(19) United States
(12) Patent Application Publication
(10) Pub. No.: US 2011/0112100 A1
(43) Pub. Date: May 12, 2011

(54) Hepatitis C Virus Inhibitors

(75) Inventors: Jared Bruce John Milbank, Sandwich (GB); David Cameron Pryde, Sandwich (GB); Thien Duc Tran, Sandwich (GB)

(73) Assignee: Pfizer Inc.

(21) Appl. No.: 12/868,342

(22) Filed: Aug. 25, 2010

Related U.S. Application Data
(63) Continuation of application No. PCT/IB2010/052734, filed on Jun. 17, 2010.
(60) Provisional application No. 61/223,142, filed on Jul. 6, 2009.
(60) Provisional application No. 61/265,880, filed on Dec. 2, 2009.

(51) Int. Cl. A61K 31/498; C07D 241/36; A61P 31/14

(52) U.S. Cl. 514/249; 544/353

(57) ABSTRACT

The present invention relates to compounds of the formula (I)

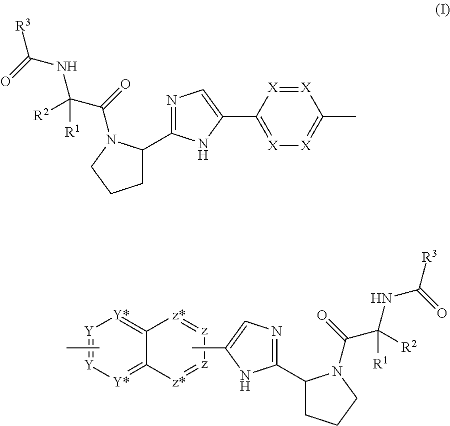

and pharmaceutically acceptable salts thereof, to compositions containing such compounds and to the use of such compounds as inhibitors of HCV replication.

The present invention is directed to certain compounds and pharmaceutically acceptable salts or solvates thereof and their use as inhibitors of the replication of hepatitis C virus (HCV). The compounds of the present invention are useful for directly or indirectly inhibiting the activity of one or more HCV proteins and for treating diseases or conditions mediated by HCV such as, for example, hepatitis C. Whilst not wishing to be bound by any specific theory, it is believed that the compounds of the present invention inhibit HCV replication by direct or indirect inhibition of the non-structural 5A (NS5A) protein. For a discussion of the NS5A protein as a target for HCV therapy and a review of the patent literature on inhibitors of NS5A, see Schmitz and Tan, Recent Patents on Ani-Infective Drug Discovery, 3, 77-92 (2008) and Holler, Parkinson and Pryde, Expert Op. Drug Disc., 4(3), 2009, 293-314.

Despite the large amount of research already performed in this area, there remains a need for inhibitors of HCV replication to treat hepatitis C. In particular, there is a need for HCV inhibitors which show activity against multiple HCV genotypes. Balanced activity against both genotype 1a and 1b is particularly desirable. Furthermore, preferred compounds should exhibit potent inhibition of the NS5A protein whilst showing little affinity for other receptors and show functional activity as inhibitors of HCV replication. They should be well absorbed from the gastrointestinal tract, be metabolically stable and possess favourable pharmacokinetic properties. They should be non-toxic and demonstrate few side-effects. In particular, good cardiovascular, liver and cell based safety profiles are important features of preferred compounds. Furthermore, the ideal drug candidate will exist in a physical form that is stable, non-hygroscopic and easily formulated.

In a first aspect, the present invention provides a compound of formula (I)

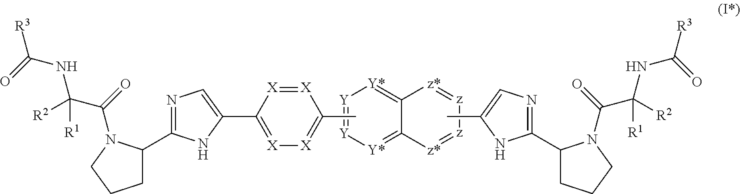

or a pharmaceutically acceptable salt thereof, wherein:

each X is independently selected from CH, CR or N, provided that the total number of N atoms in the 6-membered aromatic ring may not exceed 2 and provided that the total number of R substituents on the 6-membered aromatic ring may not exceed 2;

each Y is independently selected from C, in which case it is bonded to the 6-membered aromatic ring, CH, CR or N and each Y* is independently selected from CH, CR or N, provided that the total number of N atoms in this half of the 10-membered bicyclic aromatic ring may not exceed 2 and provided that the total number of R substituents on this half of the 10-membered bicyclic aromatic ring may not exceed 2;

each Z is independently selected from C, in which case it is bonded to the imidazole ring, CH, CR or N and each Z* is independently selected from CH, CR or N provided that the total number of N atoms in this half of the 10-membered bicyclic aromatic ring may not exceed 2 and provided that the total number of R substituents on this half of the 10-membered bicyclic aromatic ring may not exceed 2;

each R is independently selected from OH, C₁₋₄ alkoxy, CN, NH₂ or C₁₋₄ alkylsulfonyl;

each R¹ is independently selected from H, C₁₋₄ alkyl, halogen, C₁₋₄ alkoxyalkyl, C₃₋₆ cycloalkyl, phenyl, a 5- or 6-membered monocyclic heteroaryl and a 5- or 6-membered monocyclic saturated heterocyclyl;
said phenyl being optionally substituted with up to 2 halogen atoms;
said C₁₋₄ alkyl being optionally substituted with a group selected from OH, C₁₋₄ alkoxy, C₁₋₄ alkoxybenzyl, C₃₋₆ cycloalkyl, C₁₋₄ alkylsulfonyl, —NR^{a}R^{b}, —CONR^{a}R^{b}, phenyl, pyridinyl, or indolyl;
   said R^{a} and R^{b} being each independently selected from H, C₁₋₄ alkyl, C₁₋₄ alkoxyalkyl, C₁₋₄ alkylcarbonyl, or C₁₋₄ alkoxycarbonyl;

each R² is independently selected from H, C₁₋₄ alkyl, halogen, or C₁₋₄ alkoxyalkyl;
said C₁₋₄ alkyl being optionally substituted by NR^{c}R^{d};
   said R^{b} and R^{d} being each independently selected from H, C₁₋₄ alkyl, C₁₋₄ alkoxyalkyl, C₁₋₄ alkylcarbonyl, or C₁₋₄ alkoxycarbonyl; or

R¹ and R², together with the C atom to which they are attached, form a 4-, 5- or 6-membered saturated ring optionally containing 1 or 2 heteroatoms selected from O, S and NR^{e};
said R^{e} being selected from H, C₁₋₄ alkyl, C₁₋₄ alkylcarbonyl, C₁₋₄ alkoxycarbonyl or C₁₋₄ alkylsulfonyl;

each R³ is independently selected from C₁₋₄ alkyl, C₁₋₄ alkoxy, C₁₋₄ alkoxyalkyl, NH₂, NH(C₁₋₄ alkyl), N(C₁₋₄ alkyl)₂ or Ar;
said C₁₋₄ alkyl being optionally substituted with Ar or NR^{f}R^{g};
   said R^{f} and R^{g} being each independently selected from H, C₁₋₄ alkyl, C₁₋₄ alkoxyalkyl, C₁₋₄ alkylcarbonyl, or C₁₋₄ alkoxycarbonyl; and
each Ar being independently selected from isoxazolyl, pyrazinyl, dihydrobenzimidazolyl, indazolyl, and tetrahydroquinolinyl, optionally substituted with C₁₋₄ alkyl or a carbonyl group;
   provided that when a Y and a Y* both represent N then the X-containing 6-membered ring cannot represent pyrimidinyl.

In a further embodiment of the first aspect, the present invention provides a compound of formula (I*)

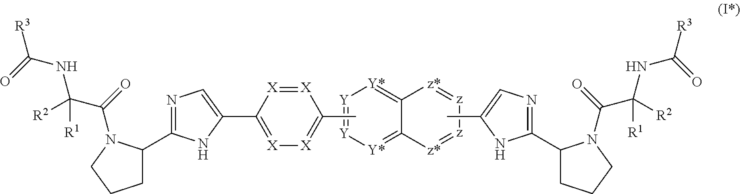

or a pharmaceutically acceptable salt or solvate thereof;
wherein:

each X is independently selected from CH, CR or N, provided that the total number of N atoms in the 6-membered aromatic ring may not exceed 2 and provided that the total number of R substituents on the 6-membered aromatic ring may not exceed 2;

each Y is independently selected from C, in which case it is bonded to the 6-membered aromatic ring, CH, CR or N and each Y* is independently selected from CH, CR or N, provided that the total number of N atoms in this half of the 10-membered bicyclic aromatic ring may not exceed 2 and provided that the total number of R substituents on this half of the 10-membered bicyclic aromatic ring may not exceed 2;

each Z is independently selected from C, in which case it is bonded to the imidazole ring, CH, CR or N and each Z* is independently selected from CH, CR or N provided that the total number of N atoms in this half of the 10-membered bicyclic aromatic ring may not exceed 2 and provided that the total number of R substituents on this half of the 10-membered bicyclic aromatic ring may not exceed 2;

each R is independently selected from OH, C₁₋₄ alkoxy, CN, NH₂ or C₁₋₄ alkylsulfonyl;

each R¹ is independently selected from H, C₁₋₄ alkyl, halogen, C₁₋₄ alkoxyalkyl, phenyl or a 5- or 6-membered monocyclic heteroaryl, wherein said phenyl is optionally substituted with up to 2 halogen atoms and said C₁₋₄ alkyl is optionally substituted with 1 NR^{a}R^{b} group wherein R^{a} and R^{b} are each independently selected from H, C₁₋₄ alkyl, C₁₋₄ alkoxyalkyl, C₁₋₄ alkylcarbonyl, and C₁₋₄ alkoxycarbonyl; and

each R² is independently selected from H, C₁₋₄ alkyl, halogen, C₁₋₄ alkoxyalkyl, wherein said C₁₋₄ alkyl is optionally substituted with 1 NR^{a}R^{b} group wherein R^{a} and R^{b} are as defined above; or

R¹ and R², together with the C atom to which they are attached, form a 4-, 5- or 6-membered saturated ring optionally containing 1 or 2 heteroatoms selected from O, S and NR^{C} wherein R^{c} is selected from H, C₁₋₄ alkyl, C₁₋₄ alkylcarbonyl, C₁₋₄ alkoxycarbonyl and C₁₋₄ alkylsulfonyl; and

each R³ is independently selected from C₁₋₄ alkyl, C₁₋₄ alkoxy, C₁₋₄ alkoxyalkyl, NH₂, NH(C₁₋₄ alkyl) or N(C₁₋₄ alkyl)₂, wherein said C₁₋₄ alkyl is optionally substituted with 1 NR^{a}R^{b} group wherein R^{a} and R^{b} are as defined above.

In a further embodiment of the first aspect, the present invention provides a compound of formula (Ia)

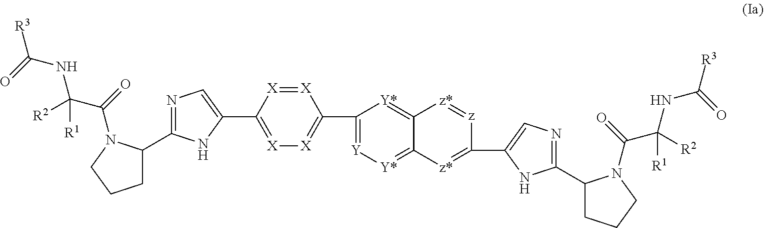

or a pharmaceutically acceptable salt thereof, wherein:
X, Y*, Z*, R¹, R² and R³ are as defined above for formula (I) or (I*), Y is selected from CH, CR and N, and Z is selected from CH, CR and N.

In a further embodiment, the present invention provides a compound of formula (Ib)

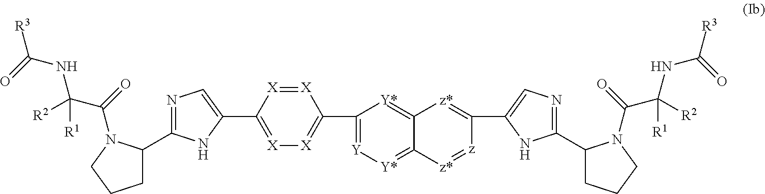

or a pharmaceutically acceptable salt thereof, wherein:
X, Y*, Z*, R¹, R² and R³ are as defined above for formula (I) or (I*), Y is selected from CH, CR and N, and Z is selected from CH, CR and N.

In a further embodiment, the present invention provides a compound of the formula

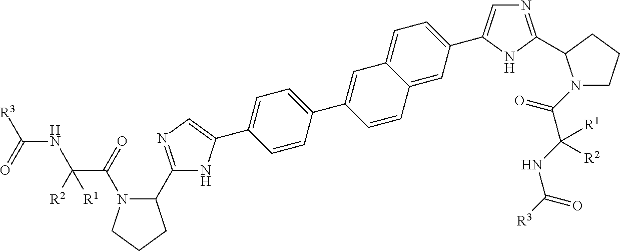

or a pharmaceutically acceptable salt thereof, wherein:
R¹, R² and R³ are as defined above for formula (I) or (I*).

In a further embodiment of the first aspect, the present invention provides compounds of the formulae:

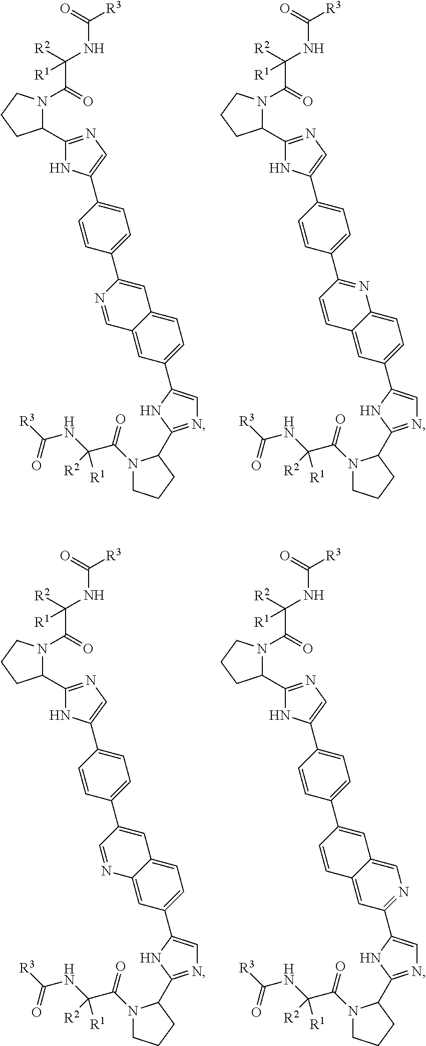

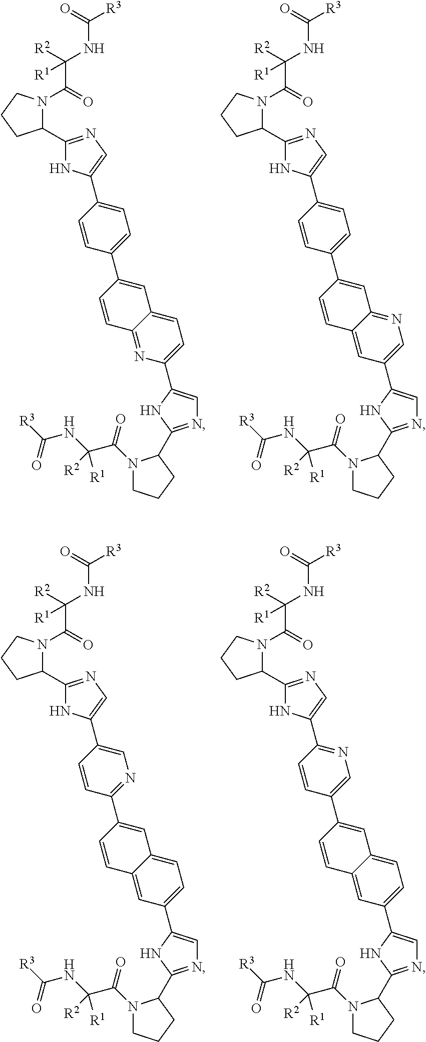

or pharmaceutically acceptable salts thereof, wherein:
R¹, R² and R³ are as defined above for formula (I) or (I*).

In a further embodiment of the first aspect, the present invention provides compounds of the formulae:

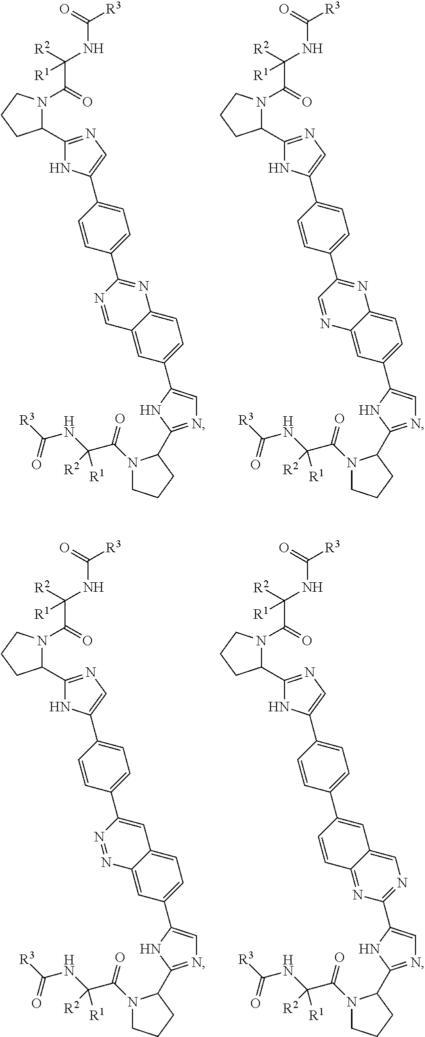

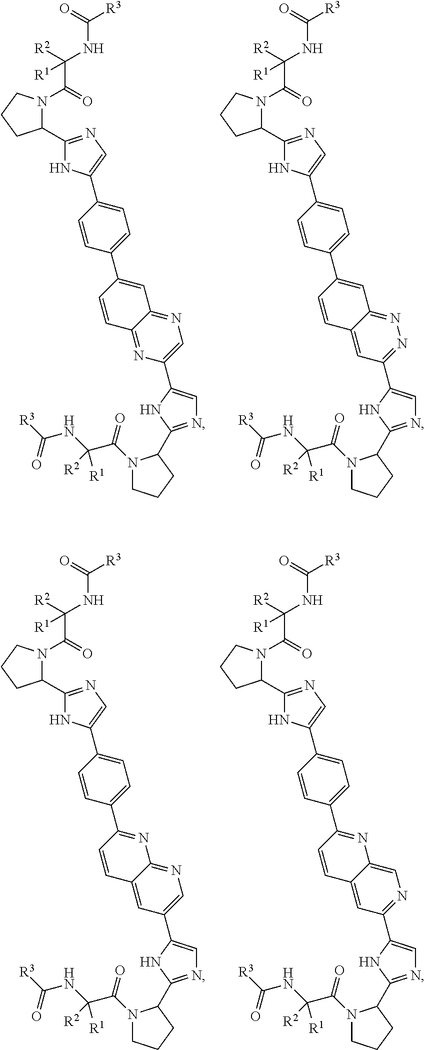

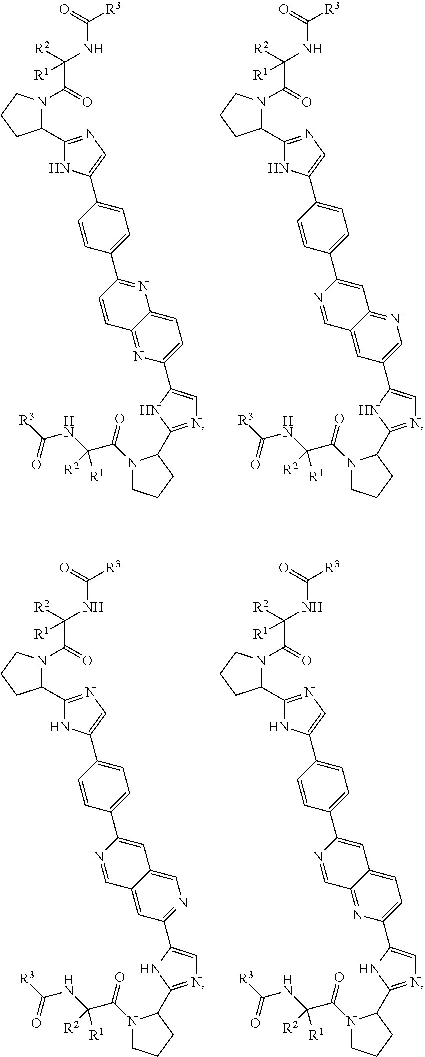

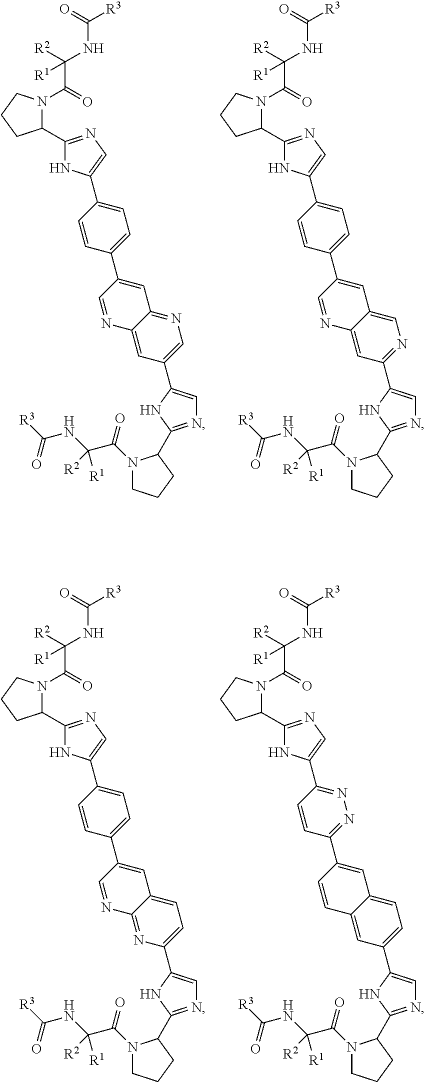

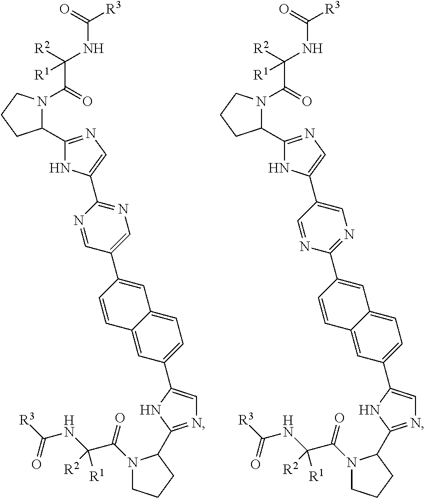

or pharmaceutically acceptable salts thereof,
wherein:
R¹, R² and R³ are as defined above for formula (I) or (I*).

In a further embodiment of the first aspect, the present invention provides compounds of the formulae:

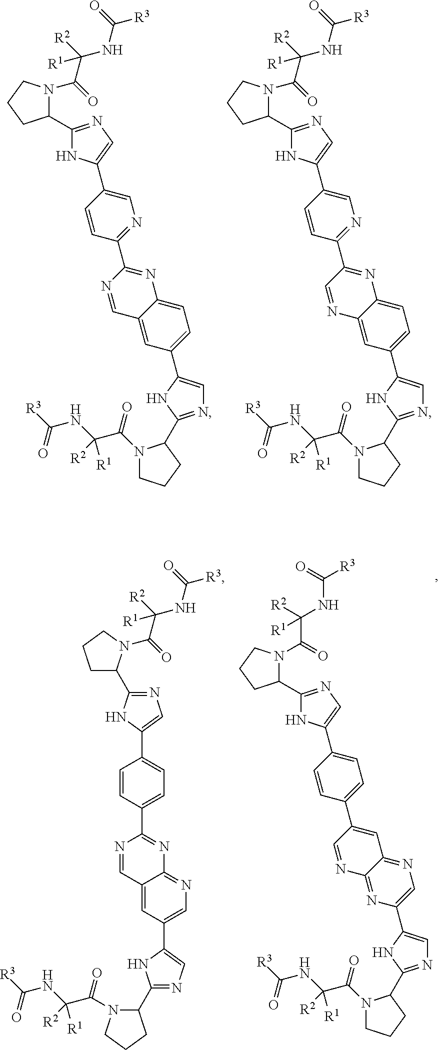

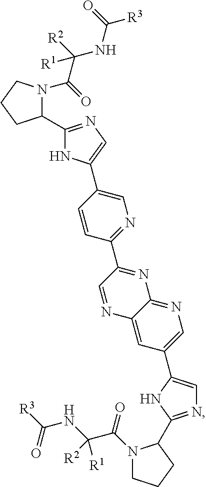

or pharmaceutically acceptable salts thereof, wherein:
R¹, R² and R³ are as defined above for formula (I) or (I*).

In a further embodiment, the present invention provides compounds of the formulae:

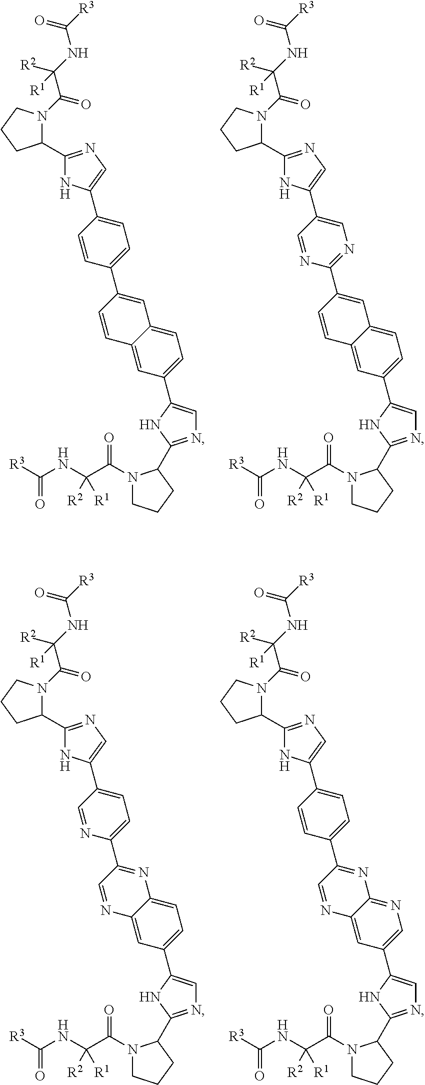

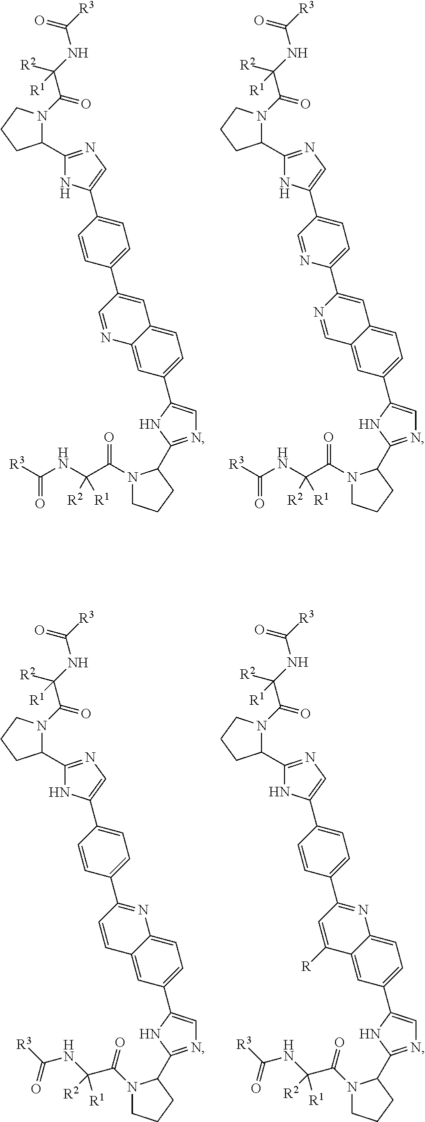

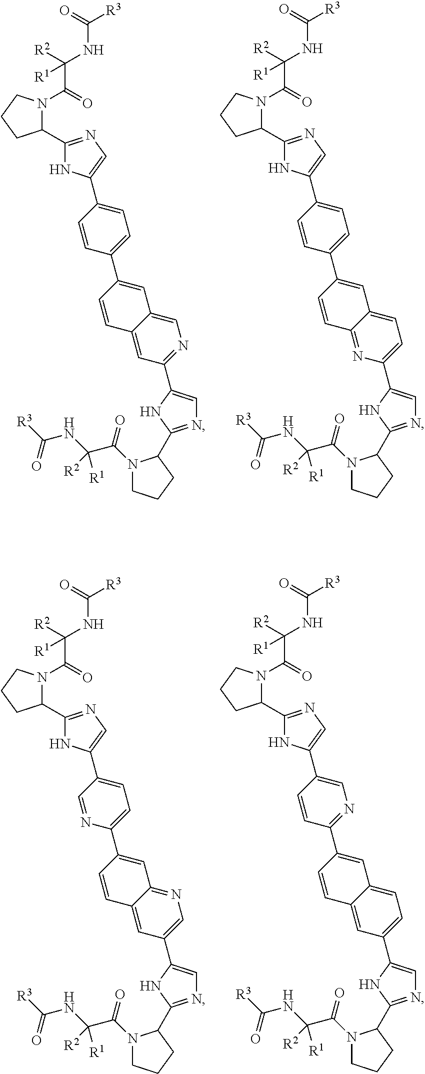

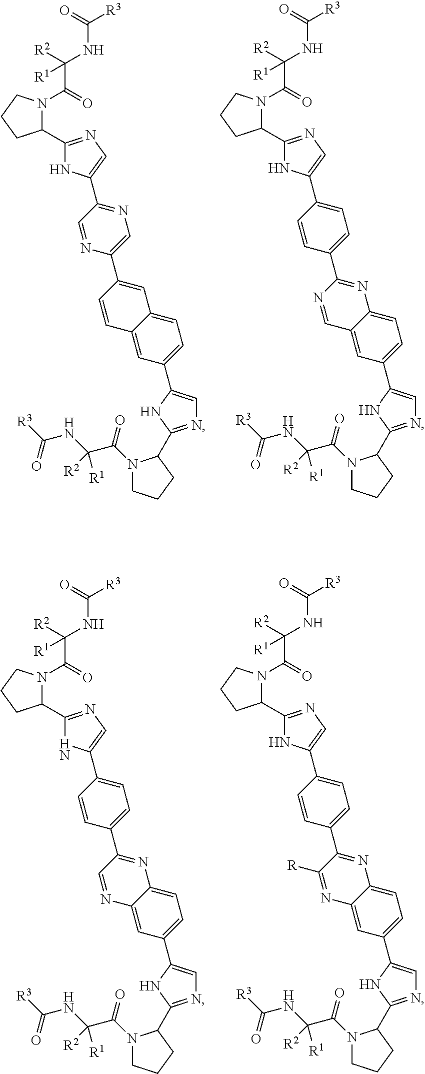

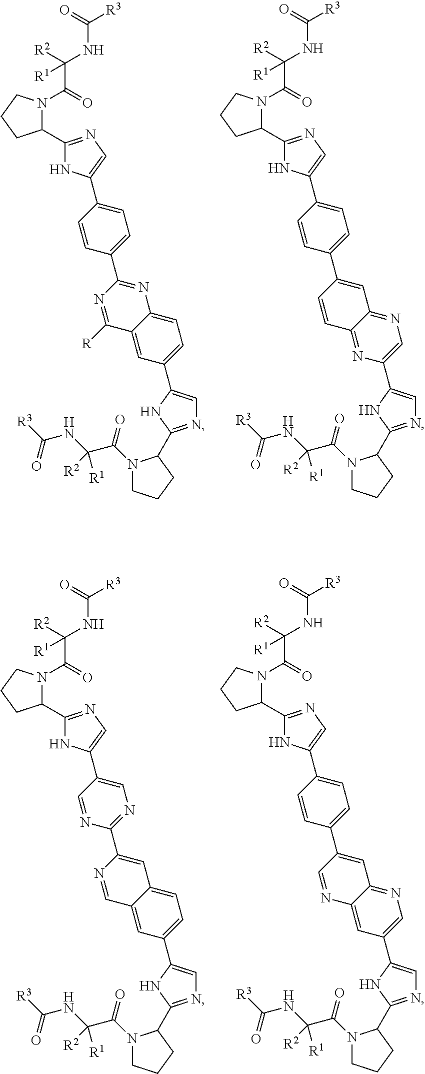

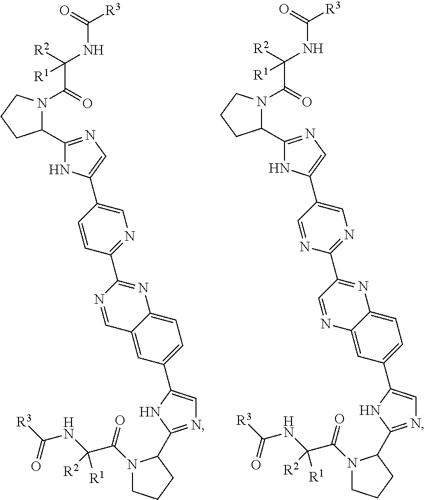

or pharmaceutically acceptable salts thereof, wherein:
R, R¹, R² and R³ are as defined above for formula (I) or (I*).

In further embodiments, the following are preferred:

(i) compounds of formulae (I), (I*), (Ia) and (Ib) wherein: each X is independently selected from CH or N, provided that the total number of N atoms in the 6-membered aromatic ring may not exceed 2;
(ii) compounds of formulae (I), (I*), (Ia) and (Ib), and embodiments (i), wherein: each Z is independently selected from C, in which case it is bonded to the imidazole ring, CH or N and each Z* is independently selected from CH or N provided that the total number of N atoms in this half of the 10-membered bicyclic aromatic ring may not exceed 2;
(iii) compounds of formulae (I), (I*), (Ia) and (Ib), and embodiment (i) and (ii), wherein: each R is independently selected from OH, C₁₋₄alkyloxy and CN;
(iv) compounds of formulae (I), (I*), (Ia) and (Ib), and embodiments (i) to (iii), wherein: each R¹ is independently selected from H, C₁₋₄ alkyl, C₃₋₆ cycloalkyl, phenyl and a 6-membered monocyclic saturated heterocyclyl;
(v) compounds of embodiment (iv), wherein: when R¹ is C₃₋₆ cycloalkyl, it is preferably C₅₋₆ cycloalkyl, more preferably C₅ cycloalkyl;
(vi) compounds of embodiment (iv), wherein: when R¹ is a 6-membered monocyclic saturated heterocyclyl, it is preferably tetrahydropyran;
(vii) compounds of embodiments (iv) to (vi), wherein: said C₁₋₄ alkyl is optionally substituted with a group selected from OH, C₁₋₄ alkoxy, C₁₋₄ alkoxybenzyl, C₃₋₆ cycloalkyl, C₁₋₄ alkylsulfonyl, —CONR^{a}R^{b}, phenyl, pyridinyl, and indolyl;
(viii) compounds of embodiment (vii), wherein: when said C₁₋₄ alkyl is substituted with C₃₋₆ cycloalkyl, it is preferably substituted with C₅₋₆ cycloalkyl, more preferably C₆ cycloalkyl;
(ix) compounds of embodiment (vii), wherein: when said C₁₋₄ alkyl is substituted with C₁₋₄ alkylsulfonyl, it is preferably substituted with C₁ alkylsulfonyl;
(x) compounds of embodiment (vii), wherein: when said C₁₋₄ alkyl is substituted with CONR^{a}R^{b}, said R^{a} and R^{b} each represent H;
(xi) compounds of formulae (I), (I*), (Ia) and (Ib), and embodiments (i) to (iv), wherein: each R¹ is independently selected from H, and C₁₋₄ alkyl;
(xii) compounds of formulae (I), (I*), (Ia) and (Ib), and embodiments (i) to (xi), wherein: each R² is independently selected from H, and C₁₋₄ alkyl;
(xiii) compounds of formulae (I), (I*), (Ia) and (Ib), and embodiments (i) to (xii), wherein: when R¹ and R², together with the carbon atom to which they are attached, form a 4-, 5- or 6-membered saturated ring, the ring contains 4 atoms, more preferably the ring contains 4 carbon atoms;
(xiv) compounds of formulae (I), (I*), (Ia) and (Ib), and embodiments (i) to (xiii), wherein: each R³ is independently selected from C₁₋₄ alkyl, C₁₋₄ alkoxy, and Ar;
(xv) compounds of embodiment (xiv), wherein: when said C₁₋₄ alkyl is optionally substituted with NR^{f}R^{g}, R^{f} and R^{g} are each independently selected from H, and C₁₋₄ alkylcarbonyl;
(xvi) compounds of embodiment (xiv), wherein: when R³ is Ar, Ar is independently selected from pyrazinyl and isoxazolyl, more preferably, Ar is substituted by C₁₋₄ alkyl and represents methyl-pyrazinyl or methyl-isoxazolyl;
(xvii) compounds of embodiment (xiv), wherein: when R³ is Ar, Ar is independently selected from indazolyl or dihydrobenzimidazole;
(xviii) compounds of formulae (I), (I*), (Ia) and (Ib), and embodiments (i) to (xiv), wherein: each R³ is independently selected from C₁₋₄ alkoxy.

For all of the formulae and embodiments depicted above, it is preferred that each Z* represents CH and each Y* represents N.

For all of the formulae and embodiments depicted above, it is preferred that each R¹ is independently selected from H or C₁₋₄ alkyl; each R² is independently selected from H or C₁₋₄ alkyl; and each R³ is independently selected from C₁₋₄ alkoxy.

For all of the formulae and embodiments depicted above, it is preferred that R¹ is H, R² is isopropyl and R³ is methoxy.

In a further embodiment the present invention provides the compounds:
[(S)-1-((S)-2-{5-[4-(6-{2-[(S)-1-((S)-2-methoxycarbonylamino-3-methylbutyryl)-pyrrolidin-2-yl]-3H-imidazol-4-yl}-quinazolin-2-yl)-phenyl]-1H-imidazol-2-yl}-pyrrolidine-1-carbonyl)-2-methyl-propyl]carbamic acid methyl ester;
methyl {(2S)-1-[(2S)-2-{5-[6-(4-{2-[(2S)-1-{(2S)-2-[(methoxycarbonyl)amino]-3-methylbutanoyl}pyrrolidin-2-yl]-1H-imidazol-4-yl}phenyl)naphthalen-2-yl]-1H-imidazol-2-yl}pyrrolidin-1-yl]-3-methyl-1-oxobutan-2-yl}carbamate;
[(S)-1-((S)-2-{4-[2-(4-{2-[(S)-1-((S)-2-methoxycarbonylamino-3-methyl-butyryl)-pyrrolidin-2-yl]-3H-imidazol-4-yl}-phenyl)-quinolin-6-yl]-1H-imidazol-2-yl}-pyrrolidine-1-carbonyl)-2-methyl-propyl]carbamic acid methyl ester;
methyl {(2S)-1-[(2S)-2-{5-[4-(6-{2-[(2S)-1-{(2S)-2-[(methoxycarbonyl)amino]-3-methylbutanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{5-[4-(2-{2-[(2S)-1-{(2S)-2-[(methoxycarbonyl)amino]-3-methyl-butanoyl}pyrrolidin-2-yl]-1H-imidazol-5-yl}quinoxalin-6-yl)phenyl]-1H-imidazol-2-yl}pyrrolidin-1-yl]-3-methyl-1-oxo butan-2-yl}carbamate;
methyl {(2S)-1-[(2S)-2-{5-[6-(5-{2-[(2S)-1-{(2S)-2-[(methoxycarbonyl)amino]-3-methyl-butanoyl}pyrrolidin-2-yl]-1H-imidazol-4-yl}pyridin-2-yl)naphthalen-2-yl]-1H-imidazol-2-yl}pyrrolidin-1-yl]-3-methyl-1-oxobutan-2-yl}carbamate;
methyl {(2S)-1-[(2S)-2-{5-[6-(5-{2-[(2S)-1-{(2S)-2-[(methoxycarbonyl)amino]-3-methyl-butanoyl}pyrrolidin-2-yl]-1H-imidazol-5-yl}pyrimidin-2-yl)naphthalen-2-yl]-1H-imidazol-2-yl}pyrrolidin-1-yl]-3-methyl-1-oxobutan-2-yl}carbamate;
methyl {(2S)-1-[(2S)-2-{5-[4-(3-{2-[(2S)-1-{(2S)-2-[(methoxycarbonyl)amino]-3-methyl-butanoyl}pyrrolidin-2-yl]-1H-imidazol-5-yl}isoquinolin-7-yl)phenyl]-1H-imidazol-2-yl}pyrrolidin-1-yl]-3-methyl-1-oxobutan-2-yl}carbamate;
methyl {(2S)-1-[(2S)-2-{5-[6-(6-{2-[(2S)-1-{(2S)-2-[(methoxycarbonyl)amino]-3-methyl-butanoyl}pyrrolidin-2-yl]-1H-imidazol-5-yl}quinoxalin-2-yl)pyridin-3-yl]-1H-imidazol-2-yl}pyrrolidin-1-yl]-3-methyl-1-oxobutan-2-yl}carbamate;
methyl {(2S)-1-[(2S)-2-{5-[6-(3-{2-[(2S)-1-{(2S)-2-[(methoxycarbonyl)amino]-3-methyl-butanoyl}pyrrolidin-2-yl]-1H-imidazol-5-yl}quinolin-7-yl)pyridin-3-yl]-1H-imidazol-2-yl}pyrrolidin-1-yl]-3-methyl-1-oxobutan-2-yl}carbamate;
methyl {(2S)-1-[(2S)-2-{4-[6-(7-{2-[(2S)-1-{(2S)-2-[(methoxycarbonyl)amino]-3-methyl-butanoyl}pyrrolidin-2-yl]-1H-imidazol-5-yl}isoquinolin-3-yl)pyridin-3-yl]-1H-imidazol-2-yl}pyrrolidin-1-yl]-3-methyl-1-oxobutan-2-yl}carbamate;
methyl {(2S)-1-[(2S)-2-{4-[4-(7-{2-[(2S)-1-{(2S)-2-[(methoxycarbonyl)amino]butanoyl}pyrrolidin-2-yl]-1H-imidazol-5-yl}-1,5-naphthyridin-3-yl)phenyl]-1H-imidazol-2-yl}pyrrolidin-1-yl]-1-oxobutan-2-yl}carbamate;
methyl {(2S)-1-[(2S)-2-{5-[4-(2-{2-[(2S)-1-{(2S)-2-[(methoxycarbonyl)amino]-3-methyl-butanoyl}pyrrolidin-2-yl]-1H-imidazol-4-yl}quinolin-6-yl)phenyl]-1H-imidazol-2-yl}pyrrolidin-1-yl]-3-methyl-1-oxobutan-2-yl}carbamate;
methyl {(2S)-1-[(2S)-2-{5-[3-(4-{2-[(2S)-1-{(2S)-2-[(methoxycarbonyl)amino]-3-methyl-butanoyl}pyrrolidin-2-yl]-1H-imidazol-5-yl}phenyl)pyrido[2,3-b]pyrazin-7-yl]-1H-imidazol-2-yl}pyrrolidin-1-yl]-3-methyl-1-oxobutan-2-yl}carbamate;
methyl {(2S)-1-[(2S)-2-{5-[4-(4-hydroxy-6-{2-[(2S)-1-{(2S)-2-[(methoxycarbonyl)amino]-3-methyl-butanoyl}pyrrolidin-2-yl]-1H-imidazol-5-yl}quinazolin-2-yl)phenyl]-1H-imidazol-2-yl}pyrrolidin-1-yl]-3-methyl-1-oxobutan-2-yl}carbamate;
methyl {(2S)-1-[(2S)-2-{5-[4-(4-ethoxy-6-{2-[(2S)-1-{(2S)-2-[(methoxycarbonyl)amino]-3-methyl-butanoyl}pyrrolidin-2-yl]-1H-imidazol-5-yl}quinazolin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(3-methoxy-6-{2-[(2S)-1-{(2S)-2-[(methoxycarbonyl)amino]-3-methyl-butanoyl}pyrrolidin-2-yl]-1H-imidazol-4-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(3-hydroxy-6-{2-[(2S)-1-{(2S)-2-[(methoxycarbonyl)amino]-3-methyl-butanoyl}pyrrolidin-2-yl]-1H-imidazol-4-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{5-[7-(4-{2-[(2S)-1-{(2S)-2-[(methoxycarbonyl)amino]-3-methylbutanoyl}pyrrolidin-2-yl]-1H-imidazol-4-yl}phenyl)-1,5-naphthyridin-3-yl]-1H-imidazol-2-yl}pyrrolidin-1-yl]-3-methyl-1-oxobutan-2-yl}carbamate;
methyl {(2S)-1-[(2S)-2-{5-[2-(5-{2-[(2S)-1-{(2S)-2-[(methoxycarbonyl)amino]-3-methylbutanoyl}pyrrolidin-2-yl]-1H-imidazol-5-yl}pyridin-2-yl)quinazolin-6-yl]-1H-imidazol-2-yl}pyrrolidin-1-yl]-3-methyl-1-oxobutan-2-yl}carbamate;
methyl {(2S)-1-[(2S)-2-{5-[2-(5-{2-[(2S)-1-{(2S)-2-[(methoxycarbonyl)amino]-3-methylbutanoyl}pyrrolidin-2-yl]-1H-imidazol-4-yl}pyrimidin-2-yl)quinoxalin-6-yl]-1H-imidazol-2-yl}pyrrolidin-1-yl]-3-methyl-1-oxobutan-2-yl}carbamate;
methyl {(2S)-1-[(2S)-2-{4-[2-(7-{2-[(2S)-1-{(2S)-2-[(methoxycarbonyl)amino]-3-methylbutanoyl}pyrrolidin-2-yl]-1H-imidazol-5-yl}isoquinolin-3-yl)pyrimidin-5-yl]-1H-imidazol-2-yl}pyrrolidin-1-yl]-3-methyl-1-oxobutan-2-yl}carbamate;
methyl {(2S)-1-[(2S)-2-{5-[4-(4-cyano-6-{2-[(2S)-1-{(2S)-2-[(methoxycarbonyl)amino]-3-methyl-butanoyl}pyrrolidin-2-yl]-1H-imidazol-4-yl}quinolin-2-yl)phenyl]-1H-imidazol-2-yl}pyrrolidin-1-yl]-3-methyl-1-oxobutan-2-yl}carbamate;
methyl {(2S)-1-[(2S)-2-{5-[6-(5-{2-[(2S)-1-{(2S)-2-[(methoxycarbonyl)amino]-3-methylbutanoyl}pyrrolidin-2-yl]-1H-imidazol-4-yl}pyrazin-2-yl)naphthalen-2-yl]-1H-imidazol-2-yl}pyrrolidin-1-yl]-3-methyl-1-oxobutan-2-yl}carbamate;
methyl {(2S)-1-[(2S)-2-{5-[4-(6-{2-[(2S)-1-{(2S)-2-[(methoxycarbonyl)amino]butanoyl}pyrrolidin-2-yl]-1H-imidazol-5-yl}naphthalen-2-yl)phenyl]-1H-imidazol-2-yl}pyrrolidin-1-yl]-1-oxobutan-2-yl}carbamate;
methyl {(2S)-1-[(2S)-2-{5-[4-(6-{2-[(2S)-1-{(2S)-2-[(methoxycarbonyl)amino]butanoyl}pyrrolidin-2-yl]-1H-imidazol-5-yl}naphthalen-2-yl)phenyl]-1H-imidazol-2-yl}pyrrolidin-1-yl]-3-methyl-1-oxobutan-2-yl}carbamate;
methyl {(2S)-1-[(2S)-2-(4-{4-[6-(2-{(2S)-1-[N-(methoxycarbonyl)-L-alanyl]pyrrolidin-2-yl}-1H-imidazol-5-yl)naphthalen-2-yl]phenyl}-1H-imidazol-2-yl)pyrrolidin-1-yl]-3-methyl-1-oxobutan-2-yl}carbamate;
methyl {1-[(2S)-2-{5-[6-(4-{2-[(2S)-1-{(2S)-2-[(methoxycarbonyl)amino]-3-methylbutanoyl}pyrrolidin-2-yl]-1H-imidazol-4-yl}phenyl)naphthalen-2-yl]-1H-imidazol-2-yl}pyrrolidin-1-yl]-2-methyl-1-oxopropan-2-yl}carbamate;
methyl {2-[(2S)-2-{5-[6-(4-{2-[(2S)-1-{(2S)-2-[(methoxycarbonyl)amino]-3-methylbutanoyl}pyrrolidin-2-yl]-1H-imidazol-4-yl}phenyl)naphthalen-2-yl]-1H-imidazol-2-yl}pyrrolidin-1-yl]-2-oxoethyl}carbamate;
methyl {(2S)-1-[(2S)-2-{5-[4-(7-{2-[(2S)-1-{(2S)-2-[(methoxycarbonyl)amino]propanoyl}pyrrolidin-2-yl]-1H-imidazol-5-yl}quinolin-3-yl)phenyl]-1H-imidazol-2-yl}pyrrolidin-1-yl]-3-methyl-1-oxobutan-2-yl}carbamate;
methyl {(2S)-1-[(2S)-2-{5-[4-(7-{2-[(2S)-1-{(2S)-2-[(methoxycarbonyl)amino]butanoyl}pyrrolidin-2-yl]-1H-imidazol-5-yl}quinolin-3-yl)phenyl]-1H-imidazol-2-yl}pyrrolidin-1-yl]-3-methyl-1-oxobutan-2-yl}carbamate;
methyl {(2S)-1-[(2S)-2-{5-[4-(7-{2-[(2S)-1-{[(methoxycarbonyl)amino]acetyl}pyrrolidin-2-yl]-1H-imidazol-5-yl}quinolin-3-yl)phenyl]-1H-imidazol-2-yl}pyrrolidin-1-yl]-3-methyl-1-oxobutan-2-yl}carbamate;
methyl {(2S)-3-methyl-1-[(2S)-2-(4-{4-[6-(2-{(2S)-1-[N-(3-methylbutanoyl)glycyl]pyrrolidin-2-yl}-1H-imidazol-5-yl) quinoxalin-2-yl]phenyl}-1H-imidazol-2-yl)pyrrolidin-1-yl]-1-oxobutan-2-yl}carbamate;
methyl {(2S)-1-[(2S)-2-{4-[4-(6-{2-[(2S)-1-(N²-acetyl-L-glutaminyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-(N-acetylglycyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-(N-acetylglycylglycyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-(N-acetyl-L-norvalyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-(4-{4-[6-(2-{(2S)-1-[(2R)-2-(acetylamino)butanoyl]pyrrolidin-2-yl}-1H-imidazol-5-yl) quinoxalin-2-yl]phenyl}-1H-imidazol-2-yl)pyrrolidin-1-yl]-3-methyl-1-oxobutan-2-yl}carbamate;
methyl {(2S)-1-[(2S)-2-(4-{4-[6-(2-{(2S)-1-[(2S)-2-(acetylamino)butanoyl]pyrrolidin-2-yl}-1H-imidazol-5-yl)quinoxalin-2-yl]phenyl}-1H-imidazol-2-yl)pyrrolidin-1-yl]-3-methyl-1-oxobutan-2-yl}carbamate;
methyl {(2S)-1-[(2S)-2-{4-[4-(6-{2-[(2S)-1-(N-acetyl-L-threonyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-3-methyl-1-[(2S)-2-{4-[4-(6-{2-[(2S)-1-{N-[(5-methylpyrazin-2-yl) carbonyl]glycyl}pyrrolidin-2-yl]-1H-imidazol-5-yl}quinoxalin-2-yl)phenyl]-1H-imidazol-2-yl}pyrrolidin-1-yl]-1-oxobutan-2-yl}carbamate;
methyl {(2S)-3-methyl-1-[(2S)-2-{4-[4-(6-{2-[(2S)-1-{N-[(5-methylisoxazol-3-yl)carbonyl]glycyl}pyrrolidin-2-yl]-1H-imidazol-5-yl}quinoxalin-2-yl)phenyl]-1H-imidazol-2-yl}pyrrolidin-1-yl]-1-oxobutan-2-yl}carbamate;
methyl {(2S)-1-[(2S)-2-(4-{4-[6-(2-{(2S)-1-[N-(1H-benzimidazol-5-ylcarbonyl)glycyl]pyrrolidin-2-yl}-1H-imidazol-5-yl)quinoxalin-2-yl]phenyl}-1H-imidazol-2-yl)pyrrolidin-1-yl]-3-methyl-1-oxobutan-2-yl}carbamate;
methyl {(2S)-1-[(2S)-2-(4-{4-[6-(2-{(2S)-1-[N-(2H-indazol-3-ylcarbonyl)glycyl]pyrrolidin-2-yl}-1H-imidazol-5-yl) quinoxalin-2-yl]phenyl}-1H-imidazol-2-yl)pyrrolidin-1-yl]-3-methyl-1-oxobutan-2-yl}carbamate;
methyl {(2S)-3-methyl-1-oxo-1-[(2S)-2-{4-[4-(6-{2-[(2S)-1-{N-[(2-oxo-3,4-dihydro quinolin-1-(2H)-yl)acetyl]glycyl}pyrrolidin-2-yl]-1H-imidazol-5-yl}quinoxalin-2-yl)phenyl]-1H-imidazol-2-yl}pyrrolidin-1-yl]butan-2-yl}carbamate;
methyl {(2S)-1-[(2S)-2-{4-[4-(6-{2-[(2S)-1-(N-acetyl-D-alanyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-(N-acetyl-L-phenylalanyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2S)-3-(benzyloxy)-2-[(methoxycarbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate carbamate;
methyl {(2S)-1-[(2S)-2-{5-[2-(4-{2-[(2S)-1-{(2S)-2-[(methoxycarbonyl)amino]-3-methylbutanoyl}pyrrolidin-2-yl]-1H-imidazol-4-yl}phenyl)quinoxalin-6-yl]-1H-imidazol-2-yl}pyrrolidin-1-yl]-1-oxo-3-phenylpropan-2-yl}carbamate;
methyl {(2S)-1-[(2S)-2-{4-[4-(6-{2-[(2S)-1-({1-[(methoxycarbonyl)amino]cyclobutyl}carbonyl)pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{2-[(methoxycarbonyl)amino]-2-methylpropanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2S)-2-[(methoxycarbonyl)amino]butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-(4-{4-[6-(2-{(2S)-1-[N-(methoxycarbonyl)-D-alanyl]pyrrolidin-2-yl}-1H-imidazol-5-yl) quinoxalin-2-yl]phenyl}-1H-imidazol-2-yl)pyrrolidin-1-yl]-3-methyl-1-oxobutan-2-yl}carbamate;
methyl {(2R)-1-[(2S)-2-{5-[2-(4-{2-[(2S)-1-{(2S)-2-[(methoxycarbonyl)amino]-3-methylbutanoyl}pyrrolidin-2-yl]-1H-imidazol-4-yl}phenyl)quinoxalin-6-yl]-1H-imidazol-2-yl}pyrrolidin-1-yl]-4-methyl-1-oxopentan-2-yl}carbamate;
methyl {(2S)-1-[(2S)-2-{5-[2-(4-{2-[(2S)-1-{(2S)-2-[(methoxycarbonyl)amino]-3-methylbutanoyl}pyrrolidin-2-yl]-1H-imidazol-4-yl}phenyl)quinoxalin-6-yl]-1H-imidazol-2-yl}pyrrolidin-1-yl]-1-oxopentan-2-yl}carbamate;
methyl {(2S)-1-[(2S)-2-{4-[4-(6-{2-[(2S)-1-{(2S)-2-[(methoxycarbonyl)amino]-2-phenylacetyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-(4-{4-[6-(2-{(2S)-1-[N-(methoxycarbonyl)-L-alanyl]pyrrolidin-2-yl}-1H-imidazol-5-yl) quinoxalin-2-yl]phenyl}-1H-imidazol-2-yl)pyrrolidin-1-yl]-3-methyl-1-oxobutan-2-yl}carbamate;
methyl {(2S)-1-[(2S)-2-{4-[4-(6-{2-[(2S)-1-{(2S,3S)-2-[(methoxycarbonyl)amino]-3-methyl pentanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2R)-2-[(methoxycarbonyl)amino]butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2S,3R)-3-hydroxy-2-[(methoxy carbonyl)amino]butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2R)-2-[(methoxycarbonyl)amino]-3-methyl butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2R)-1-[(2S)-2-{5-[2-(4-{2-[(2S)-1-{(2S)-2-[(methoxycarbonyl)amino]-3-methyl butanoyl}pyrrolidin-2-yl]-1H-imidazol-4-yl}phenyl)quinoxalin-6-yl]-1H-imidazol-2-yl}pyrrolidin-1-yl]-1-oxo-3-phenylpropan-2-yl}carbamate;
methyl {(2S)-1-[(2S)-2-{4-[4-(6-{2-[(2S)-1-{(2R)-3-(benzyloxy)-2-[(methoxy carbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2S)-3-hydroxy-2-[(methoxycarbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {2-[(2S)-2-{5-[2-(4-{2-[(2S)-1-{(2S)-2-[(methoxycarbonyl)amino]-3-methylbutanoyl}pyrrolidin-2-yl]-1H-imidazol-4-yl}phenyl)quinoxalin-6-yl]-1H-imidazol-2-yl}pyrrolidin-1-yl]-2-oxoethyl}carbamate;
methyl {(2S)-1-[(2S)-2-{4-[4-(6-{2-[(2S)-1-{cyclopentyl[(methoxycarbonyl)amino]acetyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl [(2S)-1-[(2S)-2-{5-[2-(4-{2-[(2S)-1-{(2S)-2-[(methoxycarbonyl)amino]-3-methyl butanoyl}pyrrolidin-2-yl]-1H-imidazol-4-yl}phenyl)quinoxalin-6-yl]-1H-imidazol-2-yl}pyrrolidin-1-yl]-4-(methyl sulfonyl)-1-oxobutan-2-yl]carbamate;
methyl {(2S)-1-[(2S)-2-{5-[2-(4-{2-[(2S)-1-{(2S)-2-[(methoxycarbonyl)amino]-3-methylbutanoyl}pyrrolidin-2-yl]-1H-imidazol-4-yl}phenyl)quinoxalin-6-yl]-1H-imidazol-2-yl}pyrrolidin-1-yl]-4-methyl-1-oxopentan-2-yl}carbamate;
methyl {(2S)-1-[(2S)-2-{4-[4-(6-{2-[(2S)-1-{(2S)-3-methoxy-2-[(methoxycarbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2S)-2-[(methoxycarbonyl)amino]-3,3-dimethyl butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{[(methoxycarbonyl)amino](tetrahydro-2H-pyran-4-yl)acetyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2R)-1-[(2S)-2-{5-[2-(4-{2-[(2S)-1-{(2S)-2-[(methoxycarbonyl)amino]-3-methyl butanoyl}pyrrolidin-2-yl]-1H-imidazol-4-yl}phenyl)quinoxalin-6-yl]-1H-imidazol-2-yl}pyrrolidin-1-yl]-1-oxo-4-phenylbutan-2-yl}carbamate;
methyl {(2S)-1-[(2S)-2-{4-[4-(6-{2-[(2S)-1-{(2R)-2-[(methoxycarbonyl)amino]-3,3-dimethyl butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2S,3R)-3-tert-butoxy-2-[(methoxy carbonyl)amino]butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2R,3R)-3-tert-butoxy-2-[(methoxy carbonyl)amino]butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2R)-3-tert-butoxy-2-[(methoxy carbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2R)-1-[(2S)-2-{5-[2-(4-{2-[(2S)-1-{(2S)-2-[(methoxycarbonyl)amino]-3-methyl butanoyl}pyrrolidin-2-yl]-1H-imidazol-4-yl}phenyl)quinoxalin-6-yl]-1H-imidazol-2-yl}pyrrolidin-1-yl]-1-oxopentan-2-yl}carbamate;
methyl {(2R)-1-[(2S)-2-{5-[2-(4-{2-[(2S)-1-{(2S)-2-[(methoxycarbonyl)amino]-3-methyl butanoyl}pyrrolidin-2-yl]-1H-imidazol-4-yl}phenyl)quinoxalin-6-yl]-1H-imidazol-2-yl}pyrrolidin-1-yl]-1-oxohexan-2-yl}carbamate;
methyl {(2S)-1-[(2S)-2-{5-[2-(4-{2-[(2S)-1-{(2S)-2-[(methoxycarbonyl)amino]-3-methylbutanoyl}pyrrolidin-2-yl]-1H-imidazol-4-yl}phenyl)quinoxalin-6-yl]-1H-imidazol-2-yl}pyrrolidin-1-yl]-1-oxo-4-phenylbutan-2-yl}carbamate;
methyl {(2S)-1-[(2S)-2-(4-{4-[6-(2-{(2S)-1-[3-cyclohexyl-N-(methoxycarbonyl) alanyl]pyrrolidin-2-yl}-1H-imidazol-5-yl) quinoxalin-2-yl]phenyl}-1H-imidazol-2-yl)pyrrolidin-1-yl]-3-methyl-1-oxobutan-2-yl}carbamate;
methyl {(2S)-1-[(2S)-2-{4-[4-(6-{2-[(2S)-1-{(2S)-3-tert-butoxy-2-[(methoxy carbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-{4-[4-(6-{2-[(2S)-1-{(2R)-3-(1H-indol-3-yl)-2-[(methoxy carbonyl)amino]propanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
methyl {(2S)-1-[(2S)-2-(4-{4-[6-(2-{(2S)-1-[(2R)-2-[(methoxycarbonyl)amino]-3-(pyridin-2-yl) propanoyl]pyrrolidin-2-yl}-1H-imidazol-5-yl)quinoxalin-2-yl]phenyl}-1H-imidazol-2-yl)pyrrolidin-1-yl]-3-methyl-1-oxobutan-2-yl}carbamate;
methyl {(2S)-1-[(2S)-2-{4-[4-(6-{2-[(2S)-1-{(2R)-2-[(methoxycarbonyl)amino]-2-phenylacetyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate;
   or pharmaceutically acceptable salts thereof.

In a further embodiment, the present invention provides the compounds:
[(S)-1-((S)-2-{5-[4-(6-{2-[(S)-1-((S)-2-methoxycarbonylamino-3-methylbutyryl)-pyrrolidin-2-yl]-3H-imidazol-4-yl}-quinazolin-2-yl)-phenyl]-1H-imidazol-2-yl}-pyrrolidine-1-carbonyl)-2-methyl-propyl]carbamic acid methyl ester;
methyl {(2S)-1-[(2S)-2-{5-[4-(6-{2-[(2S)-1-{(2S)-2-[(methoxycarbonyl)amino]-3-methylbutanoyl}pyrrolidin-2-yl]-1H-imidazol-5-yl}quinoxalin-2-yl)phenyl[1H-imidazol-2-yl}pyrrolidin-1-yl]-3-methyl-1-oxobutan-2-yl}carbamate;
   or pharmaceutically acceptable salts thereof.

In a further embodiment, the present invention provides the compound methyl {(2S)-1-[(2S)-2-{5-[4-(6-{2-[(2S)-1-{(2S)-2-[(methoxycarbonyl)amino]-3-methylbutanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate or a pharmaceutically acceptable salt thereof.

Hereinafter, all references to a compound of formula (I) include compounds of formulae (I), (I*), (Ia) and (Ib) as described above.

‘C₁₋₄ alkyl’ means a monovalent unsubstituted saturated straight-chain or branched-chain hydrocarbon radical having from 1 to 4 carbon atoms. ‘C₁₋₂ alkyl’ and ‘C₁₋₃ alkyl’ have analogous meanings.

‘C₃₋₆cycloalkyl’ means an unsubstituted saturated monocyclic hydrocarbon radical having from 3 to 6 carbon atoms.

‘C₁₋₄ alkoxy’ means —O—C₁₋₄ alkyl (C₁₋₄ alkyl being as defined above).

‘C₁₋₄ alkylsulfonyl’ means —(SO₂)—C₁₋₄ alkyl (C₁₋₄ alkyl being as defined above).

‘C₁₋₄ alkoxyalkyl’ means C₁₋₃ alkyl-O—C₁₋₃ alkyl C₁₋₃ alkyl being as defined above), provided that the total number of C atoms does not exceed 4.

‘C₁₋₄ alkylcarbonyl’ means—(C═O)—C₁₋₃ alkyl C₁₋₃ alkyl being as defined above).

‘C₁₋₄ alkoxycarbonyl’ means —(C═O)—O—C₁₋₃ alkyl (C₁₋₃ alkyl being as defined above).

‘C₁₋₄ alkoxybenzyl’ means PhCH₂O—C₁alkyl.

‘Halogen’ means a fluorine, chlorine, bromine or iodine atom.

‘5- or 6-membered monocyclic heteroaryl’ means a monocyclic aromatic group with a total of 5 atoms in the ring wherein from 1 to 4 of those atoms are each independently selected from N, O and S; or a monocyclic aromatic group with a total of 6 atoms in the ring wherein from 1 to 3 of those atoms are N. Preferred 5-membered monocyclic heteroaromatic groups have from 1 to 3 atoms in the ring which are each independently selected from N, O and S. 5-membered monocyclic heteroaromatic groups include pyrrolyl (also called azolyl), furanyl, thienyl (also called thiophenyl), pyrazolyl (also called 1H-pyrazolyl and 1,2-diazolyl), imidazolyl, oxazolyl (also called 1,3-oxazolyl), isoxazolyl (also called 1,2-oxazolyl), thiazolyl (also called 1,3-thiazolyl), isothiazolyl (also called 1,2-thiazolyl), triazolyl, oxadiazolyl, thiadiazolyl, tetrazolyl, oxatriazolyl and thiatriazolyl. 6-membered monocyclic heteroaromatic groups include pyridinyl, pyrimidinyl, pyrazinyl, pyridazinyl and triazinyl.

‘Pharmaceutically acceptable salts’ of the compounds of formula (I) include the acid addition and base salts thereof.

Suitable acid addition salts may be formed from acids which form non-toxic salts. Examples may include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts may be formed from bases which form non-toxic salts. Examples may include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of the compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water. A currently accepted classification system for organic hydrates is one that defines isolated site, channel, or metal-ion coordinated hydrates—see “Polymorphism in Pharmaceutical Solids” by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’and that resulting from the addition of a second component, such as water or another solvent, is described as ‘lyotropic’. Compounds that have the potential to form lyotropic mesophases are described as ‘amphiphiliO and consist of molecules which possess an ionic (such as —COO⁻Na’, —COO⁻K⁺, or —SO₃Na⁺) or non-ionic (such as —N⁻N⁺(CH₃)₃) polar head group. For more information, see “Crystals and the Polarizing Microscope” by N. H. Hartshorne and A. Stuart, 4^{th} Edition (Edward Arnold, 1970).

Hereinafter all references to a compound of formula (I) include references to salts, solvates, polymorphs, crystal habits, multi-component complexes and liquid crystals thereof and to solvates, polymorphs, crystal habits, multi-component complexes and liquid crystals of salts thereof.

Compounds of formula (I) contain at least two asymmetric carbon atoms (on the pyrrolidine rings) and can therefore exist as two or more stereoisomers. Compounds of formula (I) also contain aromatic moieties, such as the imidazole rings, wherein tautomeric isomerism (‘tautomerism’) can occur. This can take the form of proton tautomerism (for example in the imidazole rings) as well as valence tautomerism (for example in the other aromatic moieties). It follows that a single compound may exhibit more than one type of isomerism.

Included within the scope of the claimed compounds of the present invention are all stereoisomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluant affords the enriched mixture.

Mixtures of stereoisomers may be separated by conventional techniques known to those skilled in the art. See, for example, “Stereochemistry of Organic Compounds” by E L Eliel (Wiley, New York, 1994).

Due to their structure, compounds of the present invention may also exist in different stable conformational forms which may be separable. Torsional asymmetry due to restricted rotation about a single bond may permit separation of different conformers. Certain conformers which are preferred for biological activity may also be selected for through intramolecular hydrogen bonding. Included within the scope of the claimed compounds of the present invention are all conformers of the compounds of formula (I), including compounds exhibiting more than one type of conformation, and mixtures of one or more thereof.

The compounds of the invention also includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H; carbon, such as ¹¹C, ¹³C and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O and ¹⁸O; and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

In one embodiment, the compounds of formula (I) are comprised of atoms such that the average atomic mass or mass number for each atom of each element present corresponds to the average atomic mass or mass number for that element as it occurs in nature.

Pharmaceutically acceptable solvates in accordance with the invention include those wherein the solvent of crystallization may be isotopically substituted, e.g. D₂O, d₆-acetone, and d₆-DMSO.

The following schemes depict general methods for obtaining compounds of formula (I).

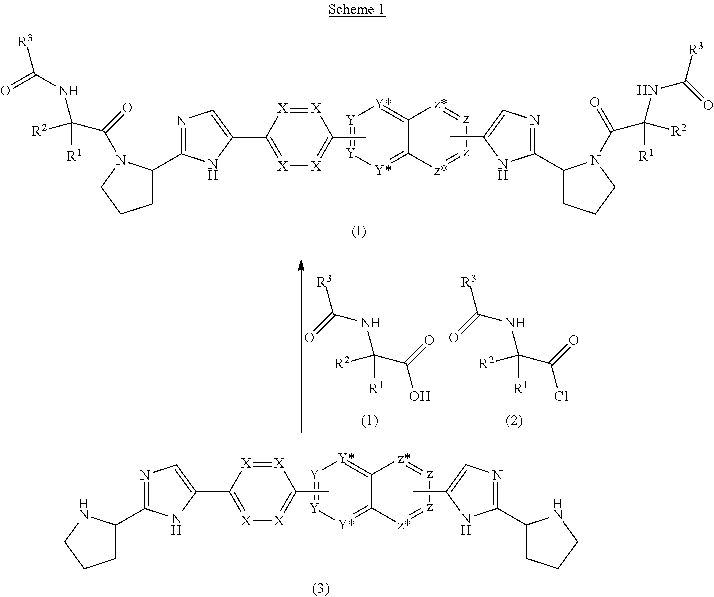

Amide coupling is carried out using standard literature conditions. The acid (1) can be converted to the acid chloride (2) using a suitable chlorinating agent, such as oxalyl chloride or thionyl chloride, in a suitable solvent, such as dichloromethane or toluene, optionally in the presence of catalytic DMF, at a suitable temperature, typically between 0° C. and room temperature. The acid chloride (2) can then be reacted with a suitable amine (3) in the presence of a base, such as triethylamine or diisopropylethylamine, in a suitable solvent, such as dichloromethane or toluene, at a temperature between 0° C. and room temperature. Alternatively the acid (1) can be converted to a suitable activated species with a coupling agent, such as

EDCI.HCl, EDCI.MeI, HBTU, HATU, PyBop, DCC, or CDI, in a suitable solvent, such as dichloromethane, acetonitrile or DMF. In the presence of EDCI.HCl or EDCI.MeI, HOBT is optionally added. A suitable base such as triethylamine or diisopropylethylamine is also used and the reaction is typically carried out at room temperature.

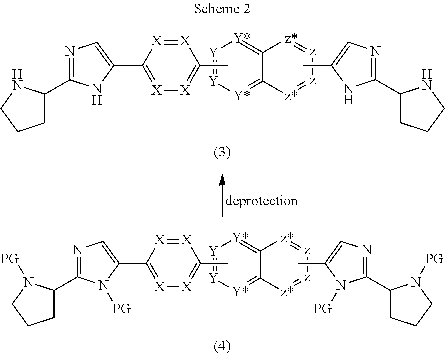

Amine (3) may be formed from protected amine (4) wherein in one or more of the N moieties are protected by a suitable protecting group (PG). Note that, depending on the chemistry used to produce amine (4), protection of one or both imidazole N moieties may not be necessary. Suitable protecting groups for the pyrrolidine N moiety include, for example, t-butyloxycarbonyl (t-BOC). Suitable protecting groups for the imidazole N moiety include, for example, (trimethylsilyl)ethoxymethyl (SEM). Deprotection is carried out using known literature methods, such as reaction with an acid such as hydrochloric acid or trifluoracetic acid, in a suitable solvent, such as methanol or 1,4-dioxane, at a temperature typically between room temperature and reflux (the temperature required depending on the nature of the protecting group).

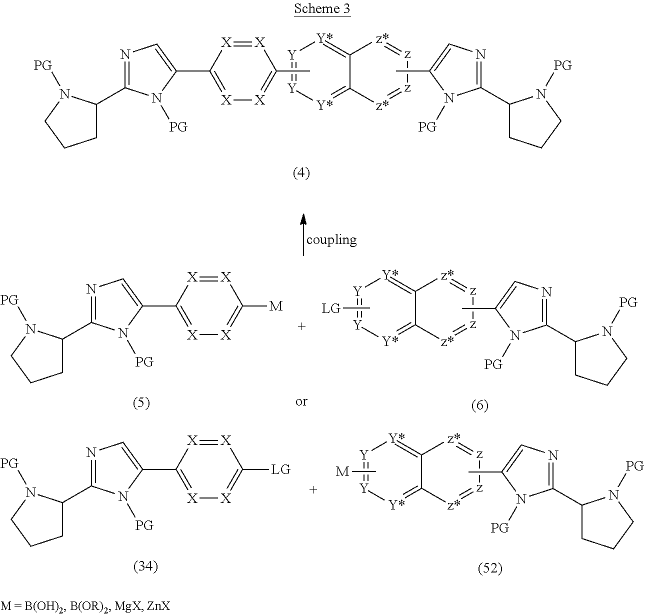

Protected amine (4) may be formed by a palladium catalysed coupling between metallated species (5) and triflate, or halide, (6) (i.e. LG=OSO₂CF₃ or Cl/Br/I). Preferably, the reaction may be carried out between the boronate (5) (M=B(OR)₂) and a halide (6) (LG=Br or I) using a suitable palladium catalyst such as palladium acetate, palladium bis(triphenylphosphine)dichloride, tetrakis(triphenylphosphine)palladium, tris(dibenzylideneacetone)dipalladium, bis(dibenzylideneacetone)palladium or (1,1′-bis(diphenylphosphino)ferrocene)dichloropalladium and a suitable base, such as sodium carbonate, sodium hydrogen carbonate, potassium acetate or potassium phosphate, in a suitable solvent, such as 1,4-dioxane or DME, at a temperature of typically around 80 to 120° C. A phosphine ligand such as tricyclohexylphosphine or 2-dicyclohexylphosphino-2′,6′-dimethoxylbiphenyl may be required with palladium acetate, tris(dibenzylideneacetone)dipalladium or bis(dibenzylideneacetone)palladium. Alternatively, protected amine (4) can be formed by reaction between metallated species (52) (preferably the reaction is carried out with a boronic acid or ester) and halide (34) under palladium coupling conditions such as those described above.

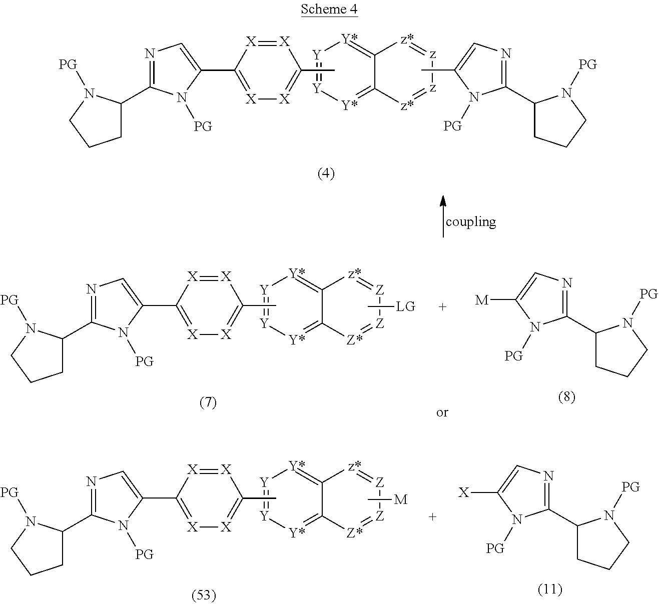

Alternatively, protected amine (4) may be formed by a palladium catalysed coupling between a triflate, or halide, (7) (i.e. LG=OSO₂CF₃ or Cl/Br/I) and a metallated imidazole (8) (the boronic acid is preferred) using conditions similar to those described for Scheme 3 above. It can also be formed using metallated species (53) (the boronic acid or ester is preferred) and haloimidazole (11) using palladium coupling conditions similar to those described for Scheme 3 above.

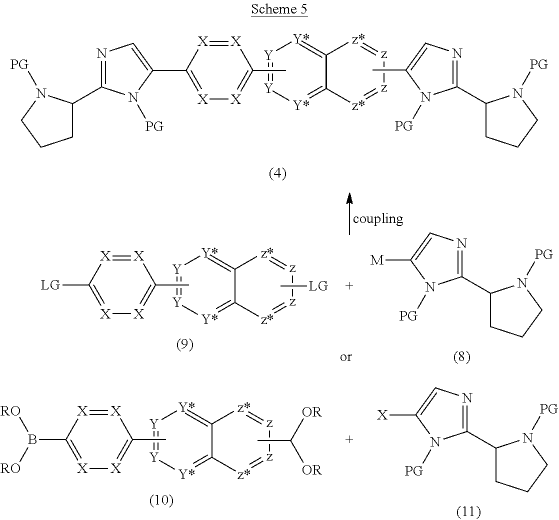

Alternatively, protected amine (4) may be formed by a palladium catalysed coupling between a ditriflate, or dihalide, (9) (i.e. LG=OSO₂CF₃ or Cl/Br/I) and a metallated imidazole (8) (the boronic acid is preferred) or between a diboronate (10) and haloimidazole (11) using palladium catalysed coupling. Preferably, the reaction may be carried out between the imidazole boronic acid (8) (M=B(OH)₂) and dihalide (9) (LG=Br or I) using a suitable palladium catalyst, such as tris(dibenzylideneacetone)dipalladium, palladium acetate or bis(dibenzylideneacetone)palladium, and a suitable phosphine base, such as tricyclohexyl-phosphine or 2-dicyclohexylphosphino-2′,6′-dimethoxylbiphenyl. A copper (I) source, such as copper (I) chloride, may optionally be added, and a suitable base, such as potassium phosphate, is required in the presence of a suitable solvent, such as 1,4-dioxane, DME or THF/water, at a temperature of typically around reflux (or at temperatures above the boiling point of the solvent, e.g. 120° C., using microwave conditions). Extra charges of catalyst and metallated species may be required to get the double addition.

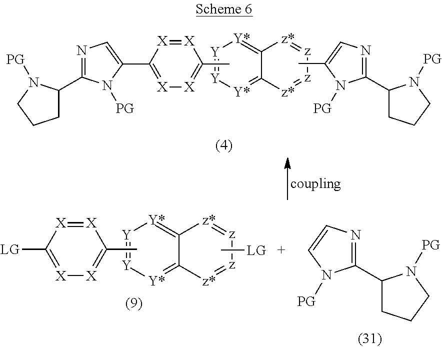

Alternatively, protected amine (4) may be also be formed by a palladium catalysed CH activation between imidazole (31) and dihalide, or ditriflate, (9) (i.e. LG=OSO₂CF₃ or Cl/Br/I). The reaction is typically carried out using a suitable palladium catalyst, such as palladium acetate, a suitable phosphine base, such as tricyclohexylphosphine (typically used as the tetrafluoroborate salt), an acid source, such as 2,2-dimethylpropionic acid (pivalic acid), and a suitable base, such as potassium carbonate, in the presence of a suitable solvent, such as N,N-dimethylacetamide or N,N-dimethylformamide, at a temperature of typically around 140° C.

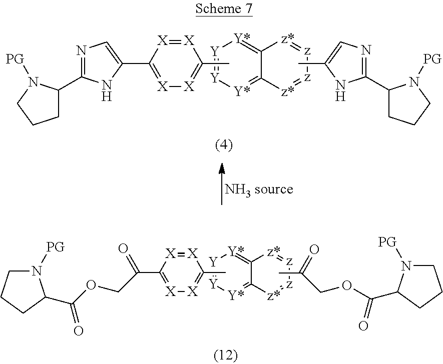

Alternatively, protected amine (4) (in this case without imidazole protecting groups) may be formed from diester (12) by an imidazole formation reaction using a suitable ammonia source, typically ammonium acetate, in a solvent, such as toluene or xylene, at a temperature of typically around 100 to 150° C.

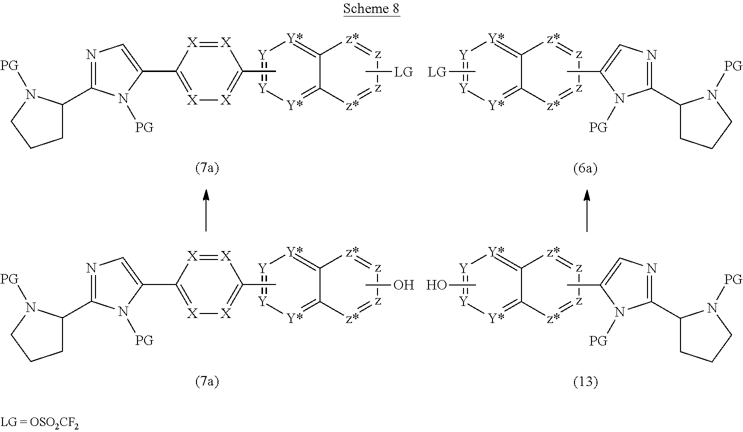

Triflates (6a) and (7a) may be formed from the corresponding phenols (13) and (14) under standard literature conditions, such as by using trifluoromethanesulfonic anhydride or phenylditrifluoromethanesulfonyl-amide and a suitable base, such as triethylamine or diisopropylethylamine, in a suitable solvent, such as dichloromethane or acetonitrile, at a temperature of typically around −40° C. to room temperature.

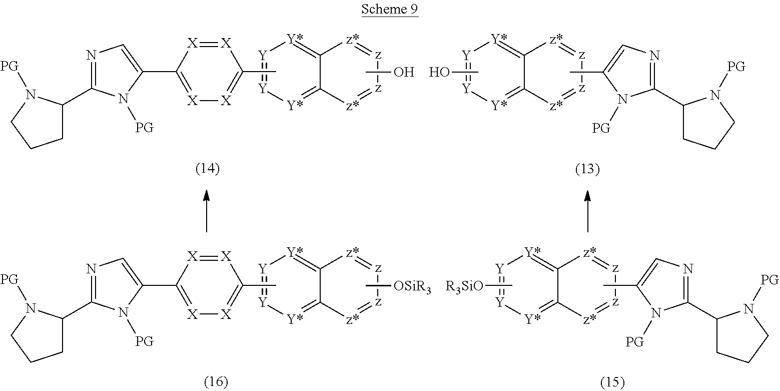

Phenols (13) and (14) may be formed by deprotection of the corresponding protected phenols (15) and (16) (a trialkylsilyl group is preferably used as the protecting group) under standard literature conditions, such as using tetrabutylammonium fluoride in a suitable solvent, such as THF, at a temperature of typically around 0° C. to room temperature.

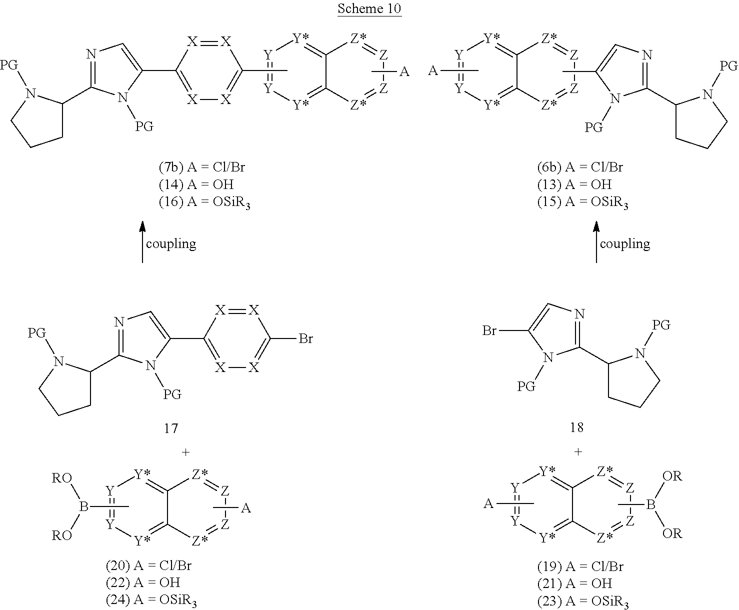

Protected phenols (15) and (16), certain unprotected phenols (13) and (14), and halides (6b) and (7b) (i.e. LG=Cl/Br) may be formed by palladium catalysed coupling reactions between halides (17) and (18) and boronates (19) to (24) under the conditions described for Scheme 3 above.

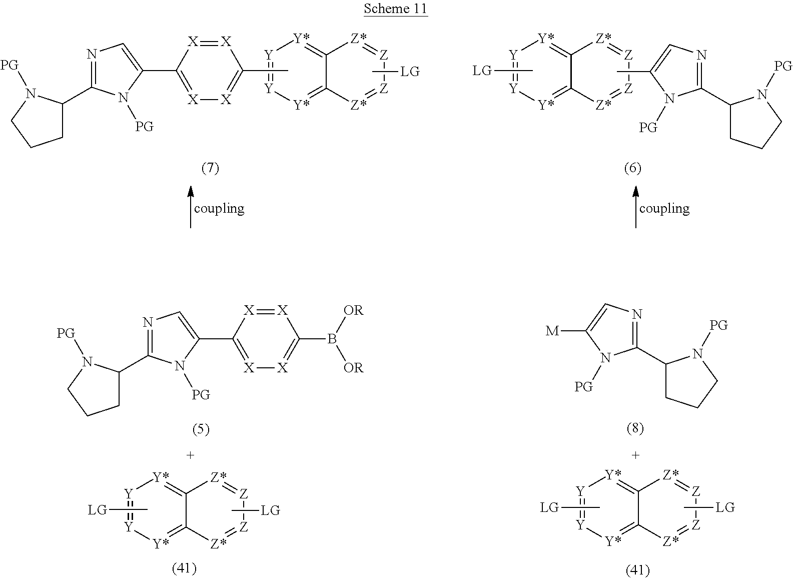

Alternatively, compounds (7) and (6) may be formed by palladium catalysed coupling reactions between ditriflate, halotriflate or dihalide (41) (LG=OSO₂CF₃ or Cl/Br/I) and metallated species (5), (preferably the boronic ester is used) under the conditions described for Scheme 3. Metallated imidazole (8) (the boronic acid is preferably used) may also be reacted with species 41 under similar conditions as described for Scheme 3 above (it is assumed that one of the two groups LG of (41) is more prone to oxidative addition and thus reaction than the other one).

Alternatively, compound (7) may be formed by palladium catalysed coupling reactions between ditriflate, or dihalide, (41) and a stannane derived from compound (34). For example formation of the stannane is typically carried out using compound (34), hexamethylditin and a palladium catalyst, such as palladium bis(triphenylphosphine)dichloride or tetrakis(triphenylphosphine)palladium, in a suitable solvent, such as DMF, 1,4-dioxane, at a temperature of typically between 80° C. and 120° C.

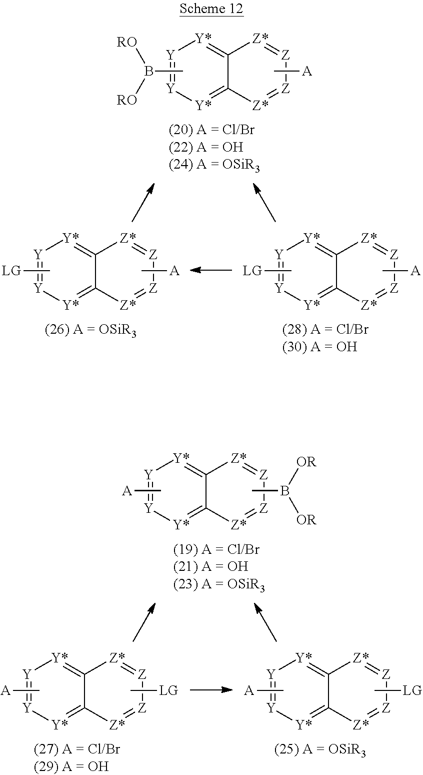

Boronates (19) to (24) may be formed from the corresponding halides, or triflates, (25) to (30) (i.e. LG=OSO₂CF₃ or Cl/Br/I) using standard literature conditions. For example, reaction can be carried out with a strong base, such as butyllithium or isopropylmagnesium chloride (for LG=Br), in a suitable solvent, such as THF, at a temperature of typically between −78° C. and room temperature, followed by quenching on a trialkylborate, such as trimethylborate or triisopropylborate; alternatively, reaction is carried out using a suitable boron source, such as bis(pinacolato)diboron, using a palladium catalyst, such as palladium acetate, palladium bis(triphenylphosphine)dichloride, tetrakis(triphenylphosphine)palladium, or (1,1′-bis(diphenyl-phosphino)ferrocene)dichloropalladium, and a suitable base, such as sodium carbonate, sodium bicarbonate, potassium acetate or potassium phosphate, in a suitable solvent, such as 1,4-dioxane or DME, at a temperature of typically around 80 to 110° C. Protected phenols (25) and (26) may be formed from compounds (29) and (30) with a suitable silyl chloride, such as triisopropylsilyl chloride or t-butyldimethyl silyl chloride, in the presence of a suitable base, such as triethylamine, DMAP or imidazole, in a suitable solvent, such as DMF or dichloromethane (it is assumed that LG is more prone to oxidative addition than A and thus reaction occurs here).

Starting materials (27) to (30) are either commercially available or may be synthesised by standard literature procedures such as, for example, those described in Eur. J. Org. Chem., 2000, 491-497; J. Org. Chem., 2008, 73, 8901-8920; and Bioorganic and Med. Chem. Letters, 2007, 6525-6528. Some examples are presented in Scheme 13.

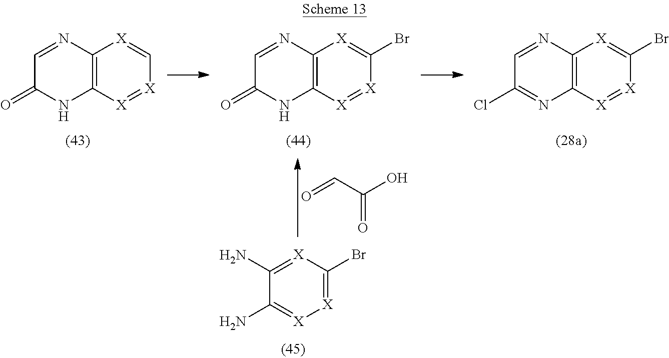

Bromide (44) may be prepared via bromination of compound (43) using standard literature methods, such as using bromine in sulphuric acid with sliver sulphate followed by the addition of carbon tetrachloride or chloroform, at a temperature of typically around 25 to 50° C.

Alternatively, bromide (44) may be prepared from diamine (45) and oxoacetic acid in a suitable solvent, such as methanol, at a temperature of typically around 0 to 25° C.

Chlorination of (44) is typically carried out using known methods, such as phosphorus oxychloride with or without catalytic DMF at a temperature of typically around 120° C., to give compound (28a).

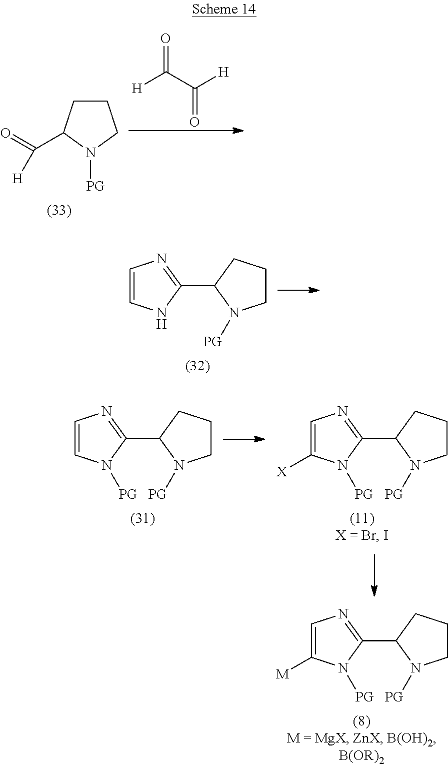

Metallated imidazole (8) is formed from haloimidazole (11) using a suitable organometallic reagent, such as butyllithium or isopropylmagnesium chloride (optionally, and preferably, used as the lithium chloride complex) in a suitable solvent, such as THF or diethylether, at a temperature of between −78° C. and room temperature. The resulting species can be further converted into another metallated species, such as an organozinc species, by further reaction with zinc chloride, or preferably into an organoboronate by reaction with a trialkylborate (such as trimethylborate) followed by hydrolysis with water, dilute base or dilute acid.

Haloimidazole (11) is formed from (31) using a source of the halogen, such as bromine, iodine, N-bromosuccinimide or N-iodosuccinimide, in a suitable solvent, such as dichloromethane or acetonitrile, at a temperature of typically between 0° C. and reflux. In the case of iodine or N-iodosuccinimide, an activating agent such as (diacetoxyiodo)benzene is typically added. The imidazole (32) is preferably protected, for example with a SEM group, using standard literature methods, such as reaction with a suitable base such as sodium hydride in a solvent, such as NMP or DMF, followed by addition of 2-(triethylsilyl)ethoxymethyl chloride at a temperature of typically between 0° C. and room temperature.

Imidazole (32) formation from compound (33) is carried out with glyoxal and ammonium hydroxide in a suitable solvent, such as methanol, at a temperature of typically between 0° C. and room temperature, wherein (the pyrrolidine nitrogen is preferably protected as a Boc or CBZ derivative).

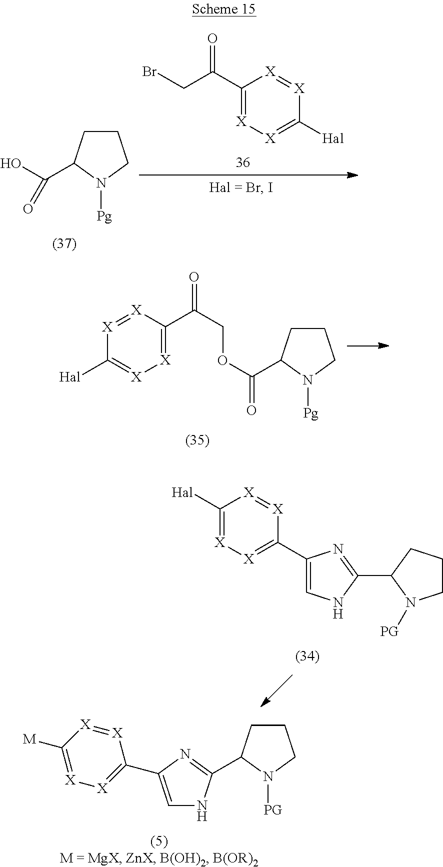

Metallated species (5) is formed from (34) using standard literature conditions as described for Scheme 12 and Scheme 14 above (the boronic acid or ester is preferably formed and used).

Imidazole (34) formation is carried out using a suitable ammonia source, typically ammonium acetate, in a solvent, such as toluene or xylene, at a temperature of typically around 100 to 150° C.

Ester (35) formation is carried out using the bromoketone (36) and a suitable base, such as triethylamine or diisopropylethylamine, in a suitable solvent, such as dichloromethane, acetonitrile or methyl-THF, at a temperature of typically between 0° C. and room temperature.

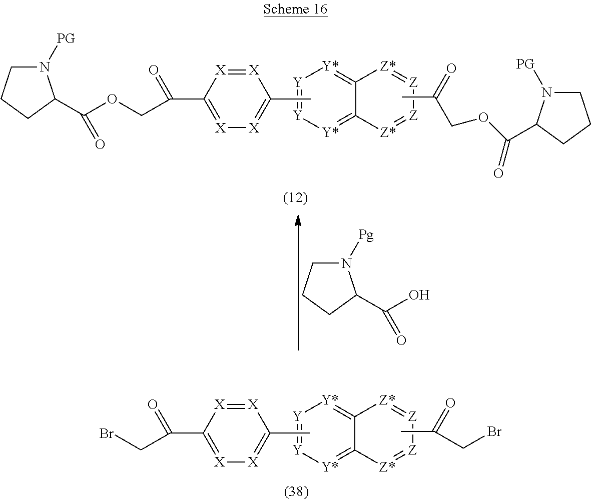

Ester (12) formation is carried out by reaction of the bromoketone (38) with the protected proline using a suitable base, such as triethylamine or diisopropylethylamine, in a suitable solvent, such as dichloromethane, acetonitrile or methyl-THF, at a temperature of typically between 0° C. and room temperature.

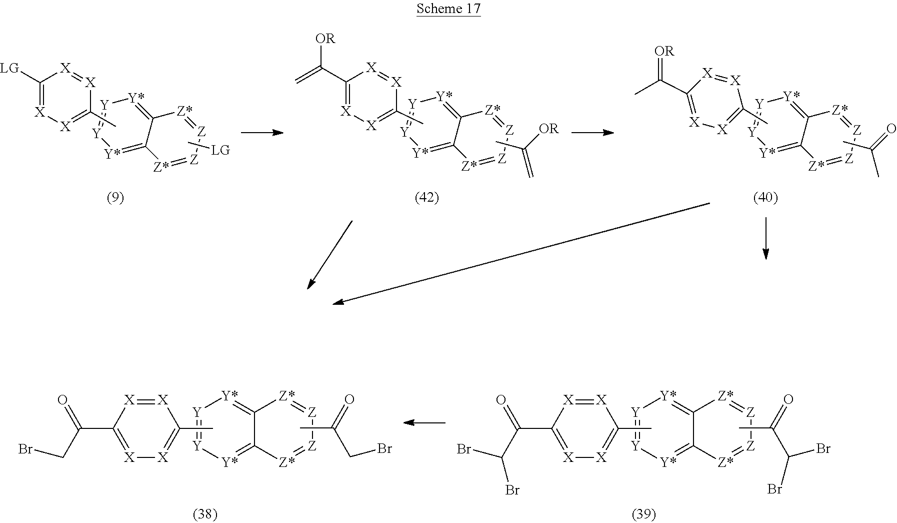

Bromoketone (38) may be formed by reduction of (39) using a suitable reducing agent, such as diethyl phosphite, in a suitable solvent, such as THF, in the presence of a base, such as triethylamine or diisopropylethylamine, at a temperature of typically between 0° C. and room temperature.

Alternatively, dienol ether (42) can be reacted directly with bromine or N-bromosuccinimide in a suitable solvent system, such as THF/water, at a temperature of typically between 0° C. and room temperature, to provide a direct route from ditriflate, or dihalide, (9) (i.e. LG=OSO₂CF₃ or Cl/Br/I).

Alternatively, bromination of the diketone (40) may be carried out using standard literature methods, such as using bromine or N-bromosuccinimide in an acidic environment, typically acetic acid, with either hydrochloric or hydrobromic acid present. The reaction is typically carried out at room temperature. Alternatively, the reaction is carried out using tetrabutylammonium tribromide in a suitable solvent, such as acetonitrile or methanol, at a temperature of typically between room temperature and 70° C. The reaction can also be carried out using copper (II) bromide in a suitable solvent, such as 1,4-dioxane, typically at reflux.

Conversion of ditriflate, or dihalide, (9) to the diketone (40) is typically carried out via formation of a dienol ether (42) and hydrolysis. Formation of the dienol ether is typically carried out using tributyl(1-ethoxy)vinyltin and a palladium catalyst, such as palladium bis(triphenylphosphine)dichloride or tetrakis(triphenylphosphine)palladium, in a suitable solvent, such as DMF, 1,4-dioxane or acetonitrile, at a temperature of typically between 80° C. and 120° C. Alternatively, reaction with an enol ether, such as n-butylvinyl ether, under palladium coupling conditions, such as tetrakis(triphenylphosphine)palladium, palladium bis(triphenylphosphine)dichloride or using palladium acetate in the presence of a suitable phosphine ligand, such as triphenylphosphine or 1,3-bis(diphenylphosphino)propane, can be carried out. Reaction is typically carried out in the presence of a suitable base, such as sodium carbonate or triethylamine, and in a solvent, such as DME, DMF, acetonitrile or ethylene glycol. Hydrolysis of the resulting enol ether under acidic conditions (for example with hydrochloric acid) gives the desired diketone.

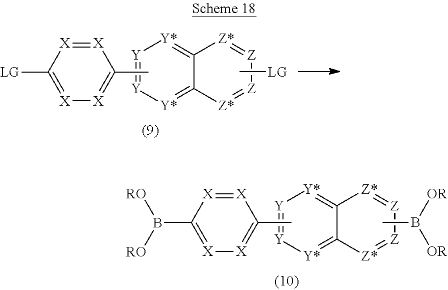

Diboronate (10) is formed from ditriflate, or dihalide, (9) (i.e. LG=OSO₂CF₃ or Cl/Br/I) by metalation using a suitable organometallic reagent, such as butyllithium or isopropylmagnesium chloride (optionally and preferably used as the lithium chloride complex) in a suitable solvent, such as THF or diethylether, at a temperature of between −78° C. and room temperature. The resulting species is then reacted with a trialkylborate, such as trimethylborate or triisopropylborate, followed by hydrolysis with water or dilute acid.

The dihalide/ditriflate (9) may be constructed by coupling the 6- and 10-membered rings using palladium catalysed coupling reactions similar to those previously described. Alternatively, depending on the identity of the 10-membered ring, it is possible to construct these compounds by ring formation reactions known to those skilled in the art. Some examples are presented in Schemes 19 to 24 below.

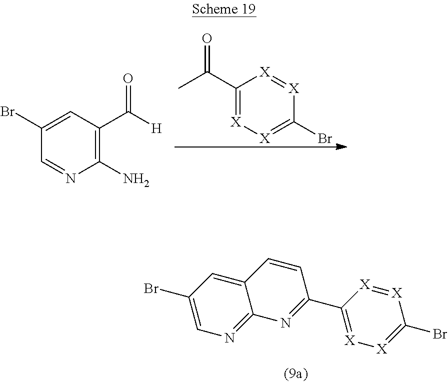

Generation of the dibromide (9a) is carried out by reaction with a ketone in the presence of a suitable amino acid, such as L-proline, in a suitable solvent, such as ethanol, typically at reflux.

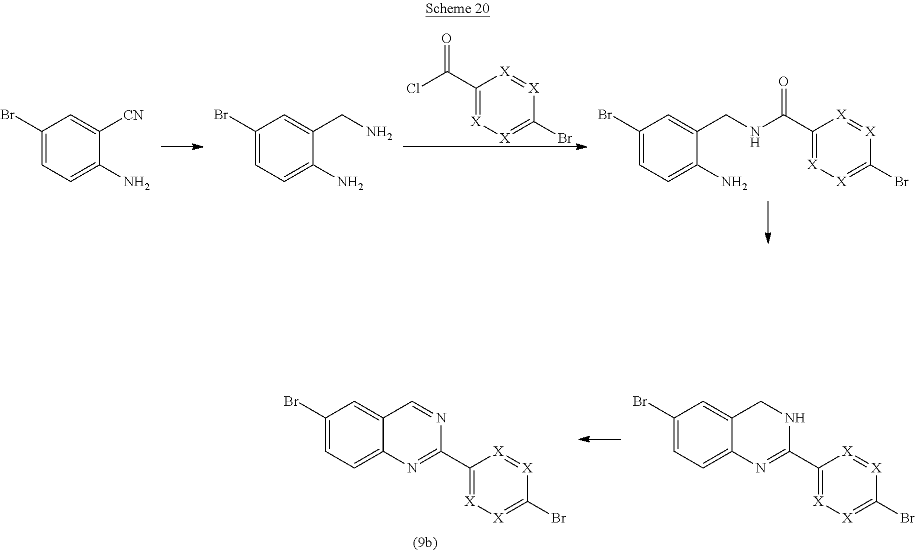

Nitrile reduction is carried out using standard literature conditions with a suitable reducing agent, such as borane or alane, in a suitable solvent, such as THF, at a temperature of typically between 0° C. and room temperature.

Amide formation is typically carried out using the acid chloride and a suitable base, such as triethylamine, diisopropylethylamine or pyridine, in a suitable solvent, such as dichloromethane or acetonitrile, at a temperature of typically between 0° C. and room temperature.

Cyclisation is typically carried out using a suitable activating agent, such as phosphorus oxychloride, typically at reflux.

Oxidation is typically carried out using known methods, such as using chloranil or DDQ, in a suitable solvent, such as toluene, at a temperature of typically around reflux.

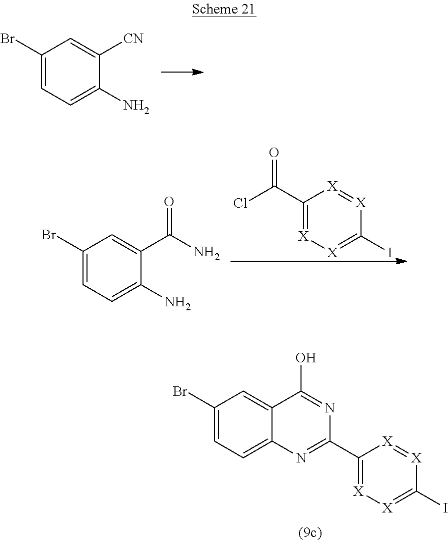

Alternatively nitrile hydrolysis is carried out using standard literature conditions, for example using sodium hydroxide solution and sodium peroxide in a suitable solvent, such as methanol, at a temperature of typically around 0° C. and room temperature.

Amide formation is typically carried out using the acid chloride and a suitable base, such as triethylamine, diisopropylethylamine or pyridine, in a suitable solvent, such as dichloromethane or acetonitrile, at a temperature of typically between 0° C. and room temperature.

Cyclisation is typically carried out using standard literature conditions, for example using aqueous potassium hydroxide in a suitable solvent, such as ethanol, at a temperature of typically around 80° C.

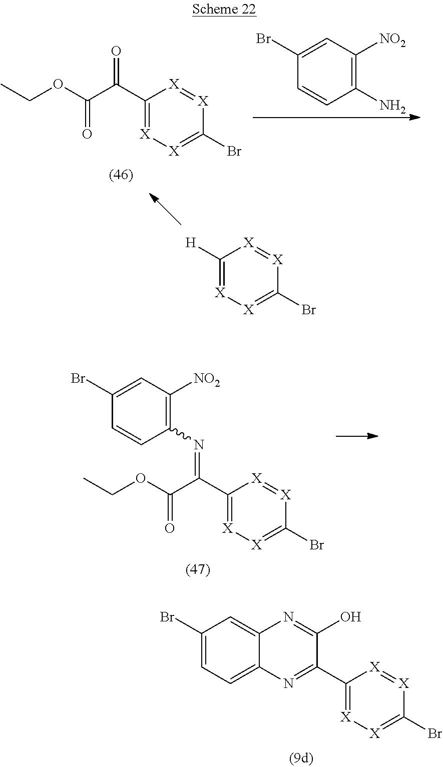

Intermediate (46) may be prepared by Friedel-Crafts acylation of a dihalo-substituted heterocycle using standard literature conditions, for example using aluminium trichloride and ethyl oxalyl chloride in a suitable solvent, such as dichloromethane, at a temperature of typically between 0° C. and room temperature.

Intermediate (47) may be prepared by imine formation using standard literature conditions, for example using titanium(IV) chloride, 4-bromo-2-nitroaniline and a suitable base, such as triethylamine, in a suitable solvent, such as dichloromethane, at a temperature of typically between 0° C. and room temperature.

Nitro reduction is typically carried out using standard literature conditions, for example using iron powder in a suitable solvent, such as acetic acid, at a temperature of typically around 80° C. Cyclisation is carried out in aqueous sodium hydroxide and hydrogen peroxide at a temperature of typically around reflux.

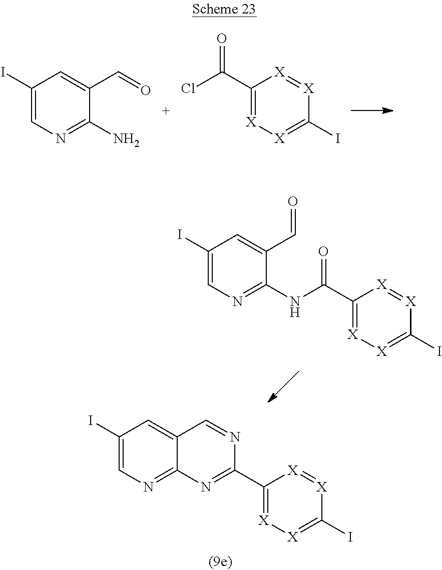

Amide coupling is typically carried out using known methods such as using a suitable base, such as triethylamine, diisopropylethylamine, (4-dimethylamino)pyridine or pyridine, in a suitable solvent, such as dichloromethane, DMF or acetonitrile, at a temperature of typically between 0° C. and reflux.

Cyclisation is typically carried out using ammonia in a suitable solvent, such as ethanol, isopropanol, THF, toluene or xylene.

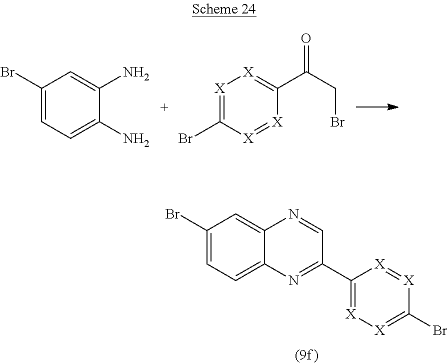

Cyclisation is typically carried out in a suitable solvent, such as ethanol, isopropanol or acetonitrile, at a temperature of typically between 50° C. and reflux.

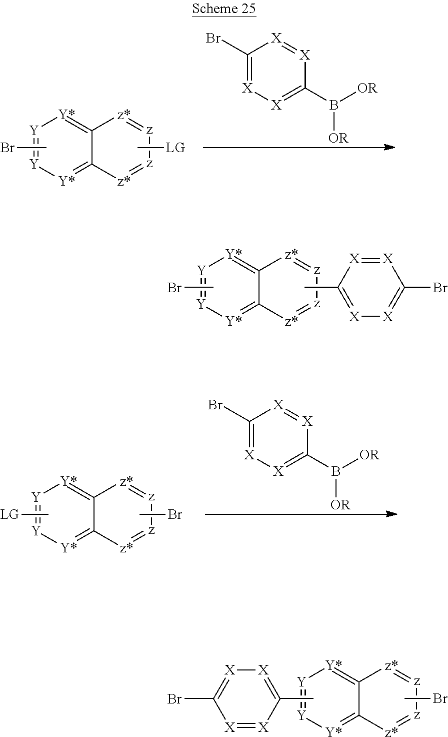

Transition metal mediated coupling is typically carried out using a palladium catalyst, such as palladium acetate, palladium bis(triphenylphosphine)dichloride, tetrakis(triphenylphosphine)palladium, or (1,1′-bis(diphenylphosphino)ferrocene)dichloropalladium, and a suitable base, such as sodium carbonate, sodium bicarbonate, potassium acetate or potassium phosphate, in a suitable solvent, such as 1,4-dioxane or DME, at a temperature of typically around 80 to 110° C. (it is assumed that LG is more prone to oxidative addition than Br and thus reaction occurs preferably here).

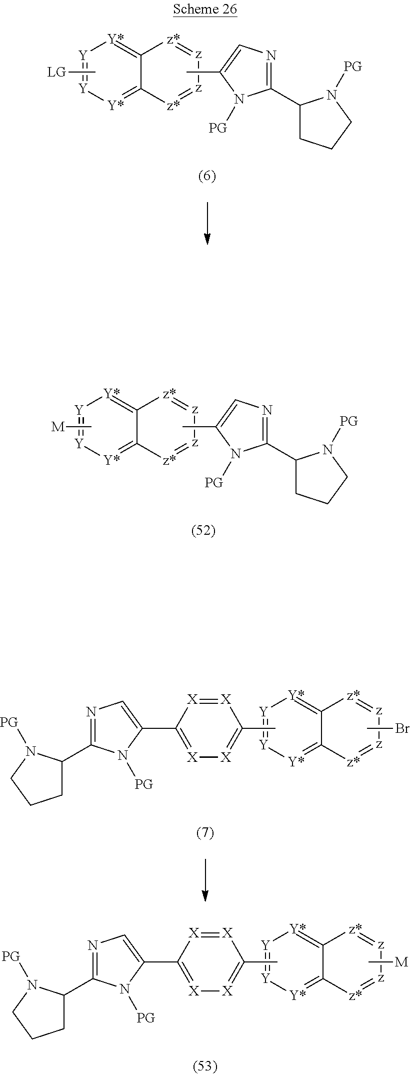

Metallated species (52) and (53) are formed from halides (6) and (7) respectively using standard literature conditions similar to described for Scheme 12 and Scheme 14 (the boronic acid or ester is preferably formed and used).

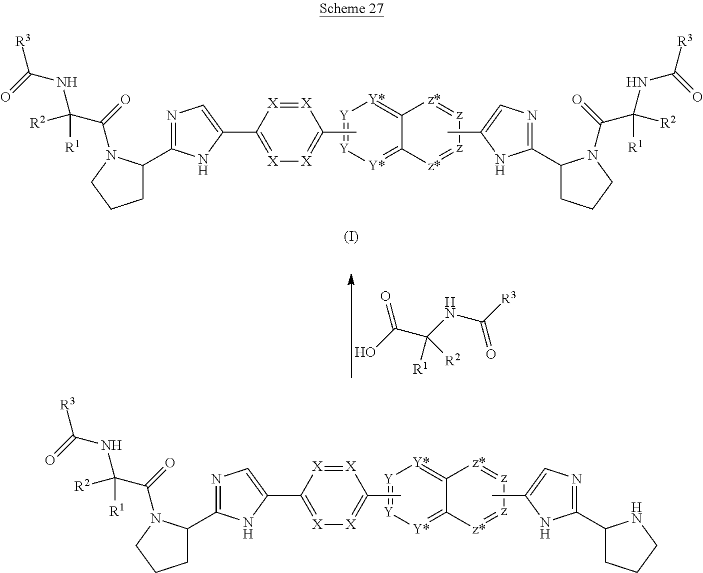

Compound (I) may also be formed by a mono amide coupling using conditions similar to those described for Scheme 1.

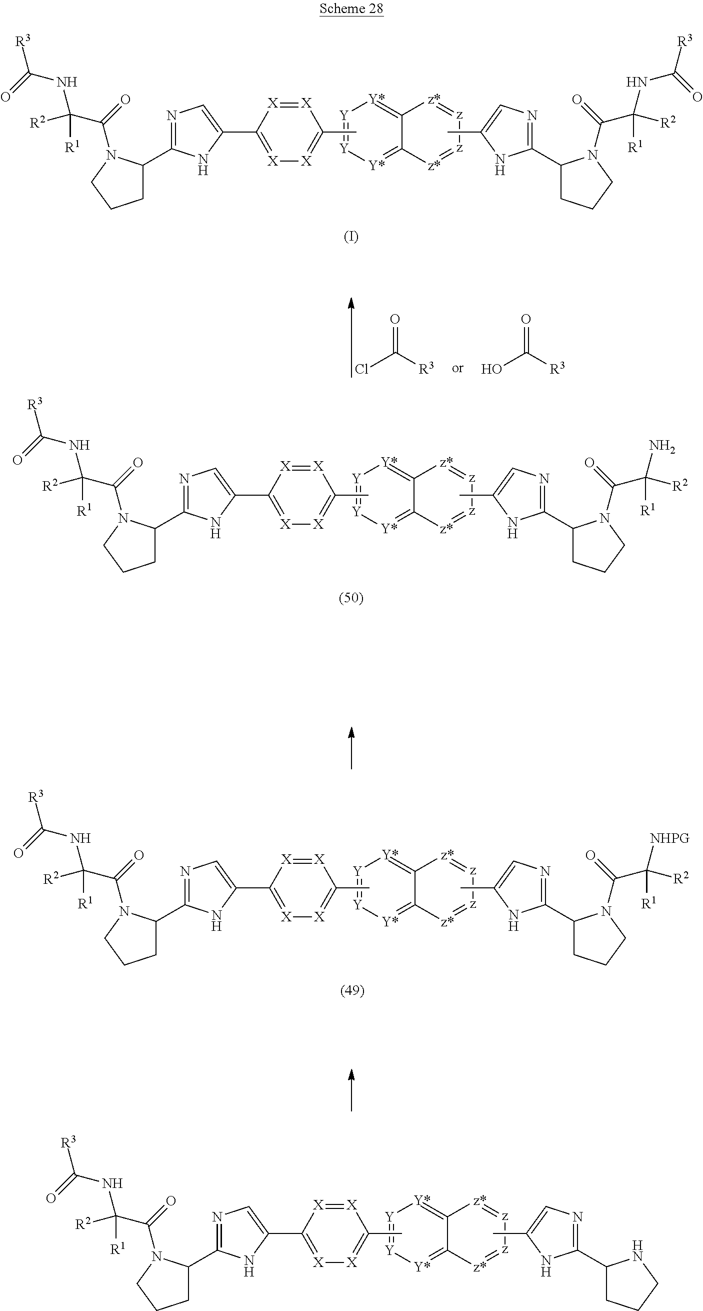

Alternatively, compound (I) may be formed by a mono amide coupling, using conditions similar to those described for Scheme 1, to give an intermediate such as compound (49) (for example, where PG=t-BOG).

Deprotection is carried out using known literature methods, such as reaction with an acid, such as hydrochloric acid or trifluoracetic acid, in a suitable solvent, such as methanol or 1,4-dioxane, at a temperature of typically between room temperature and reflux.

Acylation of compound (50) is carried out using known literature methods, such as reaction with an acid chloride in the presence of a suitable base, such as triethylamine or diisopropylethylamine, in a suitable solvent, such as dichloromethane or toluene, at a temperature of between 0° C. and room temperature. Alternatively, the acid can be converted to a suitable activated species with a coupling agent, such as

EDCI.HCl, EDCI.MeI, HBTU, HATU, PyBop, DCC, or CDI, in a suitable solvent, such as dichloromethane, acetonitrile or DMF. In the presence of EDCI.HCl or EDCI.MeI, HOBT is optionally added. A suitable base, such as triethylamine or diisopropylethylamine, is also used and the reaction is typically carried out at room temperature.

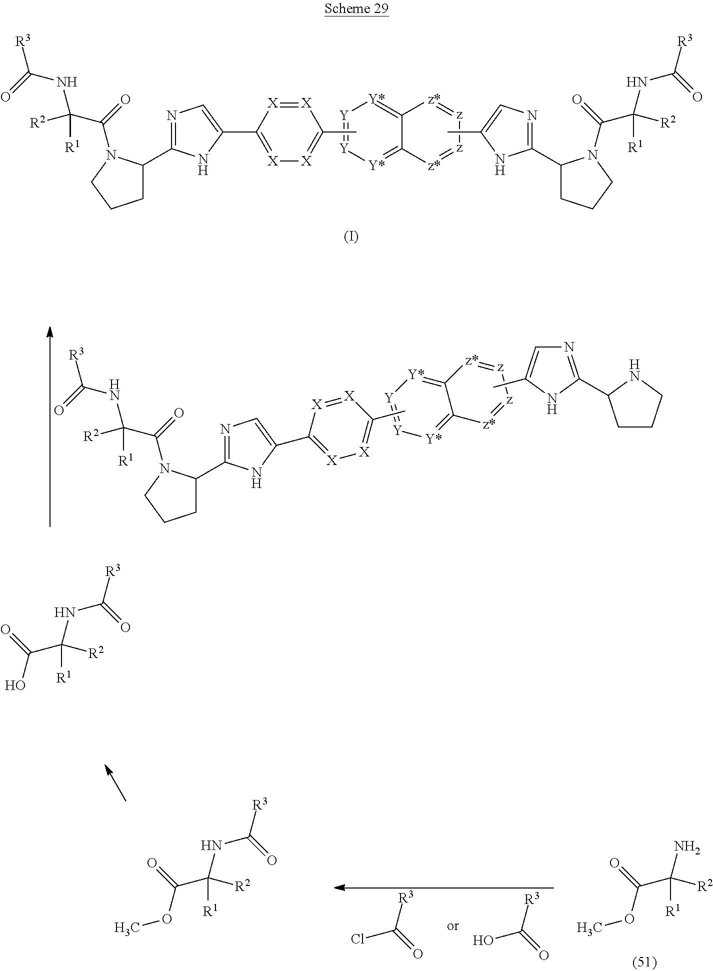

Alternatively compound (I) may be formed by acylating compound (51) using conditions similar to those described for Scheme 28.

Ester hydrolysis is carried out using known literature methods, such as reaction with lithium hydroxide, in a suitable solvent, such as THF/water mixtures, typically at room temperature.

The final amide coupling may be formed using conditions similar to those described for Scheme 1, to give (I).

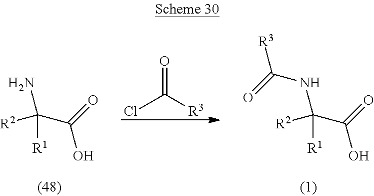

Acid (1) may be formed using standard literature conditions, for example when R³=OMe amine (48) is acylated using methyl chloroformate and a suitable base, such as sodium carbonate, in a suitable solvent, such as aqueous sodium hydroxide, at a temperature of typically around 0° C. and room temperature.

In a second aspect, the present invention also provides a pharmaceutical composition including a compound of formula (I) or a pharmaceutically acceptable salt thereof, together with a pharmaceutically acceptable excipient.

The term ‘excipient’ is used herein to describe any ingredient other than the compound of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include both solid and liquid formulations.

Solid formulations include tablets, capsules (containing particulates, liquids, or powders), lozenges (including liquid-filled lozenges), chews, multi- and nano-particulates, gels, solid solutions, liposomal preparations, films, ovules, and sprays.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11(6), 981-986, by Liang and Chen (2001).

For tablet dosage forms, depending on dose, the drug may make up from 1 weight % to 80 weight % of the dosage form, more typically from 5 weight % to 60 weight % of the dosage form.

In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate.

Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets”, Vol. 1, by H. Lieberman and L. Lachman (Marcel Dekker, New York, 1980).

Consumable oral films are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the compound of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus the compound of the invention may be formulated as a solid, semi-solid, or thixotropic liquid for administration as an implanted depot providing modified release of the active compound.

Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

As demonstrated by the results of biological testing (described in greater detail below), the compounds of formula (I) are potent inhibitors of HCV replication.

Thus, in a third aspect, the present invention provides a compound of formula (I) or a pharmaceutically acceptable salt thereof, for use as a medicament.

A specific embodiment of the invention is a compound of formula (I) or a pharmaceutically acceptable salt thereof, for use in the treatment of a disease for which an inhibitor of HCV replication is indicated.

Another specific embodiment of the invention is a compound of formula (I) or a pharmaceutically acceptable salt thereof, for use in the treatment of HCV infection.

The present invention also provides the use of a compound of formula (I) or a pharmaceutically acceptable salt thereof, for the manufacture of a medicament to treat a disease for which an inhibitor of HCV replication is indicated.

A specific embodiment of the invention is the use of a compound of formula (I) or a pharmaceutically acceptable salt thereof for the manufacture of a medicament for the treatment of HCV infection.

The present invention also provides a method of treatment of a mammal, including a human being, to treat a disease for which an inhibitor of HCV replication is indicated, including administering to said mammal an effective amount of a compound of formula (I) or a pharmaceutically acceptable salt thereof.

A specific embodiment of the invention is a method of treatment of a mammal, including a human being, to treat HCV infection, including administering to said mammal an effective amount of a compound of formula (I) or a pharmaceutically acceptable salt thereof.

The term ‘treatment’ as used herein includes both preventative and curative treatment of a disease or disorder. It also includes slowing, interrupting, controlling or stopping the progression of a disease or disorder. It also includes preventing, curing, slowing, interrupting, controlling or stopping the symptoms of a disease or disorder.

The compound of the present invention may be administered in combination with one or more additional agents for the treatment of a mammal, such as a human, that is suffering from an infection with the HCV virus, or any other disease or condition which is related to infection with the HCV virus. The agents that may be used in combination with the compounds of the present invention include, but are not limited to, cyclophilin inhibitors (such as NIM-811, Debio-025 and SCY-635), immunomodulators (such as Zadaxin, Ceplene, Cellcept, Civacir and Zadazim), TLR9 agonists (such as Actilon), antisense agents (such as ISIS14803), NS4A inhibitors (such as ACH-806), NS5A inhibitors (such as A831, BMS-790052 and A689), inosine monophosphate dehydrogenase inhibitors (such as Levovirin, Miremepodib, Viramidine and Ribavirin), inhibitors of HCV entry (such as XTL-6865), NS3 serine protease inhibitors (such as Telapravir, Bocepravir, TMC-435350, MK-7009, BI-201335, ABT-450, ITMN-191 and BILN-2061; and also compounds described in Reiser and Timm, Expert Rev. Anti Infect. Ther. 7(5), 537-547, (2009)), TLR7 agonists (such as N-(4-(4-amino-2-ethyl-1H-imidazo[4,5-c]quinolin-1-yl)butyl)methanesulfonamide, ANA-971 and ANA-773), NS5B RNA-polymerase inhibitors (such as Filibuvir, HCV-796, Valopicitabine, GL-59728, GL-60667, PSI-6130, R1626, R7128, JTK-003 GL-59728 and GS-9190; and also compounds described in Beaulieu, Expert Opin. Ther. Patents, 19(2), 145-164 (2009)), caspase inhibitors (such as IDN-6556), glucosidase inhibitors (such as Celgosivir), inhibitors of NS4B and other compounds described in Holler et al. Expert Opin. Drug Discov. 4(3), 293-314 (2009). Compounds of the present invention can also be combined with inhibitors of the hepatitis C structural proteins such as nucleocapsid or core proteins. Compounds of the present invention can also be combined with an interferon, or an interferon derivative (such as Albuferon, AlbInterferon, BLX-883 (locteron), Infergen A, Omega IFN, IFN beta, Rebif, Roferon A, Intron A, Rebetron, Actimmune, Multiferon, Wellferon, Omniferon, Pegasys, Pegasys+Ribavirin, and Pegintron+Ribavirin).

Such a combination may be administered such that the compound of the present invention is present in the same pharmaceutical composition as the additional agent(s) described above. Alternatively, such a combination may be administered such that the compound of the present invention is present in a pharmaceutical composition that is separate from the pharmaceutical composition in which the additional agent(s) is(are) found. If the compound of the present invention is administered separately from the additional agent(s), such administration may take place concomitantly or sequentially with an appropriate period of time in between.

Additionally, the compound of the present invention may be administered in combination with one or more additional agents that have the effect of increasing the exposure of the mammal to the compound of the invention. The term ‘exposure’, as used herein, refers to the concentration of the compound of the invention in the plasma of a mammal as measured over a period of time. The exposure of a mammal to a particular compound can be measured by administering the compound of the invention to a mammal in an appropriate form, withdrawing plasma samples at predetermined times, and measuring the amount of a compound of the invention in the plasma using an appropriate analytical technique, such as liquid chromatography or liquid chromatography/mass spectroscopy. The amount of the compound of the invention present in the plasma at a certain time is determined and the concentration and time data from all the samples are plotted to afford a curve. The area under this curve is calculated and affords the exposure of the mammal to the compound. The terms ‘exposure’, ‘area under the curve’, and ‘area under the concentration/time curve’ are intended to have the same meaning and may be used interchangeably.

Among the agents that may be used to increase the exposure of a mammal to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP3A4 include, but are not limited to, ritonavir, delavirdine, N-(3,4-difluorobenzyl)-2-{[(4-methoxypyridin-3-yl)amino]sulfonyl}-N-methylbenzamide, and N-(1-(5-(4-fluorobenzyl)-3-(pyridin-4-yl)-1H-pyrazole-1-carbonyl)piperidin-4-yl)methanesulfonamide.

Such a combination may be administered such that the compound of the present invention is present in the same formulation as the additional agent(s) described above. Alternatively, such a combination may be administered such that the compound of the present invention is present in a pharmaceutical composition that is separate from the pharmaceutical composition in which the additional agent(s) is(are) found. If the compound of the present invention is administered separately from the additional agent(s), such administration may take place concomitantly or sequentially with an appropriate period of time in between.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The following procedures illustrate the preparation of specific examples of compounds of the formula (I). ¹H-NMR spectra were recorded on a Varian Mercury 400 MHz or Bruker Avance 400 MHz or Jeol ECX 400 MHz NMR. NMR spectra were obtained as DMSO-d₆ solutions (reported in ppm), using chloroform as the reference standard (7.25 ppm and 77.00 ppm). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Microwave experiments were carried out using a Biotage initiator 8.

The Mass Spectra were Obtained Using:

### Waters ZQ ESCl 2 min LC-MS,

### Waters ZQ ESCl 4.5 min LC-MS,

### Waters ZQ ESCl 6 min LC-MS,

### Applied Biosystem's API-2000 5 min LC-MS (System 1),

Waters Alliance 2795 with ZQ2000 (ESI) 5 min LC-MS (System 2),

### Agilent 110 HPLC 5 min (System 5),

### Waters ZQ ESCl 8 min LC-MS,

Waters Alliance 2695 with ZQ2000 (ESI) 25 min, and
HP 1100 HPLC with Waters Micromass ZQ mass detector 12.5 min LC-MS.
HPLC mass spectra were obtained using a Waters Acquity ZQD (ESI) 1.5 min LC-MS.
2 minute LC-MS Gradient and Instrument Conditions:

### Acid Run:

A: 0.1% formic acid in water
B: 0.1% formic acid in acetonitrile
Column: C18 phase Fortis Pace 20×2.1 mm with 3 micron particle size
Gradient: 70-2% A over 1.8 min, 0.2 min hold, 1.8 mL/min
UV: 210 nm-450 nm DAD

### Temperature: 75° C.

4.5 minute LC-MS Gradient and Instrument Conditions:

### Acid Run:

A: 0.05% formic acid in water
B: 0.05% formic acid in acetonitrile
Column: C18 phase Phenomenex Gemini 50×4.6 with 3 micron particle size
Gradient: 95-5% A over 3.5 min, 1.0 min hold, 2.0 mL/min
UV: 200 nm-400 nm DAD

### Temperature: 45° C.

6 minute LC-MS Gradient and Instrument Conditions:

### Acid Run:

A: 0.1% formic acid in water
B: 0.1% formic acid in acetonitrile
Column: C18 phase Fortis 50×4.6 mm with 5 micron particle size
Gradient: 95-5% A over 3 min, 1 min hold, 1 mL/min
UV: 210 nm-450 nm DAD

### Temperature: 50° C.

5 minute LC-MS Gradient and Instrument Conditions (System 1):

### Acid Run:

A: 0.05% trifluoroacetic acid/0.05% formic acid/10 mM ammonium acetate

### B: Acetonitrile

Column: C18 phase Phenomenex Gemini 50×4.6 mm with 5 micron particle size
Gradient: 90-10% A over 3 min, 1 min hold, 1.2 mL/min

### UV: 220 nm, 260 nm

### Temperature: 25° C.

5 minute LC-MS Gradient and Instrument Conditions (System 2):

### Acid Run:

A: water

### B: Methanol

D: 2% formic acid in water
Column: XBridge C18 2.1×30 mm with 5 micron particle size
Gradient table:

| min | % A | % B | % C | % D |
|---|---|---|---|---|
| 0 | 90 | 5 | | 5 |
| 0.1 | 90 | 5 | | 5 |
| 3 | 0 | 95 | | 5 |
| 3.9 | 0 | 95 | | 5 |
| 4 | 90 | 5 | | 5 |

Flow: 1 mL/min
UV: 215 nm-350 nm DAD

### Temperature: 25° C.

### Base Run:

C: 10 mM ammonium bicarbonate @ pH10
B: methanol
Column: XBridge C18 2.1×30 mm with 5 micron particle size
Gradient table:

| min | % A | % B | % C | % D |
|---|---|---|---|---|
| 0 | | 5 | 95 | |
| 0.1 | | 5 | 95 | |
| 3 | | 95 | 5 | |
| 3.9 | | 95 | 5 | |
| 4 | | 5 | 95 | |

Flow: 0.5 mL/min
UV: 215 nm-350 nm DAD

### Temperature: 25° C.

5 minute LC-MS Gradient and Instrument Conditions (System 5)

### Acid Run:

A: 0.05% formic acid in water

### B: Acetonitrile

Column: C18 phase Phenomenex Gemini 50×4.6 mm with 3 micron particle size
Gradient: 80-50% A over 0.5 min, 50-2% over 3 min, 1.0 hold, 2 mL/min
UV: 200 nm-400 nm DAD

### Temperature: 40° C.

8 minute LC-MS Gradient and Instrument Conditions:

### Acid Run:

A: 0.05% formic acid in water
B: 0.05% formic acid in acetonitrile
Column: C18 phase Phenomenex Gemini 50×4.6 mm with 3 micron particle size
Gradient: 95-5% A over 3.5 min, 4.5 min hold, 2 mL/min
UV: 200 nm-400 nm DAD

### Temperature: 40° C.

25 minute LC-MS Gradient and Instrument Conditions:

### Acid Run:

A: water
B: acetonitrile
D: 2% formic acid in water
Column: XBridge C18 3×150 mm with 5 micron particle size
Gradient table:

| min | % A | % B | % C | % D |
|---|---|---|---|---|
| 0 | 90 | 5 | | 5 |
| 15 | 0 | 95 | | 5 |
| 25 | 0 | 95 | | 5 |

Flow: 0.5 mL/min
UV: 215 nm-350 nm DAD

### Temperature: 30° C.

### Base Run:

C: 10 mM ammonium bicarbonate @ pH10
B: acetonitrile
Column: XBridge C18 3×150 mm with 5 micron particle size
Gradient table:

| min | % A | % B | % C | % D |
|---|---|---|---|---|
| 0 | | 5 | 95 | |
| 15 | | 95 | 5 | |
| 25 | | 95 | 5 | |

Flow: 0.5 mL/min
UV: 215 nm-350 nm DAD

### Temperature: 30° C.

12.5 minute LC-MS Gradient and Instrument Conditions:

### Eluent A=Water 0.05% TFA

### Eluent B=MeCN

Flow rate 1.2 ml/min

| t = 0 | 5% B |
|---|---|
| 1 | 5% B |
| 9 | 100% B |
| 11.5 | 100% B |
| 11.6 | 5% B |
| 12.5 | 5% B |

### Temp 50° C.

Column: SB C18 3.0×50 1.8 microM
1.5 minute LC-MS Gradient Conditions:

### Acid Run:

A: 0.1% formic acid in water

### B: Acetonitrile

Column: XBridge C18 2.1×50 mm with 2.5 micron particle size
Gradient table:

| min | % A | % B |
|---|---|---|
| 0 | 98 | 2 |
| 0.8 | 2 | 98 |
| 1.2 | 2 | 98 |
| 1.25 | 98 | 2 |

Flow: 0.8 mL/min
UV: 215 nm-350 nm DAD

### Temperature: 30° C.

### Base Run:

A: 10 mM ammonium bicarbonate @ pH10
B: acetonitrile
Column: XBridge C18 2.1×50 mm with 2.5 micron particle size
Gradient table:

| min | % A | % B |
|---|---|---|
| 0 | 98 | 2 |
| 0.8 | 2 | 98 |
| 1.2 | 2 | 98 |
| 1.25 | 98 | 2 |

Flow: 0.8 mL/min
UV: 215 nm-350 nm DAD

### Temperature: 30° C.

Accurate Mass Spectra were Obtained Using a Bruker MicrOTOF Mass Spectrometer.
loop: 1 ml/min 1.5 min run time.
A: 0.1% formic acid+H20
B: 0.1% formic acid+MeCN
Sodium formate used as calibrant.
Preparative HPLC was Carried Out Using Waters Purification Systems with Either PDA/Mass Spec or Uv Detection (System 3) or (System 4).

### System 3 Eluent and Instrument Conditions:

Column: XBridge C18 19×100 mm or 19×150 mm or 30×150 mm with 5 micron particle size
Eluent: Methanol/0.1% ammonia in water
Flow: 20 or 50 mL/min

### Temperature: 25° C.

### System 4 Eluent, Gradient and Instrument Conditions:

Column: XTerra C18, 19×250 mm, 10 micron particle size
Eluent: (A) Acetonitrile/(B) 0.05% ammonia in water (pH=10.5)
Flow: 16 mL/min

### Temperature: 25° C.

### Gradient Table:

| Time | % A | % B |
|---|---|---|
| 0 | 90 | 10 |
| 3 | 70 | 30 |
| 8 | 50 | 50 |
| 18 | 20 | 80 |
| 19 | 5 | 95 |
| 20 | 5 | 95 |
| 21 | 90 | 10 |
| 25 | 90 | 10 |

### GCMS was Obtained on an Agilent 6890 Series (PLUS+) GC System (System 6).

### GCMS Conditions:

### AGILENT 5973 Network MASS SELECTIVE DETECTOR

### AGILENT 7683 Series INJECTOR

GC-MS COLUMN USED: HP-5MS (30m×0.25 mm×0.25 um)

### DILUENT: Dichloromethane

### INJECTION VOLUME: 1 μl

SPLIT RATIO: 20:1 ml/min
COLUMN FLOW: 1.2 ml/min

### FRONT INLET TEMP: 300° C.

### CARRIER GAS: He

### MS SOURCE TEMP: 230° C.

Oven Condition:

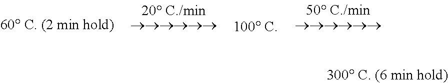

### TOTAL RUN TIME: 14 min

GCMS was obtained on a Agilent technologies 6890N GC system with Agilent 5975 inert mass selective detector (System 7)

| Column | HP-5MSi | | | |
|---|---|---|---|---|
| Column Dimensions | 30 m × 0.250 mm × 0.25 um | | | |
| Column Flow | 0.9 ml/min (constant flow mode) | | | |
| | Initial | | Final | Hold |
| | Temp | Rate | Temp | Time |
| Oven Temperature Gradient | (° C.) | (° C./min) | (° C.) | (min) |
| | 50 | — | — | 1.00 |
| | 50 | 20 | 280 | 1.00 |

| | |
|---|---|
| Run time | 13.50 min |
| Injection Volume | 1 μl |
| Inlet Mode | Split |
| Inlet temperature | 250° C. |
| Split ratio | 100:1 |
| Gas type | Helium |
| FID Temperature | 300° C. |
| FID Hydrogen Flow | 30.0 ml/min |
| FID Air flow | 400 ml/min |
| FID mode | Constant make up flow |
| FID Make up gas | Nitrogen |
| FID Make up flow | 25.0 ml/min |
| MS Acquisition mode | Scan |
| MS Solvent Delay | 4.00 min |
| MS Low Mass | 50 |
| MS High Mass | 400 |
| MS Quad temperature | 100° C. |
| MS Source Temperature | 230° C. |
| MS Transfer line temperature | 280° C. |

### ABBREVIATIONS

DIPEA: N,N-Diisopropylethylamine
EDCI.HCl: N-(3-dimethylaminopropyl)-N′-ethylcarbodiimide hydrochloride
EDCI.MeI: N-(3-dimethylaminopropyl)-N′-ethylcarbodiimide methiodide
HBTU: o-Benzotriazol-1-yl-N,N,N′,N′-tetramethyluronium hexafluorophosphate
HATU: 2-(7-Azabenzotriazol-1-yl)-1,1,3,3-tetramethyluronium hexafluorophosphate
DCC: N,N′-Dicyclohexylcarbodiimide
DCM: Dichloromethane
CDI: N,N′-Carbonyldiimidazole
HOBT: N-Hydroxybenzotriazole hydrate
PyBop: (Benzotriazol-1-yloxy)tripyrrolidinophosphonium hexafluorophosphate
APCI: Atmospheric pressure chemical ionization
ESI: Electrospray ionization
THF: Tetrahydrofuran
EtOAc: Ethyl acetate
MeOH: Methanol
EtOH: Ethanol
DMF: N,N-Dimethylformamide
DME: Dimethoxyethane
Pd₂(dba)₃: Tris(dibenzylideneacetone)dipalladium(0)
Pd(OAc)₂: Palladium acetate
PdCl₂(PPh₃)₂: Palladium bis(triphenylphosphine)dichloride
Pd(dppf)Cl₂.DCM: [1,1-Bis(diphenylphosphino)ferrocene]dichloropalladium(II), complex with dichloro-methane
Sphos: 2-Dicyclohexylphosphino-2′,6′-dimethoxybiphenyl
MgSO₄: Magnesium sulfate
Na₂SO₄: Sodium sulfate
Na₂CO₃: Sodium carbonate
K₃PO₄: Potassium phosphate
DDQ: 2,3-Dichloro-5,6-Dicyanobenzoquinone
DABCO: 1,4-Diazabicyclo[2.2.2]octane
TBME: tert-Butyl dimethyl ether
Pd(PPh₃)₄: Tetrakis(triphenylphosphine)palladium(0)
TBAF: Tetrabutyl ammonium fluoride
POBr₃: Phosphorus oxybromide
NBS: N-Bromosuccinimide

### EXAMPLE 1

### RS)-1-((S)-2-{5-[4-(6-{2-[(S)-1-((S)-2-Methoxycarbonylamino-3-methylbutyryl)-pyrrolidin-2-yl]-3H-imidazol-4-yl}-quinazolin-2-yl)-phenyl]-1H-imidazol-2-yl}-pyrrolidine-1-carbonyl)-2-methyl-propyl]carbamic acid methyl ester

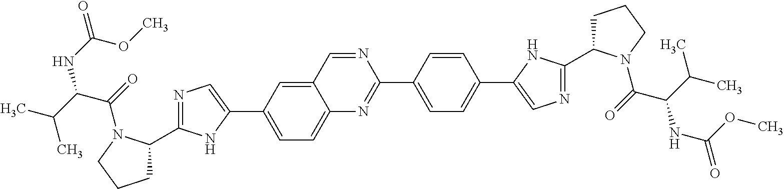

Method A: To a stirred solution of 6-((S)-2-pyrrolidin-2-yl-3H-imidazol-4-yl)-2-[4-((S)-2-pyrrolidin-2-yl-3H-imidazol-4-yl)phenyl]quinazoline hydrochloride, obtained from Preparation 10 (0.38 g, 0.7 mmol), in dry DMF (8 mL) was added N-(methoxycarbonyl)-L-valine, obtained from Preparation 22 (0.22 g, 1.26 mmol), DIPEA (0.62 mL, 3.5 mmol) and HATU (0.53 g, 1.4 mmol). The reaction mixture was stirred at room temperature for 30 minutes. The solvent was then evaporated in vacuo and the crude residue was purified by prep-HPLC to afford the title compound as a yellow solid (110 mg).

¹H NMR (400 MHz, DMSO-d₆): δ=11.87 (br s, 2H), 9.56 (1H, s), 8.52 (2H, m), 8.37 (2H, m), 7.94 (3H, m), 7.63 (1H, s), 7.50 (1H, s), 6.39 (2H, m), 5.20 (2H, m), 4.18 (2H, m), 3.78 (4H, m), 3.57 (6H, s), 2.24 (6H, m), 2.02 (4H, m), 0.90 (12H, m). LCMS (run time=6 min): Rₜ=2.13 min; m/z 791 [M+H]⁺.

Method B: To acetonitrile (1.4 L) was added HOBT (39.36 g, 257.05 mmol), and EDCI.HCl (47.31 g, 246.77 mmol) followed by N-(methoxycarbonyl)-L-valine (37.83 g, 215.92 mmol). The reaction mixture was stirred at room temperature for 1 hour. To this stirred solution was added 6-((S)-2-pyrrolidin-2-yl-3H-imidazol-4-yl)-2-[4-((S)-2-pyrrolidin-2-yl-3H-imidazol-4-yl)phenyl]quinazoline (56.5 g, 102.82 mmol) and the reaction was cooled to 0° C. To the reaction was added DIPEA (143.45 mL, 822.5 mmol). The reaction was warmed to room temperature and stirred overnight. The solvent was then evaporated in vacuo and the crude residue was dissolved in DCM (560 mL). It was washed with a saturated aqueous solution of sodium bicarbonate (560 mL) followed by H₂O (560 mL). The organic phase was dried over magnesium sulphate, filtered and concentrated to dryness. The crude residue was purified by column chromatography on silica gel (Acetone:Heptane 60:40) to afford the title compound as a yellow solid (33 g).

### EXAMPLE 2

### Methyl {(2S)-1-[(2S)-2-{5-[6-(4-{2-[(2S)-1-{(2S)-2-[(methoxycarbonyl)amino]-3-methyl-butanoyl}pyrrolidin-2-yl]-1H-imidazol-4-yl}phenyl)naphthalen-2-yl]-1H-imidazol-2-yl}pyrrolidin-1-yl]-3-methyl-1-oxobutan-2-yl}carbamate

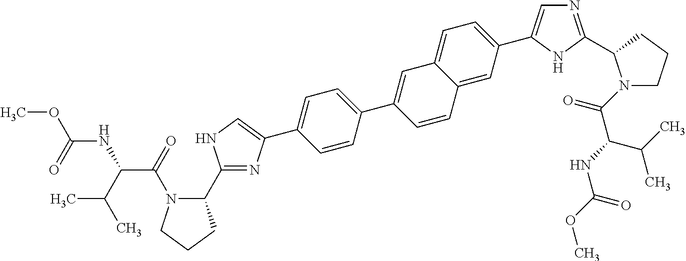

To a stirred solution of N-(methoxycarbonyl)-L-valine (41 mg, 0.23 mmol), obtained from Preparation 22, in DMF (1 mL) was added HATU (88 mg, 0.23 mmol) and DIPEA (0.04 mL, 0.23 mmol). After stirring at room temperature for 30 minutes, 2-[(2S)-pyrrolidin-2-yl]-4-[4-(6-{2-[(2S)-pyrrolidin-2-yl]-1H-imidazol-5-yl}naphthalen-2-yl)phenyl]-1H-imidazole (50 mg, 0.1 mmol), obtained from Preparation 21, was added and the mixture was stirred at room temperature for 19 hours. The reaction mixture was evaporated under reduced pressure. The resulting crude material was dry loaded onto silica and purified by column chromatography (redisep (4 g), 100% DCM-95:5:0.5 DCM: MeOH: NH₃) to give 43 mg of the title compound as a pale yellow solid. LCMS (run time=6 min): Rₜ=2.40; m/z 789 [M+H⁺]. Accurate mass spec meas. m/z 789.4098 theoretical meas. m/z 789.4088.

### EXAMPLE 3

### [(S)-1-((S)-2-{4-[2-(4-{2-[(S)-1-((S)-2-Methoxycarbonylamino-3-methyl-butyryl)-pyrrolidin-2-yl]-3H-imidazol-4-yl}-phenyl)-quinolin-6-yl]-1H-imidazol-2-yl}-pyrrolidine-1-carbonyl)-2-methyl-propyl]-carbamic acid methyl ester

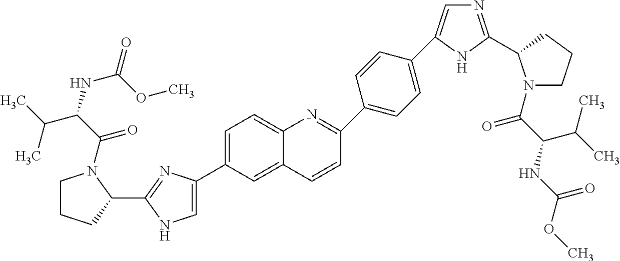

Methyl (S)-3-methyl-1-oxo-1-((S)-2-(5-(4-(4,4,5,5-tetramethyl-1,3,2-dioxaborolan-2-yl)-phenyl)-1H-imidazol-2-yl)-pyrrolidin-1-yl)butan-2-yl carbamate (30 mg, 0.06 mmol), obtained from Preparation 28, ((S)-1-{(S)-2-[4-(2-chloro-quinolin-6-yl)-1-(2-trimethylsilanyl-ethoxymethyl)-1H-imidazol-2-yl]-pyrrolidine-1-carbonyl}-2-methyl-propyl)-carbamic acid methyl ester (35 mg, 0.06 mmol), obtained from Preparation 25, and Pd(dppf)Cl₂.DCM (2.4 mg, 0.003 mmol) were added to a microwave vial, followed by 1,2-dimethoxyethane (0.5 mL) and 2N sodium bicarbonate solution (90 μL, 0.18 mmol). The mixture was heated under microwave irradiation at 120° C. for 20 minutes. The reaction mixture was filtered then partitioned between ethyl acetate and water. The organic phase was evaporated and redissolved in ethanol (3 mL). To this solution was added 4N hydrogen chloride in 1,4-dioxane (0.5 mL) and the mixture was heated to 70° C. After 15 minutes, heating was stopped and the mixture was stirred at room temperature for 16 hours. Further hydrogen chloride in 1,4-dioxane (0.5 mL) was added and the heating was resumed for a further 30 minutes. The reaction was evaporated to dryness, redissolved in methanol and applied to an SCX cartridge, eluting initially with methanol then 7N ammonia in methanol to elute the product. The resulting solution was evaporated under reduced pressure and further purified by column chromatography on silica gel (Redisep 4 g, DCM: 90:10:1 DCM:MeOH:NH3 100:0 to 0:100) to give 21 mg of the title compound as a pale orange solid after trituration with diethyl ether. LCMS (run time=6 min): Rₜ=2.44; m/z 790 [M+H⁺].

### EXAMPLE 4

### Methyl {(2S)-1-[(2S)-2-{5-[4-(6-{2-[(2S)-1-{(2S)-2-[(methoxycarbonyl)amino]-3-methyl butanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate

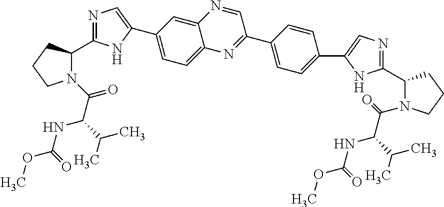

Method A: To a stirred solution of the acid obtained from Preparation 22 (13.3 mg, 0.08 mmol) in DMF (1 mL) was added PyBOP (29 mg, 0.08 mmol), and DIPEA (0.013 mL, 0.08 mmol). The mixture was stirred at room temperature for 10 minutes. The diamine, obtained from Preparation 34 (17 mg, 0.04 mmol), in DMF (1 mL) was added and the mixture was stirred at room temperature overnight. The reaction mixture was diluted with ethyl acetate (5 mL) and the organic phase was washed with water (5 mL), saturated sodium bicarbonate solution (5 mL), and brine (4×5 mL). It was dried with Na₂SO₄ and the solvent was removed under reduced pressure. The resulting crude material was purified by column chromatography on silica gel (redisep (4 g), DCM: MeOH: NH₃ 97:3:0.3-91:9:0.9) to give 17 mg of the title compound as a bright yellow solid.

¹H-NMR (400 MHz, MeOD): δ=9.29 (1H, m), 8.41-7.34 (9H, m), 5.41-5.14 (2H, m), 4.25 (2H, m), 4.01 (2H, m), 3.90 (2H, m), 3.64 (6H, m), 2.39-2.22 (4H, m), 2.18 (2H, m), 2.06 (4H, m), 1.06-0.85 (12H, m). LCMS (run time=6 min): Rₜ=2.26; m/z 791 [M+H]⁺

Method B: To a stirred solution of the acid obtained from Preparation 22 (287.1 g, 1.639 mol) in acetonitrile (13.3 L) was added HOBT (263.5 g, 1.721 mol), followed by EDCI.HCl (322.0 g, 1.680 mol). The mixture was stirred at room temperature for 90 minutes. It was then cooled to 0° C. and the diamine hydrochloride salt obtained from Preparation 34a (510 g, 0.8194 mol) was added, followed DIPEA (571.6 mL, 3.278 mol). The mixture was stirred at 0° C. for 60 minutes then allowed to warm to room temperature and stirred overnight. The reaction mixture was concentrated in vacuo and partitioned between sat. NaHCO₃ solution (3.06 L, 3.4 mol) and DCM (4.08 L). The organic phase was washed with water (4.08 L) then concentrated in vacuo. The resulting crude material was purified by column chromatography (Biotage (3×5 Kg)), Heptane:Acetone 20:80 to Acetone 100) and the appropriate fractions were concentrated in vacuo then re-concentrated from TBME (5 L). To the resulting yellow solid was added water (2 L) and the mixture was granulated overnight. The solid was collected by filtration and dried in vacuo to give 377 g of the title compound as a bright yellow powder.

### EXAMPLE 5

### Methyl {(2S)-1-[(2S)-2-{5-[4-(2-{2-[(2S)-1-{(2S)-2-[(methoxycarbonyl)amino]-3-methyl-butanoyl}pyrrolidin-2-yl]-1H-imidazol-5-yl}quinoxalin-6-yl)phenyl]-1H-imidazol-2-yl}pyrrolidin-1-yl]-3-methyl-1-oxobutan-2-yl}carbamate

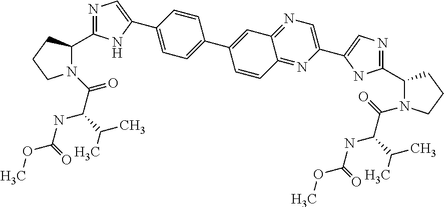

To a stirred solution of the acid obtained from Preparation 22 (41 mg, 0.23 mmol) in DMF (1 mL) was added PyBOP (87 mg, 0.23 mmol), and DIPEA (0.04 mL, 0.23 mmol). The mixture was stirred at room temperature for 10 minutes. The diamine obtained from Preparation 37 (50 mg, 0.10 mmol) in DMF (1 mL) was then added and the mixture was stirred at room temperature overnight. The reaction mixture was diluted with ethyl acetate (5 mL) and the organic phase was washed with water (5 mL), saturated sodium bicarbonate solution (5 mL) then brine (4×5 mL). It was dried with Na₂SO₄, filtered and concentrated in vacuo. The resulting crude material was purified by column chromatography on silica gel (redisep (4 g), DCM: MeOH: NH₃ 98:2:0.2 to 93:7:0.7) to give 33 mg of the title compound as a yellow solid.

¹H-NMR (400 MHz, MeOD): δ=9.39 (1H, m), 8.22 (1H, m), 8.09 (2H, m), 7.94-7.74 (5H, m), 7.37 (1H, m), 5.21 (2H, m), 4.24 (2H, m), 4.01 (2H, m), 3.90 (2H, m), 3.65 (6H, s), 2.44-2.03 (10H, m), 1.06-0.86 (12H, m).

LCMS (run time=6 min): Rₜ=2.02; m/z 791 [M+H]⁺

### EXAMPLE 6

### Methyl {(2S)-1-[(2S)-2-{5-[6-(5-{2-[(2S)-1-{(2S)-2-[(methoxycarbonyl)amino]-3-methyl-butanoyl}pyrrolidin-2-yl]-1H-imidazol-4-yl}pyridin-2-yl)naphthalen-2-yl]-1H-imidazol-2-yl}pyrrolidin-1-yl]-3-methyl-1-oxobutan-2-yl}carbamate

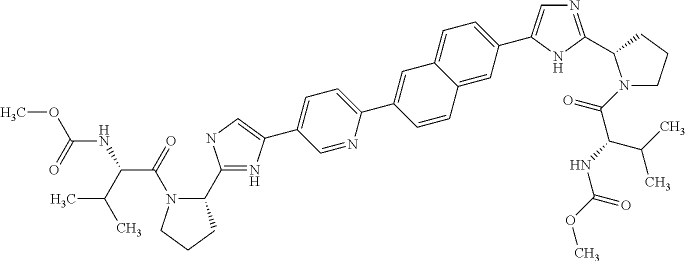

To a stirred solution of 5-{2-[(2S)-pyrrolidin-2-yl]-1H-imidazol-4-yl}-2-(6-{2-[(2S)-pyrrolidin-2-yl]-1H-imidazol-5-yl}naphthalen-2-yl)pyridine hydrochloride salt (0.37 g, 0.778 mmol), obtained from Preparation 43, in dry DMF (6 mL) was added L-valine carbamate (0.273 g, 1.557 mmol), obtained from Preparation 22, DIPEA (0.9 mL, 5.446 mmol) and HATU (0.621 g, 1.63 mmol). The reaction mixture was stirred at room temperature for 20 minutes. It was then quenched with ice-water and diluted with ethyl acetate (30 mL). The organic phase was washed with water (3×20 mL) followed by brine (20 mL), dried over Na₂SO₄ and evaporated to dryness. The crude mass was purified by prep-HPLC (System 4) to afford the title compound as an off white solid (40 mg).

¹H NMR (400 MHz, DMSO-d₆ at 100° C.): δ=11.98-11.49 (2H, m), 9.06 (1H, s), 8.54 (1H, s), 8.23-8.08 (3H, m), 8.02-7.82 (3H, m), 7.57-7.54 (2H, m), 7.35 (1H, s), 6.52 (2H, br. s), 5.19 (2H, m), 4.18 (2H, m), 3.79 (4H, m), 3.58 (6H, s), 2.33 (6H, s), 2.04-2.02 (4H, m), 0.90 (12H, m).

LCMS (System 1) (run time=5 min): Rₜ=3.17 min; 790 [M+H]⁺

### EXAMPLE 7

### Methyl {(2S)-1-[(2S)-2-{5-[6-(5-{2-[(2S)-1-{(2S)-2-[(methoxycarbonyl)amino]-3-methyl-butanoyl}pyrrolidin-2-yl]-1H-imidazol-5-yl}pyrimidin-2-yl)naphthalen-2-yl]1H-imidazol-2-yl}pyrrolidin-1-yl]-3-methyl-1-oxobutan-2-yl}carbamate

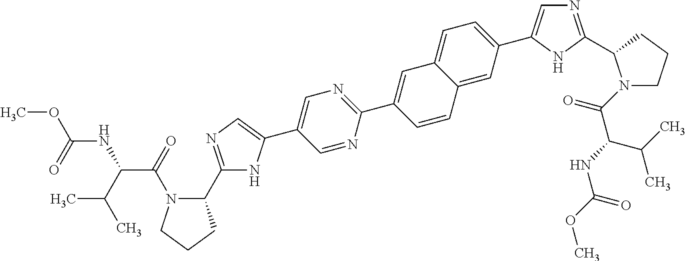

To a stirred solution of 5-{2-[(2S)-pyrrolidin-2-yl]-1H-imidazol-5-yl}-2-(6-{2-[(2S)-pyrrolidin-2-yl]-1H-imidazol-5-yl}naphthalen-2-yl)pyrimidine hydrochloride salt (0.39 g, 0.819 mmol), obtained from Preparation 50, in dry DMF (6 mL) was added L-valine carbamate (0.29 g, 1.638 mmol), obtained from Preparation 22, DIPEA (0.9 mL, 5.733 mmol) and HATU (0.65 g, 1.719 mmol). The reaction mixture was stirred at room temperature for 20 minutes. It was then quenched with ice-water and diluted with ethyl acetate (30 mL). The organic phase was washed with water (3×20 mL), and brine (20 ml). It was dried over Na₂SO₄ and then evaporated to dryness. The crude mass was purified by prep-HPLC (System 4) to give the title compound as an off white solid (25 mg).

¹H NMR (400 MHz, DMSO-d₆ at 100° C.): δ=12.08 (1H, br s), 11.85 (1H, br s), 9.19 (2H, s), 8.89 (1H, m), 8.46 (1H, m), 8.25 (1H, s), 7.98 (3H, m), 7.69 (1H, s), 7.56 (1H, s), 6.49 (2H, m), 5.19 (2H, m), 4.18 (2H, m), 3.79 (4H, m), 3.58 (6H, s), 2.23-2.02 (10H, m), 0.90 (12H, m). LCMS (System 1) (run time=5 min): Rₜ=3.13 min; 791 [M+H]⁺

### EXAMPLE 8

### Methyl {(2S)-1-[(2S)-2-{5-[4-(3-{2-[(2S)-1-{(2S)-2-[(methoxycarbonyl)amino]-3-methyl-butanoyl}pyrrolidin-2-yl]-1H-imidazol-5-yl}isoquinolin-7-yl)phenyl]-1H-imidazol-2-yl}pyrrolidin-1-yl]-3-methyl-1-oxobutan-2-yl}carbamate

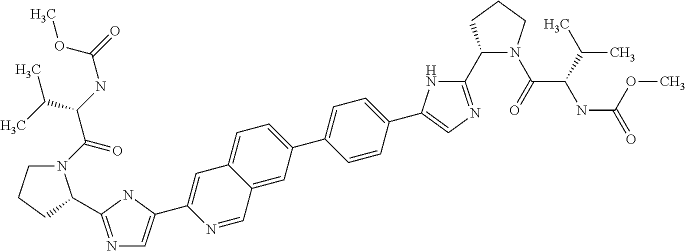

To a stirred solution of N-(methoxycarbonyl)-L-valine (20 mg, 0.116 mmol), obtained from Preparation 22, in dry acetonitrile (3 mL) was added HOBT (21 mg, 0.138 mmol) and EDCI.HCl (25 mg, 0.132 mmol). The reaction mixture was stirred at room temperature for 45 minutes. Diamine 3-{2-[(2S)-pyrrolidin-2-yl]-1H-imidazol-5-yl}-7-(4-{2-[(2S)-pyrrolidin-2-yl]-1H-imidazol-5-yl}phenyl)isoquinoline hydrochloride salt (34 mg, 0.055 mmol), obtained from Preparation 53, was added followed by DIPEA (0.096 mL, 0.55 mmol). The reaction mixture was stirred at room temperature overnight. It was then diluted with ethyl acetate (20 mL) and water was added (20 mL). The organic layer was separated and washed with saturated sodium bicarbonate solution (20 mL) and brine (20 mL). It was dried over magnesium sulphate, filtered and concentrated in vacuo. The resulting crude material was dry loaded onto silica and purified by column chromatography on silica gel (Redisep (4 g), 98:2:0.2 DCM: MeOH: NH₃ to 90:10:1 DCM: MeOH: NH₃) to give 12 mg of the title compound as a pale yellow solid.

¹H NMR (400 MHz, CDCl₃): δ=11.11-10.93 (m, 1H), 10.54-10.44 (m, 1H), 9.23-9.16 (m, 1H), 8.27-7.34 (m, 9H), 6.97-9.96 (m, 1H), 5.54-5.49 (m, 1H), 5.39-5.17 (m, 3H), 4.40-4.24 (m, 2H), 3.91-3.80 (m, 2H), 3.75-3.64 (m, 8H), 3.19-3.04 (m, 1H), 2.93-2.79 (m, 1H), 2.43-2.08 (m, 6H), 2.02-1.93 (m, 2H), 1.28-1.25 (m, 4H), 0.96-0.88 (m, 8H). LCMS (run time=6 min): Rₜ=1.87 min; m/z 790 [M+H]⁺

### EXAMPLE 9

### Methyl {(2S)-1-[(2S)-2-{5-[6-(6-{2-[(2S)-1-{(2S)-2-[(methoxycarbonyl)amino]-3-methyl-butanoyl}pyrrolidin-2-yl]-1H-imidazol-5-yl}quinoxalin-2-yl)pyridin-3-yl]-1H-imidazol-2-yl}pyrrolidin-1-yl]-3-methyl-1-oxobutan-2-yl}carbamate

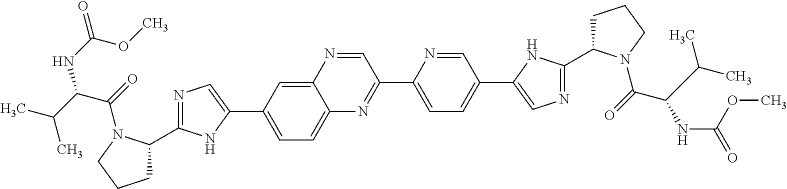

To a stirred solution of N-(methoxycarbonyl)-L-valine (0.062 g, 0.37 mmol), obtained from Preparation 22, in dry acetonitrile (2 mL) was added HOBt (0.059 g, 0.39 mmol) and EDCI.HCl (0.071 g, 0.37 mmol). This solution was stirred at room temperature for 1 hour. After this time 6-{2-[(2S)-pyrrolidin-2-yl]-1H-imidazol-5-yl}-2-(5-{2-[(2S)-pyrrolidin-2-yl]-1H-imidazol-5-yl}pyridin-2-yl)quinoxaline (0.074 g, 0.16 mmol), obtained from Preparation 58, was added and the reaction mixture was cooled to 0° C. DIPEA (0.14 mL, 0.78 mmol) and DMF (1 mL) were then added and the reaction mixture was stirred at room temperature overnight. It was then evaporated and purified by reverse phase chromatography (acetonitrile+0.1% formic acid:water+0.1% formic acid 3:97 to 85:15), to give the title compound as a yellow solid (40 mg).

¹H NMR (400 MHz, DMSO-d₆): δ=12.08 (2H, br s), 9.82 (1H, s), 9.14 (1H, s), 8.49 (1H, d), 8.39 (1H, s), 8.29 (2H, m), 8.08 (1H, d), 7.85 (1H, d), 7.79 (1H, d), 7.31 (2H, m), 5.11 (2H, m), 4.08 (2H, m), 3.83 (4H, m), 3.54 (6H, s), 2.18 (4H, m), 2.00 (6H, m), 0.90 (12H, m). LCMS (run time=8 min): Rₜ=2.07 min; m/z 792 [M+H]⁺

### EXAMPLE 10

### Methyl {(2S)-1-[(2S)-2-{5-[6-(3-{2-[(2S)-1-{(2S)-2-[(methoxycarbonyl)amino]-3-methyl-butanoyl}pyrrolidin-2-yl]-1H-imidazol-5-yl}quinolin-7-yl)pyridin-3-yl]-1H-imidazol-2-yl}pyrrolidin-1-yl]-3-methyl-1-oxobutan-2-yl}carbamate

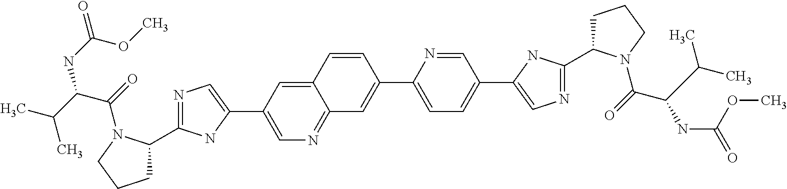

To a stirred solution of N-(methoxycarbonyl)-L-valine (0.049 g, 0.28 mmol), obtained from Preparation 22, in dry DMF (2 mL) was added PyBOP (0.11 g, 0.23 mmol) and DIPEA (0.048 mL, 0.28 mmol. This solution was stirred at room temperature for 30 minutes. After this time 3-{2-[(2S)-pyrrolidin-2-yl]1H-imidazol-5-yl}-7-(5-{2-[(2S)-pyrrolidin-2-yl]-1H-imidazol-5-yl}pyridin-2-yl)quinoline (0.06 g, 0.13 mmol), obtained from Preparation 63, was added and the reaction was allowed to stir at room temperature overnight. It was then evaporated and the crude product was purified by column chromatography on silica gel (dichloromethane: methanol:ammonia 9:1:0.5) and reverse phase chromatography (acetonitrile+0.1% formic acid:water+0.1% formic acid 5:95 to 85:15), to give the title compound as an orange solid (36 mg).

LCMS (run time=4.5 min): Rₜ=1.95 min; m/z 791 [M+H]⁺

### EXAMPLE 11

### Methyl {(2S)-1-[(2S)-2-{4-[6-(7-{2-[(2S)-1-{(2S)-2-[(methoxycarbonyl)amino]-3-methyl-butanoyl}pyrrolidin-2-yl]-1H-imidazol-5-yl}isoquinolin-3-yl)pyridin-3-yl]-1H-imidazol-2-yl}pyrrolidin-1-yl]-3-methyl-1-oxobutan-2-yl}carbamate

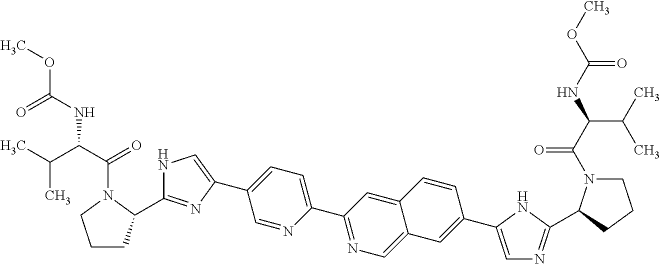

To a stirred solution of N-(methoxycarbonyl)-L-valine (0.5 g, 2.9 mmol), obtained from Preparation 22, in dry acetonitrile (5 mL) was added HOBt (0.47 g, 3.08 mmol) and EDCI.HCl (0.55 g, 2.9 mmol). This solution was stirred at room temperature for 1 hour. After this time 7-{2-[(2S)-pyrrolidin-2-yl]-1H-imidazol-5-yl}-3-(5-{2-[(2S)-pyrrolidin-2-yl]-1H-imidazol-4-yl}pyridin-2-yl)isoquinoline hydrochloride salt (0.3 g, 0.5 mmol), obtained from Preparation 66, was added followed by DIPEA (1 mL, 5.78 mmol) and the reaction mixture was allowed to stir at room temperature for 48 hours. It was then evaporated and the crude product was purified by column chromatography on silica gel (dichloromethane:methanol 95:5) to give the title compound as a yellow solid (34 mg).

¹H NMR (400 MHz, DMSO-d₆): δ=11.99 (2H, m), 9.35 (1H, s), 9.09 (1H, s), 8.65 (1H, s), 8.52 (1H, s), 8.34 (1H, d), 8.18 (2H, m), 8.02 (1H, d), 7.81 (1H, s), 7.71 (1H, s), 7.30 (2H, m), 5.12 (2H, m), 4.09 (2H, m), 3.84 (4H, m), 3.54 (6H, s), 2.17 (4H, m), 1.99 (6H, m), 0.9 (12H, m). LCMS (run time=4.5 min): Rₜ=1.94 min; m/z 791 [M+H]⁺

### EXAMPLE 12

### Methyl {(2S)-1-[(2S)-2-{4-[4-(7-{2-[(2S)-1-{(2S)-2-[(methoxycarbonyl)amino]butanoyl}pyrrolidin-2-yl]-1H-imidazol-5-yl}-1,5-naphthyridin-3-yl)phenyl]-1H-imidazol-2-yl}pyrrolidin-1-yl]-1-oxobutan-2-yl}carbamate

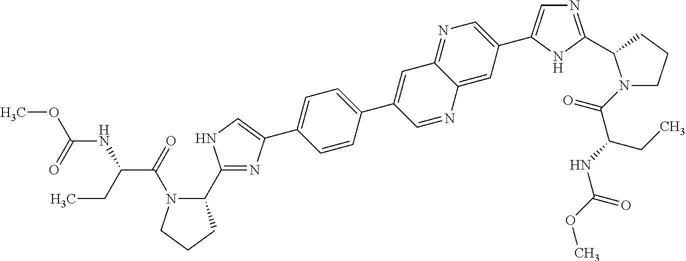

To (2S)-2-[(methoxycarbonyl)amino]butanoic acid (0.134 g, 0.83 mmol), obtained from Preparation 70, in DMF (1 mL) was added PyBOP (0.312 g, 0.83 mmol) and DIPEA (0.15 mL, 0.83 mmol). After stirring for 30 minutes, 3-{2-[(2S)-pyrrolidin-2-yl]-1H-imidazol-5-yl}-7-(4-{2-[(2S)-pyrrolidin-2-yl]-1H-imidazol-4-yl}phenyl)-1,5-naphthyridine (0.09 g, 0.19 mmol), obtained from Preparation 69, in DMF (1 mL) was added and the reaction mixture was stirred at room temperature overnight. It was then evaporated and the crude product was purified by column chromatography on silica gel (dichloromethane:methanol 95:5). The product was dissolved in a mixture of ethyl acetate and dichloromethane, and the organic phase was washed with saturated aqueous sodium bicarbonate solution (4×100 mL) and brine (100 mL). It was dried over MgSO₄ and then evaporated to give the title product as a light yellow solid (30 mg).

LCMS (System 5) (run time=5 min): Rₜ=1.27 min; m/z 763 [M+H]⁺

### EXAMPLE 13

### Methyl {(2S)-1-[(2S)-2-{5-[4-(2-{2-[(2S)-1-{(2S)-2-[(methoxycarbonyl)amino]-3-methyl-butanoyl}pyrrolidin-2-yl]-1H-imidazol-4-yl}quinolin-6-yl)phenyl]-1H-imidazol-2-yl}pyrrolidin-1-yl]-3-methyl-1-oxobutan-2-yl}carbamate

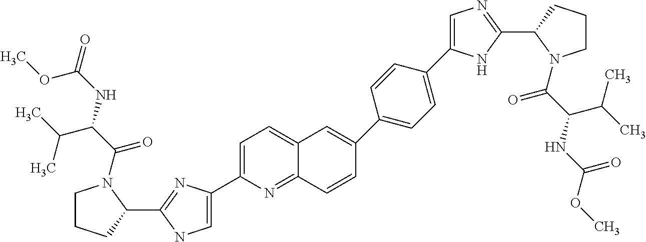

To a stirred solution of N-(methoxycarbonyl)-L-valine (18 mg, 0.104 mmol), obtained from Preparation 22, in DMF (2 mL) was added HATU (39 mg, 0.104 mmol) and DIPEA (27 μL, 0.156 mmol). After stirring at room temperature for 20 minutes, methyl {(2S)-3-methyl-1-oxo-1-[(2S)-2-{5-[4-(2-{2-[(2S)-pyrrolidin-2-yl]-1H-imidazol-5-yl}quinolin-6-yl) phenyl]-1H-imidazol-2-yl) pyrrolidin-1-yl]butan-2-yl}carbamate (66 mg, 0.104 mmol), obtained from Preparation 72, was added in DMF (3 mL) and the solution was stirred at room temperature for 2 hours. The DMF was azeotropically distilled with toluene before partitioning between ethyl acetate and water. The organic phase was evaporated and the resulting crude material was purified by column chromatography on silica gel (Redisep 4 g, eluting with a gradient of ethyl acetate: methanol 100:0 to 80:20). The product was then further purified by application to an SCX cartridge eluting initially with methanol then 7N ammonia in methanol to give the product as a yellow solid (12 mg).

LCMS (run time=6 min): Rₜ=1.87 min; m/z 790 [M+H]⁺

### EXAMPLE 14

### Methyl {(2S)-1-[(2S)-2-{5-[3-(4-{2-[(2S)-1-{(2S)-2-[(methoxycarbonyl)amino]-3-methyl-butanoyl}pyrrolidin-2-yl]-1H-imidazol-5-yl}phenyl)pyrido[2,3-b]pyrazin-7-yl]-1H-imidazol-2-yl}pyrrolidin-1-yl]-3-methyl-1-oxobutan-2-yl}carbamate

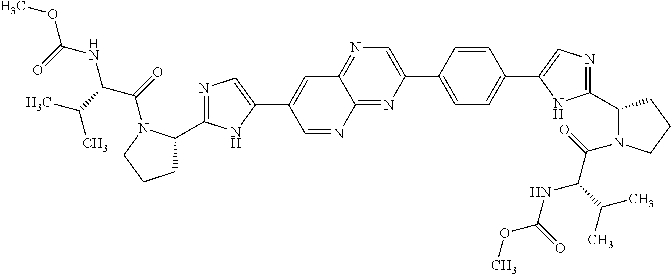

N-Methoxycarbonyl-L-valine (158 mg, 0.905 mmol), obtained from Preparation 22, HOBT (144 mg, 0.943 mmol) and EDCI.HCl (174 mg, 0.905 mmol) in acetonitrile (10 mL) were stirred at room temperature for 15 minutes. 7-{2-[(2S)-Pyrrolidin-2-yl]-1H-imidazol-5-yl}-3-(4-{2-[(2S)-pyrrolidin-2-yl]-1H-imidazol-5-yl}phenyl)pyrido[2,3-b]pyrazine (180 mg, 0.377 mmol), obtained from Preparation 77, in acetonitrile (10 mL) was added and the mixture was cooled to 0° C., followed by the dropwise addition of DIPEA (0.525 mL, 3.02 mmol). The reaction mixture was allowed to warm to room temperature and stirred for 18 hours. It was evaporated then dissolved in dichloromethane and the organic phase was washed with saturated aqueous sodium bicarbonate solution. The organic phase was dried over Na₂SO₄ and evaporated. The crude product was purified by column chromatography on silica gel (dichloromethane: methanol:ammonia 95:5:05 to 90:10:1) to give the title compound as a bright yellow solid (49 mg).

¹H NMR (400 MHz, DMSO-d₆): δ=12.15 (1H, br s), 11.90 (1H, br s), 9.64 (1H, m), 9.56 (1H, m), 8.60 (1H, m), 8.34 (2H, m), 8.00-7.90 (3H, m), 7.66 (1H, s), 7.28 (2H, m), 5.13-5.05 (2H, m), 4.07 (2H, m), 3.81 (4H, m), 3.52 (6H, s), 2.22-1.84 (10H, m), 0.95-0.90 (12H, m). LCMS (run time=6 min): Rₜ=2.24 min; m/z 792 [M+H]⁺

### EXAMPLE 15

### Methyl {(2S)-1-[(2S)-2-{5-[4-(4-hydroxy-6-{2-[(2S)-1-{(2S)-2-[(methoxycarbonyl)amino]-3-methylbutanoyl}pyrrolidin-2-yl]-1H-imidazol-5-yl}quinazolin-2-yl)phenyl]-1H-imidazol-2-yl}pyrrolidin-1-yl]-3-methyl-1-oxobutan-2-yl}carbamate

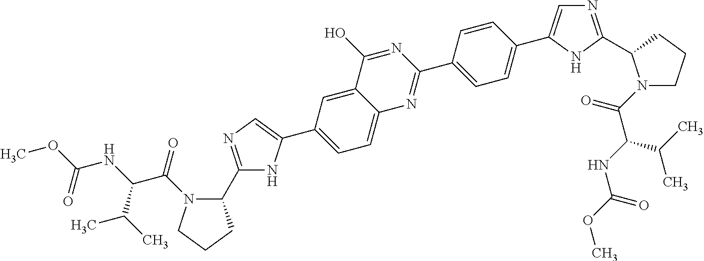

N-Methoxycarbonyl-L-valine (21 mg, 0.121 mmol), obtained from Preparation 22, HOBT (21 mg, 0.138 mmol) and EDCI.HCl (25 mg, 0.132 mmol) in acetonitrile (2 mL) were stirred at room temperature for 45 minutes. Then 6-{2-[(2S)-pyrrolidin-2-yl]-1H-imidazol-5-yl}-2-(4-{2-[(2S)-pyrrolidin-2-yl]-1H-imidazol-5-yl}phenyl)quinazolin-4-ol hydrochloride salt (35 mg, 0.055 mmol), obtained from Preparation 81, was added followed by dropwise addition of DIPEA (0.096 mL, 0.550 mmol). The reaction mixture was stirred at room temperature for 18 hours. It was then diluted with ethyl acetate, and the organic phase was washed with saturated aqueous sodium bicarbonate solution, dried over MgSO₄ and evaporated. The crude product was purified by column chromatography on silica gel (Redisep (4 g) dichloromethane: methanol:ammonia 100:0:0 to 95:5:0.5) to give the title compound as a pale yellow solid (21 mg).

¹H NMR (400 MHz, CD₃OD): δ=8.54-8.40 (1H, m), 8.20-7.98 (3H, m), 7.93-7.66 (3H, m), 7.52-7.35 (2H, m), 5.35-5.15 (2H, m), 4.24 (2H, m), 3.99 (2H, m), 3.89 (2H, m), 3.65 (6H, s), 2.40-1.94 (10H, m), 0.94 (12H, m).

LCMS (run time=6 min): Rₜ=1.44 min; m/z 807 [M+H]⁺

### EXAMPLE 16

### Methyl {(2S)-1-[(2S)-2-{5-[4-(4-ethoxy-6-{2-[(2S)-1-{(2S)-2-[(methoxycarbonyl)amino]-3-methylbutanoyl}pyrrolidin-2-yl]-1H-imidazol-5-yl}quinazolin-2-yl)phenyl]-1H-imidazol-2-yl}pyrrolidin-1-yl]-3-methyl-1-oxobutan-2-yl}carbamate

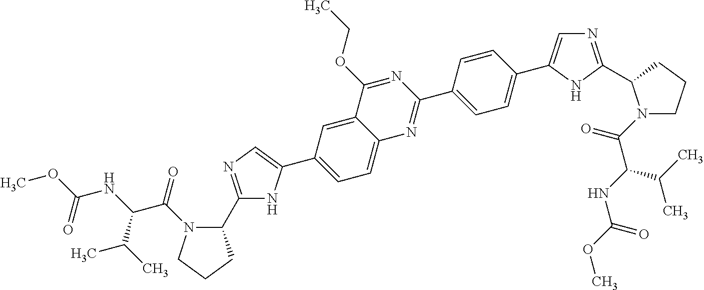

N-Methoxycarbonyl-L-valine (30 mg, 0.171 mmol), obtained from Preparation 22, HOBT (30 mg, 0.194 mmol) and EDCI.HCl (36 mg, 0.187 mmol) in acetonitrile (2 mL) were stirred at room temperature for 45 minutes. Then 4-Ethoxy-6-{2-[(2S)-pyrrolidin-2-yl]-1H-imidazol-5-yl}-2-(4-{2-[(2S)-pyrrolidin-2-yl]-1H-imidazol-5-yl}phenyl)quinazoline hydrochloride salt (52 mg, 0.078 mmol), obtained from Preparation 84, was added followed by dropwise addition of DIPEA (0.135 mL, 0.777 mmol). The reaction mixture was stirred at room temperature for 18 hours. It was then diluted with dichloromethane and the organic phase was washed with saturated aqueous sodium bicarbonate solution. The mixture was filtered through a phase-separation tube with a hydrophobic frit and the filtrate was evaporated. The crude product was purified by column chromatography on silica gel (Redisep (4 g) dichloromethane: methanol:ammonia 100:0:0 to 90:10:1) to give the title compound as a pale yellow solid (31 mg) as the predominant product which may contain traces of the methoxy variant.

¹H NMR (400 MHz, DMSO-d₆): δ=7.69-6.05 (9H, m), 4.54-4.30 (2H, m), 3.97-3.85 (2H, m), 3.43 (2H, m), 3.30-3.02 (4H, m), 2.83 (6H, s), 1.59-1.08 (10H, m), 0.75 (3H, t), 0.22-0.00 (12H, m). LCMS (run time=6 min): Rₜ=1.95 min; m/z 835 [M+H]⁺

### EXAMPLE 17

### Methyl {(2S)-1-[(2S)-2-{4-[4-(3-methoxy-6-{2-[(2S)-1-{(2S)-2-[(methoxycarbonyl)amino]-3-methylbutanoyl}pyrrolidin-2-yl]-1H-imidazol-4-yl}quinoxalin-2-yl)phenyl]-1H-imidazol-2-yl}pyrrolidin-1-yl]-3-methyl-1-oxobutan-2-yl}carbamate

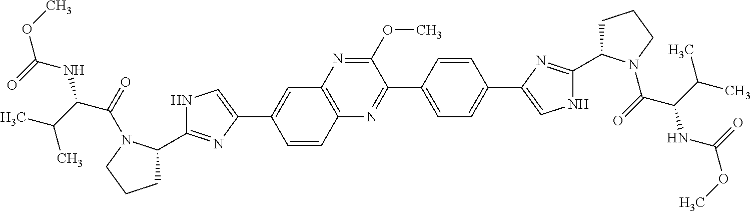

N-Methoxycarbonyl-L-valine (41.2 mg, 0.235 mmol), obtained from Preparation 22, HOBt (37.5 mg, 0.245 mMol) and EDCI.HCl (45.1 mg, 0.235 mmol) in acetonitrile (5 mL) were stirred at room temperature for 20 minutes. Then crude 3-Methoxy-6-(2-((S)-pyrrolidin-2-yl)-1H-imidazol-4-yl)-2-(4-(2-((S)-pyrrolidin-2-yl)-1H-imidazol-4-yl)phenyl)quinoxaline hydrochloride salt, directly obtained from Preparation 91, was added followed by the dropwise addition of DIPEA (0.137 mL, 0.78 mmol). The reaction mixture was stirred at room temperature for 72 hours. It was then evaporated and the residue was redissolved in ethyl acetate. The organic phase was washed with saturated aqueous sodium bicarbonate solution and brine, dried over MgSO₄ and then evaporated. The crude product was purified by preparative HPLC (System 3) (30 to 40% acetonitrile in 0.1% ammonia in water over 10 minutes, flow rate 50 mL/min) to give the title compound as a bright yellow solid (10 mg).

LCMS (run time=25 min): Rₜ=9.286 min; m/z 821 [M+H]⁺

### EXAMPLE 18

### Methyl {(2S)-1-[(2S)-2-{4-[4-(3-hydroxy-6-{2-[(2S)-1-{(2S)-2-[(methoxycarbonyl)amino]-3-methylbutanoyl}pyrrolidin-2-yl]-1H-imidazol-4-yl}quinoxalin-2-yl)phenyl]-1H-imidazol-2-yl}pyrrolidin-1-yl]-3-methyl-1-oxobutan-2-yl}carbamate

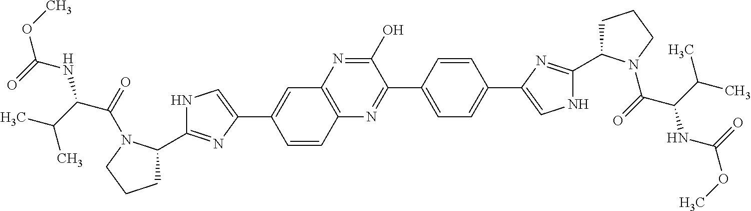

N-Methoxycarbonyl-L-valine (37.4 mg, 0.214 mmol), obtained from Preparation 22, HOBt (34 mg, 0.223 mmol) and EDCI.HCl (41 mg, 0.214 mmol) in acetonitrile (3 mL) were stirred at room temperature for 20 minutes. Then crude 7-{2-[(2S)-pyrrolidin-2-yl]-1H-imidazol-4-yl}-3-(4-{2-[(2S)-pyrrolidin-2-yl]-1H-imidazol-4-yl}phenyl)quinoxalin-2-ol hydrochloride salt, directly obtained from Preparation 92, was added followed by the dropwise addition of DIPEA (0.124 mL, 0.712 mmol). The reaction mixture was stirred at room temperature for 72 hours. It was then evaporated and the residue was redissolved in ethyl acetate. The organic phase was washed with saturated aqueous sodium bicarbonate solution and brine, dried over MgSO₄ and evaporated. The crude product was purified by preparative HPLC (System 3) (20 to 35% acetonitrile in 0.1% ammonia in water over 10 minutes, flow rate 50 mL/min) to give the title compound as a bright yellow solid (11 mg).

LCMS (run time=25 min): Rₜ=9.253 min; m/z 807 [M+H]⁺

### EXAMPLE 19

### Methyl {(2S)-1-[(2S)-2-{5-[7-(4-{2-[(2S)-1-{(2S)-2-[(methoxycarbonyl)amino]-3-methylbutanoyl}pyrrolidin-2-yl]-1H-imidazol-4-yl}phenyl)-1,5-naphthyridin-3-yl]-1H-imidazol-2-yl}pyrrolidin-1-yl]-3-methyl-1-oxobutan-2-yl}carbamate

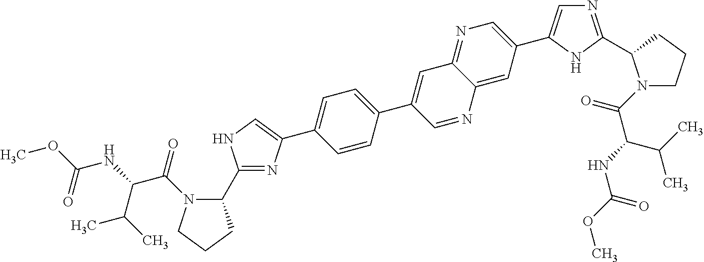

To N-methoxycarbonyl-L-valine (73 mg, 0.42 mmol), obtained from Preparation 22, in DMF (1 mL) was added PyBOP (156 mg, 0.42 mmol) and DIPEA (0.073 mL, 0.42 mmol). After stirring for 30 minutes, 3-{2-[(2S)-pyrrolidin-2-yl]-1H-imidazol-5-yl}-7-(4-{2-[(2S)-pyrrolidin-2-yl]-1H-imidazol-4-yl}phenyl)-1,5-naphthyridine (90 mg, 0.19 mmol), obtained from Preparation 69, was added and the reaction mixture was stirred at room temperature overnight. An additional solution of N-Methoxycarbonyl-L-valine (73 mg, 0.42 mmol), obtained from Preparation 22, PyBOP (156 mg, 0.42 mmol) and DIPEA (0.073 mL, 0.42 mmol) in DMF (1 mL) was prepared and, after stirring at room temperature for 10 minutes, it was added to the reaction mixture and stirring was continued for a further 4 hours. The reaction mixture was then evaporated. The crude product was dry loaded onto silica and purified by column chromatography on silica gel (dichloromethane:methanol 95:5 to 92.5:7.5). The product was dissolved in a mixture of ethyl acetate and dichloromethane and the organic phase was washed with saturated aq. sodium bicarbonate solution (4×100 mL) and brine (100 mL). It was dried over MgSO₄ and evaporated to give the title product as a light yellow solid (80 mg).

LCMS (System 5) (run time=5 min): Rₜ=1.90 min; m/z 791 [M+H]⁺

### EXAMPLE 20

### Methyl {(2S)-1-[(2S)-2-{5-[2-(5-{2-[(2S)-1-{(2S)-2-[(methoxycarbonyl)amino]-3-methylbutanoyl}pyrrolidin-2-yl]-1H-imidazol-5-yl}pyridin-2-yl)quinazolin-6-yl]-1H-imidazol-2-yl}pyrrolidin-1-yl]-3-methyl-1-oxobutan-2-yl}carbamate

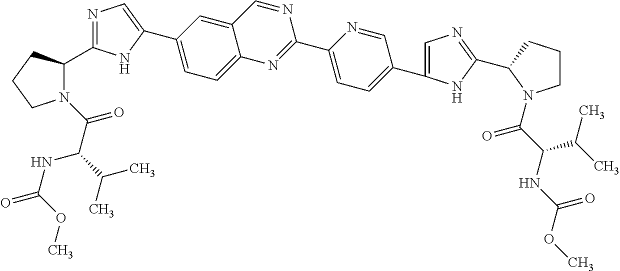

N-Methoxycarbonyl-L-valine (42 mg, 0.242 mmol), obtained from Preparation 22, HOBT (42 mg, 0.275 mmol) and EDCI.HCl (79 mg, 0.264 mmol) in acetonitrile (2 mL) were stirred at room temperature for 45 minutes. 6-{2-[(2S)-pyrrolidin-2-yl]-1H-imidazol-5-yl}-2-(5-{2-[(2S)-pyrrolidin-2-yl]-1H-imidazol-5-yl}pyridin-2-yl)quinazoline hydrochloride salt (81 mg, 0.110 mmol), obtained from Preparation 97, was added followed by the dropwise addition of DIPEA (0.192 mL, 1.10 mmol). The reaction mixture was stirred at room temperature for 18 hours. It was then concentrated and the residue was partitioned between DCM and saturated sodium bicarbonate solution. The organic layer was extracted and concentrated in vacuo. The crude product was purified by column chromatography on silica gel (dichloromethane: methanol:ammonia 100:0:0 to 90:10:1) to give the title compound as a pale yellow solid (54 mg).

LCMS (run time=2 min): Rₜ=1.11 min; m/z 792 [M+H]⁺

### EXAMPLE 21

### Methyl {(2S)-1-[(2S)-2-{5-[2-(5-{2-[(2S)-1-{(2S)-2-[(methoxycarbonyl)amino]-3-methylbutanoyl}pyrrolidin-2-yl]-1H-imidazol-4-yl}pyrimidin-2-yl)quinoxalin-6-yl]-1H-imidazol-2-yl}pyrrolidin-1-yl]-3-methyl-1-oxobutan-2-yl}carbamate

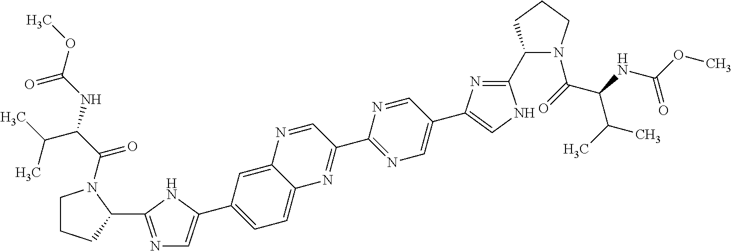

To a stirred solution of N-(methoxycarbonyl)-L-valine (0.043 g, 0.24 mmol), obtained from Preparation 22, in dry acetonitrile (0.6 mL) was added HOBt (0.039 g, 0.26 mmol) and EDCI.HCl (0.047 g, 0.24 mmol). This solution was stirred at room temperature for 1 hour. After this time, 6-{2-[(2S)-pyrrolidin-2-yl]-1H-imidazol-5-yl}-2-(5-{2-[(2S)-pyrrolidin-2-yl]-1H-imidazol-4-yl}pyrimidin-2-yl)quinoxaline hydrochloride salt (0.038 g, 0.061 mmol), obtained from Preparation 104, followed by DIPEA (0.13 mL, 0.73 mmol) was added. The reaction mixture was allowed to stir at room temperature overnight. It was then concentrated in vacuo and was purified by reverse phase chromatography (gradient, 5:95:0.1 acetonitrile: water: ammonium formate to 95:5:0.1 acetonitrile: water: ammonium formate over 30 minutes) to afford the title compound as a yellow solid (20 mg).

¹H NMR (400 MHz, DMSO-d₆): δ=12.20 (1H, s), 12.07 (1H, s), 9.80 (1H, s), 9.32 (2H, s), 8.41 (1H, d), 8.32 (1H, dd), 8.15 (1H, d), 7.90 (2H, m), 7.30 (2H, m), 5.12 (2H, m), 4.09 (2H, t), 3.84 (4H, m), 3.54 (6H, s), 2.18 (4H, m), 1.99 (6H, m), 0.90 (12H, m). LCMS (run time=4.5 min): Rₜ=2.04 min; m/z 793 [M+H]⁺

### EXAMPLE 22

### Methyl {(2S)-1-[(2S)-2-{4-[2-(7-{2-[(2S)-1-{(2S)-2-[(methoxycarbonyl)amino]-3-methylbutanoyl}pyrrolidin-2-yl]-1H-imidazol-5-yl}isoquinolin-3-yl)pyrimidin-5-yl]-1H-imidazol-2-yl}pyrrolidin-1-yl]-3-methyl-1-oxobutan-2-yl}carbamate

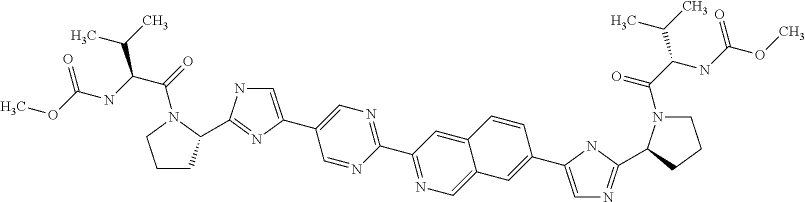

To a stirred solution of N-(methoxycarbonyl)-L-valine (0.05 g, 0.29 mmol), obtained from Preparation 22, in dry acetonitrile (1 mL) was added HOBt (0.044 g, 0.29 mmol) and EDCI.HCl (0.055 g, 0.28 mmol). This solution was stirred at room temperature for 30 minutes. After this time, 7-{2-[(2S)-pyrrolidin-2-yl]-1H-imidazol-5-yl}-3-(5-{2-[(2S)-pyrrolidin-2-yl]-1H-imidazol-4-yl}pyrimidin-2-yl)isoquinoline hydrochloride salt (0.053 g, 0.061 mmol), obtained from Preparation 106, was added as a solution in dry acetonitrile followed by DIPEA (0.2 mL, 1.0 mmol) and DMF (0.3 mL). The reaction mixture was allowed to stir at room temperature overnight. It was then concentrated in vacuo and purified by chromatography on silica gel (dichloromethane: methanol:ammonia 95:5:0.5) to afford the title compound as a yellow solid (16 mg).

¹H NMR (400 MHz, DMSO-d₆): δ=1 2.03 (2H, m), 9.37 (1H, s), 9.23 (1H, s), 8.84 (1H, m), 8.45 (1H, m), 8.21 (1H, m), 8.11 (1H, m), 7.85 (1H, m), 7.74 (1H, m), 7.71 (1H, s), 7.30 (2H, m), 5.12 (2H, m), 4.09 (2H, m), 3.83 (3H, m), 3.54 (6H, s), 2.18 (5H, m), 1.99 (6H, m), 0.9 (12H, m). LCMS (run time=4.5 min): Rₜ=1.30 min; m/z 792 [M+H]⁺

### EXAMPLE 23

### Methyl {(2S)-1-[(2S)-2-{5-[4-(4-cyano-6-{2-[(2S)-1-{(2S)-2-[(methoxycarbonyl)amino]-3-methylbutanoyl}pyrrolidin-2-yl]-1H-imidazol-4-yl}quinolin-2-yl)phenyl]-1H-imidazol-2-yl}pyrrolidin-1-yl]-3-methyl-1-oxobutan-2-yl}carbamate

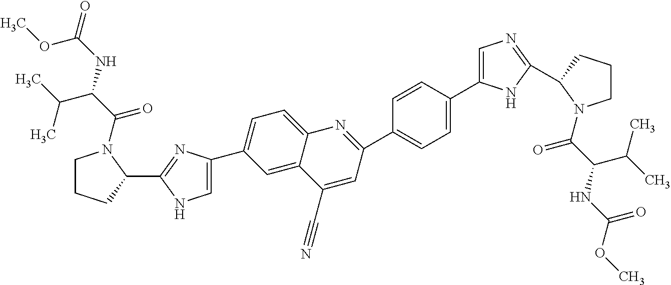

To a stirred solution of N-(methoxycarbonyl)-L-valine (2 mg, 0.071 mmol), obtained from Preparation 22, in DMF (0.5 mL) was added HATU (27 mg, 0.071 mmol) and DIPEA (19 μL, 0.106 mmol). After stirring at room temperature for 20 minutes, methyl {(2S)-1-[(2S)-2-{5-[4-(4-cyano-6-{2-[(2S)-pyrrolidin-2-yl]-1H-imidazol-5-yl}quinolin-2-yl)phenyl]-1H-imidazol-2-yl}pyrrolidin-1-yl]-3-methyl-1-oxobutan-2-yl}carbamate (47 mg, 0.071 mmol), obtained from Preparation 108, was added in DMF (1 mL) and the mixture was stirred at room temperature for 2 hours. The reaction mixture was partitioned between ethyl acetate and water, and the organic phase was evaporated. The crude material was purified by column chromatography on silica gel (Redisep 4 g, eluting with a gradient of DCM: MeOH+1% NH3 100:0 to 95:5). The product was then further purified by application to an SCX cartridge eluting initially with methanol then 7N ammonia in methanol to elute the product as a bright yellow solid (21 mg).

LCMS (run time=2 min): Rₜ=1.25 min; m/z 815 [M+H]⁺

### EXAMPLE 24

### Methyl {(2S)-1-[(2S)-2-{5-[6-(5-{2-[(2S)-1-{(2S)-2-[(methoxycarbonyl)amino]-3-methylbutanoyl}pyrrolidin-2-yl]-1H-imidazol-4-yl}pyrazin-2-yl)naphthalen-2-yl]-1H-imidazol-2-yl}pyrrolidin-1-yl]-3-methyl-1-oxobutan-2-yl}carbamate

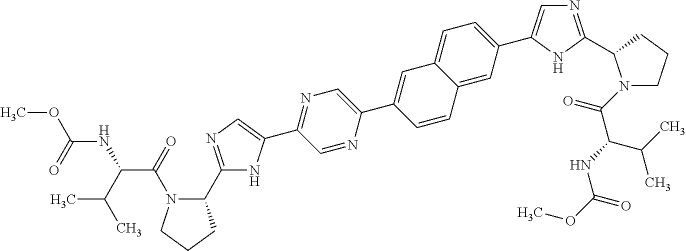

To a stirred solution of 2-{2-[(2S)-pyrrolidin-2-yl]-1H-imidazol-5-yl}-5-(6-{2-[(2S)-pyrrolidin-2-yl]-1H-imidazol-5-yl}naphthalen-2-yl)pyrazine hydrochloride salt (0.75 g, 1.20 mmol), obtained from Preparation 113, in dry DMF (10 mL) was added L-valine carbamate (0.38 g, 2.17 mmol), obtained from Preparation 22, DIPEA (1.0 mL, 6.0 mmol) and HATU (0.91 g, 2.4 mmol). The reaction mixture was stirred at room temperature for 25 minutes. It was then quenched with ice-water and was diluted with ethyl acetate (30 mL). The organic layer was washed with water (3×20 mL) followed by brine (1×20 mL), dried over Na₂SO₄ and evaporated to dryness. The crude mass was purified by prep-HPLC (system 4) to afford the title compound as an off white solid (145 mg).

¹H NMR (400 MHz, DMSO-d₆ at 100° C.): δ=11.86 (1H, br s), 11.53 (1H, br s), 9.07 (1H, s), 8.93 (1H, s), 8.65 (1H, s), 8.26 (2H, m), 7.83 (3H, m), 7.81 (1H, s), 7.57 (1H, s), 6.56 (2H, m), 5.19 (2H, m), 4.18 (2H, m), 3.79 (4H, m), 3.58 (6H, s), 2.22 (6H, m), 2.05-2.02 (4H, m), 0.93 (12H, m). LCMS (System 1) (run time=5 min): Rₜ=2.98 min, 791 [M+H]⁺

### EXAMPLE 25

### Methyl {(2S)-1-[(2S)-2-{5-[4-(6-{2-[(2S)-1-{(2S)-2-[(methoxycarbonyl)amino]butanoyl}pyrrolidin-2-yl]-1H-imidazol-5-yl}naphthalen-2-yl)phenyl]-1H-imidazol-2-yl}pyrrolidin-1-yl]-1-oxobutan-2-yl}carbamate

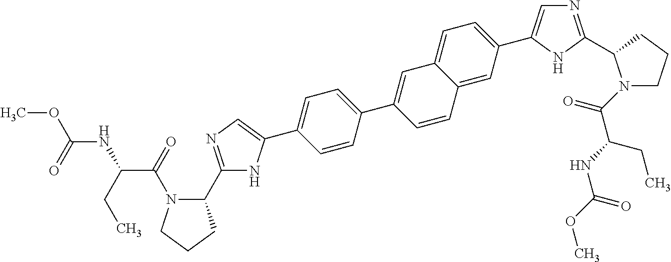

(2S)-2-[(Methoxycarbonyl)amino]butanoic acid (76 mg, 0.47 mmol), obtained from Preparation 70, in DMF (1 mL) was added PyBOP (0.178 g, 0.47 mmol) and DIPEA (0.082 mL, 0.47 mmol). After stirring for 30 minutes, 2-[(2S)-pyrrolidin-2-yl]-4-[4-(6-{2-[(2S)-pyrrolidin-2-yl]-1H-imidazol-5-yl}naphthalen-2-yl)phenyl]-1H-imidazole (0.102 g, 0.22 mmol), obtained from Preparation 21, in DMF (1 mL) was added and the reaction mixture was stirred at room temperature overnight. It was then evaporated and the crude product was purified by column chromatography on silica gel (dichloromethane: methanol:ammonia 100:0:0 to 95:5:0.5) to give the title product as a light yellow solid (130 mg).

LCMS (System 5) (run time=5 min): Rₜ=1.85 min; m/z 761 [M+H]⁺

### EXAMPLE 26

### Methyl {(2S)-1-[(2S)-2-{5-[4-(6-{2-[(2S)-1-{(2S)-2-[(methoxycarbonyl)amino]butanoyl}pyrrolidin-2-yl]-1H-imidazol-5-yl}naphthalen-2-yl)phenyl]-1H-imidazol-2-yl}pyrrolidin-1-yl]-3-methyl-1-oxobutan-2-yl}carbamate

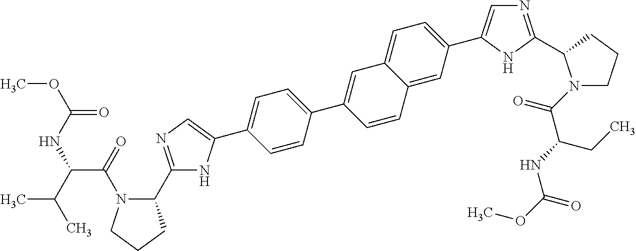

(2S)-2-[(Methoxycarbonyl)amino]butanoic acid (28 mg, 0.17 mmol), obtained from Preparation 70, in DMF (2 mL) was added PyBOP (65 mg, 0.17 mmol) and DIPEA (0.030 mL, 0.17 mmol). After stirring for 30 minutes, methyl {(2S)-3-methyl-1-oxo-1-[(2S)-2-{5-[4-(6-{2-[(2S)-pyrrolidin-2-yl]-1H-imidazol-5-yl}naphthalen-2-yl)phenyl]-1H-imidazol-2-yl}pyrrolidin-1-yl]butan-2-yl}carbamate (0.102 g, 0.22 mmol), obtained from Preparation 118, in DMF (1 mL) was added and the reaction mixture was stirred at room temperature overnight. It was then evaporated and the crude product was purified by reverse phase column chromatography (0.1% formic acid in water: 0.1% formic acid in acetonitrile 95:5 to 5:95) to give the title product as a very pale yellow solid (26 mg).

LCMS (run time=8 min): Rₜ=1.92 min; m/z 775 [M+H]⁺

### EXAMPLES 27 TO 29

Examples of compounds of the general formula:

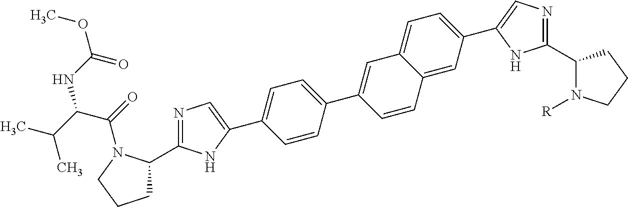

are tabulated below, and were prepared by a similar method to that described for Example 26, using the appropriate acid starting materials with the amine described in Preparation 118.

| | | LCMS | |
|---|---|---|---|
| Ex | | (run time = 8 min) | |
| No | R | m/z = | Rₜ |
| 27¹ | 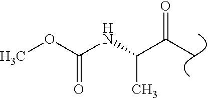 | 761 [M + H]⁺ | 1.81 |
| 28² | 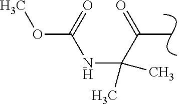 | 775 [M + H]⁺ | 1.92 |
| 29³ | 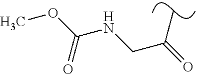 | 747 [M + H]⁺ | 1.82 |

Footnotes
¹See Preparation 114 for the preparation of the acid starting material
²See Preparation 115 for the preparation of the acid starting material
³See Preparation 116 for the preparation of the acid starting material

### EXAMPLES 30 TO 32

Examples of compounds of the general formula:

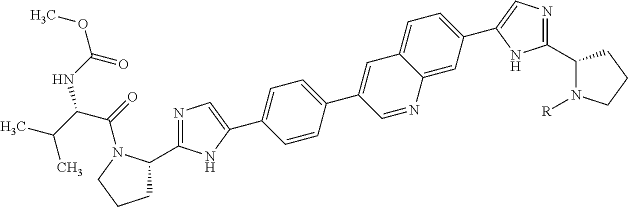

are tabulated below, and were prepared by a similar method to that described for Example 26, using the appropriate acid starting materials with the amine described in Preparation 124.

| | | LCMS | |
|---|---|---|---|
| Ex | | (run time = 4.5 min) | |
| No | R | m/z = | Rₜ |
| 30¹ | 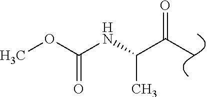 | 762 [M + H]⁺ | 1.25 |
| 31² | 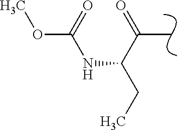 | 776 [M + H]⁺ | 1.30 |
| 32³ | 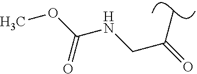 | 748 [M + H]⁺ | 1.23 |

Footnotes
¹See Preparation 114 for the preparation of the acid starting material. Purified by column chromatography (dichloromethane:methanol:ammonia 95:5:0.5 to 90:10:1)
²See Preparation 70 for the preparation of the acid starting material.
³See Preparation 116 for the preparation of the acid starting material. Purified by column chromatography (dichloromethane:methanol:ammonia 90:10:1)

### EXAMPLES 33 TO 84

Examples of compounds with the general formula:

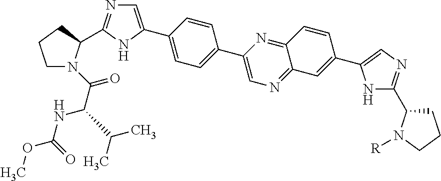

are tabulated below and have been prepared using one of the three methods (i) to (iii) presented below, as indicated in the Table. The relevant starting materials are either commercially available or can be prepared from commercially available amino acids which have been suitably protected using methods known to those skilled in the art, see for example Protective Groups In Organic Synthesis, T. W. Greene and P. G. M. Wuts, 4^{th} edition (John Wiley & Sons)

The exception to this is the acid used in Example 45 which is formed from an amide coupling reaction between commercially available (2-oxo-3,4-dihydroquinolin-1-(2H)-yl)acetic acid and glycine methyl ester, and subsequent ester hydrolysis using methods known to those skilled in the art.

### Method (i):

Method A—for Acids with Active Hydrogen:

To a solution of the amine obtained from Preparation 128 (75 μmol) in DMF (200 μL) was added a solution of the relevant acid (90 μmol) in DMF (200 μL), triethylamine (30 μl, 225 μmol), a solution of HOBT (15 μmol) in DMF (200 μL), and a solution of EDCI.HCl (90 μmol) in DMF (200 μL). The mixture was sealed and shaken at 50° C. for 3 hours. The reaction mixture was concentrated and the residue was purified by preparative HPLC.

### Method B—for Aliphatic and Amino Acids

To a solution of the amine obtained from Preparation 128 (75 μmol) in DMF (300 μL) was added a solution of the relevant acid (90 μmol) in DMF (300 μL), triethylamine (30 μl, 225 μmol), a solution of HATU (90 μmol) in DMF (200 μL). The mixture was sealed and shaken at 30° C. for 6 hours. The reaction mixture was concentrated and the residue was purified by preparative HPLC.

### Method (ii):

To a solution of the relevant acid (100 μmol) in DMF (500 μL) was added a solution of the amine obtained from Preparation 128 (100 μmol) in DMF (500 μL), a solution of HATU (100 μmol) in DMF (500 μL), and triethylamine (27 μL, 200 μmol). The mixture was sealed and shaken at 30° C. for 16 hours. The reaction mixture was concentrated and an aqueous 0.1 M solution of NaHCO₃ (1.0 mL) was added. The product was extracted with DCM (3×1.0 mL). The organic layers were combined and evaporated. The crude residue was dissolved in methanol (800 μL) and a 4.0 M solution of HCl in dioxane (400 μL) was added. The mixture was sealed and shaken at 30° C. for 2 hours. It was then concentrated and the residue was dissolved in methanol (1.0 mL) then concentrated again. Pyridine (1.2 mL) was added to the crude residue and the mixture was cooled to 0° C. Methyl chloroformate (60 μL, 400 μmol) was added and then the mixture was sealed and shaken at 50° C. for 16 hours. The reaction mixture was concentrated and the residue purified by preparative HPLC.

Method (iii):

To a solution of the relevant amino-methyl ester (150 μmol) in anhydrous DCM (300 μL) was added DIPEA (450 μmol) and a solution of methyl chloroformate (180 μmol) in anhydrous DCM (300 μL). The mixture was sealed and shaken at 30° C. for 16 hours. It was concentrated then a 0.5 M solution of LiOH in THF/H₂O (V/V=4/1) (600 μL) was added. The mixture was sealed and shaken at 30° C. for 16 hours. The reaction mixture was concentrated, then a solution of the amine obtained from Preparation 128 (75 μmol) in anhydrous DMSO (500 μL) was added, followed by the addition of a solution of HATU (75 μmol) in anhydrous DMSO (500 μL) and DIPEA (375 μmol). The mixture was sealed and was shaken at 50° C. for 16 hours. The reaction mixture was concentrated and the residue was purified by preparative HPLC.

The following analytical and preparative HPLC conditions were used in the preparation of Examples 33 to 84 and are referred to in the tables below.

| HPLC | LCMS Method A | | LCMS Method B | |
|---|---|---|---|---|
| conditions | (analytical) | | (analytical) | |
| Column | Welch XB C18 | | Welch XB C18 | |
| | 5 μm 2.1 × 50 mm | | 5 μm 2.1 × 50 mm | |
| Temperature | 50° C. | | 50° C. | |
| Injection | 2 μL | | 2 μL | |
| volume | | | | |
| Flow rate | 0.8 mL/min | | 0.8 mL/min | |
| Mobile phase | A: H2O + 0.0375% TFA | | A: H2O + 0.0375% TFA | |
| | B: MeCN + 0.1875% TFA | | B: MeCN + 0.01875% TFA | |
| | Time | | Time | |
| Gradient | (min) | % B | (min) | % B |
| | 0.00 | 1 | 0.00 | 10 |
| | 0.60 | 5 | 0.50 | 10 |
| | 4.00 | 100 | 4.00 | 100 |
| | 4.30 | 1 | 4.30 | 10 |
| | 4.70 | 1 | 4.70 | 10 |
| HPLC | LCMS Method C | | LCMS Method D | |
| conditions | (analytical) | | (analytical) | |
| Column | Welch XB C18 | | Welch XB C18 | |
| | 5 μm 2.1 × 50 mm | | 5 μm 2.1 × 50 mm | |
| Temperature | 50° C. | | 50° C. | |
| Injection | 2 μL | | 2 μL | |
| volume | | | | |
| Flow rate | 0.8 mL/min | | 0.8 mL/min | |
| Mobile phase | A: H2O + 0.05% NH4OH | | A: H2O + 0.0375% TFA | |
| | B: MeCN | | B: MeCN + 0.01875% TFA | |
| | Time | | Time | |
| Gradient | (min) | % B | (min) | % B |
| | 0.00 | 5 | 0.00 | 25 |
| | 0.50 | 5 | 0.50 | 25 |
| | 3.40 | 100 | 3.50 | 100 |
| | 4.20 | 100 | 4.00 | 25 |
| | 4.21 | 5 | 4.70 | 25 |
| | 4.70 | 5 | | |

| HPLC conditions | LCMS Method E (analytical) | | | |
|---|---|---|---|---|
| Column | Welch XB C18 | | | |
| | 5 μm 2.1 × 50 mm | | | |
| Temperature | 50° C. | | | |
| Injection volume | 2 μL | | | |
| Flow rate | 0.8 mL/min | | | |
| Mobile phase | A: H2O + 0.05% NH4OH | | | |
| | B: MeCN | | | |
| | Time | | Time | |
| Gradient | (min) | % B | (min) | % B |
| | 0.00 | 15 | | |
| | 0.50 | 15 | | |
| | 3.40 | 100 | | |
| | 3.90 | 100 | | |
| | 3.91 | 15 | | |
| | 4.70 | 15 | | |

| HPLC | LC Method A | | LC Method B | |
|---|---|---|---|---|
| conditions | (preparative) | | (preparative) | |
| Column | Agella venusil ASB C18 | | Boston Symmetrix ODS-H | |
| | 4 μm 21.2 × 150 mm | | 5 μm 30 × 150 mm | |
| Temperature | Ambient | | Ambient | |
| Detection | UV/MS | | UV | |
| Flow rate | 25 to 30 mL/min | | 30 mL/min | |
| Mobile phase | A: H2O + 0.1% TFA | | A: H2O + 0.1% TFA | |
| | B: MeCN | | B: MeCN | |
| Gradient (% B) | Begin (%) | 10-35 | Begin (%) | 20-22 |
| | End (%) | 40-63 | End (%) | 50-52 |
| | Gradient time (min) | 9-10.5 | Gradient time (min) | 8-9 |
| | Hold time (min) | 1-2 | Hold time (min) | 2 |
| HPLC | LC Method C | | LC Method D | |
| conditions | (preparative) | | (preparative) | |
| Column | Kromasil Eternity-5- C18 | | Phenomenex Gemini C18 | |
| | 5 μm 30 × 150 mm | | 10 μm 20 × 200 mm | |
| Temperature | Ambient | | Ambient | |
| Detection | UV | | UV | |
| Flow rate | 30 mL/min | | 28-30 mL/min | |
| Mobile phase | A: H2O + 0.1% TFA | | A: NH4OH(pH 10) | |
| | B: MeCN | | B: MeCN | |
| Gradient (% B) | Begin (%) | 20-24 | Begin (%) | 32-51 |
| | End (%) | 50-54 | End (%) | 62-81 |
| | Gradient time (min) | 10 | Gradient time (min) | 10-11.5 |
| | Hold time (min) | 1.5 | Hold time (min) | 1.5-2 |
| HPLC | LC Method E | | LC Method F | |
| conditions | (preparative) | | (preparative) | |
| Column | Phenomenex Gemini C18 | | Phenomenex Luna C18 | |
| | 10 μm 20 × 200 mm | | 5 μm 21.2 × 100 mm | |
| Temperature | Ambient | | Ambient | |
| Detection | UV | | UV | |
| Flow rate | 28 mL/min | | 25 mL/min | |
| Mobile phase | A: H2O + 0.1% TFA | | A: H2O + 0.1% TFA | |
| | B: MeCN | | B: MeCN | |
| Gradient (% B) | Begin (%) | 29-34 | Begin (%) | 5-20 |
| | End (%) | 59-64 | End (%) | 35-50 |
| | Gradient time (min) | 10.5 | Gradient time (min) | 7 |
| | Hold time (min) | 2 | Hold time (min) | 1.5 |
| HPLC | LC Method G | | LC Method H | |
| conditions | (preparative) | | (preparative) | |
| Column | Phenomenex Synergi C18 | | YMC-pack ODS-AQ | |
| | 4 μm 30 × 150 mm | | 5 μm 30 × 150 mm | |
| Temperature | Ambient | | Ambient | |
| Detection | UV/MS | | MS | |
| Flow rate | 17-35 mL/min | | 35 mL/min | |
| Mobile phase | A: H2O + 0.1% TFA | | A: H2O + 0.1% TFA | |
| | B: MeCN | | B: MeCN | |
| Gradient (% B) | Begin (%) | 15-30 | Begin (%) | 9-25 |
| | End (%) | 35-60 | End (%) | 49-65 |
| | Gradient time (min) | 8-16 | Gradient time (min) | 8 |
| | Hold time (min) | 1.5-2 | Hold time (min) | 1-1.5 |

| HPLC conditions | LC Method I (preparative) | |
|---|---|---|
| Column | Phenomenex Synergi C18 | |
| | 4 μm 30 × 150 mm | |
| Temperature | Ambient | |
| Detection | MS | |
| Flow rate | 35 mL/min | |
| Mobile phase | A: H2O + 0.225% FA | |
| | B: MeCN | |
| Gradient (% B) | Begin (%) | 12-13 |
| | End (%) | 42-43 |
| | Gradient time (min) | 8 |
| | Hold time (min) | 2 |

| Ex. | | | Preparative and characterising |
|---|---|---|---|
| No. | R | Name | data |
| 33 | 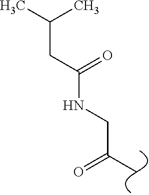 | Methyl {(2S)-3-methyl-1-[(2S)-2-(4-{4-[6- (2-{(2S)-1-[N-(3-methylbutanoyl)glycyl] pyrrolidin-2-yl}-1H-imidazol-5-yl) quinoxazolin-2-yl]phenyl}-1H-imidazol-2-yl} pyrrolidin-1-yl]-1-oxobutan-2-yl} carbamate trifluoroacetic acid salt | Prepared using Method (i) from the relevant acid and product of Preparation 128. Purified by LC Method H. Characterised by LCMS Method B: retention time 2.12 mins: LRMS m/z (ES) M+ 775 [M + H]. |
| 34 | 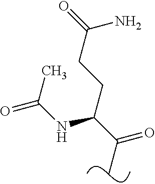 | Methyl {(2S)-1-[(2S)-2-{4-[4-(6-[2-[(2S)- 1-(N²-acetyl-L-glutaminyl)pyrrolidin-2-yl]- 1H-imidazol-5-yl}quinoxalin-2-yl)phenyl]- 1H-imidazol-2-yl}pyrrolidin-1-yl]-3- methyl-1-oxobutan-2-yl}carbamate trifluoroacetic acid salt | Prepared using Method (i) from the acid and product of Preparation 128. Purified by LC Method A. Characterised by LCMS Method C: retention time 2.40 mins: LRMS m/z (ES) M+ 804 [M + H]. |
| 35 | 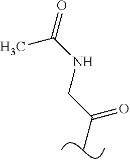 | Methyl {(2S)-1-[(2S)-2-{4-[4-(6-{2-[(2S)- 1-(N-acetylglycyl)pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (i) from the acid and product of Preparation 128. Purified by LC Method H. Characterised by LCMS Method A: retention time 2.21 mins: LRMS m/z (ES) M+ 733 [M + H]. |
| 36 | 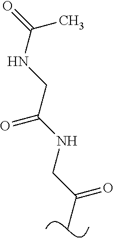 | Methyl {(2S)-1-[(2S)-2-{4-[4-(6-{2-[(2S)- 1-(N-acetylglycylglycyl)pyrrolidin-2-yl]- 1H-imidazol-5-yl}quinoxalin-2-yl)phenyl]- 1H-imidazol-2-yl}pyrrolidin-1-yl]-3- methyl-1-oxobutan-2-yl}carbamate trifluoroacetic acid salt | Prepared using Method (i) from the acid and product of Preparation 128. Purified by LC Method A. Characterised by LCMS Method A: retention time 2.18 mins: LRMS m/z (ES) M+ 790 [M + H]. |
| 37 | 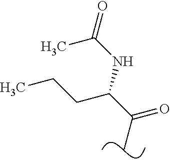 | Methyl {(2S)-1-[(2S)-2-{4-{4-[6-(2-{(2S)- 1-(N-acetyl-L-norvalyl)pyrrolidin-2-yl]-1H- imidazol-5-yl)quinoxalin-2-yl)phenyl]-1H- imidazol-2-yl}pyrrolidin-1-yl]-3-methyl-1- oxobutan-2-yl}carbamate trifluoroacetic acid salt | Prepared using Method (i) from the acid and product of Preparation 128. Purified by LC Method A. Characterised by LCMS Method C: retention time 2.64 mins: LRMS m/z (ES) M+ 775 [M + H]. |
| 38 | 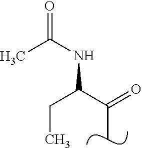 | Methyl {(2S)-1-[(2S)-2-(4-{4-[6-(2-{(2S)- 1-[(2R)-2-(acetylamino)butanoyl] pyrrolidin-2-yl}-1H-imidazol-5-yl) quinoxalin-2-yl]phenyl}-1H-imidazol-2-yl) pyrrolidin-1-yl]-3-methyl-1-oxobutan-2-yl} carbamate trifluoroacetic acid salt | Prepared using Method (i) from the acid and product of Preparation 128. Purified by LC Method A. Characterised by LCMS Method C: retention time 2.57 mins: LRMS m/z (ES) M+ 761 [M + H]. |
| 39 | 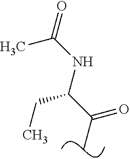 | Methyl {(2S)-1-[(2S)-2-(4-{4-[6-(2-{(2S)- 1-[(2S)-2- (acetylamino)butanoyl]pyrrolidin-2-yl}- 1H-imidazol-5-yl)quinoxalin-2-yl]phenyl}- 1H-imidazol-2-yl)pyrrolidin-1-yl]-3- methyl-1-oxobutan-2-yl}carbamate trifluoroacetic acid salt | Prepared using Method (i) from the acid and product of Preparation 128. Purified by LC Method H. Characterised by LCMS Method E: retention time 2.35 mins: LRMS m/z (ES) M+ 761 [M + H]. |
| 40 | 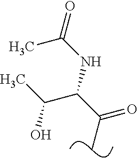 | Methyl {(2S)-1-[(2S)-2-{4-[4-(6-{2-[(2S)- 1-(N-acetyl-L-threonyl)pyrroldiin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (i) from the acid and product of Preparation 128. Purified by LC Method I. Characterised by LCMS Method C: retention time 2.43 mins: LRMS m/z (ES) M+ 777 [M + H]. |
| 41 | 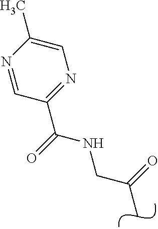 | Methyl {(2S)-3-methyl-1-[(2S)-2-{4-[4-(6- {2-[(2S)-1-{N-[(5-methylpyrazin-2-yl) carbonyl]glycyl}pyrrolidin-2-yl]-1H- imidazol-5-yl}quinoxalin-2-yl)phenyl]-1H- imidazol-2-yl}pyrrolidin-1-yl]-1-oxobutan- 2-yl}carbamate trifluoroacetic acid salt | Prepared using Method (i) from the acid and product of Preparation 128. Purified by LC Method H. Characterised by LCMS Method B: retention time 2.30 mins: LRMS m/z (ES) M+ 811 [M + H]. |
| 42 | 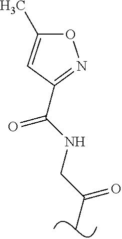 | Methyl {(2S)-3-methyl-1-[(2S)-2-{4-[4-(6- {2-[(2S)-1-{N-[(5-methylisoxazol-3-yl) carbonyl]glycyl}pyrrolidin-2-yl]-1H- imidazol-5-yl}quinoxalin-2-yl)phenyl]-1H- imidazol-2-yl}pyrrolidin-1-yl]-1-oxobutan- 2-yl}carbamate trifluoroacetic acid salt | Prepared using Method (i) from the acid and product of Preparation 128. Purified by LC Method A. Characterised by LCMS Method A: retention time 2.33 mins: LRMS m/z (ES) M+ 800 [M + H]. |
| 43 | 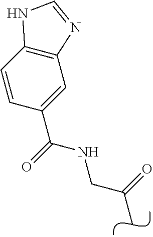 | Methyl {(2S)-1-[(2S)-2-(4-{4-[6-(2-{(2S)- 1-[N-(1H-benzimidazol-5- ylcarbonyl)glycyl]pyrrolidin-2-yl}-1H- imidazol-5-yl)quinoxalin-2-yl]phenyl}-1H- imidazol-2-yl)pyrrolidin-1-yl]-3-methyl-1- oxobutan-2-yl}carbamate trifluoroacetic acid salt | Prepared using Method (i) from the acid and product of Preparation 128. Purified by LC Method A. Characterised by LCMS Method B: retention time 1.90 mins: LRMS m/z (ES) M+ 835 [M + H]. |
| 44 | 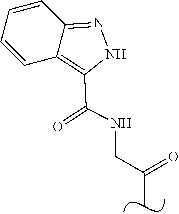 | Methyl {(2S)-1-[(2S)-2-(4-{4-[6-(2-{(2S)- 1-[N-(2H-indazol-3-ylcarbonyl)glycyl] pyrrolidin-2-yl}-1H-imidazol-5-yl) quinoxalin-2-yl]phenyl}-1H-imidazol-2-yl) pyrrolidin-1-yl]-3-methyl-1-oxobutan-2-yl} carbamate trifluoroacetic acid salt | Prepared using Method (i) from the acid and product of Preparation 128. Purified by LC Method A. Characterised by LCMS Method B: retention time 2.16 mins: LRMS m/z (ES) M+ 835 [M + H]. |
| 45 | 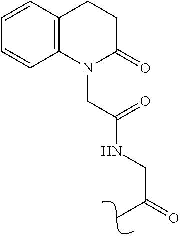 | Methyl {(2S)-3-methyl-1-oxo-1-[(2S)-2- {4-[4-(6-[2-[(2S)-1-{N-[(2-oxo-3,4-dihydro quinolin-1(2H)-yl)acetyl]glycyl}pyrrolidin- 2-yl]-1H-imidazol-5-yl}quinoxalin-2-yl) phenyl]-1H-imidazol-2-yl}pyrrolidin-1-yl] butan-2-yl}carbamate trifluoroacetic acid salt | Prepared using Method (i) from the acid and product of Preparation 128. Purified by LC Method A. Characterised by LCMS Method B: retention time 2.15 mins: LRMS m/z (ES) M+ 878 [M + H]. |
| 46 | 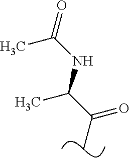 | Methyl {(2S)-1-[(2S)-2-{4-[4-(6-(2-[(2S)- 1-(N-acetyl-D-alanyl)pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (i) from the acid and product of Preparation 128. Purified by LC Method H. Characterised by LCMS Method A: retention time 2.26 mins: LRMS m/z (ES) M+ 747 [M + H]. |
| 47 | 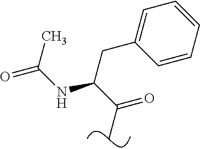 | Methyl {(2S)-1-[(2S)-2-{4-[4-(6-{2-[(2S)- 1-(N-acetyl-L-phenylalanyl)pyrrolidin-2- yl]-1H-imidazol-5-yl}quinoxalin-2-yl) phenyl]-1H-imidazol-2-yl}pyrrolidin-1-yl]- 3-methyl-1-oxobutan-2-yl}carbamate trifluoroacetic acid salt | Prepared using Method (i) from the acid and product of Preparation 128. Purified by LC Method H. Characterised by LCMS Method B: retention time 2.25 mins: LRMS m/z (ES) M+ 823 [M + H]. |
| 48 | 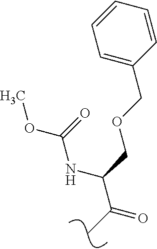 | Methyl {(2S)-1-[(2S)-2-{4-[4-(6-{2-[(2S)- 1-{(2S)-3-(benzyloxy)-2- [(methoxycarbonyl)amino]propanoyl}pyrro- lidin-2-yl]-1H-imidazol-5-yl}quinoxalin-2- yl)phenyl]-1H-imidazol-2-yl}pyrrolidin-1- yl]-3-methyl-1-oxobutan-2-yl}carbamate carbamate trifluoroacetic acid salt | Prepared using Method (ii) from the acid and product of Preparation 128. Purified by LC Method G. Characterised by LCMS Method B: retention time 2.31 mins: LRMS m/z (ES) M+ 869 [M + H]. |
| 49 | 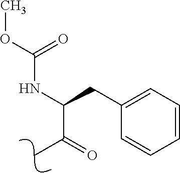 | Methyl {(2S)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-1-oxo-3- phenylpropan-2-yl}carbamate trifluoroacetic acid salt | Prepared using Method (ii) from the acid and product of Preparation 128. Purified by LC Method B. Characterised by LCMS Method B: retention time 2.27 mins: LRMS m/z (ES) M+ 839 [M + H]. |
| 50 | 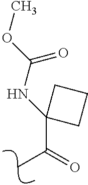 | Methyl {(2S)-1-[(2S)-2-{4-[4-(6-{2-[(2S)- 1-({1-[(methoxycarbonyl)amino] cyclobutyl}carbonyl)pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (ii) from the acid and product of Preparation 128. Purified by LC Method E. Characterised by LCMS Method B: retention time 2.15 mins: LRMS m/z (ES) M+ 789 [M + H]. |
| 51 | 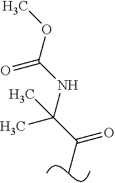 | Methyl {(2S)-1-[(2S)-2-{4-[4-(6-{2-[(2S)- 1-{2-[(methoxycarbonyl)amino]-2- methylpropanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (ii) from the acid and product of Preparation 128. Purified by LC Method A. Characterised by LCMS Method A: retention time 2.38 mins: LRMS m/z (ES) M+ 777 [M + H]. |
| 52 | 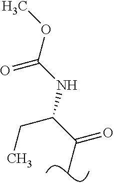 | Methyl {(2S)-1-[(2S)-2-{4-[4-(6-{2-[(2S)- 1-{(2S)-2-[(methoxycarbonyl)amino] butanoyl}pyrrolidin-2-yl]-1H-imidazol-5- yl}quinoxalin-2-yl)phenyl]-1H-imidazol-2- yl}pyrrolidin-1-yl]-3-methyl-1-oxobutan-2- yl}carbamate trifluoroacetic acid salt | Prepared using Method (ii) from the acid and product of Preparation 128. Purified by LC Method C. Characterised by LCMS Method B: retention time 2.10 mins: LRMS m/z (ES) M+ 777 [M + H]. |
| 53 | 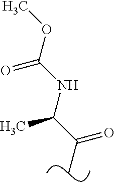 | Methyl {(2S)-1-[(2S)-2-(4-{4-[6-(2-{(2S)- 1-[N-(methoxycarbonyl)-D-alanyl] pyrrolidin-2-yl}-1H-imidazol-5-yl) quinoxalin-2-yl]phenyl}-1H-imidazol-2-yl) pyrrolidin-1-yl]-3-methyl-1-oxobutan-2-yl} carbamate trifluoroacetic acid salt | Prepared using Method (ii) from the acid and product of Preparation 128. Purified by LC Method A. Characterised by LCMS Method A: retention time 2.32 mins: LRMS m/z (ES) M+ 763 [M + H]. |
| 54 | 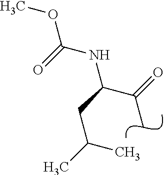 | Methyl {(2R)-1-[(2S)-2-{5-[2-(4-{2-[(2S)- 1-{(2S)-2-[(methoxycarbonyl)amino]-3- methylbutanoyl}pyrrolidin-2-yl]-1H- imidaozl-4-yl}phenyl)quinoxalin-6-yl]-1H- imidazol-2-yl}pyrrolidin-1-yl]-4-methyl-1- oxopentan-2-yl}carbamate trifluoroacetic acid salt | Prepared using Method (ii) from the acid and product of Preparation 128. Purified by LC Method A. Characterised by LCMS Method B: retention time 2.30 mins: LRMS m/z (ES) M+ 805 [M + H]. |
| 55 | 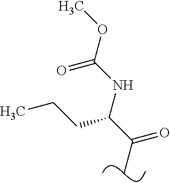 | Methyl {(2S)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-1- oxopentan-2-yl}carbamate trifluoroacetic acid salt | Prepared using Method (ii) from the acid and product of Preparation 128. Purified by LC Method H. Characterised by LCMS Method B: retention time 2.17 mins: LRMS m/z (ES) M+ 791 [M + H]. |
| 56 | 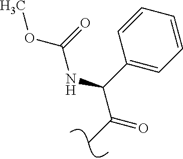 | Methyl {(2S)-1-[(2S)-2-{4-[4-(6-(2-[(2S)- 1-{(2S)-2-[(methoxycarbonyl)amino]-2- phenylacetyl}pyrrolidin-2-yl]-1H-imidazol- 5-yl}quinoxalin-2-yl)phenyl]-1H-imidazol- 2-yl}pyrrolidin-1-yl]-3-methyl-1-oxobutan- 2-yl}carbamate trifluoroacetic acid salt | Prepared using Method (ii) from the acid and product of Preparation 128. Purified by LC Method B. Characterised by LCMS Method B: retention time 2.22 mins: LRMS m/z (ES) M+ 825 [M + H]. |
| 57 | 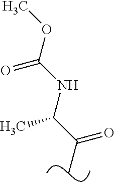 | Methyl {(2S)-1-[(2S)-2-(4-{4-[6-(2-{(2S)- 1-[N-(methoxycarbonyl)-L-alanyl] pyrrolidin-2-yl}-1H-imidazol-5-yl) quinoxalin-2-yl]phenyl}-1H-imidazol-2-yl) pyrrolidin-1-yl]-3-methyl-1-oxobutan-2-yl} carbamate trifluoroacetic acid salt | Prepared using Method (ii) from the acid and product of Preparation 128. Purified by LC Method G. Characterised by LCMS Method B: retention time 2.29 mins: LRMS m/z (ES) M+ 763 [M + H]. |
| 58 | 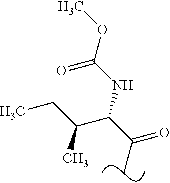 | Methyl {(2S)-1-[(2S)-2-{4-[4-(6-{2-[(2S)- 1-{(2S,3S)-2-[(methoxycarbonyl)amino]- 3-methylpentanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (ii) from the acid and product of Preparation 128. Purified by LC Method E. Characterised by LCMS Method B: retention time 2.23 mins: LRMS m/z (ES) M+ 804 [M + H]. |
| 59 | 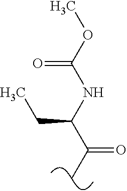 | Methyl {(2S)-1-[(2S)-2-{4-[4-(6-{2-[(2S)- 1-{(2R)-2-[(methoxycarbonyl)amino] butanoyl}pyrrolidin-2-yl]-1H-imidazol-5-yl} quinoxalin-2-yl)phenyl]-1H-imidazol-2-yl} pyrrolidin-1-yl]-3-methyl-1-oxobutan-2-yl} carbamate trifluoroacetic acid salt | Prepared using Method (ii) from the acid and product of Preparation 128. Purified by LC Method H. Characterised by LCMS Method B: retention time 2.14 mins: LRMS m/z (ES) M+ 777 [M + H]. |
| 60 | 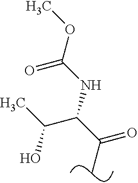 | Methyl {(2S)-1-[(2S)-2-{4-[4-(6-{2-[(2S)- 1-{(2S,3R)-3-hydroxy-2-[(methoxy carbonyl)amino]butanoyl}pyrrolidin-2-yl]- 1H-imidazol-5-yl}quinoxalin-2-yl)phenyl]- 1H-imidazol-2-yl}pyrrolidin-1-yl]-3- methyl-1-oxobutan-2-yl}carbamate trifluoroacetic acid salt | Prepared using Method (ii) from the acid and product of Preparation 128. Purified by LC Method C. Characterised by LCMS Method A: retention time 2.28 mins: LRMS m/z (ES) M+ 793 [M + H]. |
| 61 | 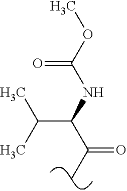 | Methyl {(2S)-1-[(2S)-2-{4-[4-(6-{2-[(2S)- 1-{(2R)-2-[(methoxycarbonyl)amino]-3- methylbutanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (ii) from the acid and product of Preparation 128. Purified by LC Method B. Characterised by LCMS Method B: retention time 2.20 mins: LRMS m/z (ES) M+ 791 [M + H]. |
| 62 | 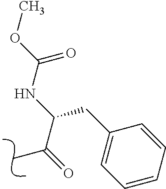 | Methyl {(2R)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-1-oxo-3- phenylpropan-2-yl}carbamate trifluoroacetic acid salt | Prepared using Method (ii) from the acid and product of Preparation 128. Purified by LC Method H. Characterised by LCMS Method B: retention time 2.33 mins: LRMS m/z (ES) M+ 839 [M + H]. |
| 63 | 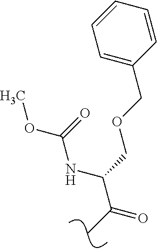 | Methyl {(2S)-1-[(2S)-2-{4-[4-(6-{2-[(2S)- 1-{(2R)-3-(benzyloxy)-2-[(methoxy carbonyl)amino]propanoyl}pyrrolidin-2- yl]-1H-imidazol-5-yl}quinoxalin-2-yl) phenyl]-1H-imidazol-2-yl}pyrrolidin-1-yl]- 3-methyl-1-oxobutan-2-yl}carbamate trifluoroacetic acid salt | Prepared using Method (ii) from the acid and product of Preparation 128. Purified by LC Method E. Characterised by LCMS Method B: retention time 2.36 mins: LRMS m/z (ES) M+ 869 [M + H]. |
| 64 | 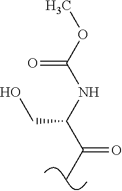 | Methyl {(2S)-1-[(2S)-2-{4-[4-(6-{2-[(2S)- 1-{(2S)-3-hydroxy-2-[(methoxycarbonyl) amino]propanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (ii) from the acid and product of Preparation 128. Purified by LC Method A. Characterised by LCMS Method A: retention time 2.25 mins: LRMS m/z (ES) M+ 779 [M + H]. |
| 65 | 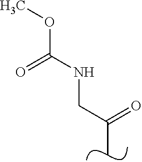 | Methyl {2-[(2S)-2-[5-[2-(4-{2-[(2S)-1- {(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-2- oxoethyl)carbamate trifluoroacetic acid salt | Prepared using Method (ii) from the acid and product of Preparation 128. Purified by LC Method F. Characterised by LCMS Method A: retention time 2.26 mins: LRMS m/z (ES) M+ 749 [M + H]. |
| 66 | 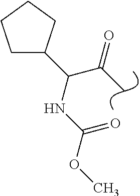 | Methyl {(2S)-1-[(2S)-2-{4-[4-(6-{2-[(2S)- 1-{cyclopentyl[(methoxycarbonyl)amino] acetyl}pyrrolidin-2-yl]-1H-imidazol-5-yl} quinoxalin-2-yl)phenyl]-1H-imidazol-2-yl} pyrrolidin-1-yl]-3-methyl-1-oxobutan-2-yl} carbamate trifluoroacetic acid salt | Prepared using Method (ii) from the acid and product of Preparation 128. Purified by LC Method A. Characterised by LCMS Method B: retention time 2.32 mins: LRMS m/z (ES) M+ 817 [M + H]. |
| 67 | 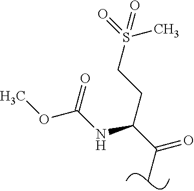 | methyl [(2S)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-4-(methyl sulfonyl)-1-oxobutan-2-yl]carbamate trifluoroacetic acid salt | Prepared using Method (ii) from the acid and product of Preparation 128. Purified by LC Method A. Characterised by LCMS Method A: retention time 2.29 mins: LRMS m/z (ES) M+ 855 [M + H]. |
| 68 | 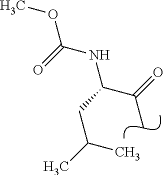 | Methyl {(2S)-1-[(2S)-2-{5-[2-(4-{2-[(2S)- 1-{(2S)-2-[(methoxycarobnyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-4-methyl-1- oxopentan-2-yl}carbamate trifluoroacetic acid salt | Prepared using Method (iii) from the acid and product of Preparation 128. Purified by LC Method E. Characterised by LCMS Method B: retention time 2.22 mins: LRMS m/z (ES) M+ 805 [M + H]. |
| 69 | 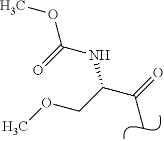 | Methyl {(2S)-1-[(2S)-2-{4-[4-(6-{2-[(2S)- 1-{(2S)-3-methoxy-2-[(methoxycarbonyl) amino]propanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (ii) from the acid and product of Preparation 128. Purified by LC Method A. Characterised by LCMS Method B: retention time 2.45 mins: LRMS m/z (ES) M+ 793 [M + H]. |
| 70 | 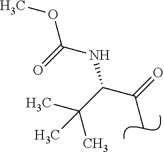 | Methyl {(2S)-1-[(2S)-2-{4-[4-(6-{2-[(2S)- 1-{(2S)-2-[(methoxycarbonyl)amino]-3,3- dimethylbutanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (iii) from the acid and product of Preparation 128. Purified by LC Method A. Characterised by LCMS Method B: retention time 2.21 mins: LRMS m/z (ES) M+ 805 [M + H]. |
| 71 | 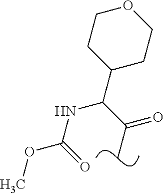 | Methyl {(2S)-1-[(2S)-2-{4-[4-(6-{2-[(2S)- 1-{[(methoxycarbonyl)amino](tetrahydro- 2H-pyran-4-yl)acetyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate | Prepared using Method (ii) from the acid and product of Preparation 128. Purified by LC Method D. Characterised by LCMS Method B: retention time 2.32 mins: LRMS m/z (ES) M+ 833 [M + H]. |
| 72 | 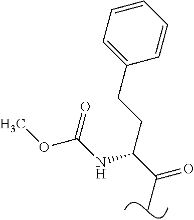 | Methyl {(2R)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-1-oxo-4- phenylbutan-2-yl}carbamate trifluoroacetic acid salt | Prepared using Method (iii) from the amine and product of Preparation 128. Purified by LC Method F. Characterised by LCMS Method B: retention time 2.310 mins: LRMS m/z (ES) M+ 853 [M + H]. |
| 73 | 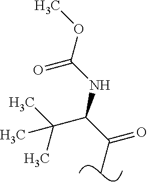 | Methyl {(2S)-1-[(2S)-2-{4-[4-(6-{2-[(2S)- 1-{(2R)-2-[(methoxycarbonyl)amino]-3,3- dimethylbutanoyl}pyrrolidin-2-yl]-1H- imidazol-5-yl}quinoxalin-2-yl)phenyl]-1H- imidazol-2-yl}pyrrolidin-1-yl]-3-methyl-1- oxobutan-2-yl}carbamate trifluoroacetic acid salt | Prepared using Method (iii) from the amine and product of Preparation 128. Purified by LC Method A. Characterised by LCMS Method A: retention time 2.31 mins: LRMS m/z (ES) M+ 805 [M + H]. |
| 74 | 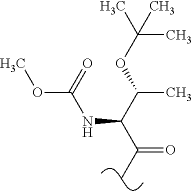 | Methyl {(2S)-1-[(2S)-2-{4-[4-(6-{2-[(2S)- 1-{(2S,3R)-3-tert-butoxy-2-[(methoxy carbonyl)amino]butanoyl}pyrrolidin-2-yl]- 1H-imidazol-5-yl}quinoxalin-2-yl)phenyl]- 1H-imidazol-2-yl}pyrrolidin-1-yl]-3- methyl-1-oxobutan-2-yl)carbamate | Prepared using Method (iii) from the amine and product of Preparation 128. Purified by LC Method E. Characterised by LCMS Method B: retention time 2.21 mins: LRMS m/z (ES) M+ 849 [M + H]. |
| 75 | 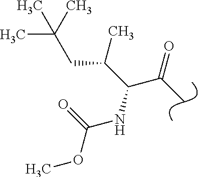 | Methyl {(2S)-1-[(2S)-2-{4-[4-(6-{2-[(2S)- 1-{(2R,3R)-3-tert-butoxy-2-[(methoxy carbonyl)amino]butanoyl}pyrrolidin-2-yl]- 1H-imidazol-5-yl}quinoxalin-2-yl)phenyl]- 1H-imidazol-2-yl}pyrrolidin-1-yl]-3- methyl-1-oxobutan-2-yl}carbamate trifluoroacetic acid salt | Prepared using Method (iii) from the amine and product of Preparation 128. Purified by LC Method E. Characterised by LCMS Method A: retention time 2.41 mins: LRMS m/z (ES) M+ 849 [M + H]. |
| 76 | 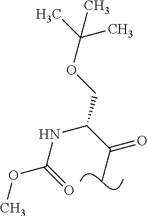 | Methyl {(2S)-1-[(2S)-2-{4-[4-(6-{2-[(2S)- 1-{(2R)-3-tert-butoxy-2-[(methoxy carbonyl)amino]propanoyl}pyrrolidin-2- yl]-1H-imidazol-5-yl]quinoxalin-2-yl) phenyl]-1H-imidazol-2-yl}pyrrolidin-1-yl]- 3-methyl-1-oxobutan-2-yl}carbamate trifluoroacetic acid salt | Prepared using Method (iii) from the amine and product of Preparation 128. Purified by LC Method A. Characterised by LCMS Method C: retention time 2.730 mins: LRMS m/z (ES) M+ 835 [M + H]. |
| 77 | 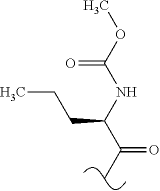 | Methyl {(2R)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-1- oxopentan-2-yl}carbamate trifluoroacetic acid salt | Prepared using Method (iii) from the amine and product of Preparation 128. Purified by LC Method A. Characterised by LCMS Method A: retention time 2.30 mins: LRMS m/z (ES) M+ 791 [M + H]. |
| 78 | 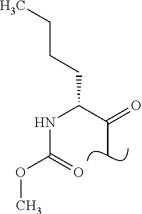 | Methyl {(2R)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl}pyrrolidin-1-yl]-1-oxohexan- 2-yl}carbamate trifluoroacetic acid salt | Prepared using Method (iii) from the amine and product of Preparation 128. Purified by LC Method A. Characterised by LCMS Method B: retention time 2.28 mins: LRMS m/z (ES) M+ 805 [M + H]. |
| 79 | 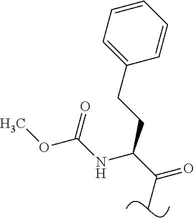 | Methyl {(2S)-1-[(2S)-2-{5-[2-(4-{2-[(2S)- 1-{(2S)-2-[(methoxycarbonyl)amino]-3- methylbutanoyl}pyrrolidin-2-yl]-1H- imidazol-4-yl}phenyl)quinoxalin-6-yl]-1H- imidazol-2-yl]pyrrolidin-1-yl]-1-oxo-4- phenylbutan-2-yl}carbamate trifluoroacetic acid salt | Prepared using Method (iii) from the amine and product of Preparation 128. Purified by LC Method E. Characterised by LCMS Method B: retention time 2.31 mins: LRMS m/z (ES) M+ 853 [M + H]. |
| 80 | 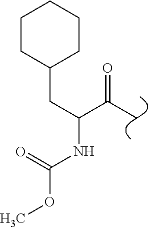 | Methyl {(2S)-1-[(2S)-2-(4-{4-[6-(2-{(2S)- 1-[3-cyclohexyl-N-(methoxycarbonyl) alanyl]pyrrolidin-2-yl}-1H-imidazol-5-yl) quinoxalin-2-yl]phenyl}-1H-imidazol-2-yl) pyrrolidin-1-yl]-3-methyl-1-oxobutan-2-yl} carbamate | Prepared using Method (iii) from the amine and product of Preparation 128. Purified by LC Method E. Characterised by LCMS Method D: retention time 1.70 mins: LRMS m/z (ES) M+ 845 [M + H]. |
| 81 | 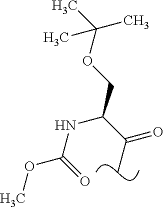 | Methyl {(2S)-1-[(2S)-2-{4-[4-(6-{2-[(2S)- 1-{(2S)-3-tert-butoxy-2-[(methoxy carbonyl)amino]propanoyl}pyrrolidin-2- yl]-1H-imidazol-5-yl}quinoxalin-2-yl) phenyl]-1H-imidazol-2-yl}pyrrolidin-1-yl]- 3-methyl-1-oxobutan-2-yl}carbamate trifluoroacetic acid salt | Prepared using Method (iii) from the amine and product of Preparation 128. Purified by LC Method E. Characterised by LCMS Method B: retention time 2.22 mins: LRMS m/z (ES) M+ 805 [M + H]. |
| 82 | 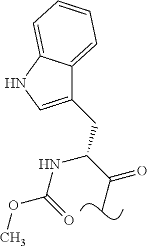 | Methyl {(2S)-1-[(2S)-2-{4-[4-(6-{2-[(2S)- 1-{(2R)-3-(1H-indol-3-yl)-2-[(methoxy carbonyl)amino]propanoyl}pyrrolidin-2- yl]-1H-imidazol-5-yl}quinoxalin-2-yl) phenyl]-1H-imidazol-2-yl}pyrrolidin-1-yl]- 3-methyl-1-oxobutan-2-yl}carbamate trifluoroacetic acid salt | Prepared using Method (iii) from the amine and product of Preparation 136. Purified by LC Method E. Characterised by LCMS Method B: retention time 2.30 mins: LRMS m/z (ES) M+ 878 [M + H]. |
| 83 | 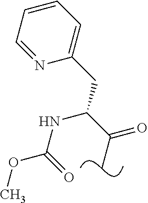 | Methyl {(2S)-1-[(2S)-2-(4-{4-[6-(2-{(2S)- 1-[(2R)-2-[(methoxycarbonyl)amino]-3- (pyridin-2-yl)propanoyl]pyrrolidin-2-yl}- 1H-imidazol-5-yl)quinoxalin-2-yl]phenyl}- 1H-imidazol-2-yl)pyrrolidin-1-yl]-3- methyl-1-oxobutan-2-yl}carbamate trifluoroacetic acid salt | Prepared using Method (iii) from the amine and product of Preparation 136. Purified by LC Method A. Characterised by LCMS Method A: retention time 2.21 mins: LRMS m/z (ES) M+ 840 [M + H]. |
| 84 | 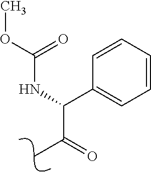 | Methyl {(2S)-1-[(2S)-2-{4-[4-(6-{2-[(2S)- 1-{(2R)-2-[(methoxycarbonyl)amino]-2- phenylacetyl}pyrrolidin-2-yl]-1H-imidazol- 5-yl}quinoxalin-2-yl)phenyl]-1H-imidazol- 2-yl}pyrrolidin-1-yl]-3-methyl-1-oxobutan- 2-yl}carbamate trifluoroacetic acid salt | Prepared using Method (iii) from the amine and product of Preparation 136. Purified by LC Method E. Characterised by LCMS Method B: retention time 2.21 mins: LRMS m/z (ES) M+ 825 [M + H]. |

### Preparation 1

### 2-Aminomethyl-4-bromophenylamine

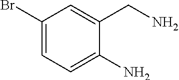

Method A: To a stirred solution of 2-amino-5-bromobenzonitrile (5.0 g, 25.3 mmol) in THF (300 mL) was added a 1M solution of BH₃.THF (32.9 mL, 32.9 mmol) slowly at 0° C. and the reaction mixture was stirred to room temperature for 72 hours. After cooling to 0° C., absolute EtOH was added followed by 4N dioxane HCl and the reaction mixture was stirred for 6 hours. The solvent was evaporated to dryness and the residue was triturated with ether (2×30 mL). It was then neutralized with methanolic-ammonia and the crude material was purified by column chromatography (methanol-dichloromethane 1:9) to afford the title compound as an off white solid (3.8 g).

¹H NMR (400 MHz, DMSO-d₆): δ=7.19 (1H, d), 7.04 (1H, dd), 6.54 (1H, d), 5.24 (2H, br s), 3.56 (2H, s), 2.04 (2H, br s).

Method B: To a stirred solution of 2-amino-5-bromobenzonitrile (500 g, 2.5 mol) in THF (10 mL/g 5 L) was added a 1M solution of BH₃. THF (5.08 L, 5.08 mol) slowly at 0° C. and the reaction mixture was stirred to room temperature for 72 hours. After cooling to 0° C., absolute EtOH (5 mL/g, 2.5 L) was added followed by 4M HCl (5 mL/g, 2.5 L) and the reaction mixture was stirred overnight. The solvent was evaporated under vacuum. To the remaining aqueous solution was added ⁱPrOAc (10 mL/g, 5 L) and the biphasic mixture was basified using solid NaOH pellets until basic pH was achieved. The phases were separated and the organic phase was washed with 2×5 mL/g H₂O and then separated. The solvent was evaporated under vacuum to dryness to afford the title compound as an off white solid (430 g).

### Preparation 2

### N-(2-Amino-5-bromobenzyl)-4-bromobenzamide

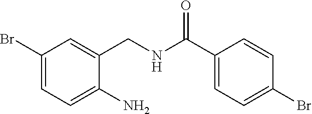

Method A: To a stirred solution of 2-aminomethyl-4-bromophenylamine (3.8 g, 18.9 mmol), obtained from Preparation 1, in dichloromethane (40 mL) was added triethylamine (3.6 mL, 28.3 mmol) followed by a solution of 4-bromobenzoyl chloride (4.1 g, 18.9 mmol) in dichloromethane (10 mL), at 0° C., slowly. The reaction mixture was stirred at room temperature for 2 hours. The organic layer was then washed with water (2×15 mL), dried over sodium sulphate and evaporated to dryness to afford the title compound as an off white solid (7.2 g).

¹H NMR (400 MHz, DMSO-d₆): δ=9.0 (1H, t), 7.8 (2H, d), 7.7 (2H, d), 7.1 (2H, m), 6.6 (1H, d), 5.3 (2H, br s), 4.3 (2H, d). LCMS (run time=5 min): Rₜ=3.38 min; m/z 385 [M+H]⁺

Method B: To a stirred solution of 2-aminomethyl-4-bromophenylamine (600 g, 2.98 mol), obtained from Preparation 1, in 2-methyltetrahydrofuran (4.8 L) was added 2N NaOH (5 mL/g; 3 L). To the biphasic solution was added a solution of 4-bromobenzoyl chloride (520 g, 2.39 mol) in 2-methyltetrahydrofuran (1.2 L) at room temperature, slowly. The reaction mixture was stirred at room temperature for 2 hours. The phases were separated and the organic phase was washed with water (3 L) followed by brine (3 L). The organic solution was stripped into TBME and the resulting slurry was isolated by filtration to afford the title compound as an off white solid (734 g).

### Preparation 3

### 6-Bromo-2-(4-bromo-phenyl)-3,4-dihydroquinazoline

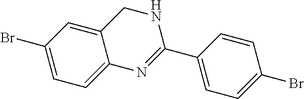

Method A: N-(2-Amino-5-bromobenzyl)-4-bromobenzamide (7.2 g, 18.7 mmol), obtained from Preparation 2, was suspended in POCl₃ (30 mL) and the mixture was heated to reflux for 2 hours. The solvent was then evaporated to dryness and the residue was neutralized with a saturated solution of sodium bicarbonate. The product was extracted into ethyl acetate (3×30 mL). The combined organic extracts were dried over sodium sulphate and concentrated in vacuo to afford the title compound as a light yellow solid (3 g).

¹H NMR (400 MHz, DMSO-d₆): δ=12.4+10.9 (1H, 2×br s), 7.9 (4H, m), 7.5 (2H, m), 7.3 (1H, d), 4.8 (2H, s).

LCMS (run time=5 min): Rₜ=2.61 min; m/z 367 [M+H]⁺

Method B: N-(2-amino-5-bromobenzyl)-4-bromobenzamide (392 g, 1.02 mol), obtained from Preparation 2, was suspended in POCl₃ (1.96 L) and the mixture was heated to reflux for 2 hours. The solvent was evaporated to dryness and restriped with toluene. The residue was suspended in toluene (2.5 L) and neutralized with 2M K₂CO_{3(aq)} solution. The biphasic slurry was granulated and isolated by filtration to afford the title compound as a light yellow solid (364.5 g).

### Preparation 4

### 6-Bromo-2-(4-bromophenyl)-quinazoline

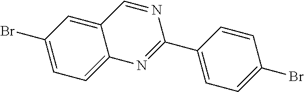

Method A: To a stirred solution of 6-bromo-2-(4-bromophenyl)-3,4-dihydroquinazoline (3 g, 8.1 mmol), obtained from Preparation 3, in toluene (50 mL) was added chloranil (2 g, 8.1 mmol) and the reaction mixture was heated to reflux for 4 hours. The mixture was concentrated in vacuo and the residue was treated with dichloromethane. The insoluble material was filtered off and was washed with dichloromethane (30 mL). The combined organic filtrates were washed with 0.1 N NaOH (1×15 mL), followed by water (2×15 mL). Then the organic layer was dried over sodium sulphate and was concentrated to dryness to afford the title compound. (2.8 g, 96% yield), (off white solid).

¹H NMR (400 MHz, DMSO-d₆): δ=9.69 (1H, s), 8.48 (3H, m), 8.16 (1H, d), 8.01 (1H, d), 7.78 (2H, d). LCMS (run time=5 min): Rₜ=2.88 min; m/z 365 [M+H]⁺

Method B: To a stirred solution of 6-bromo-2-(4-bromophenyl)-3,4-dihydroquinazoline (380 g, 1.04 mol) in 1,4-Dioxane (3.8 L) was added DDQ (280 g, 1.25 mol) and the reaction mixture was heated to reflux for 4 hours. The mixture was concentrated in vacuo and the residue was treated with a solution of 1M sodium hydroxide solution (3.8 L). The insoluble solid was filtered off to give the title compound as an off white solid (362 g).

### Preparation 5

### 1-[2-(4-Acetylphenyl)-quinazolin-6-yl]-ethanone

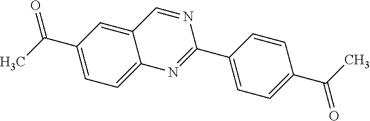

To a stirred solution of 6-bromo-2-(4-bromophenyl)-quinazoline (2.8 g, 7.6 mmol), obtained from Preparation 4, in DMF (15 mL) was added tributyl(1-ethoxy)vinyltin (6.94 g, 19 mmol), and the mixture was degassed with a stream of Ar for 30 minutes. PdCl₂(PPh₃)₂ (0.54 g, 0.76 mmol) was then added and the mixture was heated at 100° C. for 3 hours. After cooling to room temperature, the mixture was diluted with ether, treated with KF solution and stirred at room temperature for 1 hour. The mixture was filtered through celite, whereupon the organic layer was separated and the aqueous layer was extracted with ether (2×25 mL). The combined organic layers were washed with water (2×20 mL) and saturated sodium bicarbonate (1×20 mL), dried over sodium sulphate, and concentrated in vacuo. The resulting crude material was treated with 2N HCl (10 mL) and THF (20 mL) and allowed to stand at room temperature for 16 hours. The mixture was partially concentrated, diluted with water (15 mL), and extracted with ethyl acetate (3×15 mL). The combined organic layers were washed with water (2×10 mL) and brine (1×15 mL), dried (Na₂SO₄), and evaporated to dryness. The residue was purified by column chromatography on silica gel (ethyl acetate:hexane, 3:2) to afford the title compound as an off white solid (1.3 g).

¹H NMR (400 MHz, DMSO-d₆): δ=9.90 (1H, s), 8.92 (1H, s), 8.70 (2H, d), 8.47 (1H, d), 8.16 (3H, m), 2.75 (3H, s), 2.69 (3H, s). LCMS (run time=5 min): Rₜ=3.50 min; m/z 291 [M+H]⁺

### Preparation 5a

### 6-(1-Ethoxyethenyl)-2-[4-(1-ethoxyethenyl)phenyl]quinazoline

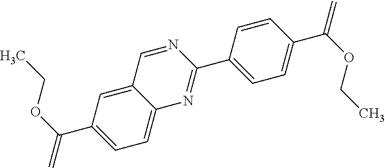

To a stirred solution of 6-bromo-2-(4-bromophenyl)-quinazoline (40 g, 109.88 mmol), obtained from Preparation 4, in DMF (240 mL) was added tributyl(1-ethoxy)vinyltin (99.21 g, 274.7 mmol), and the mixture was degassed with a stream of nitrogen_{(g)} for 30 minutes. PdCl₂(PPh₃)₂ (7.71 g, 10.99 mmol) was added and the mixture was heated at 100° C. for 3 hours. After cooling to room temperature the mixture was concentrated under vacuum to remove the DMF. The residue was dissolved in DCM (600 mL), treated with potassium fluoride solution (115 g in 600 mL H₂O) and stirred at room temperature for 1 hour. The mixture was filtered through celite, whereupon the organic layer was separated and washed with water (2×300 mL). The organic layer was dried over magnesium sulphate, and concentrated in vacuo. The resulting residue was granulated in MeCN and isolated by filtration to afford the title compound as light brown solid (32.1 g).

¹H NMR (400 MHz, DMSO-d₆): δ=9.70 (1H, s), 8.5 (2H, d), 8.35 (1H, d), 8.25 (1H, dd), 8.00 (2H, d), 5.05-4.9 (2H, dd), 4.5-3.9 (2H, dd), 3.9-4.0 (4H, m), 1.4 (6H, m).

### Preparation 6

### 2,2-Dibromo-1-{2-[4-(2,2-dibromoacetyl)phenyl]quinazolin-6-yl}ethanone

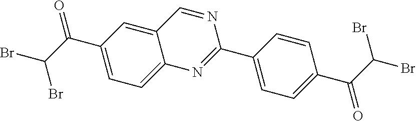

To a stirred solution of 1-[2-(4-acetylphenyl)quinazolin-6-yl]ethanone (1.3 g, 4.4 mmol), obtained from Preparation 5, in HBr (10 mL) was added a solution of Br₂ (1.4 g, 8.8 mmol) in AcOH (1 mL), dropwise at 0° C. The mixture was stirred at room temperature for 1 hour, and was then cooled to 0° C., basified with saturated sodium bicarbonate, and extracted with ethyl acetate (3×20 mL). The combined organic layers were washed with water (1×10 mL) and brine (1×10 mL), dried (Na₂SO₄) and evaporated to dryness. The resulting crude material was purified by column chromatography on silica gel (ethyl acetate:hexane, 1:4) to afford the title compound as a brown liquid (1.4 g).

¹H NMR (400 MHz, DMSO-d₆): δ=9.92 (1H, s), 9.08 (1H, s), 8.75 (2H, m), 8.57 (1H, d), 8.26 (3H, m), 8.00 (1H, s), 7.94 (1H, s).

### Preparation 7

### 2-Bromo-1-{2-[4-(2-bromoacetyl)phenyl]quinazolin-6-yl}ethanone

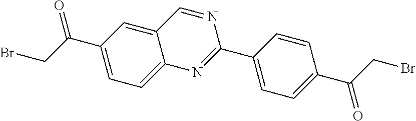

Method A: To a stirred solution of 2,2-dibromo-1-{2-[4-(2,2-dibromoacetyl)phenyl]quinazolin-6-yl}ethanone (1.4 g, 2.3 mmol), obtained from Preparation 6, in THF (40 mL) was added a solution of diethyl phosphite (0.47 g, 3.4 mmol) in THF (5 mL) and Et₃N (0.35 g, 3.4 mmol) at 0° C. The reaction mixture was stirred at room temperature for 4 hours. Water (15 mL) was added to the reaction mixture, which was then extracted with ethyl acetate (2×20 mL). The combined organic layers were washed with brine (1×20 mL), dried (Na₂SO₄), and evaporated to dryness. The crude mass was purified by column chromatography (ethyl acetate:hexane, 1:2) to afford the title compound as a yellow liquid (0.7 g).

¹H NMR (400 MHz, DMSO-d₆): δ=9.92 (1H, s), 9.00 (1H, s), 8.74 (2H, m), 8.52 (1H, d, J=8.7 Hz), 8.20 (3H, m), 5.10-5.02 (4H, m).

Method B: To a stirred solution of 6-(1-Ethoxyethenyl)-2-[4-(1-ethoxyethenyl)phenyl]quinazoline (60.6 g, 174.93 mmol), obtained from Preparation 5a, in THF (2.2 L) was added water (91 mL). To this solution was added N-bromosuccinimide (49.82 g, 279.88 mmol) and the reaction mixture was stirred at room temperature for 1 hour. The organic solution was concentrated to low volume and stripped into MeOH to provide a slurry. The slurry was granulated in MeOH (300 mL) and isolated by filtration to afford the title compound as a yellow solid (57.88 g).

### Preparation 8

### (S)-Pyrrolidine-1,2-dicarboxylic acid 1-tert-butyl ester 2-[2-(4-{6-[2-((S)-1-carboxylic acid tert-butyl-ester-pyrrolidine-2-carbonyloxy)acetyl]-quinazolin-2-yl}-phenyl)-2-oxoethyl]ester

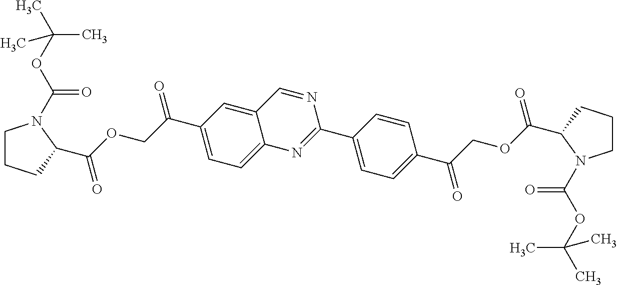

Method A: To a stirred solution of 2-bromo-1-{2-[4-(2-bromoacetyl)phenyl]quinazolin-6-yl}ethanone (0.7 g, 1.5 mmol), obtained from Preparation 7, in acetonitrile (15 mL) was added DIPEA (1.6 mL, 9 mmol) and N-boc-L-proline (0.6 g, 2.7 mmol) at 0° C. The reaction mixture was stirred at room temperature for 1.5 hours. The solvent was evaporated and the crude residue was purified by column chromatography on silica gel (ethyl acetate:hexane, 1:1) to afford the title compound as a light yellow liquid (0.7 g).

¹H NMR (400 MHz, DMSO-d₆): δ=9.92 (1H, s) 9.10 (1H, m), 8.74 (2H, m), 8.49 (1H, m), 8.20 (3H, m), 5.69 (4H, m), 4.36 (2H, m), 3.40 (2H, m), 2.21 (2H, m), 2.16 (2H, m), 1.89 (4H, m), 1.38 (20H, s). LCMS (run time=5 min): Rₜ=4.17 min; m/z 717 [M+H]⁺

Method B: To a stirred solution of 2-bromo-1-{2-[4-(2-bromoacetyl)phenyl]quinazolin-6-yl}ethanone (56.8 g, 126.93 mmol), obtained from Preparation 7, in acetonitrile (570 mL) was added DIPEA (88.55 mL, 507.7 mmol) and N-boc-L-proline (60.11 g, 279.25 mmol) at 0° C. The reaction mixture was stirred at room temperature for 1.5 hours. The solvent was evaporated and the crude residue was dissolved in EtOAc (560 mL) and washed with a saturated aqueous sodium bicarbonate solution (560 mL) and separated. The organic layer was washed with water (300 mL) and separated. The organic layer was concentrated to dryness to afford the title compound as a light orange solid (89.44 g).

### Preparation 9

### (S)-2-[5-(4-{6-[2-((S)-1-carboxylic acid tert-butyl ester-pyrrolidin-2-yl)-3H-imidazol-4-yl]quinazolin-2-yl}phenyl)-1H-imidazol-2-yl]pyrrolidine-1-carboxylic acid tert-butyl ester

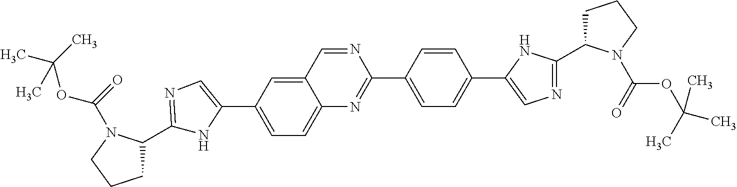

Method A: To a stirred solution of (S)-pyrrolidine-1,2-dicarboxylic acid 1-tert-butyl ester 2-[2-(4-{6-[2-((S)-1-carboxylic acid tert-butyl-ester-pyrrolidine-2-carbonyloxy)acetyl]quinazolin-2-yl}phenyl)-2-oxoethyl]ester (0.1 g, 0.13 mmol), obtained from Preparation 8, in trifluorotoluene (2 mL) was added ammonium acetate (0.24 g, 3.0 mmol). The reaction mixture was heated in a sealed tube at 120° C. for 16 hours. The solvent was removed in vacuo and the residue was partitioned between ethyl acetate (15 mL) and saturated sodium bicarbonate solution (10 mL). The organic layer was separated, washed with brine (1×5 mL), dried over sodium sulphate and was evaporated to dryness. The crude residue was purified by column chromatography on silica gel (ethyl acetate:hexane, 7:3) to afford the title compound as an off white solid (0.07 g).

¹H NMR (400 MHz, DMSO-d₆): δ=9.60 (1H, s), 8.55 (2H, d), 8.39 (2H, d), 8.00 (1H, d), 7.90 (2H, d), 7.62 (1H, s), 7.53 (1H, s), 4.92 (2H, m), 3.58 (2H, m), 3.47 (2H, m), 2.28 (2H, m), 2.09 (4H, m), 11.97 (2H, br s) 1.92 (2H, m), 1.34 (18H, s). LCMS (run time=5 min): Rₜ=2.38 min; m/z 677 [M+H]⁺

Method B: To a stirred solution of (S)-pyrrolidine-1,2-dicarboxylic acid 1-tert-butyl ester 2-[2-(4-{6-[2-((S)-1-carboxylic acid tert-butyl-ester-pyrrolidine-2-carbonyloxy)acetyl]quinazolin-2-yl}phenyl)-2-oxoethyl]ester (88.4 g, 118.39 mmol) in xylenes (1.8 L) was added ammonium acetate (45.63 g, 591.97 mmol) and the reaction mixture was heated to reflux for 8 hours. The solvent was removed in vacuo and the residue was partitioned between dichloromethane (880 mL) and saturated sodium bicarbonate solution (880 mL). The organic layer was separated, washed with brine (2×440 mL), dried over magnesium sulphate and was evaporated to dryness to afford the title compound as an orange solid (77.8 g).

### Preparation 9a

### tert-Butyl (2S)-2-(5-{2-[4-(2-[(2S)-1-(tert-butoxycarbonyl)pyrrolidin-2-yl]-1-{[2-(trimethyl silyl)ethoxy]methyl}-1H-imidazol-5-yl)phenyl]quinazolin-6-yl}-1-{[2-(trimethylsilyl)ethoxy]methyl}-1H-imidazol-2-yl)pyrrolidine-1-carboxylate

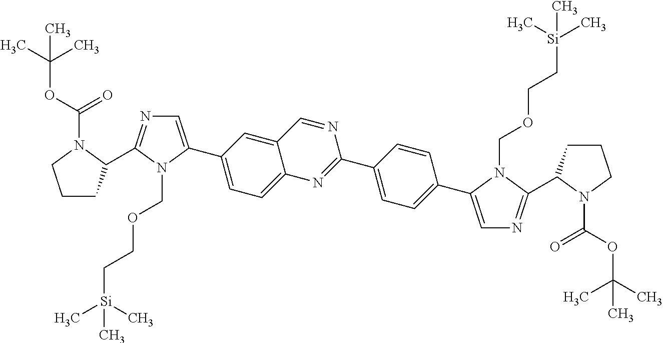

To a stirred solution of the imidazole obtained from Preparation 12 (118.8 g, 302.2 mmol) in N,N-dimethylacetamide (750 mL) was added the dibromoquinazoline obtained from Preparation 4 (50 g, 137.4 mmol), potassium carbonate (57.0 g, 412.4 mmol), Pd(OAc)₂ (3.1 g, 13.8 mmol), pivalic acid (8.5 g, 83.2 mmol) and tricyclohexylphosphine (20% w/w solution in toluene) (38.5 g, 27.5 mmol). The reaction mixture was heated at 130° C. for 16 hours then concentrated to low volume in vacuo. The residue was dissolved in toluene (500 mL), filtered and washed with water (200 mL) and saturated aqueous sodium bicarbonate solution (200 mL). The organic solution was dried (MgSO₄), filtered and concentrated in vacuo to give the desired compound as a brown oil. The crude compound contained a mixture of regioisomers and was taken through the deprotection stage without further purification.

### Preparation 10

### 6-((S)-2-Pyrrolidin-2-yl-3H-imidazol-4-yl)-2-[4-((S)-2-pyrrolidin-2-yl-3H-imidazol-4-yl)phenyl]quinazoline hydrochloride salt

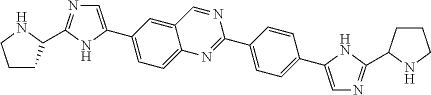

Method A: To a stirred solution of (S)-2-[5-(4-{6-[2-((S)-1-carboxylic acid tert-butyl ester-pyrrolidin-2-yl)-3H-imidazol-4-yl]quinazolin-2-yl}phenyl)-1H-imidazol-2-yl]pyrrolidine-1-carboxylic acid tert-butyl ester (0.45 g, 0.66 mmol), obtained from Preparation 9, in dioxane (2 mL) was added 4M HCl in 1,4-dioxane (10 mL) at 0° C. and the mixture was stirred at room temperature for 3 hours. The dioxane was then evaporated and the residue was triturated with ether (2×10 mL). The crude product was obtained as a light yellow solid (0.38 g) and was used directly for the next step.

¹H NMR (400 MHz, DMSO-d₆): δ=10.31 (2H, br s), 9.79 (1H, br s), 9.71 (1H, s), 9.53 (1H, br s), 8.64 (3H, m), 8.52 (1H, d), 8.13 (5H, m), 4.95 (2H, m), 3.38 (4H, m), 2.32 (3H, m), 2.20 (2H, m), 2.02 (3H, m). LCMS (run time=5 min): Rₜ=1.42 min; m/z 477 [M+H]⁺.

Method B: To a stirred solution of (S)-2-[5-(4-{6-[2-((S)-1-carboxylic acid tert-butyl ester-pyrrolidin-2-yl)-3H-imidazol-4-yl]quinazolin-2-yl}phenyl)-1H-imidazol-2-yl]pyrrolidine-1-carboxylic acid tert-butyl ester (68.28 g, 100.88 mmol), obtained from Preparation 9, in 1,4-dioxane (340 mL) was added 4M HCl in 1,4-dioxane (680 mL) at room temperature and the mixture was stirred at room temperature for 3 hours. The dioxane was then evaporated and the residue was triturated with acetonitrile to afford the crude product obtained as a light orange solid (55.44 g) as the tetra HCl salt and was used directly for the next step.

Method C: To a stirred solution of the compound obtained from Preparation 9a (32.2 g, 34.35 mmol), in absolute ethanol (150 mL), was added 4N HCl in 1,4-dioxane (101 mL, 404 mmol) and the mixture was stirred at 75° C. for 1.5 hours. It was then cooled to ambient temperature and collected by filtration, to yield the desired compound (12.5 g) as a red solid.

### Preparation 11

### tert-Butyl (2S)-2-(1H-imidazol-2-yl)pyrrolidine-1-carboxylate

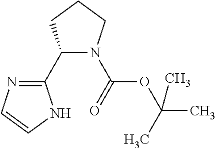

### Method A:

(i) To a solution of (S)-Boc-2-prolinol (198.7 g, 987.1 mmol) in DCM (596 mL) was added sodium bromide (12.2 g, 119 mmol), sodium bicarbonate (12.44 g, 148.1 mmol), water (257 mL) and 2,2,6,6-tetramethylpiperidine-N-oxide (TEMPO) (1.54 g, 9.87 mmol). The reaction mixture was cooled to 0° C. and 1.35M sodium hypochlorite aqueous solution (794 mL, 1066 mmol) was added over 90 minutes. The organic phase was separated and the aqueous layer was extracted with DCM (200 mL). The combined organic layers were washed with 1M sodium thiosulfate aqueous solution (1.07 L) and water (500 mL), then dried (MgSO₄), filtered and concentrated, to give 148.6 g of product as a pale orange oil. GCMS (System 7) (run time 13.5 min) Rₜ 8.61 min m/z 200 [M+H]⁺
(ii) Glyoxal (4.60 mL of 40% in water) was added dropwise over 15 minutes to a cooled (ice/water) solution of the compound obtained in (i) (2.00 g, 10.00 mmol) in ammonia, 28% w/w aqueous solution (7.82 mL) and methanol (6.00 mL). After 10 minutes the reaction mixture was allowed to warm to room temperature and stirred for a further 19 hours. The mixture was concentrated in vacuo and the residue was purified by column chromatography (silica gel, ethyl acetate) followed by a recrystallisation (ethyl acetate, room temperature) to give 1.48 g of the title compound as a white solid.

¹H-NMR (400 MHz, DMSO-d₆): δ=11.71+11.63 (1H, br s), 6.96 (1H, s), 6.77 (1H, s), 4.77 (1H, m), 3.49 (1H, m), 3.31 (1H, m), 2.19-1.80 (4H, m), 1.39/1.14 (9H, s). LRMS (APCI): m/z [M+H]⁺238.

Method B:

(i) To a solution of (S)-Boc-2-prolinol (198.7 g, 987.1 mmol) in DCM (596 mL) was added sodium bromide (12.2 g, 119 mmol), sodium bicarbonate (12.44 g, 148.1 mmol), water (257 mL) and 2,2,6,6-tetramethylpiperidine-N-oxide (TEMPO) (1.54 g, 9.87 mmol). The reaction mixture was cooled to 0° C. and 1.35M sodium hypochlorite aqueous solution (794 mL, 1066 mmol) was added over 90 minutes. The organic phase was separated and the aqueous layer was extracted with DCM (200 mL). The combined organic layers were washed with 1M sodium thiosulfate aqueous solution (1.07 L) and water (500 mL), then dried (MgSO₄), filtered and concentrated, to give 148.6 g of product as a pale orange oil. GCMS (System 7) (run time 13.5 min) Rₜ 8.61 min m/z 200 [M+H]⁺

(ii) Glyoxal (264 mL of 40% in water) was added dropwise over 15 minutes to a cooled (ice/water) solution of the compound obtained from (i) (104.4 g, 523.9 mmol) in ammonia, 28% w/w aqueous solution (230 mL) and methanol (418 mL). After 10 minutes the reaction mixture was allowed to warm to room temperature and stirred for a further 19 hours. The solution was concentrated, redissolved in DCM (200 mL), filtered through a bed of silica gel and concentrated. The residue was triturated in TBME (500 mL) and collected by filtration, then dried in vacuo to give 86.1 g of the product as a white solid.

### Preparation 12

### tert-Butyl (2S)-2-(1-{[2-(trimethylsilyl)ethoxy]methyl}-1H-imidazol-2-yl)pyrrolidine-1-carboxylate

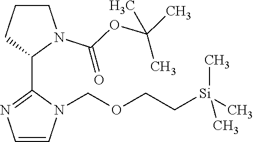

Method A: To a stirred solution of the compound obtained from Preparation 11 (1.80 g, 7.62 mmol) in DMF (18 mL) at 0° C. under nitrogen was added sodium hydride (320 mg, 8.00 mmol of a 60% dispersion in oil) portionwise and the mixture was stirred for 20 minutes. 2-(trimethylsilyl)ethoxymethyl chloride (1.48 mL, 8.38 mmol) was added dropwise. The mixture was allowed to warm to room temperature and stirred for 90 minutes. It was then quenched with water (15 mL) and the product was extracted with ethyl acetate (3×20 mL). The combined organic layers were washed with brine (3×20 mL), dried (Na₂SO₄), filtered and concentrated in vacuo. The resulting crude material was purified by column chromatography (silica gel, ethyl acetate) to give 1.98 g of the title compound as a colourless oil.

¹H-NMR (400 MHz, DMSO-d₆): δ=7.17 (1H, m), 6.81 (1H, m), 5.62/5.40 (1H, m), 5.27 (1H, d), 4.92 (1H, dd), 3.53-3.36 (4H, m), 2.15 (2H, m), 1.85 (2H, m), 1.35/1.14 (9H, s), 0.88 (2H, m), 0.00 (9H, s). LRMS (APCI): m/z [M+H]⁺368.

Method B: To a stirred solution of the compound obtained from Preparation 11 (86.1 g, 362.6 mmol) in THF (860 mL) at 0° C., was added sodium hydride (160 g, 398.9 mmol of a 60% dispersion in oil), portionwise, and the mixture was stirred for 20 minutes. 2-(Trimethylsilyl)ethoxymethyl chloride (68.8 mL, 388.0 mmol) was added dropwise. The mixture was allowed to warm to room temperature and stirred for 16 hours. It was then quenched with water (172 mL), stirred for 30 minutes, diluted with water (200 mL) and extracted with TBME (400 mL). The organic layer was washed with water (2×200 mL), dried (MgSO₄), filtered and concentrated in vacuo. The resulting crude material was redissolved in DCM (200 mL), filtered through a plug of silica and washed through with TBME (1000 mL). The TBME washings were combined and concentrated in vacuo to give 133.2 g of the product as a straw coloured oil that crystallized on standing.

### Preparation 13

### tert-Butyl (2S)-2-(5-bromo-1-{[2-(trimethylsilyl)ethoxy]methyl}-1H-imidazol-2-yl)pyrrolidine-1-carboxylate

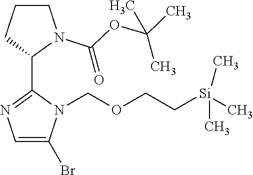

Method A: To a stirred solution of the compound obtained from Preparation 12 (1.98 g, 5.39 mmol) in DCM (50 mL) was added NBS (1.15 g, 6.46 mmol), portionwise, and the mixture was stirred at room temperature for 1 hour. Saturated NaHCO₃ (aq) (20 mL) was added and the mixture was filtered through a phase separation column. The filtrate was concentrated in vacuo. The resulting crude material was purified by column chromatography (silica gel, 15 to 30% ethyl acetate, heptane) to give 1.96 g of the title compound as a colourless oil.

¹H-NMR (400 MHz, DMSO-d₆): δ=6.96 (1H, d), 5.60+5.53 (1H, m), 5.29 (1H, m), 4.97 (1H, m), 3.62-3.35 (4H, m), 2.28-1.81 (4H, m), 1.32+1.15 (9H, s), 0.85 (2H, m), 0.00 (9H, s). LRMS (APCI): m/z [M+H]⁺446 and 448.

Method B: To a stirred solution of the compound obtained from Preparation 12 (850 g, 2.31 mol) in DCM (8.5 L) was added NBS (411.6 g, 2.31 mol) as a solution in acetonitrile (4.25 L) over 1 hour and the mixture was stirred at room temperature for an additional 1 hour. 10% wt/vol sodium metabisulfite solution (aq) (2.2 L) was added and the mixture was stirred for 30 minutes. The layers were separated and triethylamine (322.3 mL, 2.31 mol) and water (2.1 L) were added to the organic phase. The mixture was stirred for 30 minutes. The layers were separated and the organic phase was washed with water (2×2.1 L), dried (MgSO₄), filtered and concentrated in vacuo to give 1004 g of product as a yellow oil.

### Preparation 13a

### tert-Butyl (2S)-2-(5-iodo-1-{[2-(trimethylsilyl)ethoxy]methyl}-1H-imidazol-2-yl)pyrrolidine-1-carboxylate

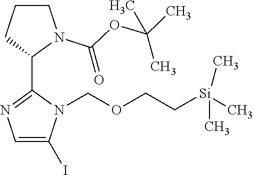

Iodine (3.08 g, 12.1 mmol) was added to a stirring solution of the compound obtained from Preparation 12 (7.44 g, 20.2 mmol) in acetonitrile (125 mL) at room temperature. (Diacetoxyiodo)benzene (7.82 g, 24.3 mmol) was added and the mixture was stirred for 16 hours in the dark. The solvent was removed under reduced pressure and the resulting orange oil was dissolved in TBME (100 mL). The mixture was washed with saturated sodium thiosulphate solution (2×100 mL), dried (MgSO₄), and the solvent was removed under reduced pressure to give a yellow oil. The compound was purified by column chromatography (SiO₂) using heptanes: ethyl acetate (9:1 to 7:3) to give 5.83 g of a yellow oil.

¹H-NMR (400 MHz, DMSO-d₆): δ=6.95 (1H, m), 5.58+5.40+5.23 (2H, 3×m), 5.02-4.92 (1H, m), 3.57-3.50 (2H, m), [3.46-3.41]+[3.37-3.30](2H, 2×m), [2.20-2.09]+[2.06-1.97](2H, 2×m), 1.89-1.77 (2H, m), 1.35+1.12 (9H, 2×s), 0.93-0.80 (2H, m), −0.03 (9H, s). LCMS (run time=4.5 min): Rₜ=3.54 min; m/z 494 [M+H]⁺

### Preparation 13b

### (2-[(2S)-1-(Tert-butoxycarbonyl)pyrrolidin-2-yl]-1-{[2 (trimethylsilyl)ethoxy]methyl}-1H-imidazol-5-yl)boronic acid

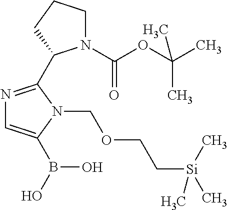

The title compound was made in two different ways (if the bromide described in Preparation 13 is used, it must be used immediately on formation):

Method A: The iodide obtained from Preparation 13a (3.28 g, 6.65 mmol) was dissolved in THF (35 mL) and cooled to 0° C. The mixture was put under nitrogen and isopropylmagnesium chloride-lithium chloride complex (14% solution in THF, 9.97 mL, 9.97 mmol) was added, dropwise. The mixture was stirred at this temperature for 1 hour. Trimethyl borate (1.19 mL, 10.6 mmol) was then added to the mixture. It was allowed to warm to room temperature and stirred for 16 hours. Water (60 mL) was added to the mixture, followed by saturated sodium bicarbonate solution (40 mL). The mixture was extracted with ethyl acetate (3×60 mL) and the combined organic fractions were washed with brine, dried (MgSO₄) and the solvent was removed under reduced pressure to give 2.72 g of the title compound as a yellow solid.

LCMS (run time=2 min): Rₜ=1.34 min; m/z 412 [M+H]⁺

Method B: The bromide obtained from Preparation 13 (4.8 g, 10.75 mmol) was dissolved in THF (120 mL) and cooled to 0° C. The mixture was put under nitrogen and isopropylmagnesium chloride-lithium chloride complex (14% solution in THF, 16.1 mL, 16.1 mmol) was added dropwise. The mixture was stirred at this temperature for 1 hour. Trimethyl borate (1.92 mL, 17.2 mmol) was added to the mixture. It was allowed to warm to room temperature and stirred for 16 hours. Water (60 mL) was added to the mixture followed by saturated sodium bicarbonate solution (40 mL). The mixture was extracted with ethyl acetate (3×60 mL) and the combined organic fractions were washed with brine and dried with MgSO₄. The solvent was removed under reduced pressure to give 3.4 g of the title compound as a yellow solid.

### Preparation 14

### (S)-2-(2-(4-Bromophenyl)-2-oxoethyl) 1-tert-butyl pyrrolidine-1,2-dicarboxylate

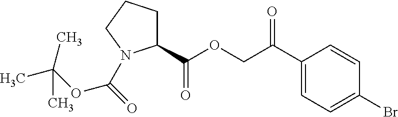

Method A: 2,4′-dibromoacetophenone (23.7 g, 85.4 mmol) was added to a stirring solution of Boc-L-proline (17.5 g, 81.3 mmol) in dichloromethane (175 mL) at 0° C. DIPEA (15.6 mL, 89.4 mmol) was added dropwise to the mixture. The resulting yellow solution was allowed to warm to room temperature and stirred for a further 2.5 hours. The mixture was washed with water (200 mL), saturated sodium bicarbonate solution (100 mL), water (200 mL) and brine (200 mL). The organic phase was dried (MgSO₄) and evaporated under reduced pressure to give a viscous yellow oil (33.6 g).

¹H NMR (400 MHz, T=90° C., DMSO-d₆): δ=7.91-7.85 (2H, m), 7.77-7.71 (2H, m), 5.48 (1H, d), 5.41 (1H, d), 4.34 (1H, dd), 3.40-3.35 (2H, m), 2.34-2.23 (1H, m), 2.14-2.05 (1H, m), 1.96-1.84 (2H, m), 1.40 (9H, s).

LRMS (APCI): m/z 312 and 314 [(M-Boc)+2H^{+].}

Method B: 2,4′-dibromoacetophenone (13.09 Kg, 47.1 mol) was added to a stirring solution of Boc-L-proline (9.67 Kg, 44.9 mol) in dichloromethane (48 L) at 5° C. DIPEA (6.38 Kg, 49.4 mol) was added dropwise to the mixture. The resulting yellow solution was allowed to warm to room temperature and stirred for a further 2.5 hours. The mixture was washed with water (25 L), saturated sodium bicarbonate solution (25 L), water (25 L) and brine (25 L). The organic phase was dried (Na₂SO₄) and evaporated under reduced pressure to give a viscous yellow oil (18.51 Kg).

### Preparation 15

### (S)-tert-Butyl 2-(5-(4-bromophenyl)-1H-imidazol-2-yl)pyrrolidine-1-carboxylate

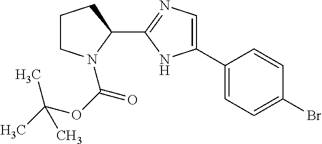

Method A: Ammonium acetate (44.6 g, 0.58 mmol) was added to a solution of the compound obtained from Preparation 14 (53 g, 0.13 mol) in xylenes (250 mL) and the resulting mixture was heated at 150° C. for 5 hours. After cooling, the mixture was filtered and the solvent was removed under reduced pressure. The resulting yellow solid was stirred in TBME (75 mL) for 1 hour and the solid that resulted was filtered off and dried to give 29.1 g of the title compound as a white solid.

¹H NMR (400 MHz, T=90° C., DMSO-d₆): δ=7.68 (2H, d), 7.55 (1H, s), 7.47 (2H, d), 4.86-4.76 (1H, m), 3.58-3.48 (1H, m), 3.45-3.34 (1H, m), 2.24-2.15 (1H, m), 2.06-1.99 (2H, m), 1.91-1.79 (1H, m), 1.29 (9H, s). LRMS (APCI): m/z [M+H]⁺392; 394.

Method B: Ammonium acetate (17.30 Kg, 224.4 mol) was added to a solution of the compound obtained from Preparation 14 (18.51 Kg, 44.9 mol) in xylenes (92.5 L) and the resulting mixture was heated at 130 to 135° C. for 5 hours. After cooling, the mixture was washed with water (22.5 L) and the aqueous layer was back extracted with ethyl acetate (22.5 L). The combined organic layers were washed with water (22.5 L), dried (Na₂SO₄), filtered and evaporated. The residue was suspended in TBME (100 L) and the resulting yellow solid was filtered off, washed with TBME (22.5 L) and dried to give 13.0 Kg of the title compound as a white solid.

### Preparation 16

### (S)-tert-Butyl 2-(5-(4-(4,4,5,5-tetramethyl-1,3,2-dioxaborolan-2-yl)phenyl)-1H-imidazol-2-yl)pyrrolidine-1-carboxylate

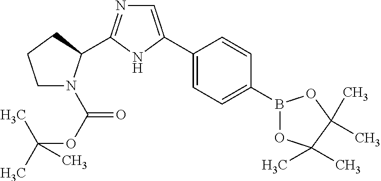

Method A: A mixture of the bromide obtained from Preparation 15 (3.00 g, 7.65 mmol), bis(pinacolato)diboron (3.88 g, 15.3 mmol), and potassium acetate (1.88 g, 19.1 mmol) were dissolved in 1,4-dioxane (15.3 mL). The mixture was degassed and then nitrogen filled three times. Pd(dppf)Cl₂.DCM (191 mg, 0.77 mmol) was added. The reaction mixture was degassed and put under nitrogen twice more. It was refluxed for 2 hours and then allowed to cool. The mixture was partitioned between ethyl acetate (100 mL) and water (100 mL). The pH of the aqueous layer was adjusted to around 8 by addition of a 2N aqueous sodium hydroxide solution and then the phases were separated. The aqueous phase was extracted again with ethyl acetate (100 mL). The combined organic layers were dried (MgSO₄) and the solvent was evaporated under reduced pressure. The crude material was purified using column chromatography on silica (50% EtOAc in Heptane to 60% EtOAc in heptane) to give the desired compound as a white foam. This material was dissolved in EtOH (10 ml) and was heated to reflux. Water (20 mL) was added and the cloudy suspension was allowed to cool to room temperature slowly. The material was filtered and washed with water and dried to give 2.54 g of the title compound as a white solid.

LCMS (run time=6 min): Rₜ=3.46 min; m/z 440 [M+H]+.

Method B: A mixture of the bromide obtained from Preparation 15 (12.1 Kg, 30.84 mol), bis(pinacolato)diboron (8.224 Kg, 32.38 mol), and potassium acetate (7.57 Kg, 77.1 mol) were dissolved in 1,4-dioxane (85 L). The mixture was degassed and then nitrogen filled three times. Pd(dppf)Cl₂.DCM (918 g, 1.124 mol) was added. The reaction mixture was degassed and put under nitrogen twice more. The mixture was refluxed for 2 hours and then allowed to cool. The solvent was evaporated and the residue was partitioned with ethyl acetate (120 L) and water (120 L). The biphasic mixture was filtered and then separated. The pH of the aqueous phase was adjusted to around 8 by the addition of 1M aqueous sodium hydroxide solution and then extracted twice with ethyl acetate (2×60 L). The combined organic phases were washed with water (60 L), dried (Na₂SO₄), and filtered. The solvent was evaporated under reduced pressure. The resulting residue was triturated in hexanes (20 L), filtered and washed with further hexanes (20 L). The product was dried to give 10.79 Kg of the title compound as a white solid.

LCMS (run time=6 min): Rₜ=3.46 min; m/z 440 [MH]⁺.

### Preparation 17

### tert-Butyl (2S)-2-[5-(6-{[tert-butyl(dimethyl)silyl]oxy}naphthalen-2-yl)-1-{[2-(trimethyl silyl)ethoxy]methyl}-1H-imidazol-2-yl]pyrrolidine-1-carboxylate

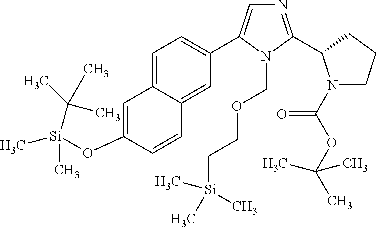

(6-{[tert-Butyl(dimethyl)silyl]oxy}naphthalen-2-yl)boronic acid (731 mg, 2.42 mmol), the bromide obtained from Preparation 13 (900 mg, 2.02 mmol) and Pd(dppf)Cl₂.DCM (100 mg, 0.40 mmol) were added to a microwave vial, followed by 1,2-dimethoxyethane (6.54 mL) and 2M saturated sodium carbonate solution (3.02 mL, 6.05 mmol). The mixture was heated under microwave irradiation at 120° C. for 20 minutes with cooling. The resulting mixture was dry loaded onto silica and purified by column chromatography on silica gel (Redisep 40 g, 0-100% ethyl acetate, heptane) to give 936 mg of the title compound as a white gum.

¹H-NMR (400 MHz, CDCl₃): δ=8.18 (1H, s), 7.78 (1H, dd), 7.73 (1H, d), 7.65 (1H, d), 7.21 (1H, s), 7.14 (1H, m), 7.03 (1H, m), 5.87+5.40 (1H, m), 5.21 (1H, d), 5.00 (1H, m), 3.78-3.51 (4H, m), 2.37-1.87 (4H, m), 1.53-0.86 (17H, m), 0.23 (9H, s), 0.00 (9H, s). LRMS (APCI): m/z [M+H⁺]624.

### Preparation 18

### tert-Butyl (2S)-2-[5-(6-hydroxynaphthalen-2-yl)-1-{[2-(trimethylsilyl)ethoxy]methyl}-1H-imidazol-2-yl]pyrrolidine-1-carboxylate

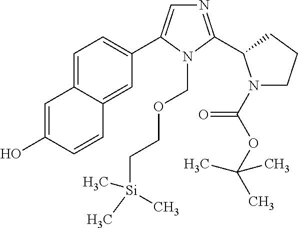

To a stirred solution of the silylated phenol obtained from Preparation 17 (842 mg, 1.35 mmol) in THF (10 mL), was added tetrabutylammonium fluoride (1.62 mL of a 1N solution in THF, 1.62 mmol). The mixture was stirred at room temperature for 5 minutes and then ethyl acetate (10 mL) was added, followed by washing with water (10 mL) and brine (10 mL). It was dried (Na₂SO₄) and then the solvent was removed under reduced pressure. The resulting crude material was purified by column chromatography on silica gel (redisep (40 g), 15 to 60% ethyl acetate, heptane) to give 652 mg of the title compound as an off white solid.

¹H-NMR (400 MHz, CDCl₃): δ=8.30-7.44 (4H, m), 7.14-6.98 (3H, m), 5.90-4.91 (3H, m), 3.80-3.48 (4H, m), 2.36, 1.89 (4H, m), 1.55-0.87 (11H, m), 0.00 (9H, s). LRMS (APCI): m/z [M+H⁺] 510.

### Preparation 19

### tert-Butyl (2S)-2-[5-(6-{[(trifluoromethyl)sulfonyl]oxy}naphthalen-2-yl)-1-{[2-(trimethyl silyl)ethoxy]methyl}-1H-imidazol-2-yl]pyrrolidine-1-carboxylate

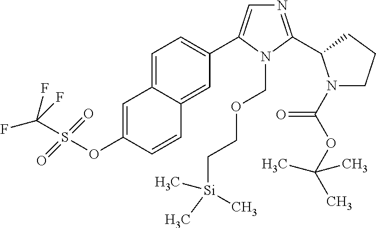

To a stirred solution of the phenol obtained from Preparation 18 (440 mg, 0.86 mmol) and triethylamine (0.27 mL, 1.90 mmol) in DCM (5 mL) at −78° C. under nitrogen was added trifluoromethanesulphonic anhydride (196 μL, 1.16 mmol). The mixture was stirred at −78° C. for 10 minutes. It was then quenched with saturated sodium bicarbonate solution (5 mL) and filtered through a phase separation column. The filtrate was concentrated under reduced pressure. The resulting crude material was purified by column chromatography on silica gel (redisep (12 g), 0 to 50% ethyl acetate, heptane) to give 550 mg of the title compound as a colourless oil.

¹H-NMR (400 MHz, CDCl₃): δ=8.48-7.33 (7H, m), 5.93-5.32 (3H, m), 3.69-3.58 (4H, m), 2.51-1.89 (4H, m), 1.55-0.86 (11H, m), 0.00 (9H, s). LRMS (APCI): m/z [M+H⁺] 642.

### Preparation 20

### tert-Butyl (2S)-2-(5-[6-(4-{2-[(2S)-1-(tert-butoxycarbonyl)pyrrolidin-2-yl]-1H-imidazol-4-yl}phenyl)naphthalen-2-yl]-1-{[2-(trimethylsilyl)ethoxy]methyl}-1H-imidazol-2-yl)pyrrolidine-1-carboxylate

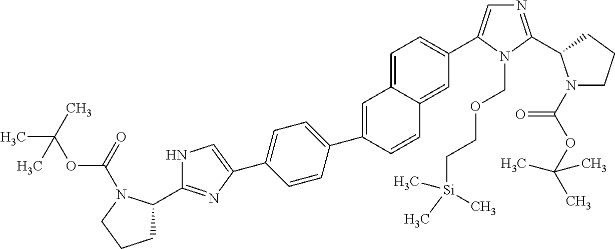

The boronic ester obtained from Preparation 16 (188 mg, 0.43 mmol), the triflate obtained from Preparation 19 (250 mg, 0.39 mmol) and Pd(dppf)Cl₂.DCM (20 mg, 0.08 mmol) were added to a microwave vial (Biotage, 2.5-5 mL), followed by 1,2-dimethoxyethane (1.27 mL) and 2M aqueous sodium carbonate solution (0.59 mL, 1.17 mmol). The mixture was heated under microwave irradiation at 120° C. for 20 minutes with cooling. The reaction was dry loaded onto silica and purified by column chromatography on silica gel (Redisep 12 g, 10-50% ethyl acetate, heptane) to give 251 mg of the title compound as a pale orange foam.

LCMS (run time=6 min): Rₜ=2.65 min; m/z [M+H]⁺805.

### Preparation 21

### 2-[(2S)-Pyrrolidin-2-yl]-4-[4-(6-{2-[(2S)-pyrrolidin-2-yl]-1H-imidazol-5-yl}naphthalen-2-yl)phenyl]-1H-imidazole

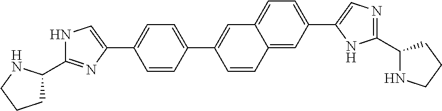

To a stirred solution of the compound obtained from Preparation 20 (378 mg, 0.47 mmol) in ethanol (3 ml), was added 2N HCl in 1,4-dioxane (3 mL, 94 mmol). The mixture was stirred at 75° C. for 5 hours. It was then concentrated under reduced pressure. The residue was dissolved in methanol (5 mL) and loaded onto an SCX column, washing with more methanol. The product was collected by flushing with 7N ammonia in methanol and the filtrate was concentrated under reduced pressure to give 192 mg of the title compound as a pale yellow foam.

¹H-NMR (400 MHz, DMSO-d₆): δ=12.12 (1H, brs), 8.25 (1H, s), 8.17 (1H, s), 7.08-7.76 (8H, m), 7.68-7.54 (2H, m), 6.23 (1H, brs), 4.36 (2H, m), 4.10 (1H, brs), 3.05 (4h, m), 2.16 (2H, m), 2.00 (2H, m), 1.84 (4H, m).

LRMS (APCI): m/z [M+H⁺] 475.

### Preparation 22

### N-(Methoxycarbonyl)-L-valine

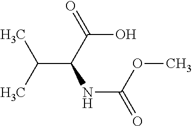

Method A: Methyl chloroformate (73.3 mL, 0.953 mol) was added dropwise to a stirring solution of sodium carbonate (45.9 g, 0.433 mol) and L-valine (101.5 g, 0.866 mol) in 1N sodium hydroxide solution (870 mL, 0.87 mol) at 0° C. The mixture was allowed to warm to room temperature and stirred for 4 hours. It was then washed with TBME (2×400 mL) and the aqueous phase was cooled to 0° C. before being acidified to pH 1 with 6N hydrochloric acid. The cloudy suspension was then extracted with DCM (6×500 mL). The combined organic fractions were dried (Na₂SO₄) and the solvent was removed under reduced pressure to give 126.5 g of the title compound as a white solid.

¹H-NMR (400 MHz, CDCl₃): δ=12.52 (1H, s), 7.30 (1H, d), 3.82 (1H, dd), 3.52 (3H, s), 2.00 (1H, m), 0.86 (6H, t).

Method B: L-valine (200 g, 1.707 mol) was added to a stirred mixture of sodium hydroxide (150.2 g, 3.755 mol), water (1000 mL) and toluene (1000 mL), then cooled to 0° C. Methylchloroformate (145.3 mL, 1.880 mol) was added over 30 minutes, and then the reaction mixture was stirred overnight at room temperature. The phases were separated. The aqueous layer was acidified with 5M sulfuric acid (800 mL, 4.0 mol) and then extracted with ethyl acetate (2×500 mL). The combined organic phases were washed with water (500 mL) and concentrated in vacuo. The solid was dried in vacuo at 45° C. to give 216 g of the desired product as a white solid.

### Preparation 23

### 2-Chloro-6-(4,4,5,5-tetramethyl-[1,3,2]dioxaborolan-2-yl)-quinoline

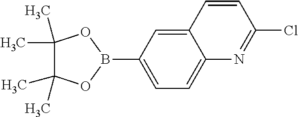

6-Bromo-2-chloro-quinoline (200 mg, 0.825 mmol), bis(pinacolato)diboron (210 mg, 0.825 mmol), potassium acetate (202 mg, 2.06 mmol) and Pd(dppf)Cl₂.DCM (21 mg, 0.083 mmol) were added to a microwave vial, followed by 1,4-dioxane (2 mL). The mixture was heated under microwave irradiation at 120° C. for 30 minutes. It was then partitioned between ethyl acetate and water. The organic layer was evaporated under reduced pressure and purified by column chromatography on silica gel (Redisep 12 g, eluting with a gradient of heptane:ethyl acetate (100:0 to 0:100) to afford 155 mg of the title compound as a pale buff solid.

LCMS (run time=2 min): Rₜ=1.90 min; m/z [M+H]⁺290.

### Preparation 24

### (S)-2-[4-(2-Chloro-quinolin-6-yl)-1-(2-trimethylsilanyl-ethoxymethyl)-1H-imidazol-2-yl]-pyrrolidine-1-carboxylic acid tert-butyl ester

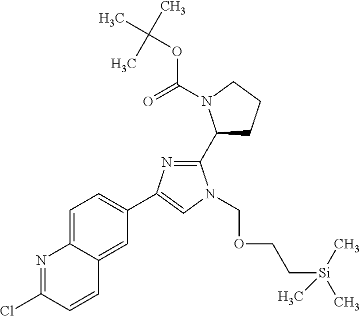

2-Chloro-6-(4,4,5,5-tetramethyl-[1,3,2]dioxaborolan-2-yl)-quinoline (80 mg, 0.28 mmol), obtained from Preparation 23, tert-butyl (2S)-2-(5-bromo-1-{[2-(trimethylsilyl)ethoxy]methyl}-1H-imidazol-2-yl)pyrrolidine-1-carboxylate (123 mg, 0.28 mmol), obtained from Preparation 13, and Pd(dppf)Cl₂.DCM (23 mg, 0.028 mmol) were added to a microwave vial, followed by 1,2-dimethoxyethane (1 mL) and 2N sodium bicarbonate solution (414 μL, 0.828 mmol). The mixture was heated under microwave irradiation at 120° C. for 20 minutes. It was then partitioned between ethyl acetate and water. The organic layer was evaporated under reduced pressure and purified by column chromatography on silica gel (Redisep 4 g, eluting with a gradient of heptane:ethyl acetate (100:0 to 0:100) to give 32 mg of the title compound as a pale brown solid.

LCMS (run time=2 min): Rₜ=1.60 min; m/z [M+H]⁺529; 531.

### Preparation 25

### ((S)-1-{(S)-2-[4-(2-Chloro-quinolin-6-yl)-1-(2-trimethylsilanyl-ethoxymethyl)-1H-imidazol-2-yl]-pyrrolidine-1-carbonyl}-2-methyl-propyl)-carbamic acid methyl ester

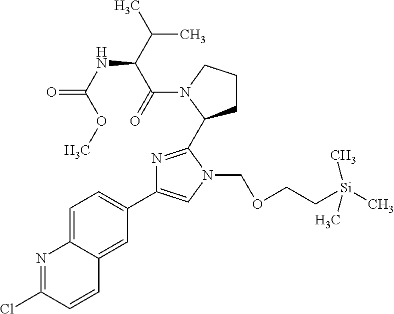

(S)-2-[4-(2-Chloro-quinolin-6-yl)-1-(2-trimethylsilanyl-ethoxymethyl)-1H-imidazol-2-yl]-pyrrolidine-1-carboxylic acid tert-butyl ester (32 mg, 0.06 mmol), obtained from Preparation 24, was treated with 1:1 dichloromethane:trifluoroacetic acid (2 mL) at room temperature for 1 hour. The reaction was concentrated and applied to an SCX cartridge, eluting initially with methanol then 7N ammonia in methanol, to elute the intermediate 2-chloro-6-[(S)-2-pyrrolidin-2-yl-1-(2-trimethylsilanyl-ethoxymethyl)-1H-imidazol-4-yl]-quinoline.

LCMS (run time=2 min): Rₜ=1.34 min; m/z [MH]⁺ 429; 431.

To a stirred solution of N-(methoxycarbonyl)-L-valine (10.5 mg, 0.06 mmol), obtained from Preparation 22, in DMF (1 mL), was added HATU (25 mg, 0.066 mmol) and diisopropylethylamine (15.5 μL, 0.12 mmol). After stirring at room temperature for 30 minutes, 2-chloro-6-[(S)-2-pyrrolidin-2-yl-1-(2-trimethylsilanyl-ethoxymethyl)-1H-imidazol-4-yl]-quinoline was added in DMF (0.5 mL) and the mixture was stirred at room temperature for 1 hour. It was then partitioned between ethyl acetate and water, and the organic layer was evaporated under reduced pressure. The resulting crude material was purified by column chromatography on silica gel (Redisep 4 g, eluting with a gradient of DCM through toDCM:MeOH:NH₃90:10:1) to give the title compound as a pale yellow solid which was used as was in the next step.

LCMS (run time=2 min): Rₜ=1.57 min; m/z [M+H]⁺ 586; 588.

### Preparation 26

### (S)-5-(4-Bromophenyl)-2-(pyrrolidin-2-yl)-1H-imidazole

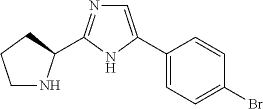

The compound obtained from Preparation 15 (10.0 g, 25.5 mmol) was added in portions to a stirring solution of trifluoroacetic acid (50 mL). The resulting yellow solution was stirred at room temperature for 1 hour and then the solvent was removed under reduced pressure. Water (25 mL) was added to the residue, then saturated aqueous sodium bicarbonate was added until the mixture was basic (pH=8). The mixture was extracted with ethyl acetate (3×200 mL) and the combined organic fractions were dried (MgSO₄). The solvent was removed under reduced pressure to give the title compound as a yellow solid.

¹H NMR (400 MHz, CDCl₃): δ=7.47 (4H, s), 7.15 (1H, s), 4.70 (1H, t), 4.11 (1H, br.s), 3.43-3.32 (1H, m), 3.29-3.16 (1H, m), 2.44-2.33 (2H, m), 2.22-2.09 (1H, m), 2.08-1.96 (1H, m). LRMS (APCI): m/z [M+H]⁺292; 294

### Preparation 27

### Methyl (S)-1-((S)-2-(5-(4-bromophenyl)-1H-imidazol-2-yl)pyrrolidin-1-yl)-3-methyl-1-oxobutan-2-yl carbamate

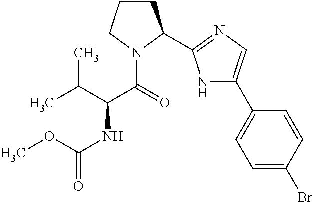

HATU (7.16 g, 18.8 mmol) was added to a stirring solution of the acid obtained from preparation 22 (3.3 g, 18.8 mmol) and DIPEA (5.96 mL, 34.2 mmol) in DMF (20 mL) at room temperature. A solution of the compound obtained from Preparation 26 (5.0 g, 17.1 mmol) in DMF (10 mL) was added to the mixture and it was stirred at room temperature for 16 hours. The solvent was removed under reduced pressure and the residue was washed with a 10% solution of aqueous potassium carbonate to give a brown gum which was taken up in dichloromethane. The solvent was removed under reduced pressure and the residue was purified by column chromatography (silica gel, 100% dichloromethane to 97:3 dichloromethane/methanol) to give the desired product contaminated with DMF. The material was taken up in diethylether (40 mL), washed with water (3×40 mL), dried (MgSO₄) and the solvent was removed under reduced pressure to give 5.55 g of the title compound as a yellow foam.

LCMS (run time=6 min): Rₜ=2.29 min; m/z [M+H]⁺ 449; 451

### Preparation 28

### Methyl (S)-3-methyl-1-oxo-1-((S)-2-(5-(4-(4,4,5,5-tetramethyl-1,3,2-dioxaborolan-2-yl)-phenyl)-1H-imidazol-2-yl)-pyrrolidin-1-yl)butan-2-yl carbamate

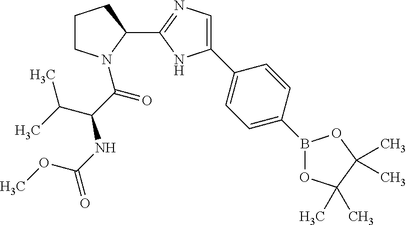

The compound obtained from Preparation 27 (3.0 g, 6 mmol), bis(pinacolato)diboron (3.05 g, 12.0 mmol), and KOAc (1.47 g, 15.0 mmol) were dissolved in 1,4-dioxane (15 mL) and put in a microwave vial. Pd(dppf)Cl₂.DCM (150 mg, 0.60 mmol) was added. The reaction mixture was degassed and put under nitrogen. The mixture was heated under microwave irradiation at 120° C. for 3 hours. The resulting mixture was diluted with dichloromethane (20 mL) and filtered through celite. The resulting solution was evaporated under reduced pressure to give a black tar which was purified by column chromatography (silica gel, 8:2 heptane:ethyl acetate) to give 2.5 g of the title compound as a pale yellow solid.

LRMS (ES): m/z [M+H]⁺ 497.

### Preparation 29

### 6-Bromoquinoxalin-2(1H)-one

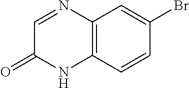

Method A: Quinoxalin-2(1H)-one (5 g, 34.2 mmol) was stirred in sulphuric acid (50 mL) and silver sulphate was added (5.3 g, 17.1 mmol). The mixture was vigorously stirred until complete dissolution, whereupon bromine (1.76 mL, 34.2 mmol) was added dropwise. The resulting mixture was stirred at room temperature under nitrogen overnight. Carbon tetrachloride (50 mL) was added and the mixture was heated to 50° C. for 30 minutes, then filtered. The solid was washed with carbon tetrachloride and the filtrate was collected. This was poured into a beaker of crushed ice and slurried to give a thick off-white suspension. The crude material was filtered and the resulting solid was triturated in methanol and re-filtered. The solid pulled dry to give 5.44 g of the title compound as a pale brown solid.

¹H-NMR (400 MHz, DMSO-d₆): δ=12.52 (1H, bs), 8.17 (1H, s), 7.94 (1H, d), 7.68 (1H, dd), 7.22 (1H, d). LCMS (run time=6 min): Rₜ=2.29 min; m/z 225; 227 [M+H]⁺

Method B: Quinoxalin-2 (1H)-one (1000 g, 6.842 mol) was stirred in sulfuric acid (5.6 L) and silver sulfate was added (1060 g, 3.400 mol). The mixture was vigorously stirred until complete dissolution, then bromine (350 mL, 6.842 mol) was added dropwise. The resulting mixture was stirred at 45° C. for 2 hours. Chloroform (10 L) was added and the mixture was heated to 50° C. for 30 minutes, then filtered. The solid was washed sequentially with sulfuric acid (2 L), chloroform (2 L), water (2 L), methanol (2 L) and TBME (2 L). The resulting solid was dried in vacuo to give 1.18 Kg of the title compound as a pale brown solid.

The title compound can also be made using the procedures detailed in Heterocycles, 1985, 23, 143-151.

### Preparation 30

### 6-Bromo-2-chloroquinoxaline

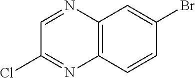

Method A: To a stirred solution of the compound obtained from Preparation 29 (2 g, 8.89 mmol) in phosphorus oxychloride (15 mL), was added DMF (1 mL). The mixture was stirred at 120° C. for 1.5 hours then allowed to cool to room temperature. The dark solution was concentrated in vacuo and cautiously quenched with crushed ice. The aqueous suspension was neutralised with 10% potassium carbonate solution and extracted with DCM (2×30 mL). The combined organic phases were dried (Na₂SO₄), filtered and concentrated to give 1.94 g of the title compound as a brown solid.

¹H-NMR (400 MHz, DMSO-d₆): δ=9.02 (1H, s), 8.40 (1H, d), 8.06 (1H, dd), 7.98 (1H, d). LCMS (run time=2 min): Rₜ=1.69 min; m/z 243; 245 [M+H]⁺

Method B: To a stirred solution of the compound obtained from Preparation 29 (1.0 Kg, 4.44 mol) in phosphorus oxychloride (3.3 L), was added DMF (0.160 L). The mixture was stirred at 120° C. for 1.5 hours and then allowed to cool to 80° C. The dark solution was cautiously quenched into 10% wt/wt NaHCO_{3 (aq)}(44 Kg) at 10 to 15° C. The solid was collected by filtration and washed with water (2 L). The material was triturated in hexanes (4 L), filtered, washed with hexanes (1 L) and dried in vacuo to give 893 g of the title compound as a brown solid.

### Preparation 31

### tert-Butyl (2S)-2-{5-[4-(6-bromoquinoxalin-2-yl)phenyl]-1H-imidazol-2-yl}pyrrolidine-1-carboxylate

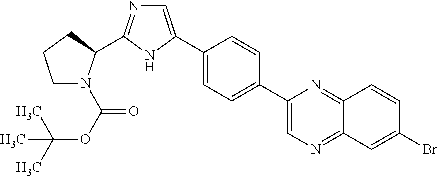

Method A: To a stirred suspension of the boronic ester obtained from Preparation 16 (217 mg, 0.49 mmol) in 1,2-dimethoxyethane (1.50 mL), was added the compound obtained from Preparation 30 (100 mg, 0.41 mmol), Pd(dppf)Cl₂.DCM (34 mg, 0.04 mmol) and 2M sodium carbonate solution (0.62 mL, 1.23 mmol). The mixture was degassed and put under nitrogen three times. It was then stirred at 30° C. overnight. The resulting dark brown mixture was partitioned between ethyl acetate (5 mL) and saturated sodium bicarbonate solution (5 mL). The organic phases were extracted and the aqueous phase was washed with more EtOAc (5 mL). The organic phases were combined, dried (Na₂SO₄), filtered and concentrated in vacuo. The resulting crude material was purified by column chromatography on silica gel (dry loaded redisep 4 g, 0 to 100% ethyl acetate, heptane) to give 163 mg of the title compound as a yellow solid.

¹H-NMR (400 MHz, DMSO-d₆): δ=12.02 (1H, m), 9.64 (1H, m), 8.42-8.33 (3H, m), 8.09 (1H, d), 8.02-7.95 (3H, m), 4.89-4.75 (1H, m), 3.56 (1H, m), 3.38 (1H, m), 2.22 (1H, m), 2.03-1.80 (3H, m), 1.45-1.11 (9H, m).

LCMS (run time=6 min): Rₜ=2.76 min; m/z 520; 522 [M+H]⁺

Method B: To a stirred suspension of the boronic ester obtained from Preparation 16 (2.13 Kg, 4.85 mol) in 1,2-dimethoxyethane (15 L), was added the compound obtained from Preparation 30 (1.0 Kg, 4.11 mol), Pd(dppf)Cl₂.DCM (0.16 Kg, 0.196 mol) and 2M sodium carbonate solution (7.75 L, 15.5 mol). The mixture was degassed and put under nitrogen three times and then stirred at 45° C. overnight. The resulting dark brown mixture was cooled to room temperature, diluted with water (12 L), filtered, washed with water (6 L) and dried in vacuo. The resulting crude material was purified by column chromatography (dry loaded on 2 Kg silica gel, 8 Kg silica gel, 0 to 60% ethyl acetate, hexanes). The product was triturated in hexanes (10 L), filtered, washed with hexanes (2 L) and dried in vacuo to give 686 g of the title compound as a yellow solid.

### Preparation 32

### tert-Butyl (2S)-2-(5-{4-[6-(4,4,5,5-tetramethyl-1,3,2-dioxaborolan-2-yl)quinoxalin-2-yl]phenyl}-1H-imidazol-2-yl)pyrrolidine-1-carboxylate

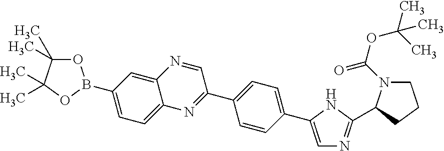

A mixture of the bromide obtained from Preparation 31 (889 mg, 1.71 mmol), bis(pinacolato)diboron (868 mg, 3.42 mmol), potassium acetate (419 mg, 4.273 mmol), and Pd(dppf)Cl₂.DCM (43 mg, 0.17 mmol) in dioxane (10 mL) was degassed by evacuating then refilling with nitrogen 3 times. The mixture was heated under gentle reflux for 1.5 hours and then stood at room temperature overnight. The resulting dark brown mixture was partitioned between ethyl acetate (5 mL) and saturated sodium bicarbonate solution (5 mL). The organic phases were extracted and the aqueous phase was washed with more ethyl acetate (5 mL). The organic phases were then combined, dried (Na₂SO₄), filtered and concentrated in vacuo. The resulting crude material was purified by column chromatography (dry loaded redisep (80 g), 20 to 100% ethyl acetate, heptane) to give 370 mg of the title compound as a yellow solid.

¹H-NMR (400 MHz, MeOD): δ=9.48 (1H, s), 8.49 (1H, s), 8.34 (2H, m), 8.13 (2H, s), 7.95 (2H, m), 7.50 (1H, m), 5.02 (1H, m), 3.70 (1H, m), 3.53 (1H, m), 2.40-1.90 (4H, m), 1.52-1.37 (9H, m), 1.29-1.18 (12H, m).

LCMS (run time=6 min): Rₜ=3.06 min; m/z 568 [M+H]⁺

### Preparation 33

### tert-Butyl (2S)-2-(5-[2-(4-{2-[(2S)-1-(tert-butoxycarbonyl)pyrrolidin-2-yl]-1H-imidazol-5-yl}phenyl)quinoxalin-6-yl]-1-{[2-(trimethylsilyl)ethoxy]methyl}-1H-imidazol-2-yl)pyrrolidine-1-carboxylate

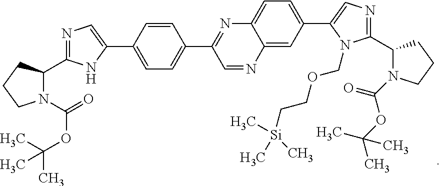

Method A: The bromo imidazole obtained from Preparation 13 (41 mg, 0.09 mmol), the compound obtained from Preparation 32 (52 mg, 0.09 mmol), and Pd(dppf)Cl₂.DCM (4.5 mg, 0.018 mmol) were added to a microwave vial (Biotage, 0.5-2.0 mL), followed by 1,2-dimethoxyethane (0.5 mL) and 2M Na₂CO_{3(aq)}(0.14 mL, 0.28 mmol). The mixture was heated under microwave irradiation at 120° C. for 20 minutes. The resulting mixture was partitioned between ethyl acetate (5 mL) and saturated sodium bicarbonate solution (5 mL). The organic phases were extracted and the aqueous phase was washed with more EtOAc (5 mL). The organic phases were combined, dried (Na₂SO₄), filtered and concentrated in vacuo. The resulting crude material was purified by column chromatography on silica gel (dry loaded redisep (4 g), 20 to 100% ethyl acetate, heptane) to give 32 mg of the title compound as a yellow oil.

¹H-NMR (400 MHz, MeOD): δ=9.41 (1H, m), 8.45 (1H, m), 8.36-8.20 (3H, m), 8.12 (1H, m), 7.84 (2H, m), 7.80 (1H, m), 7.48 (1H, m), 5.79-5.54 (1H, m), 5.39 (1H, m), 5.05 (1H, m), 4.93 (1H, m), 3.81-3.47 (6H, m), 2.47-1.92 (8H, m), 1.51-1.20 (18H, m), 0.97 (2H, m), 0.04 (9H, s). LCMS (run time=6 min): Rₜ=3.39 min; m/z 807 [M+H]⁺

Method B: To the compound obtained from Preparation 31 (17.7 g, 34.0 mmol) in 1,2-dimethoxyethane (425 mL), was added 1N Na₂CO_{3 (aq)}(104 mL, 104 mmol) and heated to 80° C. The imidazole boronic acid obtained from Preparation 13b (22.4 g, 54.4 mmol) was added as a solution in 1,2-dimethoxyethane (104 mL). Pd(dppf)Cl₂.DCM (0.22 g, 0.27 mmol) was added and heating continued for 5 hours. The resulting mixture was concentrated in vacuo, and partitioned with ethyl acetate (354 mL) and water (354 mL). The aqueous phase was extracted with further ethyl acetate (354 mL), then the organic phases were combined, dried (MgSO₄), filtered and concentrated in vacuo. The resulting crude material was purified by column chromatography on silica gel (Biotage snap (4×340 g), 0 to 10% methanol: ethyl acetate) to give the title compound as a yellow foam (15.5 g).

Method C: To the compound obtained from Preparation 31 (200 g, 384 mmol) in 1,4-dioxane (2000 mL), was added potassium acetate (113 g, 1.15 mol) and bis(pinacolato)diboron (97.6 g, 384 mmol). The reaction mixture was heated to 60° C. Pd(dppf)Cl2.DCM (3.14 g, 3.84 mmol) was then added and the reaction mixture was heated further to 110° C. for 5 hours. It was then cooled to 85° C. and a solution of the bromo imidazole obtained from Preparation 13 (204 g, 456.9 mmol) in 1,4-dioxane (500 mL) was added, followed by 2M Na₂CO₃ (aq) (577 mL, 1154 mmol) and Pd(dppf)Cl2.DCM (3.14 g, 3.84 mmol). The resulting mixture was heated to 110° C. for 16 hours. It was then concentrated in vacuo, the residue dissolved in toluene (2000 mL) and filtered. The filtrate was concentrated in vacuo and the resulting crude material was purified by column chromatography (Biotage 3×5 Kg), 0-10% ethanol, TBME) to give the title compound (248 g).

### Preparation 34

### 6-{2-[(2S)-Pyrrolidin-2-yl]-1H-imidazol-5-yl}-2-(4-{2-[(2S)-pyrrolidin-2-yl]-1H-imidazol-5-yl}phenyl)quinoxaline

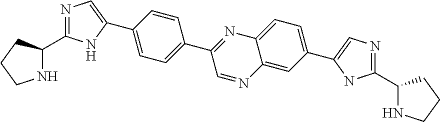

To a stirred solution of the compound obtained from Preparation 33 (32 mg, 0.04 mmol), in absolute ethanol (1 mL), was added 2N HCl in dioxane (1 mL, 2 mmol). The mixture was stirred at 75° C. for 1.5 hours. It was then concentrated in vacuo. The residue was dissolved in water and basified to pH˜11 with 2N sodium hydroxide solution. The product was extracted with 20% MeOH/DCM (4×10 mL). The organic phases were combined, dried (Na₂SO₄), filtered, concentrated in vacuo and then azeotroped with toluene to give 17 mg of title compound as a yellow oil.

¹H-NMR (400 MHz, MeOD): δ=9.41 (1H, s), 8.40 (1H, s), 8.30 (2H, d), 8.24 (1H, d), 8.12 (1H, d), 7.94 (2H, d), 7.66 (1H, s), 7.52 (1H, s), 4.37 (2H, m), 3.23 (2H, m), 3.06 (2H, m), 2.32 (2H, m), 2.12-1.91 (6H, m).

LCMS (run time=6 min): Rₜ=2.06 min; m/z 477 [M+H]⁺

### Preparation 34a

### 6-{2-[(2S)-Pyrrolidin-2-yl]-1H-imidazol-5-yl}-2-(4-{2-[(2S)-pyrrolidin-2-yl]-1H-imidazol-5-yl}phenyl)quinoxaline tetra hydrochloride salt

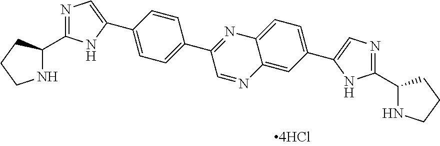

To a stirred solution of the compound obtained from Preparation 33 (1093.9 g, 1.355 mol), in absolute ethanol (10 L) at 80° C., was added 11.6N aqueous HCl (915.4 mL, 10.62 mol) over 2 hours. The mixture was stirred at 80° C. for 4 hours. It was then cooled to ambient temperature and the solid was collected by filtration. The cake was washed with TBME (1 L) and dried in vacuo to give 902.7 g of the title compound as an orange solid.

¹H-NMR (400 MHz, DMSO-d₆): δ=9.66 (1H, s), 8.69 (1H, s), 8.47-8.45 (3H, m), 8.37-8.35 (2H, m), 8.17-8.15 (3H, m), 5.19-5.13 (2H, m), 3.56-3.39 (4H, m), 2.58-2.55 (4H, m), 2.26-2.22 (2H, m), 2.04-1.99 (2H, m) LCMS (run time=12.5 min): Rₜ=3.66 min; m/z 477 [M+H]⁺

### Preparation 35

### tert-Butyl (2S)-2-[5-(6-bromoquinoxalin-2-yl)-1-{[2-(trimethylsilyl)ethoxy]methyl}-1H-imidazol-2-yl]pyrrolidine-1-carboxylate

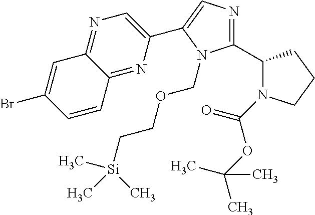

To a stirred suspension of (2-[(2S)-1-(tert-butoxycarbonyl)pyrrolidin-2-yl]-1-{[2 (trimethylsilyl)ethoxy]methyl}-1H-imidazol-5-yl)boronic acid (150 mg, 0.26 mmol), obtained from Preparation 13b, in 1,2-dimethoxyethane (1 mL), was added 6-bromo-2-chloroquinoxaline (62 mg, 0.26 mmol), obtained from Preparation 30, Pd(dppf)Cl₂.DCM (13 mg, 0.05 mmol) and 2M Na₂CO_{3 (aq)} (0.38 mL, 0.77 mmol). The mixture was degassed, then put under nitrogen three times and then stirred at 30° C. for 3 hours. The resulting dark brown mixture was partitioned between ethyl acetate (5 mL) and water (5 mL). The organic phases were extracted and the aqueous phase was washed with more EtOAc (5 mL). The organic phases were combined, dried (Na₂SO₄), filtered and concentrated in vacuo. The resulting crude material was purified by column chromatography (dry loaded redisep (4 g), 20 to 80% ethyl acetate, heptane) to give 98 mg of the title compound as a orange foam.

¹H-NMR (400 MHz, MeOD): δ=9.28 (1H, d), 8.22 (1H, d), 7.95 (3H, m), 6.70 (1H, m), 5.95 (1H, dd), 5.16 (1H, m), 3.75-3.52 (4H, m), 2.42 (1H, m), 2.24-1.91 (3H, m), 1.46-1.21 (9H, m), 0.86 (2H, m), −0.17 (9H, d).

LCMS (run time=6 min): Rₜ=4.38 min; m/z 574; 576 [M+H]⁺

### Preparation 36

### tert-Butyl (2S)-2-(5-[6-(4-{2-[(2S)-1-(tert-butoxycarbonyl)pyrrolidin-2-yl]-1H-imidazol-5-yl}phenyl)quinoxalin-2-yl]-1-{[2-(trimethylsilyl)ethoxy]methyl}-1H-imidazol-2-yl)pyrrolidine-1-carboxylate

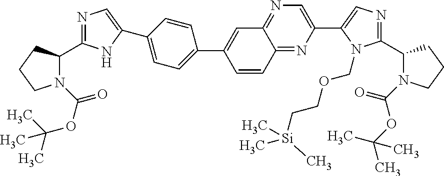

(S)-tert-Butyl 2-(5-(4-(4,4,5,5-tetramethyl-1,3,2-dioxaborolan-2-yl)phenyl)-1H-imidazol-2-yl)pyrrolidine-1-carboxylate (90 mg, 0.21 mmol), obtained from Preparation 16, tert-butyl (2S)-2-[5-(6-bromoquinoxalin-2-yl)-1-{[2-(trimethylsilyl)ethoxy]methyl}-1H-imidazol-2-yl]pyrrolidine-1-carboxylate (98 mg, 0.17 mmol), obtained from Preparation 35, and Pd(dppf)Cl₂.DCM (9 mg, 0.03 mmol) were added to a microwave vial (Biotage, 0.5-2.0 mL), followed by 1,2-dimethoxyethane (2 mL) and 2M Na₂CO_{3(aq)} (0.26 mL, 0.51 mmol). The mixture was heated under microwave irradiation at 120° C. for 20 minutes. The resulting mixture was partitioned between ethyl acetate (5 mL) and water (5 mL). The organic phases were extracted and the aqueous phase was washed with more EtOAc (5 mL). The organic phases were combined, dried (Na₂SO₄), filtered and concentrated in vacuo. The resulting crude material was purified by column chromatography (dry loaded redisep (4 g), 40 to 100% ethyl acetate, heptane continued into 0 to 20% methanol, ethyl acetate) to give 85 mg of the title compound as a bright yellow foam.

¹H-NMR (400 MHz, MeOD): δ=9.28 (1H, d), 8.31 (1H, m), 8.21 (1H, m), 8.14 (1H, d), 7.97-7.84 (5H, m), 7.43 (1H, m), 6.75 (1H, m), 5.98 (1H, dd), 5.17 (1H, m), 4.94 (1H, m), 3.70 (4H, m), 3.56 (2H, m), 2.41 (2H, m), 2.25-1.94 (6H, m), 1.52-1.23 (18H, m), 0.89 (2H, m), −0.15 (9H, d). LCMS (run time=6 min): Rₜ=2.83 min; m/z 807 [M+H]⁺

### Preparation 37

### 2-{2-[(2S)-Pyrrolidin-2-yl]-1H-imidazol-5-yl}-6-(4-{2-[(2S)-pyrrolidin-2-yl]-1H-imidazol-5-yl}phenyl)quinoxaline

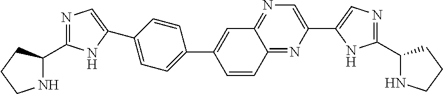

To a stirred solution of tert-butyl (2S)-2-(5-[6-(4-{2-[(2S)-1-(tert-butoxycarbonyl)pyrrolidin-2-yl]-1H-imidazol-5-yl}phenyl)quinoxalin-2-yl]-1-{[2-(trimethylsilyl)ethoxy]methyl}-1H-imidazol-2-yl)pyrrolidine-1-carboxylate (85 mg, 0.1 mmol), obtained from Preparation 36, in absolute ethanol (1 mL), was added 2N HCl in dioxan (1 mL, 30 mmol) and the mixture was stirred at 75° C. for 1.5 hours. It was then concentrated in vacuo. The residue was dissolved in water and basified to pH˜11 with 2N NaOH aq. The product was extracted with 20% MeOH/DCM (4×10 mL). The organic phases were combined, dried (Na₂SO₄), filtered, concentrated in vacuo and then azeotroped with toluene. The residue was dissolved in methanol (5 mL) and loaded onto an SCX column washing with more methanol. The product was collected by flushing with 7N ammonia in methanol and the filtrate was concentrated in vacuo to give 50 mg of title compound as a yellow solid.

¹H-NMR (400 MHz, MeOD): δ=9.30 (1H, m), 8.13 (1H, m), 8.03 (1H, m), 7.98 (1H, m), 7.89 (1H, m), 7.81-7.73 (4H, m), 7.35 (1H, s), 4.20 (2H, m), 3.13 (2H, m), 2.87 (2H, m), 2.31-2.08 (3H, m), 1.99-1.79 (5H, m).

LCMS (run time=6 min): Rₜ=1.50 min; m/z 477 [M+H]⁺

### Preparation 38

### 5-Bromo-2-[6-(tert-butyl-dimethyl-silanyloxy)-naphthalen-2-yl]-pyridine

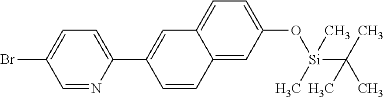

To a stirred solution of 2,5-dibromo pyridine (0.5 g, 2.11 mmol) in toluene (18 mL) was added 6-(tert-butyl-dimethyl-silanyloxy)-naphthalen-2-yl-boronic acid (0.765 g, 2.52 mmol), ethanol (6 mL), water (4 mL) and Na₂CO₃ (0.671 g, 6.33 mmol). Argon was bubbled through the reaction mixture for 30 minutes. Then Pd(PPh₃)₄ (0.121 g, 0.104 mmol) was added and the mixture was heated in a sealed tube at 90° C. for 16 hours. The solvent was evaporated under vacuum and the residue was diluted with ethyl acetate (25 mL). The reaction mixture was filtered through celite. The organic layer was washed with water (15 mL) followed by brine (15 mL), dried over Na₂SO₄ and was evaporated to dryness. The crude mass was purified by column chromatography on silica gel by using ethyl acetate:hexane (1:9) mixture to afford the title compound as a white solid (300 mg).

¹H NMR (400 MHz, CDCl₃): δ=8.75 (1H, s), 8.37 (1H, s), 8.04 (1H, m), 7.88 (1H, m), 7.80 (1H, m), 7.74 (1H, m), 7.20 (1H, s), 7.11 (1H, m), 1.02 (9H, s), 0.26 (6H, s). LCMS (System 1) (run time=5 min): Rₜ=3.46 min; 414; 416 [M+H]⁺

### Preparation 39

### tert-Butyl (2S)-2-(5-[6-(6-{[tert-butyl(dimethyl)silyl]oxy}naphthalen-2-yl)pyridin-3-yl]-1-{[2-(trimethylsilyl)ethoxy]methyl}-1H-imidazol-2-yl)pyrrolidine-1-carboxylate

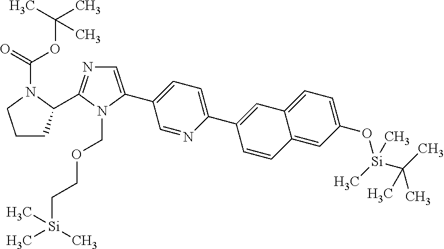

To a stirred solution of 5-bromo-2-[6-(tert-butyl-dimethyl-silanyloxy)-naphthalen-2-yl]-pyridine (0.150 g, 0.363 mmol), obtained from Preparation 38, in toluene (6 mL) was added (2-[(2S)-1-(tert-butoxycarbonyl)pyrrolidin-2-yl]-1-{[2 (trimethylsilyl)ethoxy]methyl}-1H-imidazol-5-yl)boronic acid (0.239 g, 0.581 mmol), obtained from Preparation 13b, ethanol (2 mL), water (1 mL) and Na₂CO₃ (0.061 g, 0.581 mmol). Argon was bubbled through the reaction mixture for 30 minutes. Then Pd(PPh₃)₄ (0.042 g, 0.036 mmol) was added and the mixture was heated in a sealed tube at 90° C. for 16 hours. The solvent was evaporated under vacuum and the residue was diluted with ethyl acetate (15 mL). The reaction mixture was filtered through celite. The organic layer was washed with water (10 mL), followed by brine (10 mL), then dried over Na₂SO₄ and evaporated to dryness. The crude mass was purified by column chromatography on silica gel by using ethyl acetate:hexane (1:4) mixture to afford the title compound as alight yellow liquid (150 mg).

LCMS (System 1) (run time=5 min): Rₜ=1.93 min; m/z 701 [M+H]⁺

### Preparation 40

### tert-Butyl (2S)-2-(5-[6-(6-hydroxynaphthalen-2-yl)pyridin-3-yl]-1-{[2-(trimethylsilyl)ethoxy]methyl}-1H-imidazol-2-yl)pyrrolidine-1-carboxylate

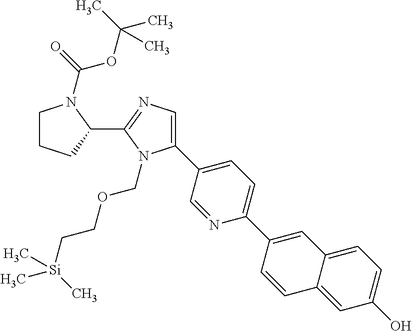

To a stirred solution of tert-butyl (2S)-2-(5-[6-(6-{[tert-butyl(dimethyl)silyl]oxy}naphthalen-2-yl)pyridin-3-yl]-1-{[2-(trimethylsilyl)ethoxy]methyl}-1H-imidazol-2-yl)pyrrolidine-1-carboxylate (0.8 g, 1.14 mmol), obtained from Preparation 39, in THF (30 mL) was added 1M solution of TBAF (1.14 mL). The reaction mixture was stirred at room temperature for 0.5 hours. The solvent was then evaporated and the reaction mixture was diluted with ethyl acetate (30 mL). The organic layer was washed with water (30 mL), followed by brine (30 mL), then dried over Na₂SO₄ and evaporated to dryness. The crude mass was purified by column chromatography on silica gel by using ethyl acetate:hexane (2:5) mixture to afford the title compound as a light yellow liquid (600 mg).

### Preparation 41

### tert-Butyl (2S)-2-(5-[6-(6-{[(trifluoromethyl)sulfonyl]oxy}naphthalen-2-yl)pyridin-3-yl]-1-{[2-(trimethylsilyl)ethoxy]methyl}-1H-imidazol-2-yl)pyrrolidine-1-carboxylate

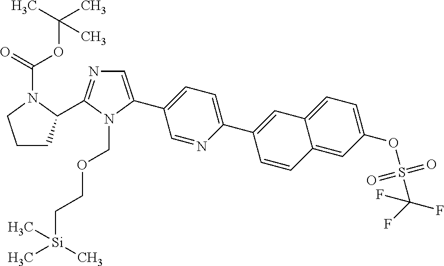

To a stirred solution of tert-butyl (2S)-2-(5-[6-(6-hydroxynaphthalen-2-yl)pyridin-3-yl]-1-{[2-(trimethyl silyl)ethoxy]methyl}-1H-imidazol-2-yl)pyrrolidine-1-carboxylate (0.14 g, 0.238 mmol), obtained from Preparation 40, in DCM (5 mL) was added triethylamine (0.139 mL, 0.523 mmol) and triflic anhydride (0.06 mL, 0.358 mmol) at −40° C. The mixture was stirred at this temperature for 10 minutes. It was then quenched with ice water and diluted with DCM (10 mL). The DCM layer was washed with water (10 mL), followed by brine (10 mL), then dried over Na₂SO₄ and evaporated to dryness. The crude material was purified by column chromatography on silica gel by using ethyl acetate:hexane (2:5) mixture to afford the title compound as a light yellow liquid (120 mg).

LCMS (System 1) (run time=5 min): Rₜ=1.56 min; m/z 719 [M+H]⁺

### Preparation 42

### tert-Butyl (2S)-2-(5-{6-[6-(2-[(2S)-1-(tert-butoxycarbonyl)pyrrolidin-2-yl]-1-{[2-(trimethyl silyl)ethoxy]methyl}-1H-imidazol-5-yl)naphthalen-2-yl]pyridin-3-yl}-1-{[2-(trimethylsilyl)ethoxy]methyl}-1H-imidazol-2-yl)pyrrolidine-1-carboxylate

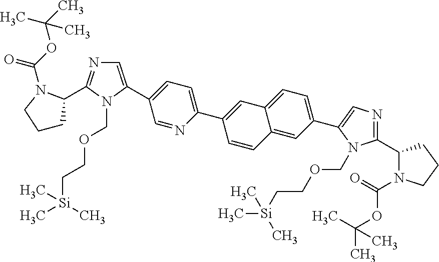

To a stirred solution of tert-butyl (2S)-2-(5-[6-(6-{[(trifluoromethyl)sulfonyl]oxy}naphthalen-2-yl)pyridin-3-yl]-1-{[2-(trimethylsilyl)ethoxy]methyl}-1H-imidazol-2-yl)pyrrolidine-1-carboxylate (0.5 g, 0.696 mmol), obtained from Preparation 41, in acetonitrile (10 mL) was added (2-[(2S)-1-(tert-butoxycarbonyl)pyrrolidin-2-yl]-1-{[2 (trimethylsilyl)ethoxy]methyl}-1H-imidazol-5-yl)boronic acid (0.572 g, 1.39 mmol), obtained from Preparation 13b, and CsF (0.951 g, 6.264 mmol). Argon was bubbled through the reaction mixture for 30 minutes. Tricyclohexyl phosphine (0.064 g, 0.229 mmol) and Pd(OAc)₂ (0.031 g, 0.2 mmol) were added to the reaction mixture and it was heated in a sealed tube at 80° C. for 16 hours. The acetonitrile was evaporated, and the reaction mixture was diluted with ethyl acetate (40 mL). The ethyl acetate layer was washed with water (15 mL), followed by brine (15 mL), then dried over Na₂SO₄ and evaporated to dryness. The crude material was purified by column chromatography on silica gel by using ethyl acetate:hexane (4:5) mixture to afford the title compound as a light yellow solid (540 mg).

LCMS (System 1) (run time=5 min): Rₜ=1.08 min; m/z 936 [M+H]⁺

### Preparation 43

### 5-{2-[(2S)-Pyrrolidin-2-yl]-1H-imidazol-4-yl}-2-(6-{2-[(2S)-pyrrolidin-2-yl]-1H-imidazol-5-yl}naphthalen-2-yl)pyridine hydrochloride salt

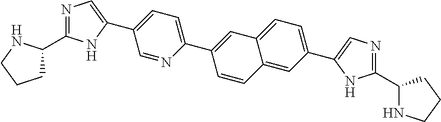

To a stirred solution of tert-butyl (2S)-2-(5-{6-[6-(2-[(2S)-1-(tert-butoxycarbonyl)pyrrolidin-2-yl]-1-{[2-(trimethyl silyl)ethoxy]methyl}-1H-imidazol-5-yl)naphthalen-2-yl]pyridin-3-yl}-1-{[2-(trimethylsilyl)ethoxy]methyl}-1H-imidazol-2-yl)pyrrolidine-1-carboxylate (0.540 g, 0.577 mmol), obtained from Preparation 42, in dioxane (4 mL) was added 4M HCl in 1,4-dioxane (30 mL) at 0° C. The mixture was stirred at room temperature for 16 hours. Then the dioxane was evaporated and the residue was triturated with ether (2×10 mL) to give the title compound as a light yellow solid (400 mg).

LCMS (System 1) (run time=5 min): Rₜ=3.13 min; m/z 476 [M+H]⁺

### Preparation 44

### 2,5-Dibromo-pyrimidine

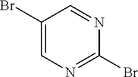

To a stirred solution of 2-hydroxy-5-bromo pyrimidine (2 g, 11.43 mmol) in PBr₃ (6 mL) was added POBr₃ (1.4 mL, 13.72 mmol) at 0° C. The mixture was stirred for 10 minutes and then refluxed for 2 hours, after which time it was quenched with a saturated solution of sodium bicarbonate at 0° C. It was then diluted with ethyl acetate (40 mL), washed with water (30 mL), followed by brine (20 mL). The organic phase was dried over sodium sulphate and evaporated to dryness. The crude mass was purified by column chromatography on silica gel by using ethyl acetate:hexane (1:9) mixture to give the title compound as a white solid (1.8 g).

¹H NMR (400 MHz, DMSO-d₆): δ=8.93 (s, 2H). GCMS (System 7) (run time=13.5 min): Rₜ=6.53 min; m/z 238 [M+H]⁺

### Preparation 45

### 5-Bromo-2-(6-{[tert-butyl(dimethyl)silyl]oxy}naphthalen-2-yl)pyrimidine

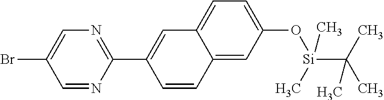

To a stirred solution of 2,5 dibromo pyrimidine (0.32 g, 1.332 mmol), obtained from Preparation 44, in toluene (6 mL) was added 6-(tert-butyl-dimethyl-silanyloxy)-naphthalen-2-yl-boronic acid (0.48 g, 1.585 mmol), EtOH (2 mL), water (1 mL) and Na₂CO₃ (0.35 g, 3.33 mmol). Argon was bubbled through the reaction mixture for 30 minutes. Then Pd(PPh₃)₄ (0.075 g, 0.065 mmol) was added and the mixture was heated in a sealed tube at 90° C. for 16 hours. The solvent was evaporated under vacuum and the reaction mixture was diluted with ethyl acetate (15 mL). The ethyl acetate layer was filtered through celite then washed with water (10 mL) and brine (10 mL). It was then dried over Na₂SO₄ and evaporated to dryness. The crude mass was purified by column chromatography on silica gel by using ethyl acetate:hexane (1:9) mixture to give the title compound as a white solid (546 mg).

¹H NMR (400 MHz, CDCl₃): δ=8.86 (s, 1H), 8.83 (s, 1H), 8.40 (d, 1H), 7.87 (d, 1H), 7.76 (d, 1H), 7.31 (s, 1H), 7.20 (s, 1H), 7.10 (dd, 1H), 1.02 (s, 9H), 0.26 (s, 6H). LCMS (System 1) (run time=5 min): Rₜ=3.57 min; m/z 415; 417 [M+H]⁺

### Preparation 46

### tert-Butyl (2S)-2-(5-[2-(6-{[tert-butyl(dimethyl)silyl]oxy}naphthalen-2-yl)pyrimidin-5-yl]-1-{[2-(trimethylsilyl)ethoxy]methyl}-1H-imidazol-2-yl)pyrrolidine-1-carboxylate

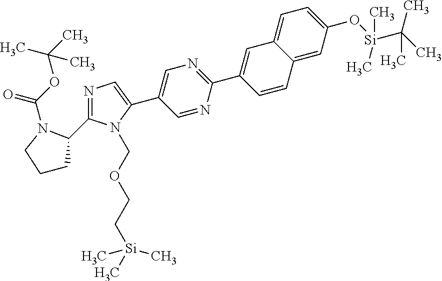

To a stirred solution of 5-bromo-2-(6-{[tert-butyl(dimethyl)silyl]oxy}naphthalen-2-yl)pyrimidine (0.25 g, 0.485 mmol), obtained from Preparation 45, in toluene (6 mL) was added (2-[(2S)-1-(tert-butoxycarbonyl)pyrrolidin-2-yl]-1-{[2 (trimethylsilyl)ethoxy]methyl}-1H-imidazol-5-yl)boronic acid (0.3 g, 0.728 mmol), obtained from Preparation 13b, EtOH (2 mL), water (1 mL) and Na₂CO₃ (82 mg, mmol). Argon was bubbled through the reaction mixture for 30 minutes. Pd(PPh₃)₄ (0.056 g, 0.048 mmol) was added and the mixture was heated in a sealed tube at 90° C. for 16 hours. The solvent was evaporated under vacuum and the reaction mixture was diluted with ethyl acetate (15 mL). It was then filtered through celite and washed with water (10 mL), then brine (10 mL). The organic phase was dried over Na₂SO₄ and evaporated to dryness. The crude mass was purified by column chromatography on silica gel by using ethyl acetate:hexane (1:4) mixture to give the title compound as a light yellow liquid (300 mg).

LCMS (System 1) (run time=5 min): Rₜ=2.14 min; m/z 702 [M+H]⁺

### Preparation 47

### tert-Butyl (2S)-2-(5-[2-(6-hydroxynaphthalen-2-yl)pyrimidin-5-yl]-1-{[2-(trimethyl silyl)ethoxy]methyl}-1H-imidazol-2-yl)pyrrolidine-1-carboxylate

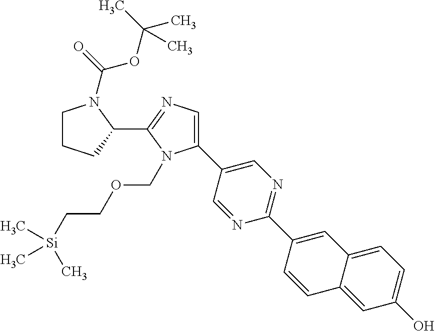

To a stirred solution of tert-butyl (2S)-2-(5-[2-(6-{[tert-butyl(dimethyl)silyl]oxy}naphthalen-2-yl)pyrimidin-5-yl]-1-{[2-(trimethylsilyl)ethoxy]methyl}-1H-imidazol-2-yl)pyrrolidine-1-carboxylate (1.1 g, 1.56 mmol), obtained from Preparation 46, in THF (20 mL) was added 1M solution of TBAF (1.6 mL). The reaction mixture was stirred at room temperature for 0.5 hours. The solvent was evaporated and the reaction mixture was diluted with ethyl acetate (30 mL). The organic layer was washed with water (30 mL), brine (30 mL), then dried over Na₂SO₄ and evaporated to dryness. The crude mass was purified by column chromatography on silica gel by using ethyl acetate:hexane (2:5) mixture to give the title compound as a light yellow liquid (700 mg).

LCMS (System 1) (run time=5 min): Rₜ=3.18 min: m/z 588 [M+H]⁺

### Preparation 48

### tert-Butyl (2S)-2-(5-[2-(6-{[(trifluoromethyl)sulfonyl]oxy}naphthalen-2-yl)pyrimidin-5-yl]-1-{[2-(trimethylsilyl)ethoxy]methyl}-1H-imidazol-2-yl)pyrrolidine-1-carboxylate

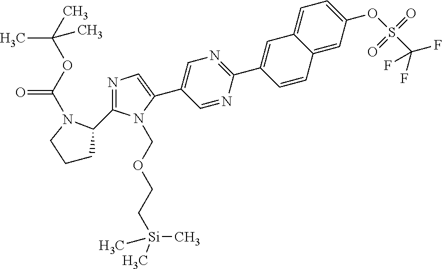

To a stirred solution of tert-butyl (2S)-2-(5-[2-(6-hydroxynaphthalen-2-yl)pyrimidin-5-yl]-1-{[2-(trimethyl silyl)ethoxy]methyl}-1H-imidazol-2-yl)pyrrolidine-1-carboxylate (0.47 g, 0.801 mmol), obtained from Preparation 47, in DCM (10 mL) was added triethylamine (0.245 mL, 1.76 mmol) and triflic-anhydride (0.182 mL, 1.08 mmol) at −78° C. The mixture was stirred at this temperature for 10 minutes. It was then quenched with a saturated solution of sodium bicarbonate and diluted with DCM (10 mL). The organic layer was washed with water (10 mL), and brine (10 mL), then dried over Na₂SO₄ and evaporated to dryness, to give the title compound as a light yellow liquid (600 mg). LCMS (System 1) (run time=5 min): Rₜ=3.55 min; m/z 720 [M+H]⁺

### Preparation 49

### tert-Butyl (2S)-2-(5-{2-[6-(2-[(2S)-1-(tert-butoxycarbonyl)pyrrolidin-2-yl]-1-{[2-(trimethyl silyl)ethoxy]methyl}-1H-imidazol-5-yl)naphthalen-2-yl]pyrimidin-5-yl}-1-{[2-(trimethylsilyl)ethoxy]methyl}-1H-imidazol-2-yl)pyrrolidine-1-carboxylate

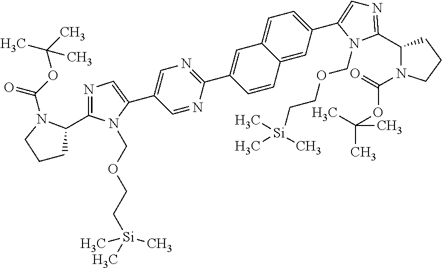

To a stirred solution of tert-butyl (2S)-2-(5-[2-(6-{[(trifluoromethyl)sulfonyl]oxy}naphthalen-2-yl)pyrimidin-5-yl]-1-{[2-(trimethylsilyl)ethoxy]methyl}-1H-imidazol-2-yl)pyrrolidine-1-carboxylate (0.6 g, 0.834 mmol), obtained from Preparation 48, in acetonitrile (10 mL) was added (2-[(2S)-1-(tert-butoxycarbonyl)pyrrolidin-2-yl]-1-{[2 (trimethylsilyl)ethoxy]methyl}-1H-imidazol-5-yl)boronic acid (0.69 g, 1.668 mmol), obtained from Preparation 13b, and CsF (1.14 g, 7.506 mmol). Argon was bubbled through the reaction mixture for 30 minutes. Tricyclohexyl phosphine (0.077 g, 0.275 mmol) and Pd(OAc)₂ (0.04 g, 0.1668 mmol) were then added to the reaction mixture and it was heated in a sealed tube at 80° C. for 16 hours. The solvent was evaporated and the residue was diluted with ethyl acetate (40 mL). The solution was washed with water (15 mL), and brine (15 mL), then dried over Na₂SO₄ and evaporated to dryness. The crude material was purified by column chromatography on silica gel by using ethyl acetate:hexane (4:5) mixture to give the title compound as a light yellow solid (600 mg).

LCMS (System 1) (run time=5 min): Rₜ=1.24 min; m/z 937 [M+H]⁺

### Preparation 50

### 5-{2-[(2S)-Pyrrolidin-2-yl]-1H-imidazol-5-yl}-2-(6-{2-[(2S)-pyrrolidin-2-yl]-1H-imidazol-5-yl}naphthalen-2-yl)pyrimidine hydrochloride salt

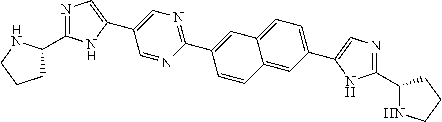

To a stirred solution of tert-butyl (2S)-2-(5-{2-[6-(2-[(2S)-1-(tert-butoxycarbonyl)pyrrolidin-2-yl]-1-{[2-(trimethyl silyl)ethoxy]methyl}-1H-imidazol-5-yl)naphthalen-2-yl]pyrimidin-5-yl}-1-{[2-(trimethylsilyl)ethoxy]methyl}-1H-imidazol-2-yl)pyrrolidine-1-carboxylate (0.06 g, 0.640 mmol), obtained from Preparation 49, in dioxane (4 mL) was added 4M HCl in 1,4-dioxane (30 mL) at 0° C. The mixture was stirred at room temperature for 16 hours. The solvent was then evaporated and the residue was triturated with ether (2×10 mL) to give the title compound as a light yellow solid (400 mg).

LCMS (System 1) (run time=5 min): Rₜ=1.59 min; m/z 477 [M+H]⁺

### Preparation 51

### tert-Butyl (2S)-2-{5-[4-(3-chloroisoquinolin-7-yl)phenyl]-1H-imidazol-2-yl}pyrrolidine-1-carboxylate

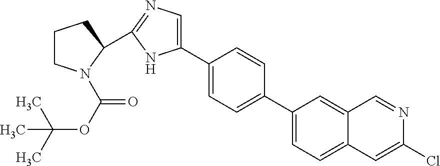

7-Bromo-3-chloro-isoquinoline (50 mg, 0.210 m mol), (S)-tert-butyl 2-(5-(4-(4,4,5,5-tetramethyl-1,3,2-dioxaboro Ian-2-yl)phenyl)-1H-imidazol-2-yl)pyrrolidine-1-carboxylate (108 mg, 0.247 mmol), obtained from Preparation 16, and Pd(ddpf)Cl₂ (17 mg, 0.021 mmol) were dissolved in DME (1 mL) in a microwave vial. 2M Na₂CO₃ (aq) (0.309 mL, 0.618 mmol) was then added and the vial was sealed and heated at 120° C. for 20 minutes in the microwave. The reaction mixture was then diluted with ethyl acetate (20 mL) and washed with water (10 mL) followed by brine (10 mL). The organic extracts were concentrated in vacuo to give the crude product, which was dry loaded onto silica. Purification by column chromatography on silica gel (redisep 4 g, 30:70 to 70:30 EtOAc: Heptane) gave 95 mg of the title compound as a brown glass.

¹H NMR (CDCl3, 400 MHz): δ=9.12 (s, 1H), 8.16 (s, 1H), 8.00 (d, 1H), 7.84 (d, 1H), 7.81-7.71 (m, 5H), 5.04 (m, 1H), 3.47 (m, 2H), 3.02 (m, 1H), 2.22 (m, 2H), 2.05-1.96 (m, 2H), 1.25 (s, 9H). LCMS (run time=2 min): R=1.42 min; m/z 475; 477 [M+H]⁺

### Preparation 52

### tert-Butyl (2S)-2-(5-[7-(4-{2-[(2S)-1-(tert-butoxycarbonyl)pyrrolidin-2-yl]-1H-imidazol-5-yl}phenyl)isoquinolin-3-yl]-1-{[2-(trimethylsilyl)ethoxy]methyl}-1H-imidazol-2-yl)pyrrolidine-1-carboxylate

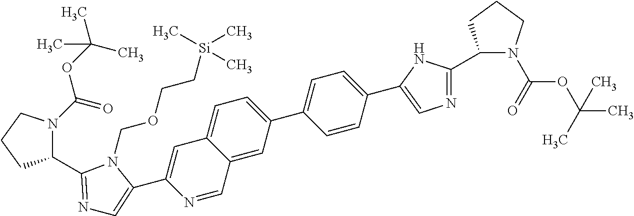

A mixture of tert-butyl (2S)-2-{5-[4-(3-chloroisoquinolin-7-yl)phenyl]-1H-imidazol-2-yl}pyrrolidine-1-carboxylate (75 mg, 0.160 mmol), obtained from Preparation 51, (2-[(2S)-1-(tert-butoxycarbonyl)pyrrolidin-2-yl]-1-{[2 (trimethylsilyl)ethoxy]methyl}-1H-imidazol-5-yl)boronic acid (100 mg, 0.243 mmol), obtained from Preparation 13b, SPhos (13 mg, 0.032 mmol) and Pd₂(dba)₃ (7 mg, 0.008 mmol) were dissolved in dioxane (3 mL). The mixture was evacuated and placed under nitrogen three times. A solution of K₃PO₄ (67 mg, 0.316 mmol) in water (0.249 mL) was added and the mixture was evacuated and placed under nitrogen three times. The mixture was heated at 110° C. overnight and then allowed to cool to room temperature. A further charge of (2-[(2S)-1-(tert-butoxycarbonyl)pyrrolidin-2-yl]-1-{[2 (trimethylsilyl)ethoxy]methyl}-1H-imidazol-5-yl)boronic acid (100 mg, 0.243 mmol), obtained from Preparation 13b, SPhos (13 mg, 0.032 mmol) and Pd₂(dba)₃ (7 mg, 0.008 mmol) were added and the mixture was evacuated and placed under nitrogen three times. It was then heated at 110° C. for 4 hours. The reaction mixture was concentrated in vacuo and purified by column chromatography on silica gel (Redisep (4 g), 99:1:0.1 DCM:MeOH, NH₃ to 90:10:1 DCM:MeOH:NH₃) to afford 58 mg of the title compound as a brown glass.

¹H NMR (400 MHz, CDCl₃): δ=11.07+10.58 (1H, 2×brs), 9.30 (1H, s), 8.17 (1H, s), 8.03-7.96 (1H, m), 7.93-7.91 (3H, m), 7.77-7.71 (2H, m), 7.61-7.57 (1H, m), 7.42-7.40 (1H, m), 7.32 (1H, s), 6.32-5.69 (2H, 2×dd), 5.21-4.97 (2H, m), 3.82-3.37 (6H, m), 2.45-1.77 (8H, m), 1.70-1.38 (18H, m), 0.94-0.77 (2H, m), −0.09- −0.13 (9H, m). LCMS (run time=2 min): Rₜ=1.69 min; m/z 806 [M+H]⁺

### Preparation 53

### 3-{2-[(2S)-Pyrrolidin-2-yl]-1H-imidazol-5-yl}-7-(4-{2-[(2S)-pyrrolidin-2-yl]-1H-imidazol-5-yl}phenyl)isoquinoline hydrochloride salt

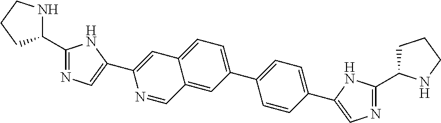

To a stirred solution of tert-butyl (2S)-2-(5-[7-(4-{2-[(2S)-1-(tert-butoxycarbonyl)pyrrolidin-2-yl]-1H-imidazol-5-yl}phenyl)isoquinolin-3-yl]-1-{[2-(trimethylsilyl)ethoxy]methyl}-1H-imidazol-2-yl)pyrrolidine-1-carboxylate (55 mg, 0.068 mmol), obtained from Preparation 52, in EtOH (1.5 mL) was added 4N hydrogen chloride in dioxane (1.53 mL, 6.12 mmol). The mixture was then heated at 75° C. for 2 hours and allowed to cool to room temperature overnight. The resulting suspension was diluted with TBME (10 mL) and the solid was collected by filtration. It was dried in vacuo at 60° C. to give 34 mg of the title compound as an orange/brown solid.

¹H NMR (400 MHz, CD₃OD): δ=9.75 (1H, s), 8.83 (1H, s), 8.81 (1H, s), 8.55 (1H, d), 8.36 (1H, d), 8.25 (1H, s), 8.14 (1H, s), 8.08 (4H, s), 5.21 (1H, t), 5.01 (1H, t, 1H), 3.69-3.52 (4H, m), 2.78-2.69 (1H, m), 2.66-2.53 (2H, m), 2.46-2.20 (5H, m). LCMS (run time=2 min): Rₜ=0.98 min; m/z 476 [M+H]⁺

### Preparation 54

### 2-[2-(6-Bromopyridin-3-yl)-2-oxoethyl]1-tert-butyl (2S)-pyrrolidine-1,2-dicarboxylate

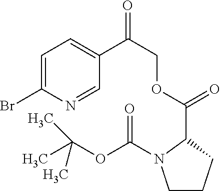

To a solution of Boc-L-proline (3.64 g, 16.9 mmol) in dichloromethane (50 mL), cooled to 0° C. and stirred, was added 2-bromo-1-(6-bromopyridin-3-yl)ethanone (4.96 g, 17.8 mmol) followed by DIPEA (6.2 mL, 35.5 mmol).

The reaction mixture was allowed to warm to room temperature and was stirred overnight. It was then washed with water (100 mL), saturated aqueous sodium bicarbonate solution (150 mL), water (150 mL) and brine (150 mL). The organic phase was dried over MgSO₄ and evaporated to give a yellow oil (7.1 g).

¹H NMR (400 MHz, CDCl₃): δ=8.86 (1H, s), 8.03 (1H, dt), 7.66 (1H, dd), 5.32 (2H, m), 4.45 (1H, m), 3.57 (1H, m), 3.44 (1H, m), 2.29 (2H, m), 2.05 (1H, m), 1.94 (1H, m) 1.46 (9H, m).

### Preparation 55

### tert-Butyl (2S)-2-[5-(6-bromopyridin-3-yl)-1H-imidazol-2-yl]pyrrolidine-1-carboxylate

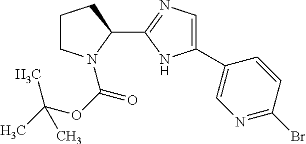

To a stirred solution of 2-[2-(6-bromopyridin-3-yl)-2-oxoethyl] 1-tert-butyl (2S)-pyrrolidine-1,2-dicarboxylate (7.0 g, 16.9 mmol), obtained from Preparation 54, in xylenes (40 mL) was added ammonium acetate (6.5 g, 84.7 mmol). The reaction mixture was brought to reflux for 6 hours, after which time it was cooled to room temperature and diluted with ethyl acetate (100 mL). The organic phase was washed with water (3×100 mL) and brine (150 mL). It was then dried over MgSO₄ and evaporated. The crude material was purified by column chromatography on silica gel (ethyl acetate:heptane 1:1 to 7:3) to give the title compound as an off-white foam (5.3 g).

¹H NMR (400 MHz, DMSO-d₆): δ=12.06 (1H, m), 8.74 (1H, d), 8.03 (1H, dd), 7.68 (1H, m), 7.58 (1H, d), 4.80 (1H, m), 3.52 (1H, m), 3.35 (1H, m), 2.21 (1H, m), 1.91 (3H, m), 1.25 (9H, m).

### Preparation 56

### tert-Butyl (2S)-2-{5-[6-(6-bromoquinoxalin-2-yl)pyridin-3-yl]-1H-imidazol-2-yl}pyrrolidine-1-carboxylate

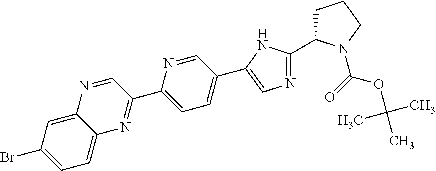

To a stirred solution of tert-butyl (2S)-2-[5-(6-bromopyridin-3-yl)-1H-imidazol-2-yl]pyrrolidine-1-carboxylate (1.5 g, 3.8 mmol), obtained from Preparation 55, in dry 1,4-dioxane (25 mL) was added hexamethylditin (1.25 g, 3.8 mmol), followed by Pd(PPh₃)₄ (1.1 g, 0.95 mmol). The reaction mixture was degassed, put under nitrogen three times and then heated at reflux for 2 hours. The reaction mixture was then allowed to cool to room temperature and diluted with ethyl acetate (50 mL). The organic phase was washed with saturated aqueous ammonium chloride solution (50 mL), water (50 mL) and brine (50 mL). The organic portion was then dried over sodium sulphate and evaporated. The residue was then dissolved in DMF (25 mL). 6-Bromo-2-chloroquinoxaline (0.93 g, 3.8 mmol) was added, followed by cesium fluoride (1.1 g, 6.9 mmol), copper (I) chloride (0.34 g, 13.8 mmol) and Pd(PPh₃)₄ (1.0 g, 0.86 mmol). The reaction mixture was degassed three times and then heated to 110° C. for 5 hours. It was allowed to cool to room temperature and then was poured into ethyl acetate (200 mL). The resulting suspension was washed with 0.880 ammonia solution (200 mL). The aqueous layer was extracted with more ethyl acetate (2×50 mL) and the combined organic layers were dried over sodium sulphate and evaporated. The crude product was purified by column chromatography (ethyl acetate:heptane 1:3 to 1:1) to afford the title compound as a yellow solid (0.73 g).

¹H NMR (400 MHz, DMSO-d₆): δ=12.16 (1H, m), 9.90 (1H, s), 9.19 (1H, d), 8.51 (1H, d), 8.38 (1H, d), 8.34 (1H, dd), 8.11 (1H, d), 8.03 (1H, dd), 7.82 (1H, m), 4.85 (1H, m), 3.56 (1H, m), 3.38 (1H, m), 2.24 (2H, m), 1.94 (2H, m), 1.30 (9H, br s, 9H).

### Preparation 57

### tert-Butyl (2S)-2-(5-[2-(5-{2-[(2S)-1-(tert-butoxycarbonyl)pyrrolidin-2-yl]-1H-imidazol-4-yl}pyridin-2-yl)quinoxalin-6-yl]-1-{[2-(trimethylsilyl)ethoxy]methyl}-1H-imidazol-2-yl)pyrrolidine-1-carboxylate

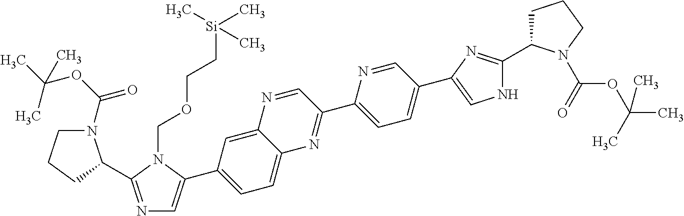

tert-Butyl (2S)-2-{5-[6-(6-bromoquinoxalin-2-yl)pyridin-3-yl]-1H-imidazol-2-yl}pyrrolidine-1-carboxylate (0.2 g, 0.38 mmol), obtained from Preparation 56, was added to a microwave vial followed by (2-[(2S)-1-(tert-butoxycarbonyl)pyrrolidin-2-yl]-1-{[2 (trimethylsilyl)ethoxy]methyl}-1H-imidazol-5-yl)boronic acid (0.2 g, 0.5 mmol), obtained from Preparation 13b, and Pd(dppf)Cl₂.DCM (0.02 g, 0.08 mmol). The reaction mixture was then dissolved in dimethoxyethane (3 mL) and 2 M sodium carbonate solution (0.6 mL) was added. The reaction was sealed and heated under microwave irradiation at 120° C. for 40 minutes. The reaction mixture was then evaporated and the crude product was purified by reverse phase chromatography (acetonitrile+0.1% formic acid:water+0.1% formic acid 3:97 to 85:15) to give the title compound as a brown solid (150 mg).

LCMS (run time=4.5 min): Rₜ=2.86 min; m/z 808 [M+H]⁺

### Preparation 58

### 6-{2-[(2S)-Pyrrolidin-2-yl]-1H-imidazol-5-yl}-2-(5-{2-[(2S)-pyrrolidin-2-yl]-1H-imidazol-4-yl}pyridin-2-yl)quinoxaline

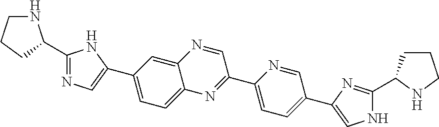

To a stirred solution of tert-butyl (2S)-2-(5-[2-(5-{2-[(2S)-1-(tert-butoxycarbonyl)pyrrolidin-2-yl]-1H-imidazol-4-yl}pyridin-2-yl)quinoxalin-6-yl]-1-{[2-(trimethylsilyl)ethoxy]methyl}-1H-imidazol-2-yl)pyrrolidine-1-carboxylate (0.15 g, 0.19 mmol), obtained from Preparation 57, in methanol (2 mL) was added 4N HCl in dioxane solution (0.93 mL). The reaction mixture was heated to 75° C. for 1.5 hours. After this time, it was evaporated and the crude product was purified by SCX column (methanol to ammonia in methanol) to afford the title compound as a brown solid (74 mg).

LCMS (run time=4.5 min): Rₜ=1.50 min; m/z 478 [M+H]⁺

### Preparation 59

### tert-Butyl (2S)-2-[5-(7-hydroxyquinolin-3-yl)-1-{[2-(trimethylsilyl)ethoxy]methyl}-1H-imidazol-2-yl]pyrrolidine-1-carboxylate

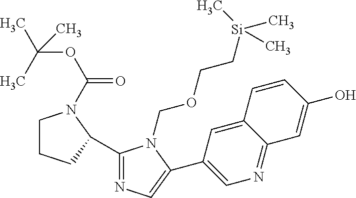

3-Bromoquinolin-7-yl tert-butyl carbonate (0.25 g, 0.77 mmol) was added to a microwave vial followed by (2-[(2S)-1-(tert-butoxycarbonyl)pyrrolidin-2-yl]-1-{[2 (trimethylsilyl)ethoxy]methyl}-1H-imidazol-5-yl)boronic acid (0.7 g, 0.85 mmol), obtained from Preparation 13b, and Pd(dppf)Cl₂.DCM (0.04 g, 0.15 mmol). The reaction mixture was dissolved in dimethoxyethane (2 mL), and then 2M sodium carbonate solution (1.2 mL) was added. The reaction was sealed and then heated under microwave irradiation at 120° C. for 90 minutes. The reaction mixture was diluted with dichloromethane (10 mL) then placed in a sonic bath for 5 minutes. It was then passed through a phase separation cartridge and the filtrate was evaporated. The crude product was purified by column chromatography on silica gel (ethyl acetate:methanol 8:2) to give the title compound as a light brown solid (204 mg).

LCMS (run time=4.5 min): Rₜ=2.54 min; m/z 511 [M+H]⁺

### Preparation 60

### tert-Butyl (2S)-2-[5-(7-{[(trifluoromethyl)sulfonyl]oxy}quinolin-3-yl)-1-{[2-(trimethyl silyl)ethoxy]methyl}-1H-imidazol-2-yl]pyrrolidine-1-carboxylate

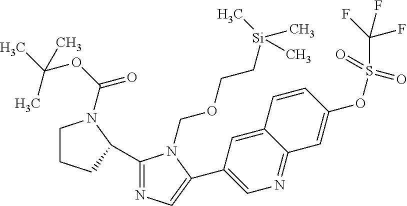

To a stirred solution of tert-butyl (2S)-2-[5-(7-hydroxyquinolin-3-yl)-1-{[2-(trimethylsilyl)ethoxy]methyl}-1H-imidazol-2-yl]pyrrolidine-1-carboxylate (0.2 g, 0.4 mmol), obtained from Preparation 59, in dry dichloromethane (5 mL) was added triethylamine (0.22 mL, 1.6 mmol). The mixture was cooled to −60° C. and trifluoromethanesulfonic anhydride (0.1 mL, 0.6 mmol) was then added. It was stirred at −60° C. for 5 minutes, after which time, it was diluted with dichloromethane (20 mL) and washed with saturated sodium bicarbonate solution (20 mL). The organic layer was dried over sodium sulphate and evaporated. The crude material was purified by column chromatography on silica gel (dichloromethane: methanol 97.5:2.5) to give the title compound as an orange oil (164 mg).

LCMS (run time=4.5 min): Rₜ=3.58 min; m/z 643 [M+H]⁺

### Preparation 61

### tert-Butyl (2S)-2-(5-[7-(4,4,5,5-tetramethyl-1,3,2-dioxaborolan-2-yl)quinolin-3-yl]-1-{[2-(trimethylsilyl)ethoxy]methyl}-1H-imidazol-2-yl)pyrrolidine-1-carboxylate

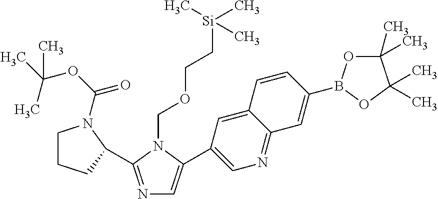

tert-Butyl (2S)-2-[5-(7-{[(trifluoromethyl)sulfonyl]oxy}quinolin-3-yl)-1-{[2-(trimethylsilyl)ethoxy]methyl}-1H-imidazol-2-yl]pyrrolidine-1-carboxylate (0.16 g, 0.24 mmol), obtained from Preparation 60, was added to a microwave vial followed by bis-pinacolatodiborane (0.12 g, 0.49 mmol), potassium acetate (0.06 g, 0.61 mmol) and Pd(dppf)Cl₂.DCM (0.006 g, 0.024 mmol). The reaction mixture was then dissolved in 1,4-dioxane (2 mL). The vessel was sealed and then heated under microwave irradiation at 120° C. for 2 hours. The reaction mixture was diluted with dichloromethane (10 mL) and filtered through a pad of celite, which was washed thoroughly with more dichloromethane (25 mL). The solvent was evaporated and the crude product was purified by column chromatography on silica gel (dichloromethane:methanol 97:3) to give the title compound as a yellow oil (114 mg). Hydrolysis to the boronic acid was observed in the LCMS.

LCMS (run time=4.5 min): Rₜ=3.56 min; m/z 621 [M+H]⁺

### Preparation 62

### tert-Butyl (2S)-2-(5-[7-(5-{2-[(2S)-1-(tert-butoxycarbonyl)pyrrolidin-2-yl]-1H-imidazol-5-yl}pyridin-2-yl)quinolin-3-yl]-1-{[2-(trimethylsilyl)ethoxy]methyl}-1H-imidazol-2-yl)pyrrolidine-1-carboxylate

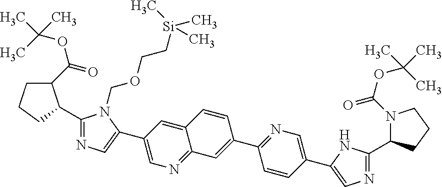

tert-Butyl (2S)-2-[5-(6-bromopyridin-3-yl)-1H-imidazol-2-yl]pyrrolidine-1-carboxylate (0.065 g, 0.16 mmol), from Preparation 55, tert-butyl (2S)-2-(5-[7-(4,4,5,5-tetramethyl-1,3,2-dioxaborolan-2-yl)quinolin-3-yl]-1-{[2-(trimethylsilyl)ethoxy]methyl}-1H-imidazol-2-yl)pyrrolidine-1-carboxylate (0.12 g, 0.18 mmol), obtained from Preparation 61, and Pd(dppf)Cl₂.DCM (0.01 g, 0.03 mmol) were added to a microwave vial. The reaction mixture was dissolved in dimethoxyethane (0.6 mL) and 2M sodium carbonate solution (0.25 mL) was added.

The microwave vial was sealed and then heated under microwave irradiation at 120° C. for 40 minutes. Silica (1 g) was added to the reaction mixture and the solvent was evaporated. The crude product was purified by column chromatography on silica gel (dichloromethane:methanol 9:1) to give the title product as a pale green solid (94 mg).

LCMS (run time=4.5 min): Rₜ=2.66 min; m/z 807 [M+H]⁺

### Preparation 63

### 3-{2-[(2S)-Pyrrolidin-2-yl]-1H-imidazol-5-yl}-7-(5-{2-[(2S)-pyrrolidin-2-yl]-1H-imidazol-5-yl}pyridin-2-yl)quinoline

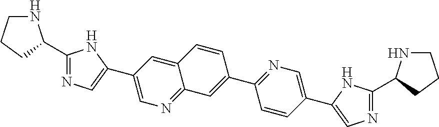

To a stirred solution of tert-butyl (2S)-2-(5-[7-(5-{2-[(2S)-1-(tert-butoxycarbonyl)pyrrolidin-2-yl]-1H-imidazol-5-yl}pyridin-2-yl)quinolin-3-yl]-1-{[2-(trimethylsilyl)ethoxy]methyl}-1H-imidazol-2-yl)pyrrolidine-1-carboxylate (0.094 g, 0.12 mmol), obtained from Preparation 62, in dry methanol (2 mL) was added 4 N HCl in dioxane solution (1 mL). The reaction mixture was heated to 75° C. for 1 hour. After this time, the reaction mixture was evaporated and the crude product was purified by SCX column (gradient methanol to ammonia in methanol) to give the title compound as a brown solid (60 mg).

LCMS (run time=4.5 min): Rₜ=1.37 min; m/z 477 [M]⁺

### Preparation 64

### tert-Butyl (2S)-2-[5-(3-chloroisoquinolin-7-yl)-1-{[2-(trimethylsilyl)ethoxy]methyl}-1H-imidazol-2-yl]pyrrolidine-1-carboxylate

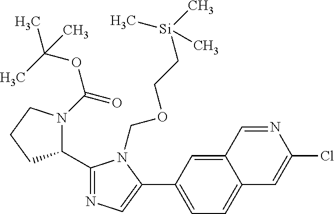

7-Bromo-3-chloroisoquinoline (0.6 g, 2.5 mmol), (2-[(2S)-1-(tert-butoxycarbonyl)pyrrolidin-2-yl]-1-{[2(trimethylsilyl)ethoxy]methyl}-1H-imidazol-5-yl)boronic acid (0.68 g, 1.5 mmol), obtained from Preparation 13b, and Pd(dppf)Cl₂.DCM (0.2 g, 0.25 mmol) were added to a microwave vial. The reaction mixture was dissolved in dimethoxyethane (6 mL) and 2M sodium carbonate solution (1.9 mL) was added. The reaction vessel was sealed and heated under microwave radiation at 120° C. for 1 hour. Dichloromethane (40 mL) was added to the reaction mixture and it was filtered through Arbocel, which was washed with more dichloromethane (80 mL). The solvent was evaporated and the crude product was purified by column chromatography on silica gel (ethyl acetate:heptane 2:3 to 3:2) to give the title compound as a yellow gum (750 mg).

LCMS (run time=4.5 min): Rₜ=3.11 min; m/z 529; 531 [M+H]⁺

### Preparation 65

### tert-Butyl (2S)-2-(5-[3-(5-{2-[(2S)-1-(tert-butoxycarbonyl)pyrrolidin-2-yl]-1H-imidazol-4-yl}pyridine-2-yl)isoquinolin-7-yl]-1-{[2-(trimethylsilyl)ethoxy]methyl}-1H-imidazol-2-yl)pyrrolidine-1-carboxylate

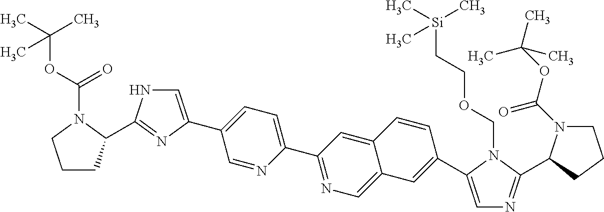

To a solution of tert-butyl (2S)-2-[5-(6-bromopyridin-3-yl)-1H-imidazol-2-yl]pyrrolidine-1-carboxylate (0.5 g, 1.27 mmol), obtained from Preparation 55, in dry 1,4-dioxane (5 mL), was added hexamethyl di-tin (0.42 g, 1.27 mmol) followed by Pd(PPh₃)₄ (0.37 g, 0.32 mmol). The reaction mixture was degassed and put under nitrogen three times. It was then heated at reflux for 2 hours, after which time, it was allowed to cool to room temperature and diluted with ethyl acetate (20 mL). The organic phase was washed with saturated aqueous ammonium chloride solution (20 mL), water (20 mL) and brine (20 mL). The organic phase was then dried over sodium sulphate and evaporated. The residue was dissolved in dry DMF (5 mL) and tert-butyl (2S)-2-[5-(3-chloroisoquinolin-7-yl)-1-{[2-(trimethylsilyl)ethoxy]methyl}-1H-imidazol-2-yl]pyrrolidine-1-carboxylate (0.75 g, 1.27 mmol), obtained from Preparation 64, was added. Cesium fluoride (0.35 g, 2.29 mmol), copper (I) chloride (0.13 g, 1.27 mmol) and Pd(PPh₃)₄ (0.16 g, 0.14 mmol) were also added. The reaction mixture was then degassed and put under nitrogen three times. The reaction was stirred at 110° C. for 5 hours. It was allowed to cool to room temperature and then diluted with ethyl acetate (50 mL). The resulting suspension was washed with 0.880 ammonia solution (50 mL). The aqueous layer was washed with more ethyl acetate (2×50 mL). The combined organic phases were dried over sodium sulphate and evaporated. The crude product was purified by column chromatography on silica gel (ethyl acetate:methanol 1:0 to 95:5) to give the title product as a yellow solid (400 mg).

LCMS (run time=4.5 min): Rₜ=2.69 min; m/z 807 [M+H]⁺

### Preparation 66

### 7-{2-[(2S)-Pyrrolidin-2-yl]-1H-imidazol-5-yl}-3-(5-{2-[(2S)-pyrrolidin-2-yl]-1H-imidazol-4-yl}pyridine-2-yl)isoquinoline hydrochloride salt

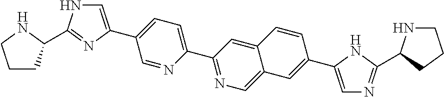

To a stirred solution of tert-butyl (2S)-2-(5-[3-(5-{2-[(2S)-1-(tert-butoxycarbonyl)pyrrolidin-2-yl]-1H-imidazol-4-yl}pyridine-2-yl)isoquinolin-7-yl]-1-{[2-(trimethylsilyl)ethoxy]methyl}-1H-imidazol-2-yl)pyrrolidine-1-carboxylate (0.4 g, 0.5 mmol), obtained from Preparation 65, in dry methanol (2 mL) was added 4N HCl in 1,4-dioxane (2.5 mL). The reaction mixture was stirred at 75° C. for 90 minutes. It was then allowed to cool to room temperature and evaporated to give the title compound as an orange solid (300 mg).

LCMS (run time=4.5 min): Rₜ=1.36 min; m/z 477 [M+H]⁺

### Preparation 67

### tert-Butyl (2S)-2-{4-[4-(7-bromo-1,5-naphthyridin-3-yl)phenyl]-1H-imidazol-2-yl}pyrrolidine-1-carboxylate

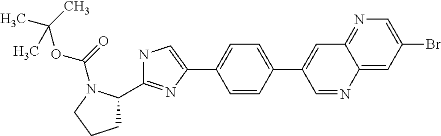

tert-Butyl (2S)-2-{4-[4-(4,4,5,5-tetramethyl-1,3,2-dioxaborolan-2-yl)phenyl]-1H-imidazol-2-yl}pyrrolidine-1-carboxylate (275 mg, 0.63 mmol), obtained from Preparation 16,3,7-dibromo-1,5-naphthyridine (198 mg, 0.69 mmol) and Pd(dppf)Cl₂.DCM (31.2 mg, 0.13 mmol) were added to a microwave vial, followed by dimethoxyethane (6 mL) and 2M aq. sodium carbonate solution (2 mL). The mixture was heated under microwave irradiation for 40 minutes at 120° C. It was then evaporated and the crude product was purified by column chromatography on silica gel (ethyl acetate; heptane 1:1 to 1:0) to give the title compound as an off-white solid (170 mg).

LCMS (run time=4.5 min): Rₜ=2.25 min; m/z 520; 522 [M+H]⁺

### Preparation 68

### tert-Butyl (2S)-2-(5-[7-(4-{2-[(2S)-1-(tert-butoxycarbonyl)pyrrolidin-2-yl]-1H-imidazol-4-yl}phenyl)-1,5-naphthyridin-3-yl]-1-{[2-(trimethylsilyl)ethoxy]methyl}-1H-imidazol-2-yl)pyrrolidine-1-carboxylate

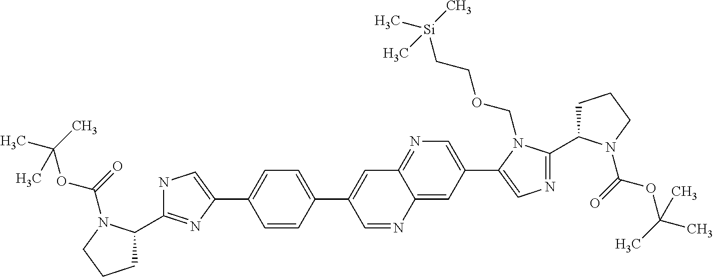

tert-Butyl (2S)-2-{4-[4-(7-bromo-1,5-naphthyridin-3-yl)phenyl]-1H-imidazol-2-yl}pyrrolidine-1-carboxylate (289 mg, 0.56 mmol), obtained from Preparation 67, (2-[(2S)-1-(tert-butoxycarbonyl)pyrrolidin-2-yl]-1-{[2-(trimethylsilyl)ethoxy]methyl}-1H-imidazol-5-yl)boronic acid (359 mg, 0.61 mmol), obtained from Preparation 13b, and Pd(dppf)Cl₂.DCM (28 mg, 0.11 mmol) were added to a microwave vial, followed by dimethoxyethane (4 mL) and 2M aq. sodium carbonate solution (0.8 mL). The mixture was heated under microwave irradiation for 40 minutes at 120° C. It was then evaporated and the crude product was purified by column chromatography on silica gel (dichloromethane:methanol 96:4) to give the title product as a yellow oil (289 mg).

LCMS (run time=4.5 min): Rₜ=2.54 min; m/z 807 [M]⁺

### Preparation 69

### 3-{2-[(2S)-Pyrrolidin-2-yl]-1H-imidazol-5-yl}-7-(4-{2-[(2S)-pyrrolidin-2-yl]-1H-imidazol-4-yl}phenyl)-1,5-naphthyridine

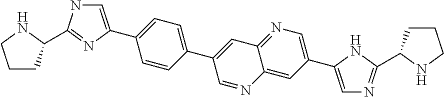

tert-Butyl (2S)-2-(5-[7-(4-{2-[(2S)-1-(tert-butoxycarbonyl)pyrrolidin-2-yl]-1H-imidazol-4-yl}phenyl)-1,5-naphthyridin-3-yl]-1-{[2-(trimethylsilyl)ethoxy]methyl}-1H-imidazol-2-yl)pyrrolidine-1-carboxylate (289 mg, 0.358 mmol), obtained from Preparation 68, was dissolved in ethanol (5 mL), and then 4M HCl in 1,4-dioxane (2.24 mL) was added. The reaction mixture was heated to 75° C. for 90 minutes. It was allowed to cool to room temperature and then the solvent was evaporated. The crude product was purified by SCX column (gradient methanol to ammonia in methanol) to give the title compound as a yellow solid (184 mg).

LCMS (run time=4.5 min): Rₜ=1.43 min; m/z 477 [M+H]⁺

### Preparation 70

### (2S)-2-[(Methoxycarbonyl)amino]butanoic acid

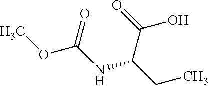

To a stirred solution L-2-aminobutryic acid (2.00 g, 19.40 mmol) and Na₂CO₃ (1.03 g, 9.70 mmol) in 1M NaOH (20 mL, 19.5 mmol), at 0° C., was added methyl chloroformate (1.64 mL, 21.3 mmol), dropwise. The reaction mixture was stirred at room temperature for 4 hours. It was then washed with TBME (2×40 mL) and the aqueous phase was cooled to 0° C. before being acidified to pH 1 with 6 M HCl. The cloudy suspension was then extracted with DCM (6×50 mL). The combined organic extracts were dried (Na₂SO₄), filtered and evaporated to give the title compound as a white solid (1.792 g).

¹H NMR (400 MHz, DMSO-d₆): δ=3.53 (3H, s), 1.76-1.66 (1H, m), 1.63-1.52 (1H, m), 0.88 (3H, t).

### Preparation 71

### Methyl {(2S)-1-[(2S)-2-{5-[4-(2-chloroquinolin-6-yl)phenyl]-1H-imidazol-2-yl}pyrrolidin-1-yl]-3-methyl-1-oxobutan-2-yl}carbamate

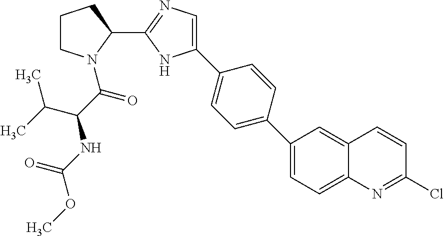

6-Bromo-2-chloro-quinoline (350 mg, 1.44 mmol), methyl {(2S)-3-methyl-1-oxo-1-[(2S)-2-{5-[4-(4,4,5,5-tetramethyl-1,3,2-dioxaborolan-2-yl)phenyl]-1H-imidazol-2-yl}pyrrolidin-1-yl]butan-2-yl}carbamate (716 mg, 1.44 mmol), obtained from Preparation 28, 2N sodium bicarbonate (2.16 mL, 4.33 mmol) and Pd(dppf)Cl₂.DCM (58 mg, 0.072 mmol) were added to a microwave vial, followed by DME (3 mL). The mixture was heated under microwave irradiation at 120° C. for 30 minutes. It was then absorbed onto silica and purified by column chromatography on silica gel (Redisep 40 g. eluting with a gradient of heptane: ethyl acetate (100:0 to 0:100) then 100% DCM: MeOH: NH₃90:10:1 to afford 390 mg of the title compound as an orange foam.

LRMS (run time=2 min) Rₜ=1.28 min; m/z 532; 534 [M+H]⁺

### Preparation 72

### Methyl {(2S)-3-methyl-1-oxo-1-[(2S)-2-{5-[4-(2-{2-[(2S)-pyrrolidin-2-yl]-1H-imidazol-5-yl}quinolin-6-yl)phenyl]-1H-imidazol-2-yl}pyrrolidin-1-yl]butan-2-yl}carbamate

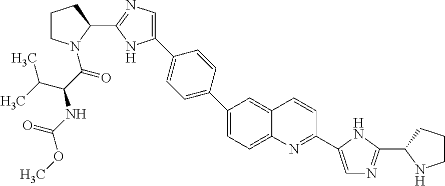

Methyl {(2S)-1-[(2S)-2-{5-[4-(2-chloroquinolin-6-yl)phenyl]-1H-imidazol-2-yl}pyrrolidin-1-yl]-3-methyl-1-oxobutan-2-yl}carbamate (65 mg, 0.12 mmol), obtained from Preparation 71, (2-[(2S)-1-(tert-butoxy carbonyl)pyrrolidin-2-yl]-1-{[2-(trimethylsilyl)ethoxy]methyl}-1H-imidazol-5-yl)boronic acid (50 mg, 0.12 mmol), obtained from Preparation 13b, 2N sodium bicarbonate (0.183 mL, 0.366 mmol) and Pd(dppf)Cl₂.DCM (5 mg, 0.006 mmol) were added to a microwave vial, followed by DME (1 mL). The mixture was heated under microwave irradiation at 120° C. for 30 minutes. It was then partitioned between ethyl acetate and water. The organic phase was dried over MgSO₄ and evaporated. The residue was redissolved in ethanol (2 mL) and treated with 4N HCl/Dioxan (2 mL) at room temperature for 3 days. The reaction was concentrated and applied to an SCX cartridge eluting initially with methanol then 7N ammonia in methanol to elute the product as a brown glass (66 mg).

LRMS (run time=2 min) Rₜ=0.95 min; m/z 633 [M+H]⁺

### Preparation 73

### 7-Bromopyrido[2,3-b]pyrazin-3(4H)-one

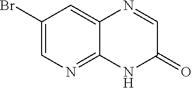

5-Bromopyridine-2,3-diamine (5.0 g, 26.6 mmol) was dissolved in methanol (100 mL) and cooled to 0° C. Oxoacetic acid (2.94 g, 31.9 mmol), in methanol (50 mL), was added dropwise and the resulting mixture was allowed to warm to room temperature, and stirred under nitrogen overnight. The solvent was then evaporated and the residue was triturated with TBME (50 mL). The resulting solid was collected by filtration, washing with TBME (20 mL), to give a mixture of the title compound and it's regioisomer as a brown solid (5.84 g).

LCMS (run time=6 min): Rₜ=1.58 min; m/z 226; 228 [M+H]⁺(77%) and Rₜ=2.02 min; m/z 226; 228 [M+H]⁺(23%)

### Preparation 74

### 7-Bromo-3-chloropyrido[2,3-b]pyrazine

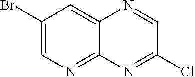

7-Bromopyrido[2,3-b]pyrazin-3(4H)-one and its regioisomer (5.84 g, 25.84 mmol), obtained from Preparation 73, and phosphorus oxychloride (30 mL) were heated at 120° C. for 1.5 hours, then cooled to room temperature. The solvent was evaporated and the residue was quenched with crushed ice. The aqueous suspension was neutralised with 10% potassium carbonate solution and extracted with ethyl acetate. The solid which remained was removed by filtration and the aqueous phase was extracted with more ethyl acetate. The organic layers were combined, dried over MgSO₄ and evaporated. The crude product was purified by column chromatography (ethyl acetate:heptane 1:1) to give the title compound as an orange oil (354 mg).

¹H NMR (400 MHz, DMSO-d₆): δ=9.26 (1H, d), 9.61 (1H, s), 8.98 (1H, d).

### Preparation 75

### tert-Butyl (2S)-2-{5-[4-(7-bromopyrido[2,3-b]pyrazin-3-yl)phenyl]-1H-imidazol-2-yl}pyrrolidine-1-carboxylate

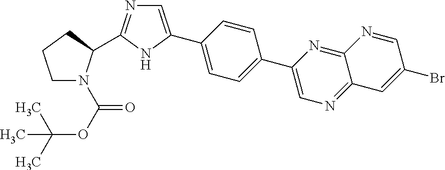

To a stirred solution of 7-bromo-3-chloropyrido[2,3-b]pyrazine (350 mg, 1.43 mmol), obtained from Preparation 74, (S)-tert-butyl 2-(5-(4-(4,4,5,5-tetramethyl-1,3,2-dioxaborolan-2-yl)phenyl)-1H-imidazol-2-yl)pyrrolidine-1-carboxylate (661 mg, 1.50 mmol), obtained from Preparation 16, and Pd(dppf)Cl₂.DCM (117 mg, 0.14 mmol), in dimethoxyethane (5 mL), was added 2 M sodium bicarbonate solution (2.15 mL). The mixture was evacuated and placed under nitrogen three times. It was then stirred at room temperature for 1 hour. The reaction mixture was evaporated and the crude product was purified by column chromatography on silica gel (ethyl acetate:heptane 1:1 to 1:0) to give the title compound as a bright yellow solid (352 mg).

¹H NMR (400 MHz, DMSO-d₆): δ=11.98 (1H, m), 9.74 (1H, m), 9.21 (1H, m), 8.87 (1H, s), 8.38 (2H, m), 7.98 (2H, m), 7.69 (1H, m), 4.82 (1H, m), 3.39-3.31 (2H, m), 2.30-1.80 (4H, m), 1.43-1.08 (9H, m). LCMS (run time=6 min): Rₜ=2.47 min; m/z 521; 523 [M+H]⁺

### Preparation 76

### tert-Butyl (2S)-2-(5-[3-(4-{2-[(2S)-1-(tert-butoxycarbonyl)pyrrolidin-2-yl]-1H-imidazol-4-yl}phenyl)pyrido[2,3-b]pyrazin-7-yl]-1-{[2-(trimethylsilyl)ethoxy]methyl}-1H-imidazol-2-yl)pyrrolidine-1-carboxylate

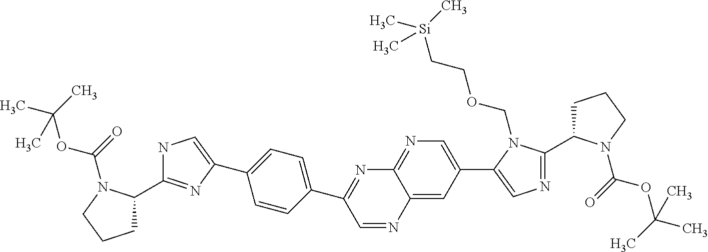

tert-Butyl (2S)-2-{5-[4-(7-bromopyrido[2,3-b]pyrazin-3-yl)phenyl]-1H-imidazol-2-yl}pyrrolidine-1-carboxylate (100 mg, 0.192 mmol), obtained from Preparation 75, (2-[(2S)-1-(tert-butoxycarbonyl)pyrrolidin-2-yl]-1-{[2 (trimethylsilyl)ethoxy]methyl}-1H-imidazol-5-yl)boronic acid (124 mg, 0.211 mmol), obtained from Preparation 13b, and Pd(dppf)Cl₂.DCM (9.5 mg, 0.038 mmol) were dissolved in dimethoxyethane (4 mL) and 2N sodium carbonate solution (0.288 mL), in a microwave vial. The vial was sealed, evacuated, and placed under nitrogen three times, before heating under microwave irradiation at 120° C. for 30 minutes. A further charge of (2-[(2S)-1-(tert-butoxycarbonyl)pyrrolidin-2-yl]-1-{[2 (trimethylsilyl)ethoxy]methyl}-1H-imidazol-5-yl) boronic acid (124 mg, 0.211 mmol), obtained from Preparation 13b, was added and heating was continued at 120° for a further 15 minutes. The solvent was then evaporated and the crude product was purified by column chromatography on silica gel (dichloromethane: methanol: ammonia 93:7:0.8) to give the title compound as a bright yellow oil (292 mg). LCMS (run time=6 min): Rₜ=2.92 min; m/z 808 [M+H]⁺

### Preparation 77

### 7-{2-[(2S)-Pyrrolidin-2-yl]-1H-imidazol-5-yl}-3-(4-{2-[(2S)-pyrrolidin-2-yl]-1H-imidazol-5-yl}phenyl)pyrido[2,3-b]pyrazine

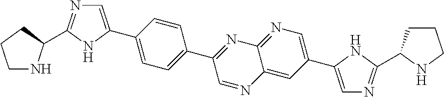

To a stirred solution of tert-butyl (2S)-2-(5-[3-(4-{2-[(2S)-1-(tert-butoxycarbonyl)pyrrolidin-2-yl]-1H-imidazol-4-yl}phenyl)pyrido[2,3-b]pyrazin-7-yl]-1-{[2-(trimethylsilyl)ethoxy]methyl}-1H-imidazol-2-yl)pyrrolidine-1-carboxylate (292 mg, 0.361 mmol), obtained from Preparation 76, in dichloromethane (2 mL), was added 4M HCl in 1,4-dioxane (5 mL, 20.0 mmol). The reaction mixture was stirred at room temperature for 1 hour. The solvent was then evaporated and the crude product was dissolved in methanol and loaded onto a pre-wetted SCX cartridge (Biotage 2 g). The cartridge was eluted with methanol, then 7 M ammonia in methanol. The filtrate was evaporated to give the title compound as a yellow oil (180 mg) which was used directly in the next step.

### Preparation 78

### 2-Amino-5-bromobenzamide

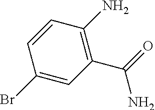

To a stirred solution of 2-amino-5-bromobenzonitrile (1.0 g, 5.0 mmol), in methanol (5 mL), at 0° C., was added 1N sodium hydroxide solution (5 mL) and hydrogen peroxide (5 mL of a 35% W/W solution in water). The mixture was allowed to warm to room temperature and stirred for 1 hour. 1N sodium hydroxide solution (1 mL) and hydrogen peroxide (1 mL of a 35% W/W solution in water) were then added and the reaction mixture was stirred for a further 10 minutes. The precipitated solid was collected by filtration and dried in vacuo to give the title compound as a white solid (929 mg).

¹H NMR (400 MHz, DMSO-d₆): δ=7.81 (1H, br s), 7.68 (1H, d), 7.23 (1H, dd), 7.15 (1H, br s), 6.69 (2H, br s), 6.63 (1H, d). LCMS (run time=6 min): Rₜ=2.17 min; m/z 215; 217 [M+H]+

### Preparation 79

### 6-Bromo-2-(4-iodophenyl)quinazolin-4-ol

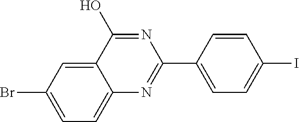

To a stirred suspension of 2-amino-5-bromobenzamide (820 mg, 3.81 mmol), obtained from Preparation 78, in dichloromethane (16 mL), was added triethylamine (1.06 mL, 2.0 mmol) then 4-iodobenzoyl chloride (1020 mg, 3.81 mmol). The mixture was stirred at room temperature under nitrogen for 2 hours. It was then filtered and the solid was then added to ethanol (9.8 mL). 5% potassium hydroxide (aq) (16 mL) was then added and the mixture was stirred at reflux for 20 minutes, then cooled to room temperature. The reaction mixture was acidified to ˜pH6 with acetic acid and the precipitate was collected by filtration, washing with water. The solid was dried in vacuo to give the title compound as a white solid (1.5 g).

¹H NMR (400 MHz, DMSO-d₆): δ=8.11 (1H, d), 8.03 (2H, m), 7.83 (2H, m), 7.77 (1H, dd), 7.53 (1H, d).

### Preparation 80

### tert-Butyl (2S)-2-(5-{4-[6-(2-[(2S)-1-(tert-butoxycarbonyl)pyrrolidin-2-yl]-1-{[2-(trimethylsilyl)ethoxy]methyl}-1H-imidazol-5-yl)-4-hydroxyquinazolin-2-yl]phenyl}-1-{[2-(trimethylsilyl)ethoxy]methyl}-1H-imidazol-2-yl)pyrrolidine-1-carboxylate

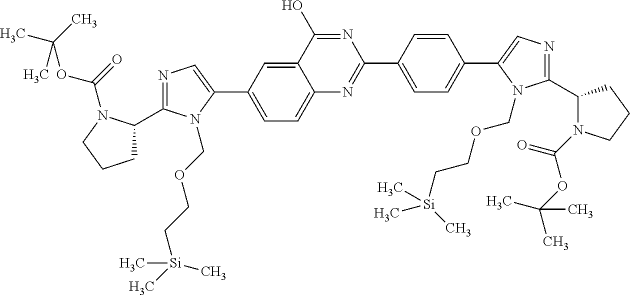

6-Bromo-2-(4-iodophenyl)quinazolin-4-ol (50 mg, 0.12 mmol), obtained from Preparation 79, (2-[(2S)-1-(tert-butoxycarbonyl)pyrrolidin-2-yl]-1-{[2 (trimethylsilyl)ethoxy]methyl}-1H-imidazol-5-yl)boronic acid (294 mg, 0.71 mmol), obtained from Preparation 13b, Pd₂(dba)₃ (0.9 mg, 1.0 μmol), tricyclohexylphosphine (0.8 mg, 3.0 μmol) and potassium phosphate (90 mg, 425 μmol) in 1,4-dioxane (1.5 mL)/water (0.335 mL) were added to a microwave vial. The vial was evacuated and placed under nitrogen three times. The reaction mixture was heated under microwave irradiation at 120° C. for 10 minutes, then a further charge of Pd₂(dba)₃ (0.9 mg, 1.0 μmol) and tricyclohexylphosphine (0.8 mg, 3.0 μmol) were added. The reaction mixture was then heated under microwave irradiation at 120° C. for a further 10 minutes. A further charge of (2-[(2S)-1-(tert-butoxycarbonyl)pyrrolidin-2-yl]-1-{[2 (trimethylsilyl)ethoxy]methyl}-1H-imidazol-5-yl)boronic acid (50 mg, 0.12 mmol), obtained from Preparation 13b, Pd₂(dba)₃ (0.9 mg, 1.0 μmol), tricyclohexylphosphine (0.8 mg, 3.0 μmol), potassium phosphate (90 mg, 425 μmol) and water (0.335 mL) were added and the reaction mixture was heated under microwave irradiation at 120° C. for a further 10 minutes. It was then diluted with ethyl acetate and washed with water. The aqueous layer was then washed again with ethyl acetate. The combined ethyl acetate extracts were dried over Na₂SO₄ and evaporated. The crude product was dry loaded onto silica and purified by column chromatography (redisep (4 g) methanol: ethyl acetate 0:100 to 10:90) to give the title compound as an yellow oil (51 mg).

¹H NMR (400 MHz, MeOD): δ=8.40 (1H, m), 8.25 (2H, m), 7.97 (1H, m), 7.90 (1H, m), 7.74 (2H, m), 7.17 (2H, m), 5.65 (1H, m), 5.47 (1H, m), 5.33 (2H, m), 5.10 (2H, m), 3.79-3.51 (8H, m), 2.48-1.73 (8H, m), 1.50-1.21 (18H, m), 0.95 (4H, m), 0.01 (18H, d). LCMS (run time=6 min): Rₜ=2.95 min; m/z 953 [M+H]⁺

### Preparation 81

### 6-{2-[(2S)-pyrrolidin-2-yl]-1H-imidazol-5-yl}-2-(4-{2-[(2S)-pyrrolidin-2-yl]-1H-imidazol-5-yl}phenyl)quinazolin-4-ol hydrochloride salt

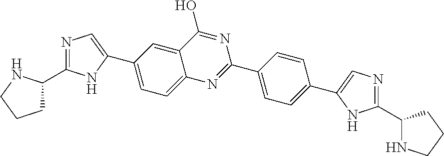

To a stirred solution of tert-butyl (2S)-2-(5-{4-[6-(2-[(2S)-1-(tert-butoxycarbonyl)pyrrolidin-2-yl]-1-{[2-(trimethylsilyl)ethoxy]methyl}-1H-imidazol-5-yl)-4-hydroxyquinazolin-2-yl]phenyl}-1-{[2-(trimethylsilyl)ethoxy]methyl}-1H-imidazol-2-yl)pyrrolidine-1-carboxylate (51 mg, 0.053 mmol), obtained from Preparation 80, in ethanol (1 mL), was added 4M HCl in 1,4-dioxane (1 mL, 30.6 mmol). The reaction mixture was stirred at 75° C. for 1 hour. It was then evaporated and azeotroped with toluene to give the title compound as a pale yellow solid (35 mg).

LCMS (run time=6 min): Rₜ=1.38 min; m/z 493 [M+H]⁺

### Preparation 82

### 6-Bromo-2-(4-iodophenyl)-4-methoxyquinazoline

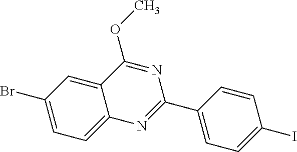

To a stirred solution of 6-bromo-2-(4-iodophenyl)quinazolin-4-ol (500 mg, 1.17 mmol), obtained from Preparation 79, in DMF (5 mL), was added sodium hydride (60% dispersion in oil) (112 mg, 2.81 mmol). The mixture was stirred at room temperature for 45 minutes. DMF (3 mL) was then added and the mixture was stirred for a further 45 minutes. Methyl iodide (0.291 mL, 4.68 mmol) was then added and the mixture was stirred for a further 30 minutes. It was then quenched with water and diluted with ethyl acetate. The organic layer was extracted, washed with brine (4×10 mL), dried over Na₂SO₄ and evaporated. Purification was carried out by column chromatography on silica gel (Redisep (40 g) ethyl acetate:heptane 0:100 to 30:70) to give the title compound as a white solid (88 mg).

¹H NMR (400 MHz, DMSO-d₆): δ=8.25 (1H, d), 7.97 (1H, dd), 7.91 (2H, m), 7.63 (1H, d), 7.47 (2H, m), 3.34 (3H, s).

### Preparation 83

### tert-Butyl (2S)-2-(5-{4-[6-(2-[(2S)-1-(tert-butoxycarbonyl)pyrrolidin-2-yl]-1-{[2-(trimethylsilyl)ethoxy]methyl}-1H-imidazol-5-yl)-4-methoxyquinazolin-2-yl]phenyl}-1-{[2-(trimethylsilyl)ethoxy]methyl}-1H-imidazol-2-yl)pyrrolidine-1-carboxylate

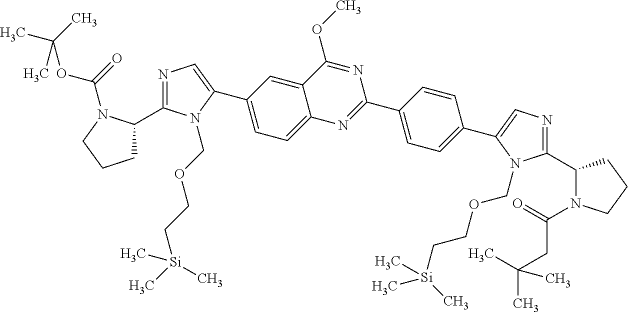

6-Bromo-2-(4-iodophenyl)-4-methoxyquinazoline (44 mg, 0.10 mmol), obtained from Preparation 82, (2-[(2S)-1-(tert-butoxycarbonyl)pyrrolidin-2-yl]-1-{[2 (trimethylsilyl)ethoxy]methyl}-1H-imidazol-5-yl)boronic acid (159 mg, 0.39 mmol), obtained from Preparation 13b, Pd₂(dba)₃ (0.9 mg, 1.0 μmol), copper (1) chloride (20 mg, 0.20 mmol), tricyclohexylphosphine (0.6 mg, 2.0 μmol) and potassium phosphate (72 mg, 340 μmol) in 1,4-dioxane (2.1 mL)/water (0.268 mL) were added to a microwave vial. The vial was evacuated and placed under nitrogen three times. The reaction mixture was heated under microwave irradiation at 120° C. for 10 minutes, then a further charge of (2-[(2S)-1-(tert-butoxycarbonyl)pyrrolidin-2-yl]-1-{[2 (trimethylsilyl)ethoxy]methyl}-1H-imidazol-5-yl)boronic acid (106 mg, 0.26 mmol), obtained from Preparation 13b, Pd₂(dba)₃ (0.9 mg, 1.0 μmol) tricyclohexylphosphine (0.6 mg, 2.0 μmol), potassium phosphate (72 mg, 340 μmol), 1,4-dioxane (0.4 mL) and water (0.268 mL) were added. The mixture was heated under microwave irradiation at 120° C. for 20 minutes. It was then diluted with ethyl acetate and washed with 880 ammonia solution. The aqueous layer was washed again with ethyl acetate. The combined ethyl acetate extracts were dried over Na₂SO₄ and evaporated. The crude product was purified by column chromatography on silica gel (redisep (12 g) ethyl acetate: heptane 50:50 to 100:0) to give the title compound as a orange oil (90 mg).

¹H NMR (400 MHz, MeOD): δ=8.71 (2H, m), 8.37 (1H, m), 8.13-8.01 (2H, m), 7.68 (2H, m), 7.23-7.06 (2H, m), 5.66 (1H, m), 5.50 (1H, m), 5.39-5.27 (2H, m), 5.16-5.05 (2H, m), 4.37 (3H, s), 3.80-3.48 (8H, m), 2.51-1.93 (8H, m), 1.51-1.18 (18H, m), 0.94 (4H, m), 0.00 (18H, m). LCMS (run time=6 min): Rₜ=3.54 min; m/z 967 [M+H]⁺

### Preparation 84

### 4-Ethoxy-6-{2-[(2S)-pyrrolidin-2-yl]-1H-imidazol-5-yl}-2-(4-{2-[(2S)-pyrrolidin-2-yl]-1H-imidazol-5-yl}phenyl)quinazoline hydrochloride salt

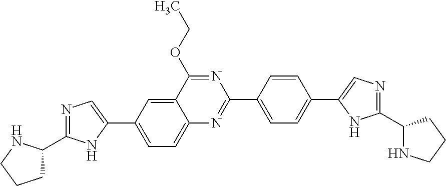

To a stirred solution of tert-butyl (2S)-2-(5-{4-[6-(2-[(2S)-1-(tert-butoxycarbonyl)pyrrolidin-2-yl]-1-{[2-(trimethyl silyl)ethoxy]methyl}-1H-imidazol-5-yl)-4-methoxyquinazolin-2-yl]phenyl}-1-{[2-(trimethylsilyl)ethoxy]methyl}-1H-imidazol-2-yl)pyrrolidine-1-carboxylate (90 mg, 0.093 mmol), obtained from Preparation 83, in ethanol (2 mL) was added 4M HCl in 1,4-dioxane (1.0 mL, 30.0 mmol). The reaction mixture was stirred at 75° C. for 3 hours. It was then evaporated and azeotroped with toluene to give the title compound as a yellow solid (51 mg) as the predominant product, which may have contained traces of the methoxy variant.

LCMS (run time=6 min): Rₜ=1.66 min; m/z 521 [M+H]⁺

### Preparation 85

### Ethyl 2-(4-bromophenyl)-2-oxoacetate

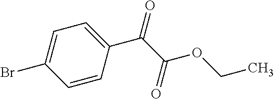

Aluminium trichloride (16.64 g, 125 mmol) was added, portionwise, to 4-bromobenzene (7.0 mL, 65 mmol) and ethyl oxalyl chloride (10.6 mL, 95 mmol) in dichloromethane (125 mL), at 0° C. The resulting mixture was allowed to warm to room temperature and stirred for a further 18 hours. It was then poured onto ice/conc. hydrochloric acid and extracted with dichloromethane. The organic layer was washed with 2N aqueous sodium hydroxide, and brine, then dried over MgSO₄ and evaporated to give the title compound as a pale yellow oil (8.68 g).

¹H NMR (400 MHz, CDCl₃): δ=7.90 (2H, dd), 7.66 (2H, dd), 4.44 (2H, q), 1.41 (3H, t).

### Preparation 86

### Ethyl 2-(4-bromo-2-nitrophenylimino)-2-(4-bromophenyl)acetate

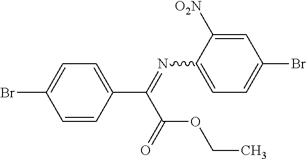

Titanium(IV) chloride (2.37 mL, 21.6 mmol) in dichloromethane (10 mL) was added dropwise to ethyl 2-(4-bromophenyl)-2-oxoacetate (7.94 g, 30.88 mmol), obtained from Preparation 85, triethylamine (25.8 mL, 185.3 mmol) and 4-bromo-2-nitroaniline (8.04 g, 37.06 mmol), in dichloromethane (300 mL), at 0° C. The resulting mixture was stirred, allowing to warm to room temperature, for 18 hours. Water (˜15 mL) was added and the reaction mixture was then filtered through celite. The filtrate was evaporated and the residue was purified by column chromatography on silica gel (heptane: ethyl acetate 85:15) to give the title compound as an orange oil (10.93 g).

¹H NMR (400 MHz, CDCl₃): δ=8.27 (1H, s), 7.79 (2H, d), 7.70-7.56 (3H, m), 6.79 (1H, d), 4.21-4.06 (2H, m), 1.10-0.97 (3H, m).

### Preparation 87

### 7-Bromo-3-(4-bromophenyl)quinoxalin-2-ol

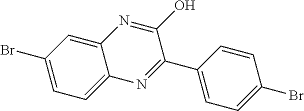

Ethyl 2-(4-bromo-2-nitrophenylimino)-2-(4-bromophenyl)acetate (593 mg, 1.3 mmol), obtained from Preparation 86, and iron powder (500 mg) in acetic acid (5 mL) were heated at 70° C. for 4 hours. Excess iron was removed using a magnet, then the reaction mixture was evaporated. The residue was suspended in sodium hydroxide (2N, aq., 30 mL), and hydrogen peroxide (30% aq., 3 mL) was added. The mixture was heated at reflux temperature for 2 hours. It was then neutralised by the addition of hydrochloric acid (2N, aq.) and extracted with ethyl acetate. The organic layer was dried over MgSO₄ and evaporated to give the title compound as a pale yellow solid (160 mg).

¹H NMR (400 MHz, DMSO-d₆): δ=12.66 (1H, br s), 8.25 (2H, d), 7.73 (1H, d), 7.67 (2H, d), 7.45 (2H, d).

### Preparation 88

### 6-Bromo-2-(4-bromophenyl)-3-chloroquinoxaline

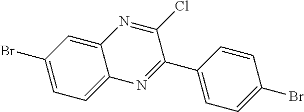

7-Bromo-3-(4-bromophenyl)quinoxalin-2-ol (250 mg, 0.66 mmol), obtained from Preparation 87, in phosphorus oxychloride (4 mL), was heated at 100° C. for 5 hours. Upon cooling, the reaction mixture was added slowly to ice-water then extracted with dichloromethane. The organic layer was washed with sodium bicarbonate (sat. aq.), dried over MgSO₄ and evaporated to give the title compound as a yellow solid (200 mg).

¹H NMR (400 MHz, CDCl₃): δ=8.23 (1H, d), 7.98 (1H, d), 7.89 (1H, d), 7.75 (2H, d), 7.67 (2H, d).

### Preparation 89

### 6-Bromo-2-(4-bromophenyl)-3-methoxyquinoxaline

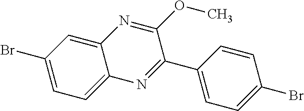

6-Bromo-2-(4-bromophenyl)-3-chloroquinoxaline (200 mg, 0.50 mmol), obtained from Preparation 88, and sodium methoxide (100 mg, 1.85 mmol), in tetrahydrofuran (10 mL) and methanol (10 mL), were stirred at room temperature for 24 hours. The solvent was partially evaporated, then the mixture was partitioned between ethyl acetate and water. The organic layer was washed with brine, dried over MgSO₄ and evaporated to give the title compound as a pale yellow solid (200 mg).

¹H NMR (400 MHz, CDCl₃): δ=8.09-7.99 (3H, m), 7.88 (1H, d), 7.70-7.58 (3H, m), 4.15 (3H, s).

### Preparation 90a

### (S)-tert-Butyl 2-(4-(4-(6-(2-((S)-1-(tert-butoxycarbonyl)pyrrolidin-2-yl)-1-(2-(trimethylsilyl)ethoxy)methyl)-1H-imidazol-4-yl)-3-methoxyquinoxalin-2-yl)phenyl)-1-((2-(trimethylsilyl)ethoxy)methyl)-1H-imidazol-2-yl)pyrrolidine-1-carboxylate

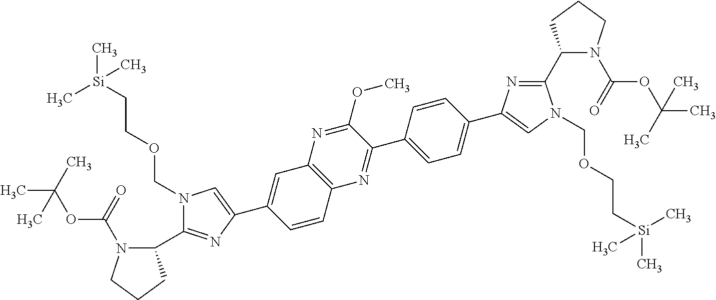

and

### Preparation 90b

### tert-Butyl (2S)-2-(4-{4-[6-(2-[(2S)-1-(tert-butoxycarbonyl)pyrrolidin-2-yl]-1-{[2-(trimethylsilyl)ethoxy]methyl}-1H-imidazol-4-yl)-3-hydroxyquinoxalin-2-yl]phenyl}-1-{[2-(trimethylsilyl)ethoxy]methyl}-1H-imidazol-2-yl)pyrrolidine-1-carboxylate

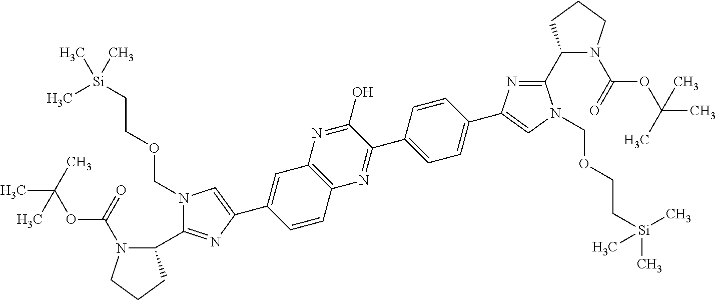

6-Bromo-2-(4-bromophenyl)-3-methoxyquinoxaline (300 mg, 0.76 mMol), obtained from Preparation 89, (S)-tert-butyl 2-(1-((2-(trimethylsilyl)ethoxy)methyl)-1H-imidazol-2-yl)pyrrolidine-1-carboxylate (839 mg, 2.28 mmol), obtained from Preparation 12, Pd(OAc)₂ (17 mg, 0.076 mmol), tricyclohexylphosphonium tetrafluoroborate (56 mg, 0.152 mmol), trimethylacetic acid (23 mg, 0.228 mmol) and potassium carbonate (315 mg, 2.28 mmol) in degassed dimethylacetamide (5 mL) were heated at 140° C. for 16 hours. The reaction mixture was then diluted with ethyl acetate and filtered through celite. The filtrate was washed with sodium bicarbonate (sat. aq.), dried over MgSO₄ and evaporated. The residue was purified by column chromatography on silica gel (dichloromethane: acetone 65:35 to 50:50) to give (S)-tert-butyl 2-(4-(4-(6-(2-((S)-1-(tert-butoxycarbonyl)pyrrolidin-2-yl)-1-((2-(trimethylsilyl)ethoxy)methyl)-1H-imidazol-4-yl)-3-methoxy quinoxalin-2-yl)phenyl)-1-((2-(trimethylsilyl)ethoxy)methyl)-1H-imidazol-2-yl)pyrrolidine-1-carboxylate (90a) as a bright yellow oil (96 mg);

¹H NMR (400 MHz, CDCl₃): δ=8.45 (2H, d), 7.96 (1H, d), 7.61-7.45 (4H, m), 7.25-7.11 (2H, m), 5.76 (1H, t), 5.22 (2H, d), 5.40-5.30+4.97-4.90 (1H, m), 5.10-5.03 (1H, m), 3.78 (3H, s), 3.78-3.40 (8H, m), 2.54-2.18 (2H, m), 2.05-1.57 (5H, m), 1.43 (9H, s), 1.24 (9H, s), 1.02-0.79 (6H, m), 0.01 (9H, s), 0.00 (9H, s). UPLC: (run time=1.5 min): Rₜ=1.26 min

and tert-butyl (2S)-2-(4-{4-[6-(2-[(2S)-1-(tert-butoxycarbonyl)pyrrolidin-2-yl]-1-{[2-(trimethylsilyl)ethoxy]methyl}-1H-imidazol-4-yl)-3-hydroxyquinoxalin-2-yl]phenyl}-1-{[2-(trimethylsilyl)ethoxy]methyl}-1H-imidazol-2-yl)pyrrolidine-1-carboxylate (90b) as a bright yellow oil (85 mg).

¹H NMR (400 MHz, CDCl₃) δ=8.51 (2H, d), 7.94 (1H, d), 7.65-7.31 (4H, m), 7.23-7.15 (2H, m), 5.74 (1H, t), 5.29-5.19 (2H, d), 5.41-5.34+4.96-4.90 (1H, m), 5.12-5.04 (1H, m), 3.80-3.41 (8H, m), 2.49-2.18 (2H, m), 2.08-1.51 (5H, m), 1.41+1.24 (18H, d+s), 1.00-0.78 (6H, m), 0.01 (9H, s), 0.00 (9H, s). UPLC: (run time=1.5 min): Rₜ=1.17 min

### Preparation 91

### 3-Methoxy-6-(2-((S)-pyrrolidin-2-yl)-1H-imidazol-4-yl)-2-(4-(2-((S)-pyrrolidin-2-yl)-1H-imidazol-4-yl)phenyl)quinoxaline hydrochloride salt

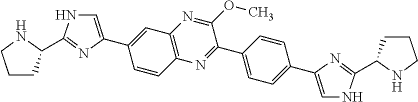

4M HCl in 1,4-dioxane (1.0 mL, 0.25 mmol) was added to (S)-tert-butyl 2-(4-(4-(6-(2-((S)-1-(tert-butoxycarbonyl)pyrrolidin-2-yl)-1-((2-(trimethylsilyl)ethoxy)methyl)-1H-imidazol-4-yl)-3-methoxyquinoxalin-2-yl)phenyl)-1-((2-(trimethylsilyl)ethoxy)methyl)-1H-imidazol-2-yl)pyrrolidine-1-carboxylate (95 mg, 98 μmol), obtained from Preparation 90a, in ethanol (5 mL) and the reaction mixture was heated at 70° C. for 4 hours. The solvent was evaporated and the residue was taken directly into the next step.

### Preparation 92

### 7-{2-[(2S)-pyrrolidin-2-yl]-1H-imidazol-4-yl}-3-(4-{2-[(2S)-pyrrolidin-2-yl]-1H-imidazol-4-yl}phenyl)quinoxalin-2-ol hydrochloride salt

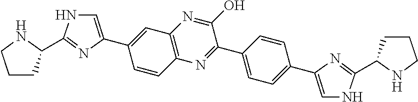

4M HCl in 1,4-dioxane (1.0 mL, 0.25 mmol) was added to tert-butyl (2S)-2-(4-{4-[6-(2-[(2S)-1-(tert-butoxycarbonyl)pyrrolidin-2-yl]-1-{[2-(trimethylsilyl)ethoxy]methyl}-1H-imidazol-4-yl)-3-hydroxyquinoxalin-2-yl]phenyl}-1-{[2-(trimethylsilyl)ethoxy]methyl}-1H-imidazol-2-yl)pyrrolidine-1-carboxylate (85 mg, 89 μmol), obtained from Preparation 90b, in ethanol (5 mL) and the reaction mixture was heated at 70° C. for 4 hours. The solvent was evaporated and the residue was taken directly into the next step.

### Preparation 93

### N-(2-Amino-5-bromobenzyl)-5-bromopyridine-2-carboxamide

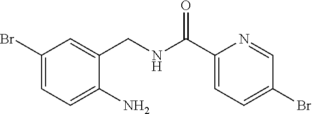

To a stirred solution of 5-bromopyridine-2-carboxylic acid (1.27 g, 6.3 mmol) in dry acetonitrile (45 mL) was added HBTU (2.61 g, 6.88 mmol). This solution was stirred at room temperature for 30 minutes. After this time, 2-(aminomethyl)-4-bromoaniline dihydrochloride salt (1.57 g, 5.73 mmol) was added, followed by DIPEA (3.29 mL, 18.9 mmol). The reaction mixture was allowed to stir at room temperature overnight. It was then concentrated in vacuo and the residue was taken up in ethyl acetate (50 mL). The organic layer was treated with saturated sodium bicarbonate solution (50 mL). At this point, a precipitate formed and was filtered to afford the title compound as a white solid (1.25 g).

¹H NMR (400 MHz, DMSO-d₆): δ=9.38 (1H, t), 8.80 (1H, dd), 8.27 (1H, dd), 7.98 (1H, dd), 7.17 (1H, d), 7.08 (1H, dd), 6.57 (1H, d), 5.39 (2H, s), 4.28 (2H, d). LCMS (run time=4.5 min): Rₜ=3.15 min; m/z 384; 386; 388 [M+H]⁺

### Preparation 94

### 6-Bromo-2-(5-bromopyridin-2-yl)-3,4-dihydroquinazoline

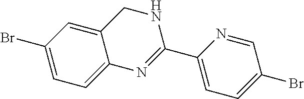

N-(2-amino-5-bromobenzyl)-5-bromopyridine-2-carboxamide (1.25 g, 2.1 mmol), obtained from Preparation 93, was dissolved in glacial acetic acid (25 mL) and the mixture was heated to reflux for 48 hours. It was then reduced in vacuo and the residue was suspended in methanol (10 mL) and saturated sodium bicarbonate solution (20 mL). The resulting precipitate was filtered and washed with ethyl acetate to afford the title compound as a yellow solid (1.06 g).

LCMS (run time=4.5 min): Rₜ=1.80 min; m/z 368 [M+H]⁺

### Preparation 95

### 6-Bromo-2-(5-bromopyridin-2-yl)quinazoline

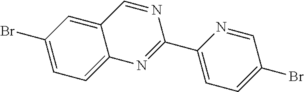

To a suspension of 6-bromo-2-(5-bromopyridin-2-yl)-3,4-dihydroquinazoline (1.06 g, 2.1 mmol), obtained from Preparation 94, in THF (20 mL), was added DDQ (0.48 g, 2.1 mmol) and the reaction mixture was stirred at room temperature for 30 minutes. 1M sodium hydroxide solution (6 mL), ethyl acetate (50 mL) and water (150 mL) were then added, in that order, and a precipitate formed, which was filtered to afford the title compound as a white solid (588 mg).

¹H NMR (400 MHz, DMSO-d₆): δ=9.75 (1H, d), 8.94 (1H, d), 8.55 (1H, d), 8.51 (1H, dd), 8.30 (1H, dd), 8.22 (1H, dd), 8.09 (1H, d). LCMS (run time=4.5 min): Rₜ=3.22 min; m/z 366 [M+H]⁺

### Preparation 96

### tert-Butyl (2S)-2-(5-{2-[5-(2-[(2S)-1-(tert-butoxycarbonyl)pyrrolidin-2-yl]-1-{[2-(trimethylsilyl)ethoxy]methyl}-1H-imidazol-5-yl)pyridin-2-yl]quinazolin-6-yl}-1-{[2-(trimethylsilyl)ethoxy]methyl}-1H-imidazol-2-yl)pyrrolidine-1-carboxylate

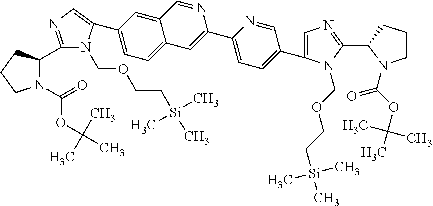

6-Bromo-2-(5-bromopyridin-2-yl)quinazoline (50 mg, 0.14 mmol), obtained from Preparation 95, (2-[(2S)-1-(tert-butoxycarbonyl)pyrrolidin-2-yl]-1-{[2 (trimethylsilyl)ethoxy]methyl}-1H-imidazol-5-yl)boronic acid (193 mg, 0.47 mmol), obtained from Preparation 13b, Pd₂(dba)₃ (0.9 mg, 1.0 μmol), copper (1) chloride (27 mg, 0.27 mmol), tricyclohexylphosphine (0.8 mg, 3.0 μmol) and potassium phosphate (99 mg, 466 μmol) in 1,4-dioxane (1.6 mL)/water (0.367 mL) were added to a microwave vial. The vial was evacuated and placed under nitrogen three times. The mixture was heated under microwave irradiation at 120° C. for 10 minutes, then a further charge of (2-[(2S)-1-(tert-butoxycarbonyl)pyrrolidin-2-yl]-1-{[2 (trimethylsilyl)ethoxy]methyl}-1H-imidazol-5-yl)boronic acid (128 mg, 0.31 mmol), obtained from Preparation 13b, in 1,4-dioxane (0.4 mL), Pd₂(dba)₃ (0.9 mg, 1.0 μmol) tricyclohexylphosphine (0.8 mg, 3.0 μmol), potassium phosphate (99 mg, 466 μmol) and water (0.367 mL) were added. The mixture was heated under microwave irradiation at 120° C. for 20 minutes. It was then diluted with ethyl acetate and washed with 880 ammonia solution. The aqueous layer was then washed again with ethyl acetate. The combined ethyl acetate extracts were dried over Na₂SO₄ and evaporated. The crude product was purified by column chromatography on silica gel (redisep (12 g) dichloromethane: methanol:ammonia 100:0:0 to 92:8:0.2) to give the title compound as a yellow gum (99 mg).

LCMS (run time=6 min): Rₜ=3.25 min; m/z 938 [M+H]⁺

### Preparation 97

### 6-{2-[(2S)-Pyrrolidin-2-yl]-1H-imidazol-5-yl}-2-(5-{2-[(2S)-pyrrolidin-2-yl]-1H-imidazol-5-yl}pyridin-2-yl)quinazoline hydrochloride salt

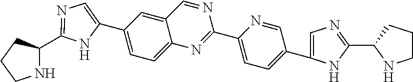

To a stirred solution of tert-butyl (2S)-2-(5-{2-[5-(2-[(2S)-1-(tert-butoxycarbonyl)pyrrolidin-2-yl]-1-{[2-(trimethylsilyl)ethoxy]methyl}-1H-imidazol-5-yl)pyridin-2-yl]quinazolin-6-yl}-1-{[2-(trimethylsilyl)ethoxy]methyl}-1H-imidazol-2-yl)pyrrolidine-1-carboxylate (99 mg, 0.11 mmol), obtained from Preparation 96, in ethanol (5 mL) was added 4M HCl in 1,4-dioxane (5 mL, 14.9 mmol) and the reaction mixture was stirred at 75° C. for 2 hours. It was then evaporated to give the title compound as a yellow solid (88 mg) which was used directly in the next step.

### Preparation 98

### 2-Chloro-5-(1-ethoxyethenyl)pyrimidine

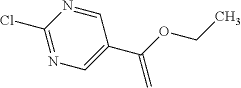

To a stirred solution of 5-bromo-2-chloropyrimidine (4.6 g, 24 mmol), in DMF (80 mL), was added tributyl(1-ethoxyvinyl) tin (8.83 mL, 26.2 mmol) and PdCl₂(PPh₃)₂ (3.34 g, 4.76 mmol). The reaction mixture was then degassed and heated to 70° C. for 2.5 hours. It was then allowed to cool to room temperature and 2M potassium fluoride (150 mL) was added. The reaction mixture was allowed to stir at room temperature overnight, after which time it was poured into water (250 mL) and extracted with ethyl acetate (3×250 mL). The combined organic phases were dried over anhydrous magnesium sulphate, filtered and the solvent was removed in vacuo. The crude material was purified by chromatography on silica gel (ethyl acetate:heptane 1:6) to afford the title compound as a white solid (2.06 g).

¹H NMR (400 MHz, CDCl₃): δ=8.81 (2H, s), 4.74 (1H, d), 4.41 (1H, d), 3.96 (2H, q), 1.44 (3H, t).

### Preparation 99

### 2-Bromo-1-(2-chloropyrimidin-5-yl)ethanone

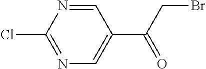

To a stirred solution of 2-chloro-5-(1-ethoxyethenyl)pyrimidine (0.5 g, 2.71 mmol), obtained from Preparation 98, in a mixture of THF and water (31.5 mL; 5% water), was added NBS (0.53 g, 2.90 mmol). The reaction mixture was stirred at room temperature for 2 hours. It was then concentrated in vacuo and the crude material was purified by chromatography on silica gel (ethyl acetate:heptane 1:6 to 1:5) to afford the title compound as a white solid (354 mg).

¹H NMR (400 MHz, CDCl₃): δ=9.17 (2H, s), 4.36 (2H, s).

### Preparation 100

### 1-tert-Butyl 2-[2-(2-chloropyrimidin-5-yl)-2-oxoethyl](2S)-pyrrolidine-1,2-dicarboxylate

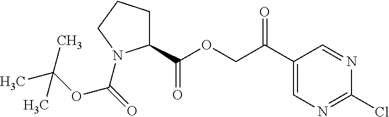

To an ice cooled stirred solution of L-BOC proline (0.3 g, 1.39 mmol), in dichloromethane (7 mL), was added 2-bromo-1-(2-chloropyrimidin-5-yl)ethanone (0.35 g, 1.46 mmol), obtained from Preparation 99, and DIPEA (0.5 mL, 2.93 mmol). The reaction mixture was allowed to warm to room temperature and was stirred for 3 hours. It was then washed with water (20 mL), saturated aqueous sodium bicarbonate solution (25 mL), water (25 mL) and brine (25 mL). The organic phase was dried over anhydrous MgSO₄, filtered and the solvent was removed in vacuo to afford the title compound as an orange solid (423 mg).

LCMS (run time=4.5 min): Rₜ=3.02 min; m/z 270; 272 [(M-Boc)+2H]⁺

### Preparation 101

### tert-Butyl (2S)-2-[5-(2-chloropyrimidin-5-yl)-1H-imidazol-2-yl]pyrrolidine-1-carboxylate

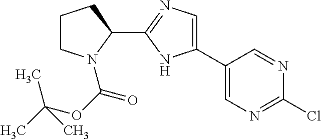

To a stirred solution of 1-tert-butyl 2-[2-(2-chloropyrimidin-5-yl)-2-oxoethyl](2S)-pyrrolidine-1,2-dicarboxylate (0.42 g, 1.14 mmol), obtained from Preparation 100, in xylenes (5 mL), was added ammonium acetate (0.44 g, 5.7 mmol). The reaction mixture was heated at reflux for 18 hours. It was then diluted with ethyl acetate (20 mL) and was washed with water (20 mL) and brine (20 mL). The organic phase was dried over anhydrous magnesium sulphate, filtered and the solvent was removed in vacuo. The residue was purified by chromatography on silica gel (ethyl acetate:heptane 3:1) to afford the title compound as a white solid (114 mg).

LCMS (run time=4.5 min): Rₜ=2.15 min; m/z 350 [M+H]⁺

### Preparation 102

### tert-Butyl (2S)-2-{4-[2-(6-bromoquinoxalin-2-yl)pyrimidin-5-yl]-1H-imidazol-2-yl}pyrrolidine-1-carboxylate

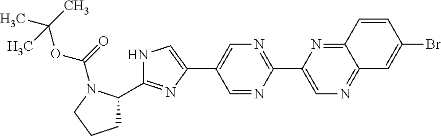

To a stirred solution of tert-butyl (2S)-2-[5-(2-chloropyrimidin-5-yl)-1H-imidazol-2-yl]pyrrolidine-1-carboxylate (0.11 g, 0.33 mmol), obtained from Preparation 101, in dry dioxane (2 mL), was added hexamethylditin (0.11 g, 0.33 mmol), followed by Pd(PPh₃)₄ (0.095 g, 0.082 mmol). The reaction mixture was degassed and then heated to reflux for 2 hours. After this time, it was cooled to room temperature and diluted with ethyl acetate (20 mL). The organic phase was washed with saturated ammonium chloride solution (20 mL), water (20 mL) and brine (20 mL); then dried over anhydrous sodium sulphate, filtered and the solvent was removed in vacuo. The residue was dissolved in DMF (2 mL) and 6-bromo-2-chloroquinoxaline (0.088 g, 0.33 mmol), obtained from Preparation 30, was added, followed by cesium fluoride (0.09 g, 0.59 mmol), copper (I) chloride (0.033 g, 0.33 mmol) and Pd(PPh₃)₄ (0.095 g, 0.082 mmol). The reaction mixture was degassed and stirred at 110° C. for 3.5 hours. It was then allowed to cool to room temperature and was diluted with ethyl acetate (20 mL). The resulting suspension was washed with 0.88 ammonia solution (50 mL) and the aqueous phase was back extracted with more ethyl acetate (4×20 mL). The combined organic phases were dried over anhydrous sodium sulphate, filtered and the solvent was removed in vacuo. The crude material was purified by chromatography on silica gel (ethyl acetate:heptane 1:5 to 1:1 to ethyl acetate:methanol 95:5) to afford the title compound as a bright yellow solid (80 mg).

LCMS (run time=4.5 min): Rₜ=2.76 min; m/z 522; 524 [M+H]⁺.

### Preparation 103

### tert-Butyl (2S)-2-(5-[2-(5-{2-[(2S)-1-(tert-butoxycarbonyl)pyrrolidin-2-yl]-1H-imidazol-4-yl}pyrimidin-2-yl)quinoxalin-6-yl]-1-{[2-(trimethylsilyl)ethoxy]methyl}-1H-imidazol-2-yl)pyrrolidine-1-carboxylate

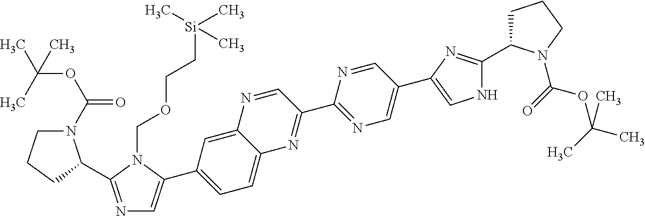

tert-Butyl (2S)-2-{4-[2-(6-bromoquinoxalin-2-yl)pyrimidin-5-yl]-1H-imidazol-2-yl}pyrrolidine-1-carboxylate (0.08 g, 0.15 mmol), obtained from Preparation 102, (2-[(2S)-1-(tert-butoxycarbonyl)pyrrolidin-2-yl]-1-{[2-(trimethylsilyl)ethoxy]methyl}-1H-imidazol-5-yl)boronic acid (0.08 g, 0.2 mmol), obtained from Preparation 13b, and Pd(dppf)Cl₂.DCM (0.008 g, 0.03 mmol) were added to a microwave vial. Dimethoxyethane (1.5 mL) and 2 M sodium carbonate solution (0.23 mL) were added. The vial was sealed and heated under microwave irradiation for 40 minutes. The reaction was allowed to cool and the solvent was removed in vacuo. The crude material was purified by chromatography on silica gel (ethyl acetate:methanol 1:0 to 95:5) to afford the title compound as a yellow solid (82 mg).

LCMS (run time=4.5 min): Rₜ=2.90 min; m/z 809 [M+H]⁺

### Preparation 104

### 6-{2-[(2S)-Pyrrolidin-2-yl]-1H-imidazol-5-yl}-2-(5-{2-[(2S)-pyrrolidin-2-yl]-1H-imidazol-4-yl}pyrimidin-2-yl)quinoxaline hydrochloride salt

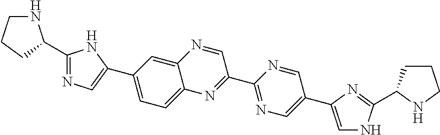

To a stirred solution of tert-butyl (2S)-2-(5-[2-(5-{2-[(2S)-1-(tert-butoxycarbonyl)pyrrolidin-2-yl]-1H-imidazol-4-yl}pyrimidin-2-yl)quinoxalin-6-yl]-1-{[2-(trimethylsilyl)ethoxy]methyl}-1H-imidazol-2-yl)pyrrolidine-1-carboxylate (0.08 g, 0.1 mmol), obtained from Preparation 103, in methanol (1 mL) was added 4 M HCl in dioxane (0.5 mL). The reaction mixture was heated to 75° C. for 2 hours. It was then allowed to cool to room temperature and the solvent was removed in vacuo to afford the title compound as a brown solid (38 mg).

LCMS (run time=4.5 min): Rₜ=0.44 min; m/z 479 [M+H]⁺

### Preparation 105

### tert-Butyl (2S)-2-(5-[3-(5-{2-[(2S)-1-(tert-butoxycarbonyl)pyrrolidin-2-yl]-1H-imidazol-4-yl}pyrimidin-2-yl)isoquinolin-7-yl]-1-{[2-(trimethylsilyl)ethoxy]methyl}-1H-imidazol-2-yl)pyrrolidine-1-carboxylate

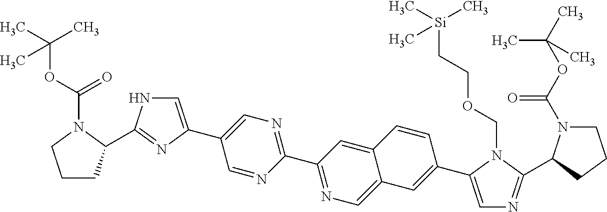

To a stirred solution of tert-butyl (2S)-2-[5-(2-chloropyrimidin-5-yl)-1H-imidazol-2-yl]pyrrolidine-1-carboxylate (0.17 g, 0.49 mmol), obtained from Preparation 101, in dry dioxane (3 mL), was added hexamethylditin (0.16 g, 0.49 mmol), followed by Pd(PPh₃)₄ (0.14 g, 0.12 mmol). The reaction mixture was degassed, put under nitrogen and then heated to reflux for 2 hours. After this time, it was cooled to room temperature and diluted with ethyl acetate (30 mL). The organic phase was washed with saturated ammonium chloride solution (30 mL) and brine (30 mL), dried over anhydrous sodium sulphate, filtered and the solvent was removed in vacuo. The residue was dissolved in DMF (1.5 mL) and was added to a solution of tert-butyl (2S)-2-[5-(3-chloroisoquinolin-7-yl)-1-{[2-(trimethylsilyl)ethoxy]methyl}-1H-imidazol-2-yl]pyrrolidine-1-carboxylate (0.263 g, 0.49 mmol), obtained from Preparation 64, followed by cesium fluoride (0.14 g, 0.9 mmol), copper (I) chloride (0.049 g, 0.49 mmol) and Pd(PPh₃)₄ (0.14 g, 0.12 mmol). The reaction mixture was degassed, put under nitrogen, and stirred at 110° C. for 3.5 hours. It was then allowed to cool to room temperature and diluted with ethyl acetate (30 mL). The resulting suspension was washed with 0.88 ammonia solution (10 mL) and the aqueous phase was back extracted with more ethyl acetate (4×20 mL). The combined organic phases were dried over anhydrous sodium sulphate, filtered and the solvent was removed in vacuo. The crude material was purified by chromatography on silica gel (dichloromethane: methanol:ammonia 95:5:0.5) to afford the title compound as a bright yellow solid (58 mg).

LCMS (run time=4.5 min): Rₜ=2.70 min; m/z 808 [M]⁺

### Preparation 106

### 7-{2-[(2S)-Pyrrolidin-2-yl]-1H-imidazol-5-yl}-3-(5-{2-[(2S)-pyrrolidin-2-yl]-1H-imidazol-4-yl}pyrimidin-2-yl)isoquinoline hydrochloride salt

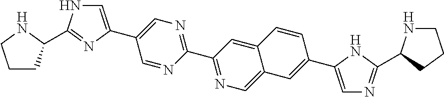

To a stirred solution of tert-butyl (2S)-2-(5-[3-(5-{2-[(2S)-1-(tert-butoxycarbonyl)pyrrolidin-2-yl]-1H-imidazol-4-yl}pyrimidin-2-yl)isoquinolin-7-yl]-1-{[2-(trimethylsilyl)ethoxy]methyl}-1H-imidazol-2-yl)pyrrolidine-1-carboxylate (0.06 g, 0.07 mmol), obtained from Preparation 105, in methanol (2 mL) was added 4 M HCl in dioxane (0.7 mL). The reaction mixture was heated to 75° C. for 45 minutes. It was then allowed to cool to room temperature and the solvent was removed in vacuo to afford the title compound as an orange solid (53 mg).

LCMS (run time=4.5 min): Rₜ=0.44 min; m/z 478 [M+H]⁺

### Preparation 107

### Methyl {(2S)-1-[(2S)-2-{5-[4-(6-bromo-4-cyanoquinolin-2-yl)phenyl]-1H-imidazol-2-yl}pyrrolidin-1-yl]-3-methyl-1-oxobutan-2-yl}carbamate

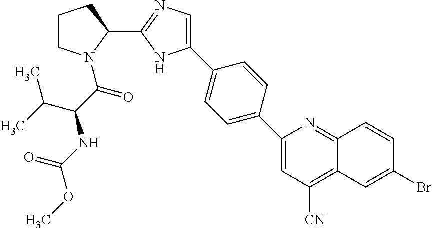

6-Bromo-2-chloroquinoline-4-carbonitrile (100 mg, 0.374 mmol), methyl {(2S)-3-methyl-1-oxo-1-[(2S)-2-{5-[4-(4,4,5,5-tetramethyl-1,3,2-dioxaborolan-2-yl)phenyl]-1H-imidazol-2-yl}pyrrolidin-1-yl]butan-2-yl}carbamate (186 mg, 0.374 mmol), obtained from Preparation 28, 2N sodium bicarbonate (0.561 mL, 1.12 mmol) and Pd(dppf)Cl₂.DCM (15 mg, 0.019 mmol) were stirred at 40° C. in DME (1 mL) for 18 hours. The reaction mixture was then partitioned between DCM and water, and the organic extract was concentrated and purified by column chromatography on silica gel (Redisep 4 g, eluting with a gradient of heptane:ethyl acetate (80:20 to 40:60) to afford 180 mg of the title compound as a bright yellow solid.

LCMS (run time=2 min): Rₜ=1.47 min; m/z 601; 603 [M+H]⁺

### Preparation 108

### Methyl {(2S)-1-[(2S)-2-{5-[4-(4-cyano-6-{2-[(2S)-pyrrolidin-2-yl]-1H-imidazol-5-yl}quinolin-2-yl)phenyl]-1H-imidazol-2-yl}pyrrolidin-1-yl]-3-methyl-1-oxobutan-2-yl}carbamate

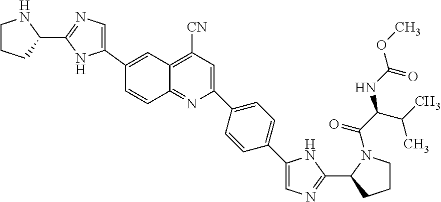

Methyl {(2S)-1-[(2S)-2-{5-[4-(6-bromo-4-cyanoquinolin-2-yl)phenyl]-1H-imidazol-2-yl}pyrrolidin-1-yl]-3-methyl-1-oxobutan-2-yl}carbamate (65 mg, 0.11 mmol), obtained from Preparation 107, (2-[(2S)-1-(tert-butoxycarbonyl)pyrrolidin-2-yl]-1-{[2-(trimethylsilyl)ethoxy]methyl}-1H-imidazol-5-yl)boronic acid (90 mg, 0.216 mmol), obtained from Preparation 13b, 2N sodium bicarbonate (0.162 mL, 0.324 mmol) and Pd(dppf)Cl₂.DCM (4 mg, 0.005 mmol) were added to a microwave vial, followed by DME (1 mL). The mixture was heated under microwave irradiation at 120° C. for 40 minutes. Further (2-[(2S)-1-(tert-butoxycarbonyl)pyrrolidin-2-yl]-1-{[2-(trimethylsilyl)ethoxy]methyl}-1H-imidazol-5-yl)boronic acid (10 mg, 0.024 mmol), obtained from Preparation 13b, was added and the mixture was heated for a further 20 minutes. It was then partitioned between ethyl acetate and water, and the organic extract was dried over MgSO₄ and evaporated. The crude material was purified by column chromatography on silica gel (Redisep 4 g, eluting with a gradient of DCM: MeOH+1% NH3 100:0 to 90:10). The purified intermediate was dissolved in ethanol (2 mL) and treated with 4N HCl/Dioxan (2 mL) at 70° C. for 2 hours. The reaction mixture was concentrated and applied to an SCX cartridge, eluting initially with methanol then 7N ammonia in methanol, to elute the product as an orange glass (47 mg).

LCMS (run time=2 min): Rₜ=1.11 min; m/z 658 [M+H]⁺

### Preparation 109

### 2-(6-{[tert-Butyl(dimethyl)silyl]oxy}naphthalen-2-yl)-5-chloropyrazine

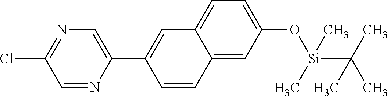

To a stirred solution of 2,5-dichloro-pyrazine (0.5 g, 3.37 mmol) in toluene (10 mL) was added 6-(tert-butyl-dimethyl-silanyloxy)-naphthalen-2-yl-boronic acid (1.02 g, 3.37 mmol), EtOH (10 mL), water (2 mL) and NaHCO₃ (0.42 g, 5.0 mmol). Argon was bubbled through the reaction mixture for 30 min. Then Pd(PPh₃)₄ (0.19 g, 0.16 mmol) was added and the mixture was heated at 45° C. for 2 hours. The EtOH was evaporated under vacuum and the mixture was diluted with ethyl acetate (35 mL). The reaction mixture was then filtered through celite. The organic layer was washed with water (1×10 mL), and brine (1×10 mL), then dried over Na₂SO₄ and evaporated to dryness. The crude mass was purified by column chromatography on silica gel (ethyl acetate:hexane, 1:19) to afford the title compound as a white solid (450 mg).

¹H NMR (400 MHz, DMSO-d₆): δ=0.28 (6H, s), 1.00 (9H, s), 7.20 (1H, d), 7.38 (1H, s), 7.99 (2H, m), 8.17 (1H, m), 8.70 (1H, s), 8.75 (1H, s), 9.40 (1H, s). LCMS (System 1) (run time=5 min): Rₜ=3.45 min, 371 [M+H]⁺

### Preparation 110

### tert-Butyl (2S)-2-(5-[5-(6-hydroxynaphthalen-2-yl)pyrazin-2-yl]-1-{[2-(trimethylsilyl)ethoxy]methyl}-1H-imidazol-2-yl)pyrrolidine-1-carboxylate

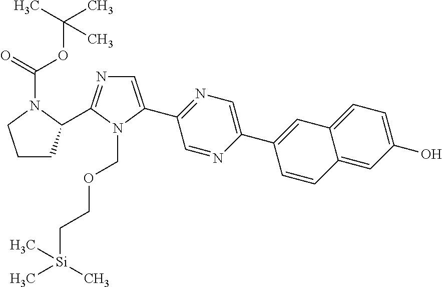

To a stirred solution of 2-[6-(tert-butyl-dimethyl-silanyloxy)-naphthalen-2-yl]-5-chloro-pyrazine (0.45 g, 1.21 mmol), obtained from Preparation 109, in toluene (8 mL) was added (2-[(2S)-1-(tert-butoxycarbonyl)pyrrolidin-2-yl]-1-{[2 (trimethylsilyl)ethoxy]methyl}-1H-imidazol-5-yl)boronic acid (0.75 g, 1.82 mmol), obtained from Preparation 13b, EtOH (8 mL), water (1.8 mL) and Na₂CO₃ (0.32 g, 3.04 mmol). Argon was bubbled through the reaction mixture for 30 minutes. Pd(PPh₃)₄ (0.069 g, 0.05 mmol) was added and the mixture was heated in a sealed tube at 100° C. for 16 hours. The EtOH was removed in vacuo and the mixture was diluted with ethyl acetate (30 mL). The reaction mixture was filtered through celite. The organic extract was washed with water (1×10 mL), and brine (1×10 mL), then dried over Na₂SO₄ and evaporated to dryness. The crude mass was purified by column chromatography on silica gel (ethyl acetate:hexane 3:7) to afford the title compound as an off white solid (550 mg).

LCMS (System 1) (run time=5 min): Rₜ=1.11 min, 588 [M+H]⁺

### Preparation 111

tert-Butyl (2S)-2-(5-[5-(6-{[(trifluoromethyl)sulfonyl]oxy}naphthalen-2-yl)pyrazin-2-yl]-1-{[2-(trimethylsilyl)ethoxy]methyl}-1H-imidazol-2-yl)pyrrolidine-1-carboxylate

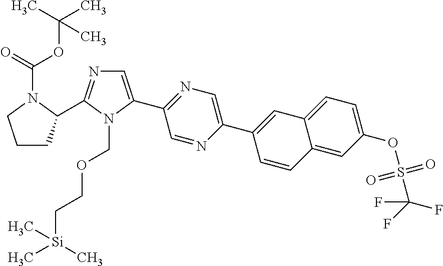

To a stirred solution of tert-butyl (2S)-2-(5-[5-(6-hydroxynaphthalen-2-yl)pyrazin-2-yl]-1-{[2-(trimethylsilyl)ethoxy]methyl}-1H-imidazol-2-yl)pyrrolidine-1-carboxylate (0.55 g, 0.93 mmol), obtained from Preparation 110, in DCM (20 mL) was added triethyl amine (0.29 mL, 2.06 mmol) and triflic anhydride (0.24 mL, 1.40 mmol) at −78° C. The reaction mixture was stirred at this temperature for 10 minutes. It was then quenched with a saturated solution of sodium bicarbonate and extracted with DCM (2×20 mL). The combined organic fractions were washed with water (1×10 mL, and brine (1×10 mL), then dried over Na₂SO₄ and evaporated to dryness. The crude material was isolated as a yellow solid (620 mg) and was used for the next step without further purification.

LCMS (System 1) (run time=5 min): Rₜ=2.63 min, 720 [M+H]⁺

### Preparation 112

### tert-Butyl (2S)-2-(5-{5-[6-(2-[(2S)-1-(tert-butoxycarbonyl)pyrrolidin-2-yl]-1-{[2-(trimethylsilyl)ethoxy]methyl}-1H-imidazol-5-yl)naphthalen-2-yl]pyrazin-2-yl}-1-{[2-(trimethylsilyl)ethoxy]methyl}-1H-imidazol-2-yl)pyrrolidine-1-carboxylate

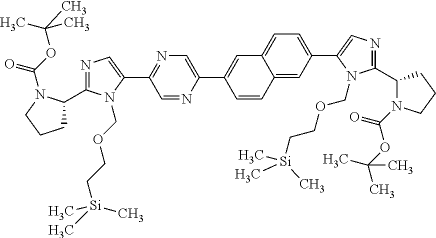

To a stirred solution of tert-butyl (2S)-2-(5-[5-(6-{[(trifluoromethyl)sulfonyl]oxy}naphthalen-2-yl)pyrazin-2-yl]-1-{[2-(trimethylsilyl)ethoxy]methyl}-1H-imidazol-2-yl)pyrrolidine-1-carboxylate (0.6 g, 0.83 mmol), obtained from Preparation 111, in acetonitrile (15 mL), was added (2-[(2S)-1-(tert-butoxycarbonyl)pyrrolidin-2-yl]-1-{[2 (trimethylsilyl)ethoxy]methyl}-1H-imidazol-5-yl)boronic acid (0.69 g, 1.66 mmol), obtained from Preparation 13b, and CsF (1.14 g, 7.51 mmol). Argon was bubbled through the reaction mixture for 30 minutes. Tricyclohexyl phosphine (0.078 g, 0.275 mmol) and Pd(OAc)₂ (0.037 g, 0.166 mmol) were added to the reaction mixture and it was heated in a sealed tube at 90° C. for 16 hours. The acetonitrile was evaporated under reduced pressure and the residue was taken up in ethyl acetate (40 mL). The ethyl acetate layer was washed with water (1×15 mL), and brine (1×15 mL), then dried over Na₂SO₄ and evaporated to dryness. The crude material was purified by column chromatography on silica gel (ethyl acetate:hexane 2:3) to afford the title compound as an off white solid (600 mg).

LCMS (System 1) (run time=5 min): Rₜ=1.13 min, 937 [M+H]⁺

### Preparation 113

### 2-{2-[(2S)-Pyrrolidin-2-yl]-1H-imidazol-5-yl}-5-(6-{2-[(2S)-pyrrolidin-2-yl]-1H-imidazol-5-yl}naphthalen-2-yl)pyrazine hydrochloride salt

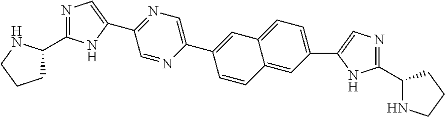

To a stirred solution of tert-butyl (2S)-2-(5-{-[6-(2-[(2S)-1-(tert-butoxycarbonyl)pyrrolidin-2-yl]-1-{[2-(trimethylsilyl)ethoxy]methyl}-1H-imidazol-5-yl)naphthalen-2-yl]pyrazin-2-yl}-1-{[2-(trimethylsilyl)ethoxy]methyl}-1H-imidazol-2-yl)pyrrolidine-1-carboxylate (1.2 g, 1.28 mmol), obtained from Preparation 112, in dioxane (5 mL), was added 4M HCl in 1,4-dioxane (20 mL), at 0° C., and the mixture was stirred at room temperature for 16 hours. Then the dioxane was removed by evaporation and the residue was triturated with ether (2×10 mL). The crude product was isolated as an off white solid (750 mg).

LCMS (System 1) (run time=5 min): Rₜ=1.55 min, 477 [M+H]⁺

### Preparation 114

### N-(Methoxycarbonyl)-L-alanine

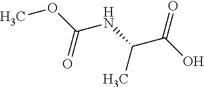

Methyl chloroformate (1.9 mL, 24.7 mmol) was added dropwise to an ice-cooled, clear solution of Na₂CO₃ (1.19 g, 11.2 mmol) and L-alanine (2 g, 20 mmol) in 1M NaOH (22.5 mL, 22.5 mmol). The mixture was allowed to warm to room temperature and stirred overnight. It was then washed with TBME (2×40 mL) and the aqueous phase was cooled to 0° C. before being acidified to pH˜1 with 6 M HCl. The cloudy suspension was then extracted with DCM (6×50 mL). The organic phases were combined, dried over Na₂SO₄ and the solvent was evaporated to give the title compound as a colourless oil (1.6 g).

¹H NMR (400 MHz, DMSO-d₆): δ=12.49 (1H, br s); 7.45-7.43 (1H, d), 4.01-3.94 (1H, q), 3.52 (3H, s), 1.24 (3H, d).

### Preparation 115

### N-(methoxycarbonyl)-2-methylalanine

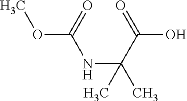

Methyl chloroformate (1.64 mL, 21.3 mmol) was added dropwise to an ice-cooled, clear solution of Na₂CO₃ (1.03 g, 9.70 mmol) and 2-aminoisobutyric acid (2 g, 19.4 mmol) in 1M NaOH (19.5 mL, 19.5 mmol). The mixture was allowed to warm to room temperature and stirred overnight. It was then washed with TBME (2×40 mL) and the aqueous phase was cooled to 0° C. before being acidified to pH˜1 with 6M HCl. The cloudy suspension was then extracted with DCM (6×50 mL). The organic phases were combined, dried over Na₂SO₄ and the solvent was evaporated to give the title compound as a white solid (1.29 g).

¹H NMR (400 MHz, DMSO-d₆): δ=12.27 (1H, br s), 7.39 (1H, br s), 3.49 (3H, s), 1.32 (6H, s).

### Preparation 116

### N-(methoxycarbonyl)glycine

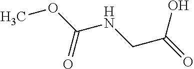

Methyl chloroformate (2.26 mL, 29.3 mmol) was added dropwise to an ice-cooled, clear solution of Na₂CO₃ (1.41 g, 13.3 mmol) and glycine (2 g, 30 mmol) in 1M NaOH (26.8 mL, 26.8 mmol). The mixture was allowed to warm to room temperature and stirred overnight. It was then washed with TBME (2×40 mL) and the aqueous phase was cooled to 0° C. before being acidified to pH˜1 with 6M HCl. The solution was extracted with DCM: MeOH (9:1) (14×20 mL). The organic phases were combined, dried over Na₂SO₄ and the solvent was evaporated to give the title compound as a white solid (2.35 g).

¹H NMR (400 MHz, DMSO-d₆): δ=12.5 (1H, br s), 7.26 (1H, t), 3.59 (2H, d), 3.53 (3H, s).

### Preparation 117

### tert-Butyl (2S)-2-(5-{6-[4-(2-{(2S)-1-[N-(methoxycarbonyl)-L-valyl]pyrrolidin-2-yl}-1H-imidazol-4-yl)phenyl]naphthalen-2-yl}-1-{[2-(trimethylsilyl)ethoxy]methyl}-1H-imidazol-2-yl)pyrrolidine-1-carboxylate

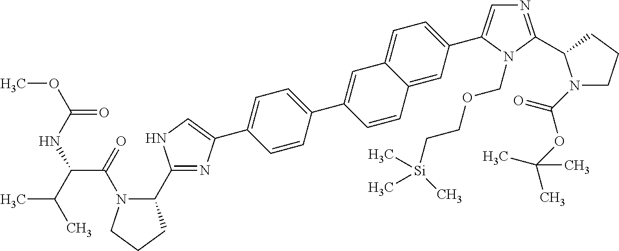

tert-Butyl (2S)-2-[5-(6-{[(trifluoromethyl)sulfonyl]oxy}naphthalen-2-yl)-1-{[2-(trimethylsilyl)ethoxy]methyl}-1H-imidazol-2-yl]pyrrolidine-1-carboxylate (571 mg, 0.89 mmol), obtained from Preparation 19, methyl (S)-3-methyl-1-oxo-1-((S)-2-(5-(4-(4,4,5,5-tetramethyl-1,3,2-dioxaborolan-2-yl)-phenyl)-1H-imidazol-2-yl)-pyrrolidin-1-yl)butan-2-ylcarbamate (624 mg, 1.07 mmol), obtained from Preparation 28, and Pd(dppf)Cl₂.DCM (44 mg, 0.178 mmol) were added to a microwave vial, followed by 1,2-dimethoxyethane (3 mL) and 2M sodium carbonate solution (1.34 mL, 2.67 mmol). The vial was sealed and the reaction mixture was stirred in a microwave at 120° C. for 20 minutes. The solvent was then removed by evaporation and the residue was purified by column chromatography on silica gel (dichloromethane; methanol 100:0 to 95:5), then by reverse phase chromatography (acetonitrile:0.1% formic acid in water) to give the title compound as an off-white solid (380 mg).

LCMS (run time=8 min): Rₜ=2.55 min; m/z 862 [M+H]⁺

### Preparation 118

### Methyl {(2S)-3-methyl-1-oxo-1-[(2S)-2-{5-[4-(6-{2-[(2S)-pyrrolidin-2-yl]-1H-imidazol-5-yl}naphthalen-2-yl)phenyl]-1H-imidazol-2-yl}pyrrolidin-1-yl]butan-2-yl}carbamate

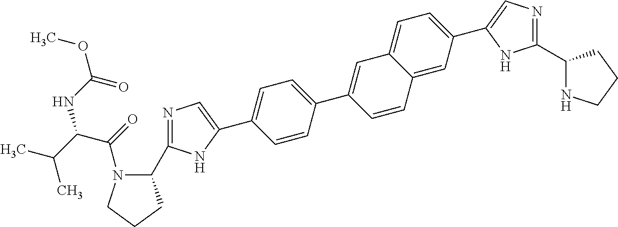

To a stirred solution of tert-butyl (2S)-2-(5-{6-[4-(2-{(2S)-1-[N-(methoxycarbonyl)-L-valyl]pyrrolidin-2-yl}-1H-imidazol-4-yl)phenyl]naphthalen-2-yl}-1-{[2-(trimethylsilyl)ethoxy]methyl}-1H-imidazol-2-yl)pyrrolidine-1-carboxylate (380 mg, 0.44 mmol), obtained from Preparation 117, in dry EtOH (5 mL), was added 4N HCl, in dioxane (3 mL). The mixture was stirred at 75° C. for 1 hour. It was then evaporated and the residue was dissolved in MeOH and loaded onto an SCX column, washing with more methanol. The product was collected by flushing with 20% ammonia in methanol and the filtrate was concentrated in vacuo to give the title compound as an off-white solid (218 mg).

LCMS (run time=8 min): Rₜ=1.78 min; m/z 632 [M+H]⁺

### Preparation 119

### tert-Butyl quinolin-7-yl carbonate

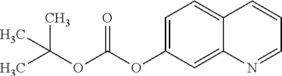

To a stirred solution of quinolin-7-ol (2.86 g, 19.7 mmol), in DMF (20 mL), was added BOC-anhydride (10.6 g, 48.5 mmol) and DMAP (318 mg, 2.60 mmol). The mixture was stirred at room temperature over night. It was then diluted with a 1:1 v/v mixture of TBME-EtOAc (400 mL) and washed with 1M NaOH (400 mL). The organic phase was separated, washed with water (400 mL), and brine (400 mL), then dried over Na₂SO₄ and evaporated. The crude material was purified by column chromatography on silica gel (1:1 EtOAc:heptane) to give the title product as a pale yellow oil which solidified on standing (4.36 g).

¹H NMR (400 MHz, CDCl₃): δ=8.92 (1H, dd), 8.16 (1H, m), 7.89 (1H, d), 7.83 (1H, d), 7.41 (1H, dd), 7.39 (1H, dd), 1.59 (9H, s). LCMS (run time=4.5 min): Rₜ=2.27 min; m/z 246 [M+H]⁺

### Preparation 120

### 3-Bromoquinolin-7-yl tert-butyl carbonate

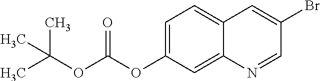

Bromine (1.05 ml, 20.4 mmol) was added to a stirred solution of tert-butyl quinolin-7-yl carbonate (4.36 g, 17.8 mmol), obtained from Preparation 119, in carbon tetrachloride (40 mL), and heated to reflux. Pyridine (1.58 ml, 19.6 mmol) was added over 10 minutes and the solution was then stirred at reflux for 18 hours. The reaction mixture was decanted and the brown solid which remained was dissolved in MeOH (20 mL). This solution was then combined with the rest of the reaction mixture, which was then diluted with EtOAc (240 mL) and washed with sat aq NaHCO₃ (200 mL). The aqueous phase was separated and washed with EtOAc (3×200 mL). The combined organic extracts were dried over Na₂SO₄, filtered and evaporated. The crude material was purified by flash chromatography on silica gel (EtOAc: heptane, 1:4) to give the title product as a pale yellow oil which solidified on standing (3.15 g).

¹H NMR (400 MHz, CDCl₃): δ=8.91 (1H, d), 8.31 (1H, d), 7.88 (1H, d), 7.75 (1H, d), 7.44 (1H, dd), 1.59 (9H, s). LCMS (run time=4.5 min): Rₜ=3.62 min; m/z 324; 326 [M+H]⁺

### Preparation 121

### Methyl {(2S)-1-[(2S)-2-{4-[4-(7-hydroxyquinolin-3-yl)phenyl]-1H-imidazol-2-yl}pyrrolidin-1-yl]-3-methyl-1-oxobutan-2-yl}carbamate

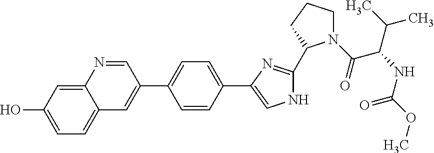

3-Bromoquinolin-7-yl tert-butyl carbonate of (150 mg, 0.46 mmol), obtained from Preparation 120, methyl (S)-3-methyl-1-oxo-1-((S)-2-(5-(4-(4,4,5,5-tetramethyl-1,3,2-dioxaborolan-2-yl)-phenyl)-1H-imidazol-2-yl)-pyrrolidin-1-yl)butan-2-yl carbamate (325 mg, 0.56 mmol), obtained from Preparation 28, and Pd(dppf)Cl₂.DCM (23 mg, 0.093 mmol), in 1,2-dimethoxyethane (0.75 mL) and 2M sodium carbonate solution (0.70 mL, 1.39 mmol), were stirred in a microwave at 120° C. for 40 minutes. The reaction mixture was then diluted with MeOH (10 mL), absorbed onto silica and purified by column chromatography (dichloromethane: methanol:ammonia 95:5:0.5) to give the title compound as a yellow glass (135 mg).

LCMS (System 5) (run time=5 min): Rₜ=1.67 min; m/z 514 [M+H]⁺

### Preparation 122

### 3-[4-(2-{(2S)-1-[N-(methoxycarbonyl)-L-valyl]pyrrolidin-2-yl}-1-[(trifluoromethyl) sulfonyl]-1H-imidazol-4-yl)phenyl]quinolin-7-yl trifluoromethanesulfonate

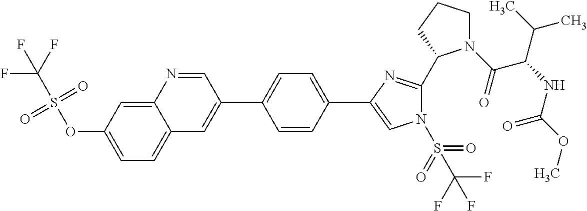

To a stirred solution of methyl {(2S)-1-[(2S)-2-{4-[4-(7-hydroxyquinolin-3-yl)phenyl]-1H-imidazol-2-yl}pyrrolidin-1-yl]-3-methyl-1-oxobutan-2-yl}carbamate (215 mg, 0.42 mmol), obtained from Preparation 121, and triethylamine (0.23 mL, 1.68 mmol), in DCM (5 mL), at −60° C., was added triflic anhydride (0.11 mL, 0.63 mmol). The mixture was stirred at −60° C. for 5 minutes. It was then diluted with DCM (20 mL) and washed with sat aq NaHCO₃ solution (20 mL). The organic layer was separated, dried over Na₂SO₄, filtered and evaporated. The crude material was purified by flash chromatography on silica gel (EtOAc:heptanes 1:1) to give the title compound as a colourless foam (208 mg).

¹H NMR (400 MHz, CDCl₃): δ=9.21 (1H, d), 8.31 (1H, d), 8.00 (1H, d), 7.92 (1H, d), 7.81 (2H, m), 7.68 (2H, d), 7.50 (1H, s), 7.46 (1H, dd), 5.44 (1H, dd), 5.29 (1H, d), 4.33 (1H, dd), 3.94-3.84 (2H, m), 3.60 (3H, s), 2.39-2.30 (1H, m), 2.20-2.14 (1H, m), 2.09-1.97 (3H, m), 1.07 (3H, d), 0.88 (3H, d). LCMS (run time=8 min): Rₜ=5.19 min; m/z 778 [M+H]⁺

### Preparation 123

### tert-Butyl (2S)-2-(5-{3-[4-(2-{(2S)-1-[N-(methoxycarbonyl)-L-valyl]pyrrolidin-2-yl}-1H-imidazol-4-yl)phenyl]quinolin-7-yl}-1-{[2-(trimethylsilyl)ethoxy]methyl}-1H-imidazol-2-yl)pyrrolidine-1-carboxylate

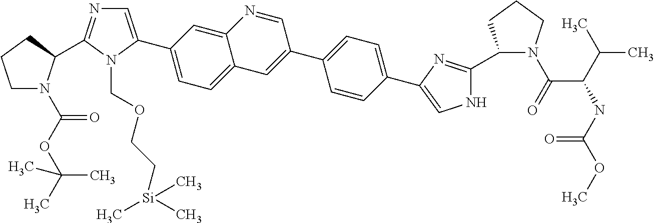

3-[4-(2-{(2S)-1-[N-(methoxycarbonyl)-L-valyl]pyrrolidin-2-yl}-1-[(trifluoromethyl)sulfonyl]-1H-imidazol-4-yl)phenyl]quinolin-7-yl trifluoromethanesulfonate (208 mg, 0.27 mmol), obtained from Preparation 122, (2-[(2S)-1-(tert-butoxycarbonyl)pyrrolidin-2-yl]-1-{[2 (trimethylsilyl)ethoxy]methyl}-1H-imidazol-5-yl)boronic acid (173 mg, 0.29 mmol), obtained from Preparation 13b, and Pd(dppf)Cl₂.DCM (13 mg, 0.053 mmol), in 1,2-dimethoxyethane (1 mL) and 2M sodium carbonate solution (0.40 mL, 0.80 mmol), were stirred in a microwave at 120° C. for 40 minutes. The reaction mixture was diluted with MeOH (10 mL), absorbed onto silica and purified by column chromatography on silica gel (dichloromethane: methanol:ammonia 95:5:0.5) to give the title compound as a yellow gum (120 mg).

LCMS (run time=4.5 min): Rₜ=2.20 min; m/z 863 [M+H]⁺

### Preparation 124

### Methyl {(2S)-3-methyl-1-oxo-1-[(2S)-2-{4-[4-(7-{2-[(2S)-pyrrolidin-2-yl]-1H-imidazol-5-yl}quinolin-3-yl)phenyl]-1H-imidazol-2-yl}pyrrolidin-1-yl]butan-2-yl}carbamate

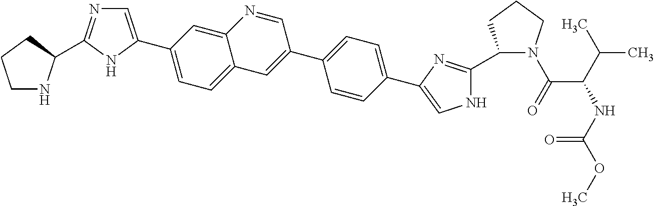

To a stirred solution of tert-butyl (2S)-2-(5-{3-[4-(2-{(2S)-1-[N-(methoxycarbonyl)-L-valyl]pyrrolidin-2-yl}-1H-imidazol-4-yl)phenyl]quinolin-7-yl}-1-{[2-(trimethylsilyl)ethoxy]methyl}-1H-imidazol-2-yl)pyrrolidine-1-carboxylate (120 mg, 0.14 mmol), obtained from Preparation 123, in MeOH (2 mL), was added 4M HCl in dioxane (0.70 mL, 2.78 mmol). The reaction mixture was stirred at 75° C. for 45 minutes. The solvent was then evaporated. The residue was dissolved in MeOH and loaded onto a 2 g SCX column, washing with more methanol. The product was collected by flushing with 7N ammonia in methanol and the filtrate concentrated in vacuo to give the title compound as yellow gum (82 mg).

LCMS (run time=4.5 min): Rₜ=1.17 min; m/z 633 [M+H]⁺

### Preparation 125

### 6-Bromo-2-(4-{2-[(2S)-pyrrolidin-2-yl]-1H-imidazol-5-yl}phenyl)quinoxaline hydrochloride salt

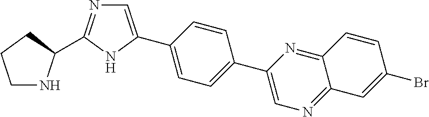

To a stirred solution of tert-butyl (2S)-2-{5-[4-(6-bromoquinoxalin-2-yl)phenyl]-1H-imidazol-2-yl}pyrrolidine-1-carboxylate (58 g, 0.11 mol), obtained from Preparation 31, in dioxane (600 mL), was added a solution of 4N HCl in dioxane (111.4 mL), at 10° C. The reaction mixture was stirred at room temperature overnight. The resulting precipitate was filtered and the filtered cake was dried in vacuum to give the title compound as a yellow solid (62.3 g) which was used directly in the next step.

### Preparation 126

### Methyl {(2S)-1-[(2S)-2-{5-[4-(6-bromoquinoxalin-2-yl)phenyl]-1H-imidazol-2-yl}pyrrolidin-1-yl]-3-methyl-1-oxobutan-2-yl}carbamate

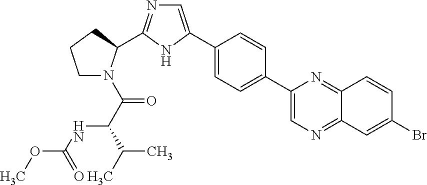

To a stirred suspension of 6-bromo-2-(4-{2-[(2S)-pyrrolidin-2-yl]-1H-imidazol-5-yl}phenyl)quinoxaline hydrochloride salt (62 g, 0.15 mol), obtained from Preparation 125, in dry DMF (600 mL), was added L-valine carbamate (28.4 g, 0.16 mol), obtained from Preparation 22, DIPEA (76.3 g, 0.59 mmol) and HATU (61.7 g, 0.16 mol), at 0° C. The reaction mixture was allowed to warm room temperature and stirred overnight. It was then poured into ice-water and extracted with EtOAc (3×600 mL). The combined organic layers were washed with brine (300 mL), dried over Na₂SO₄ and concentrated to give the crude product, which was purified by column chromatography (petroleum ether: ethyl acetate, 5:1 to 1:2) to give the title compound as an orange solid (44.2 g).

¹H NMR (400 MHz, CDCl₃): 9.28 (1H, s), 8.24-8.15 (3H, m), 8.00-7.81 (4H, m), 7.32 (1H, s), 5.55-5.52 (1H, m), 5.31-5.28 (1H, m), 4.37 (1H, t), 3.90-3.88 (1H, m), 3.77-3.68 (4H, m), 2.98 (1H, s), 2.40-2.35 (1H, m), 2.28-2.23 (1H, m), 2.18-2.10 (1H, m), 2.06-1.99 (2H, m), 0.95-0.91 (6H, t).

### Preparation 127

### tert-Butyl (2S)-2-(5-{2-[4-(2-{(2S)-1-[N-(methoxycarbonyl)-L-valyl]pyrrolidin-2-yl}-1H-imidazol-5-yl)phenyl]quinoxalin-6-yl}-1-{[2-(trimethylsilyl)ethoxy]methyl}-1H-imidazol-2-yl)pyrrolidine-1-carboxylate

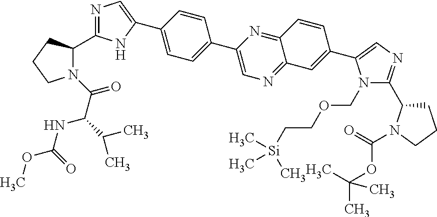

A mixture of methyl {(2S)-1-[(2S)-2-{5-[4-(6-bromoquinoxalin-2-yl)phenyl]-1H-imidazol-2-yl}pyrrolidin-1-yl]-3-methyl-1-oxobutan-2-yl}carbamate (15 g, 26.0 mmol), obtained from Preparation 126, (2-[(2S)-1-(tert-butoxycarbonyl)pyrrolidin-2-yl]-1-{[2 (trimethylsilyl)ethoxy]methyl}-1H-imidazol-5-yl)boronic acid (12.8 g, 31.2 mmol), obtained from Preparation 13b, CsF (9.9 g, 64.9 mmol) and CuI (0.5 g, 2.6 mmol), in DMF (150 mL), was degassed and backfilled with nitrogen five times. Pd(PPh₃)₄ (1.5 g) was added and the vessel was degassed and backfilled with nitrogen five times again. The reaction mixture was then heated to 90° C. overnight. The cooled reaction mixture was filtered. The filtrate was poured into ice-water (250 mL) and extracted with EtOAc (3×300 mL). The combined organic layers were washed with brine (2×200 mL), dried over Na₂SO₄ and concentrated to give the crude product, which was purified by column chromatography (petroleum ether: ethyl acetate, 5:1 to 1:3) to give the title compound as a yellow solid (15.2 g) which was used directly in the next step.

### Preparation 128

### Methyl {(2S)-3-methyl-1-oxo-1-[(2S)-2-{5-[4-(6-{2-[(2S)-pyrrolidin-2-yl]-1H-imidazol-5-yl}quinoxalin-2-yl)phenyl]-1H-imidazol-2-yl}pyrrolidin-1-yl]butan-2-yl}carbamate trifluoroacetate salt

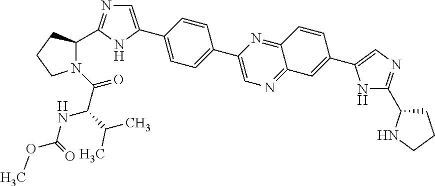

To a stirred solution of tert-butyl (2S)-2-(5-{2-[4-(2-{(2S)-1-[N-(methoxycarbonyl)-L-valyl]pyrrolidin-2-yl}-1H-imidazol-5-yl)phenyl]quinoxalin-6-yl}-1-{[2-(trimethylsilyl)ethoxy]methyl}-1H-imidazol-2-yl)pyrrolidine-1-carboxylate (17 g, 19.7 mmol), obtained from Preparation 127, in EtOH (170 mL), was added a solution of 4N HCl in dioxane (196.7 mL), dropwise. After the addition, the reaction mixture was heated to 75° C. for 1.5 hours. The solvent was removed under reduced pressure to give the crude HCl salt of the product, which was purified by prep. HPLC to give the title compound as a yellow solid (15.2 g).

¹H NMR (400 MHz, D₂O): 8.75 (1H, s), 7.65-7.54 (5H, m), 7.46 (1H, s), 7.40 (1H, s), 7.33-7.31 (2H, d), 5.10-5.06 (1H, t), 4.84-4.80 (1H, m), 4.18-4.16 (1H, d), 3.93 (1H, s), 3.82-3.75 (1H, m), 3.56 (3H, s), 3.44-3.40 (2H, m), 2.42-2.35 (2H, m), 2.18-1.86 (7H, m), 0.84-0.76 (6H, m).

The compounds of the invention may have the advantage that they are more potent, have a longer duration of action, have a broader range of activity, are more stable, have fewer side effects or are more selective, or have other more useful properties than the compounds of the art.

### Determination of HCv Replicon Inhibitory Activity

The ability of the compounds of the formula (I) to inhibit HCV replication may be measured using the assays described below.

### Test Compound Preparation

Test compounds were solubilised to 4 mM in 100% DMSO. Dilutions were made to the desired starting concentration in 100% DMSO and then 1 in 3 dilutions made, again in 100% DMSO. 0.5 μl of each sample was added to 384-well assay plates. White Lumitrac (Greiner) plates were used for the 1a and 1b replicon assays and black, clear bottomed (Greiner) plates were used for the WST-1 cytotoxity assay.

### Determination of HCV 1a Replicon Inhibitory Activity

1a-H77 replicon cells (licensed from Apath LLC) were resuspended to a concentration of 1.4×10⁵ cells/ml by addition of pre-warmed assay medium (Dulbecco's Modified Eagle's Medium (DMEM)+10% fetal calf serum (FCS)). 50 μl of this suspension was added to each well of a 384-well assay plate (Lumitrac, Greiner), already containing 0.5 μl of test compound.

All plates were covered with gas permeable seals and incubated at 37° C., 5% CO₂ for 48 hours. After 48 hours, the assay plate was removed from the incubator and left to cool to room temperature for 15 to 30 mins. Medium was removed from the wells and 5 μl 1× lysis buffer (from Promega's Renilla Luciferase assay kit, E2820) was added to each well. The plate was incubated at room temperature for 15 mins then 15 μl 1×Assay Substrate (from the same kit) was added to each well. Luminescence was read immediately using an EnVision (Perkin Elmer) plate reader. The half maximal effective concentration (1050) values were calculated by constructing log concentration-response curves for each compound.

### Determination of HCV 1b Replicon Inhibitory Activity

1b (con1) replicon cells (pFK1389lucubineo/NS3-3′/ET/9B, licensed from Ralf Bartenschlager, University of Heidelberg) were resuspended to a concentration of 1.4×10⁵ cells/ml by addition of pre-warmed medium (DMEM+10% FCS). 50 μl of this suspension was added to each well of a 384-well assay plate (Lumitrac, Greiner) already containing 0.5 μl of test compound.

All plates were covered with gas permeable seals and incubated at 37° C., 5% CO₂ for 48 hours. After 48 hours, the plate was removed from the incubator and left to cool to room temperature for 15-30 mins. 35 ul of reconstituted Lyophilised Britelite Plus Substrate (PerkinElmer, 6016767) to medium was added to each well. Luminescence was read after 1 min but before 15 mins on an EnVision (Perkin Elmer) plate reader. The half maximal effective concentration (1050) values were calculated by constructing log concentration-response curves for each compound.

### Determination of Compound Induced Cytotoxicity in HCV Replicon Cell Lines (as Measured Using WST-1 Reagent)

1b (con1) replicon cells were resuspended to a concentration of 1.4×10⁵ cells/ml by addition of pre-warmed medium (DMEM+10% FCS). 50 μl of this suspension was added to each well of a 384-well assay plate (Black, clear bottomed, Greiner) already containing 0.5 μl of test compound.

All plates were covered with gas permeable seals and incubated at 37° C., 5% CO₂ for 48 hours. After 48 hours, 5 μl WST-1 reagent (Roche, 11 644 807 001) was added to each well and the plate returned to the incubator for 3 hours. After this incubation period absorbance was read at 450 nm on an EnVision (Perkin Elmer) plate reader. The half maximal cytotoxic concentration (CC₅₀) values were calculated for each compound by constructing log concentration-response curves.

All of the exemplified compounds have been screened in a cell proliferation assay (WST-1). A majority have CC₅₀ values>μM in at least 2 independent experiments, the exceptions being the compounds of examples 1, 10, 12, 23, 25, 37, 47, 48, 49, 52, 56, 57, 58, 60, 67, 68, 69, 70, 71, 72, 73, 74, 79, 81 and 84 which have CC₅₀ values>2 μM in 1 experiment, and example 20 where the CC₅₀ is reported as >10 nM (in this instance the top concentration tested in the assay was 10 nM).

TABLE 1
HCV Replicon Inhibition

| Ex | IC50 value (95% Confidence Intervals) pM | |
|---|---|---|
| No. | Replicon 1A assay | Replicon 1B assay |
| 1 | 36.0 (29.1, 44.6) 22 | 5.89 (4.82, 7.19) 20 |
| 2 | 22.3 (15.8, 31.5) 25 | 2.37 (1.68, 3.34) 19 |
| 3 | 74.6 (6.41, 869) 7 | 9.31 (1.43, 60.5) 8 |
| 4 | 17.4 (13.2, 22.8) 21 | 7.70 (5.55, 10.7) 21 |
| 5 | 387 (136, 1100) 8 | 8.12 (4.98, 13.3) 5 |
| 6 | 56.4 (11.1, 287) 5 | 3.51 (1.74, 7.05) 4 |
| 7 | 68.4 (38.1, 123) 11 | 21.4 (2.00, 228) 13 |
| 8 | 91.2 (64.0, 130) 6 | 20.5 (14.9, 28.4) 6 |
| 9 | 42.9 (33.6, 54.7) 17 | 12.7 (10.6, 15.2) 11 |
| 10 | 1320 (259, 6770) 6 | 49.4 (18.7, 130) 4 |
| 11 | 42.1 (17.0, 104) 6 | 20.8 (18.5, 23.5) 7 |
| 12 | 3100 (657, 14600) 5 | 358 (104, 1230) 5 |
| 13 | 78.1 (65.5, 93.1) 6 | 12.0 (6.64, 21.6) 6 |
| 14 | 882 (193, 4030) 8 | 189 (62.5, 571) 8 |
| 15 | 194 (157, 240) 17 | 102 (56.9, 183) 11 |
| 16 | 35.3 (24.8, 50.3) 11 | 5.28 (3.86, 7.23) 8 |
| 17 | 560 (231, 1360) 5 | 22.3 (19.4, 25.6) 4 |
| 18 | 536 (447, 642) 10 | 35.8 (31.4, 41.0) 6 |
| 19 | 468 (174, 1260) 13 | 38.3 (11.1, 132) 11 |
| 20 | 1720 (343, 8640) 5 | 127 (56.8, 286) 5 |
| 21 | 671 (459, 982) 6 | 222 (167, 296) 6 |
| 22 | 2190 (1820, 2640) 6 | 152 (128, 181) 6 |
| 23 | 5450 (n = 1 9100, n = 2 3260) | 58.4 (n = 1 61.7, n = 2 55.3) |
| 24 | 19900 (2050, 194000) 5 | 2010 (598, 6750) 9 |
| 25 | 88.8 (16.8, 469) 5 | 10.8 (9.89, 11.9) 4 |
| 26 | 64.1 (32.3, 127) 11 | 1.92 (0.464, 7.98) 8 |
| 27 | 148 (48.1, 456) 9 | 2.34 (0.254, 21.6) 3 |
| 28 | 4290 (462, 39800) 8 | 384 (150, 983) 10 |
| 29 | 3020 (1170, 7820) 14 | 188 (74.7, 472) 9 |
| 30 | 1020 (22.2, 46500) 6 | 123 (1.25, 12000) 5 |
| 31 | 511 (15.8, 16500) 5 | 120 (1.18, 12100) 4 |
| 32 | 20300 (2580, 161000) 3 | 634 (30.9, 13000) 4 |
| 33 | 2590 (610, 11000) 4 | 32.2 (2.06, 504) 3 |
| 34 | 5930 (1090, 32400) 5 | 1430 (630, 3250) 4 |
| 35 | 2300 (95.8, 55400) 3 | 254 (23.2, 2780) 3 |
| 36 | 29800 (1140, 777000) 3 | 1740 (1160, 2600) 7 |
| 37 | 284 (88.0, 914) 4 | 42.9 (36.0, 51.1) 3 |
| 38 | 1500 (329, 6820) 4 | 71.9 (44.5, 116) 3 |
| 39 | 1240 (193, 7960) 4 | 48.1 (8.94, 259) 3 |
| 40 | 353 (42.2, 2940) 4 | 55.8 (15.7, 198) 3 |
| 41 | 1950 (726, 5230) 4 | 128 (47.7, 346) 3 |
| 42 | 2870 (1840, 4470) 6 | 247 (107, 571) 5 |
| 43 | 787 (25.5, 24300) 3 | 388 (75.2, 2000) 3 |
| 44 | 3510 (1860, 6590) 6 | 100 (14.3, 700) 4 |
| 45 | 20000 (2510, 160000) 4 | 507.0 (106, 2420) 3 |
| 46 | 803 (347, 1860) 4 | 44.2 (21.9, 89.4) 3 |
| 47 | 6850 (2580, 18200) 3 | 52.8 (6.84, 408) 3 |
| 48 | 27.8 (18.9, 40.9) 6 | 6.84 (4.78, 9.78) 6 |
| 49 | 73.0 (3.26, 1640) 4 | 7.27 (2.96, 17.8) 3 |
| 50 | 356 (159, 795) 7 | 77.2 (26.0, 229) 6 |
| 51 | 1850 (468, 7300) 5 | 144 (17.4, 1180) 4 |
| 52 | 22.1 (15.3, 32.1) 3 | 16.6 (10.3, 26.7) 3 |
| 53 | 646 (48.6, 8570) 4 | 21.1 (5.76, 77.3) 3 |
| 54 | 61.2 (37.5, 99.9) 3 | 14.9 (10.7, 20.7) 3 |
| 55 | 18.1 (11.2, 29.1) 5 | 8.85 (6.31, 12.4) 5 |
| 56 | 26.3 (10.3, 67.3) 3 | 10.2 (5.07, 20.6) 3 |
| 57 | 21.8 (15.8, 30.1) 6 | 21.9 (15.2, 31.7) 5 |
| 58 | 6.71 (2.67, 16.9) 3 | 5.07 (1.69, 15.2) 3 |
| 59 | 2390 (90.1, 63600) 3 | 97.5 (2.32, 4100) 3 |
| 60 | 22.3 (5.93, 83.7) 3 | 23.7 (7.66, 73.5) 3 |
| 61 | 6400 (2320, 17600) 3 | 83.5 (0.732, 9540) 3 |
| 62 | 2570 (489, 13500) 4 | 44.1 (3.76, 517) 3 |
| 63 | 4770 (874, 26100) 4 | 400 (73.8, 2170) 4 |
| 64 | 44.9 (10.7, 188) 3 | 42.0 (17.5, 101) 3 |
| 65 | 700 (255, 1910) 5 | 54.5 (17.4, 170) 3 |
| 66 | 1720 (314, 9410) 4 | 21.7 (12.1, 38.8) 3 |
| 67 | 27.5 (13.9, 54.5) 3 | 74.8 (28.2, 198) 3 |
| 68 | 23.2 (10.3, 52.7) 3 | 8.15 (4.36, 15.3) 3 |
| 69 | 22.3 (3.93, 126) 3 | 19.6 (9.32, 41.4) 3 |
| 71 | 30.7 (10.6, 88.8) 3 | 23.7 (8.55, 65.4) 3 |
| 70 | 9.13 (1.73, 48.2) 3 | 5.17 (1.88, 14.2) 3 |
| 72 | 53.6 (20.9, 138) 3 | 7.81 (4.26, 14.3) 3 |
| 73 | 13.3 (6.05, 29.2) 5 | 8.11 (2.53, 26.0) 4 |
| 74 | 43.7 (33.5, 56.9) 7 | 14.2 (8.96, 22.6) 7 |
| 75 | 4190 (2080, 8420) 6 | 143 (51.3, 398) 4 |
| 76 | 157 (60.5, 40) 6 | 58.8 (34.7, 99.6) 4 |
| 77 | 186 (71.5, 482) 3 | 31.40 (19.0, 51.9) 3 |
| 78 | 443 (354, 555) 5 | 45.5 (40.0, 51.9) 4 |
| 79 | 47.6 (22.1, 103) 3 | 11.7 (5.25, 26.3) 3 |
| 80 | 117 (37.6, 362) 5 | 11.4 (3.09, 42.3) 4 |
| 81 | 44.0 (34.2, 56.5) 3 | 13.7 (7.1, 26.4) 3 |
| 82 | 490 (142, 1690) 4 | 190 (70.7, 509) 3 |
| 83 | 1030 (475, 2250) 4 | 53.4 (33.6, 84.9) 3 |
| 84 | 19.0 (10.1, 35.8) 3 | 10.9 (4.50, 26.2) 3 |

Table 1. Data for Replicon 1a and Replicon 1b in vitro pharmacology assays detailing geometric means from IC₅₀ values generated.
95% confidence intervals are shown in parentheses followed by the respective n value.

### Estimation of Fᵤ—Equilibrium Dialysis.

Plasma binding free fractions (fᵤ) were determined using a proprietary validated Rapid Equilibrium Dialysis (RED) device (Thermofisher). The analyte was spiked to a concentration of 2 μM in pre-prepared male CD rat plasma and 220 μl aliquots (n=4) were loaded into a RED device. The samples were dialysed vs. 350 μl of dulbeccos phosphate buffered saline (dPBS) for 4 h at 37° C. in a CO₂ incubator in order to reach equilibrium. After 4 hours, 45 μl buffer and 15 μl plasma aliquots were taken and added to a 200 μl 384-well polypropylene plate. Respective volumes of control buffer were added to the plasma or control plasma was added to the buffer samples to give an identical matrix between samples. The samples were then crashed in 120 μl of acetonitrile and analysed via LC-MS/MS.

Compounds of the present invention may exhibit a high degree of binding to human plasma proteins (for example, the plasma protein binding for Examples 1, 2 and 3 is 99.85%, 99.9% and >99.85% respectively). For highly bound compounds in the potency assay, which includes 10% foetal calf serum, measured potencies may not reflect real intrinsic potency due to a significant degree of compound binding. True in vitro activity for compounds of this type could be significantly greater, when adjustments are made for unbound compound.

1-8. (canceled)

8. A compound of the formula: 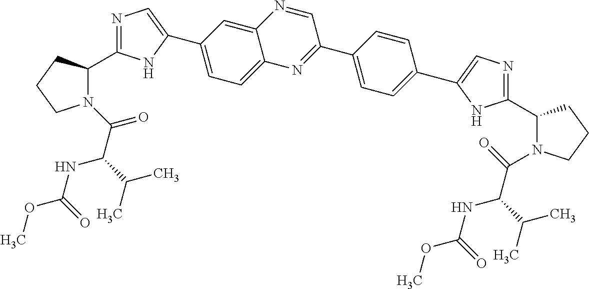
or a pharmaceutically acceptable salt thereof.

9. A pharmaceutical composition comprising a compound of claim 8 together with at least one pharmaceutically acceptable excipient.

10. (canceled)

11. A compound of claim 8 for use in the treatment of a disease for which an inhibitor of HCV replication is indicated.

12. A compound of claim 8 for use in the treatment of HCV infection.

13. A method of treatment of a disease in a mammal, for which inhibition of HCV replication is indicated, comprising administering to said mammal an effective amount of a compound of claim 8

14. A method of treating an HCV infection in a mammal comprising administering to said mammal an effective amount of a compound of claim 8.

15. A compound of claim 8, in combination with one or more other pharmacologically active agents.

16. A compound of claim 8, in combination with one or more other agents which are useful for the treatment of HCV infection.

17. A compound according to claim 16, wherein at least one of the one or more other agents is a NS5B RNA-polymerase inhibitor.

18. A compound according to claim 17, wherein the NS5B RNA-polymerase inhibitor is selected from Filibuvir, HCV-796, Valopicitabine, GL-59728, GL-60667, PSI-6130, R1626, R7128, JTK-003 GL-59728 and GS-9190.

19. A compound according to claim 18, wherein the NS5B RNA-polymerase inhibitor is Filibuvir.

20. A product comprising a compound of claim 8 and one or more other pharmacologically active agents as a combined preparation for simultaneous, separate or sequential use in therapy.

21. A kit comprising two or more pharmaceutical compositions, at least one of which comprises a compound of claim 8 together with a pharmaceutically acceptable excipient, and means for separately retaining said compositions.

22. Methyl {(2S)-1-[(2S)-2-{5-[4-(6-{2-[(2S)-1-{(2S)-2-[(methoxycarbonyl)amino]-3-methylbutanoyl}pyrrolidin-2-yl]-1H-imidazol-5-yl}quinoxalin-2-yl)phenyl]-1H-imidazol-2-yl}pyrrolidin-1-yl]-3-methyl-1-oxobutan-2-yl}carbamate, or a pharmaceutically acceptable salt of said compound.
